SEQUENCE LISTING (33 sequences)

SEQ ID NO: 1 - DNA, 2808 bp, synthetic construct
  source 1..2808: mol_type = other DNA
atggctttcctgtggctgctgagctgctgggctctgctgggcaccacctttgggctgctggtgcctagggagctgtctgggtctagccctgtgctggaggagactcaccctgcccatcagcagggggctagcaggcctggccccagggatgctcaggcccaccctggcaggcccagggctgtgcccacccagtgtgatgtgccccccaacagcaggtttgactgtgcccctgacaaggccattacccaggagcagtgtgaggccaggggctgctgctacattccagctaagcagggcctgcagggggcccagatgggccagccctggtgcttcttcccccccagctatcctagctataaactggagaacctgagcagctctgagatgggctatactgccaccctgactaggactactcccaccttttttcctaaggatatcctgaccctgaggctggatgtgatgatggagactgagaacaggctgcacttcactattaaggaccctgccaataggaggtatgaagtgcctctggagactcctcatgtgcactctagggcccccagccccctgtattctgtggagttctctgaggagccctttggggtgattgtgaggaggcagctggatggcagggtgctgctgaacaccactgtggcccccctgttctttgctgaccagttcctgcagctgagcaccagcctgcccagccagtacatcactgggctggctgagcatctgagccctctgatgctgagcacctcttggaccaggatcaccctgtggaatagggatctggcccccacccctggggctaatctgtatggctctcatcccttttacctggccctggaggatgggggctctgcccatggggtgtttctgctgaacagcaatgccatggatgtggtgctgcagccctctcctgccctgagctggaggagcactgggggcatcctggatgtgtacatcttcctgggccctgagcccaagtctgtggtccagcagtatctggatgtggtgggctacccctttatgcccccctattggggcctgggcttccacctgtgcaggtgggggtattcttctactgctatcaccaggcaggtggtggagaacatgaccagggctcacttccccctggatgtgcagtggaatgacctggactatatggactctaggagggatttcaccttcaacaaggatggcttcagggacttccctgctatggtccaggagctgcatcaggggggcaggaggtacatgatgattgtggaccctgccatcagcagctctggccctgctggcagctataggccctatgatgagggcctgaggaggggggtgtttatcactaatgaaactgggcagcccctgattggcaaggtgtggcctggctctactgccttccctgacttcaccaaccccactgctctggcctggtgggaggacatggtggctgagttccatgaccaggtgccttttgatggcatgtggattgacatgaatgagcccagcaacttcatcaggggctctgaggatgggtgccccaataatgagctggagaaccccccctatgtgcctggggtggtggggggcaccctgcaggctgccactatttgtgccagctctcaccagttcctgagcacccactacaacctgcacaatctgtatggcctgactgaggccattgccagccacagggccctggtgaaggccaggggcactaggccctttgtgatctctagaagcacctttgctggccatgggaggtatgctggccactggactggggatgtgtggagctcttgggagcagctggccagctctgtgcctgagatcctgcagttcaacctgctgggggtgcccctggtgggggctgatgtgtgtggcttcctgggcaacacctctgaagagctgtgtgtgaggtggacccagctgggggccttctaccctttcatgaggaaccacaacagcctgctgagcctgcctcaggagccttactctttctctgagcctgcccagcaggccatgaggaaggccctgaccctgaggtatgctctgctgccccacctgtacaccctgttccaccaggcccatgtggctggggagactgtggccaggcccctgttcctggagtttcctaaggatagcagcacctggactgtggaccaccagctgctgtggggggaggccctgctgattacccctgtgctgcaggctggcaaggctgaggtgactggctacttccccctgggcacttggtatgacctgcagactgtgcctgtggaagccctgggcagcctgcctcccccccctgctgcccccagggagcctgccatccactctgagggccagtgggtgaccctgcctgcccccctggacaccattaatgtgcatctgagggctgggtatattatccccctgcaggggcctgggctgactaccactgagagcaggcagcagcctatggccctggctgtggctctgactaaggggggggaggccaggggggagctgttctgggatgatggggagagcctggaggtgctggagaggggggcctacacccaggtgattttcctggccaggaacaacaccattgtgaatgagctggtgagggtgacctctgagggggctggcctgcagctgcagaaagtgactgtgctgggggtggccactgccccccagcaggtgctgagcaatggggtgcctgtgagcaacttcacctacagccctgacaccaaggtgctggatatttgtgtgagcctgctgatgggggagcagttcctggtgagctggtgctga

SEQ ID NO: 2 - DNA, 2808 bp, synthetic construct
  source 1..2808: mol_type = other DNA
atggctttcctgtggctgctgtcttgctgggccctgctggggactacctttggcctgctggtgcccagggaactgtctggctctagcccagtgctggaggagacccaccctgcccaccagcagggggcttctaggcctggccccagggatgcccaggcccaccctggcaggccaagggctgtgcccacccagtgtgatgtgccccccaactctagatttgattgtgcccctgataaggccatcacccaggagcagtgtgaggctaggggctgctgctacatccctgctaagcagggcctgcagggggctcagatgggccagccctggtgcttcttcccccccagctatccctcttacaagctggagaatctgagcagctctgagatgggctacactgccaccctgaccaggactactcccaccttcttccccaaggacatcctgaccctgaggctggatgtgatgatggagactgagaacaggctgcatttcaccatcaaggatcctgccaacaggaggtatgaggtgcctctggagaccccccatgtgcacagcagggctccttctcccctgtactctgtggagttctctgaggaaccctttggggtgattgtgaggaggcagctggatggcagggtcctgctgaacaccactgtggcccccctgttctttgctgatcagttcctgcagctgtccacttctctgcctagccagtacatcactgggctggctgagcacctgagccctctgatgctgagcacctcttggactaggatcaccctgtggaacagggacctggcccccacccctggggccaacctgtatggcagccaccccttctatctggccctggaggatgggggctctgcccatggggtgttcctgctgaatagcaatgctatggatgtggtgctgcagcccagccctgccctgtcttggaggagcactgggggcatcctggatgtgtacattttcctggggcctgagcccaagtctgtggtgcagcagtacctggatgtggtgggctaccccttcatgcctccctactggggcctgggcttccacctgtgcaggtggggctacagctctactgccatcaccaggcaggtggtggagaatatgaccagggcccacttccccctggatgtgcagtggaatgacctggactacatggactctaggagggacttcaccttcaataaggatggcttcagagacttccctgccatggtgcaggagctgcatcaggggggcaggaggtacatgatgattgtggaccctgccatcagctcttctggccctgctggctcttacaggccctatgatgagggcctgaggaggggggtgttcatcaccaatgagactgggcagcccctgattgggaaggtgtggcctggctctactgccttccctgacttcaccaatcctactgccctggcctggtgggaggacatggtggctgagttccatgaccaggtgccctttgatggcatgtggattgacatgaatgagccctctaatttcatcaggggctctgaggatggctgccccaacaatgagctggagaaccccccctatgtgcctggggtggtggggggcaccctgcaggctgccaccatctgtgctagctctcaccagttcctgagcacccactacaatctgcataacctgtatggcctgactgaggccattgccagccacagggccctggtgaaggctaggggcaccaggccctttgtgatttctaggagcacttttgctggccatggcaggtatgctgggcactggactggggatgtgtggtctagctgggagcagctggcttcttctgtgcctgagatcctgcagttcaacctgctgggggtgcctctggtgggggctgatgtgtgtgggttcctgggcaacacttctgaggagctgtgtgtgaggtggacccagctgggggccttctaccctttcatgaggaaccacaacagcctgctgagcctgccccaggagccctacagcttctctgagcctgcccagcaggccatgaggaaggccctgaccctgaggtatgccctgctgccccacctgtacaccctgttccaccaggcccatgtggctggggagactgtggctaggcctctgttcctggagttccccaaggactctagcacctggactgtggaccaccagctgctgtggggggaggccctgctgatcactcctgtgctgcaggctgggaaggctgaggtgactggctatttccccctgggcacctggtatgacctgcagactgtgcctgtggaggccctggggagcctgccccccccccctgctgcccccagggagcctgccatccactctgagggccagtgggtgaccctgcctgcccctctggataccatcaatgtgcacctgagggctggctacatcattcccctgcagggccctggcctgaccactactgagtctaggcagcagcccatggccctggctgtggccctgaccaaggggggggaggctaggggggagctgttttgggatgatggggagagcctggaggtgctggagaggggggcctacactcaggtgatcttcctggccaggaacaataccattgtgaatgagctggtgagggtgacctctgagggggctggcctgcagctgcagaaggtgactgtgctgggggtggccactgccccccagcaggtgctgagcaatggggtgcctgtgagcaacttcacctatagccctgataccaaggtgctggatatttgtgtgagcctgctgatgggggagcagttcctggtgagctggtgctga

SEQ ID NO: 3 - DNA, 2808 bp, synthetic construct
  source 1..2808: mol_type = other DNA
atggctttcctgtggctgctgtcttgttgggctctgctgggcaccacctttggcctgctggtgcccagggagctgtctggcagcagccctgtgctggaggagacccaccctgctcatcagcagggggctagcaggcctggccccagggatgcccaggctcaccctgggagacccagggctgtgcccactcagtgtgatgtgccccccaacagcaggtttgactgtgctcctgacaaggctatcacccaggagcagtgtgaggccagggggtgctgctacattcctgctaagcagggcctgcagggggcccagatgggccagccctggtgcttcttccccccctcttatcccagctataagctggagaacctgagcagctctgagatgggctacactgccaccctgaccaggaccactcccaccttctttcccaaggatattctgactctgaggctggatgtgatgatggagactgagaacaggctgcacttcactatcaaggaccctgccaataggaggtatgaggtgcccctggagactcctcatgtgcatagcagggccccttctcctctgtattctgtggagttctctgaggagccctttggggtgattgtgaggaggcagctggatggcagggtgctgctgaacaccactgtggcccccctgttctttgctgaccagttcctgcagctgagcacttctctgcccagccagtacattactgggctggctgagcatctgagccccctgatgctgagcacctcttggaccaggatcaccctgtggaacagggacctggcccccactcctggggctaacctgtatggctctcaccccttttacctggccctggaggatgggggctctgcccatggggtgtttctgctgaacagcaatgctatggatgtggtgctgcagccctctccagccctgtcttggaggagcactgggggcattctggatgtgtacattttcctggggcctgaacccaagtctgtggtgcagcagtacctggatgtggtgggctaccccttcatgcccccctattgggggctggggtttcacctgtgcaggtggggctacagcagcactgccatcaccaggcaggtggtggagaacatgaccagggcccatttccccctggatgtgcagtggaatgacctggactacatggatagcaggagggatttcaccttcaacaaggatggcttcagggactttcctgccatggtgcaggagctgcaccaggggggcaggaggtatatgatgattgtggaccctgctatcagcagctctggccctgctggctcttacaggccctatgatgagggcctgaggaggggggtgtttatcactaatgaaactggccagcctctgattggcaaggtctggcctggctctactgccttccctgattttactaaccccactgccctggcctggtgggaggacatggtggctgagttccatgatcaggtgccttttgatggcatgtggattgatatgaatgaaccaagcaacttcatcagaggctctgaggatggctgccccaacaatgagctggagaaccccccctatgtgcctggggtggtggggggcactctgcaggctgccaccatttgtgctagcagccaccagttcctgagcacccactacaatctgcacaacctgtatggcctgactgaagccattgccagccatagggccctggtgaaggccaggggcactaggccttttgtgatcagcaggagcacttttgctggccatggcaggtatgctggccactggactggggatgtgtggagcagctgggagcagctggccagctctgtgcctgagattctgcagtttaacctgctgggggtgcccctggtgggggctgatgtgtgtggcttcctgggcaacacctctgaggagctgtgtgtgaggtggacccagctgggggccttttatcccttcatgaggaaccacaacagcctgctgagcctgcctcaggagccctactctttctctgagcctgcccagcaggccatgaggaaggccctgaccctgaggtatgccctgctgccccacctgtataccctgttccaccaggcccatgtggctggggagactgtggccaggcccctgttcctggagttccccaaggacagcagcacctggactgtggatcatcagctgctgtggggggaggccctgctgatcacccctgtgctgcaggctggcaaggctgaggtcactggctacttccctctgggcacctggtatgacctgcagactgtgcctgtggaggctctgggcagcctgccccccccccctgctgctcccagggagcctgccatccactctgagggccagtgggtgaccctgcctgctcccctggacaccatcaatgtgcacctgagggctggctacattatccccctgcagggcccagggctgactaccactgagagcagacagcagcccatggctctggctgtggccctgaccaaggggggggaagctaggggggagctgttctgggatgatggggagagcctggaggtgctggagaggggggcctatacccaggtgatcttcctggctaggaacaacaccattgtcaatgagctggtgagggtgacttctgagggggctgggctgcagctgcagaaggtgactgtgctgggggtggccactgctccccagcaggtgctgagcaatggggtgcctgtgagcaacttcacctacagccctgacaccaaggtgctggacatctgtgtgtctctgctgatgggggagcagttcctggtgagctggtgctga

SEQ ID NO: 4 - DNA, 2808 bp, synthetic construct
  source 1..2808: mol_type = other DNA
atggccttcctgtggctgctgtcttgctgggctctgctggggaccacctttggcctgctggtccccagggagctgtctggctcttctcctgtcctggaggagacccaccctgcccaccagcagggggctagcaggcctggccccagggatgcccaggcccaccctggcaggcccagggctgtgcccacccagtgtgatgtgcctcccaacagcaggtttgactgtgcccctgacaaggccatcacccaggagcagtgtgaggccaggggctgctgctatatccctgccaagcagggcctgcagggggctcagatgggccagccctggtgcttctttcccccctcttatcctagctataagctggagaacctgagcagctctgagatggggtacactgccaccctgaccaggaccacccccactttcttccctaaggacatcctgaccctgaggctggatgtgatgatggagactgagaataggctgcactttactatcaaggaccctgccaacaggaggtatgaggtgcctctggagaccccccatgtgcattctagggcccccagccccctgtactctgtggagttctctgaggagccctttggggtgattgtgaggagacagctggatggcagggtcctgctgaacaccactgtggctcccctgttttttgctgaccagttcctgcagctgagcaccagcctgcccagccagtacatcactgggctggctgagcacctgagccccctgatgctgagcaccagctggaccaggatcaccctgtggaacagggatctggctcctacccctggggccaacctgtatggctctcaccccttttacctggccctggaggatgggggctctgcccatggggtgttcctgctgaacagcaatgctatggatgtggtgctgcagcccagccctgccctgagctggaggtctactgggggcatcctggatgtgtacatctttctggggcctgagcccaagtctgtggtgcagcagtacctggatgtggtgggctatccttttatgcccccctattggggcctgggcttccacctgtgcaggtggggctacagcagcactgccatcaccagacaggtggtggagaacatgaccagggcccacttccccctggatgtgcagtggaatgacctggactacatggacagcaggagggacttcacctttaacaaggatggctttagggacttccctgccatggtgcaggagctgcatcaggggggcaggaggtacatgatgattgtggacccagccatcagcagctctgggcctgctgggtcttacaggccctatgatgagggcctgaggaggggggtgttcatcaccaatgagactggccagcccctgattggcaaggtgtggcctgggagcactgccttccctgattttaccaaccccactgccctggcctggtgggaggatatggtggctgagtttcatgaccaggtgccctttgatggcatgtggattgacatgaatgagcccagcaatttcatcaggggctctgaggatggctgccccaacaatgagctggagaatcctccctatgtgcctggggtggtggggggcaccctgcaggctgccaccatctgtgcctctagccaccagttcctgagcacccactataacctgcataacctgtatggcctgactgaggccattgccagccatagagccctggtgaaggccagagggaccaggccctttgtgatctctaggagcacctttgctggccatggcaggtatgctggccactggactggggatgtgtggagctcttgggagcagctggccagctctgtgccagagatcctgcagttcaacctgctgggggtgcctctggtgggggctgatgtgtgtggcttcctgggcaatacctctgaagagctgtgtgtgaggtggactcagctgggggccttctatcccttcatgaggaaccacaacagcctgctgtctctgccccaggagccctacagcttctctgagcctgctcagcaggctatgaggaaggccctgaccctgaggtatgccctgctgccccatctgtacaccctgttccaccaggcccatgtggctggggagactgtggccaggcccctgtttctggagtttcccaaggacagcagcacctggactgtggaccatcagctgctgtggggggaggctctgctgattacccctgtgctgcaggctggcaaggctgaggtgactgggtacttccccctggggacttggtatgacctgcagactgtgcctgtggaagctctgggcagcctgcccccaccccctgctgcccctagggagcctgccatccactctgagggccagtgggtgaccctgcctgcccctctggacaccatcaatgtgcacctgagggctggctatatcatccccctgcagggccctgggctgaccaccactgagagcaggcagcagcccatggccctggctgtggccctgactaaggggggggaggccaggggggagctgttctgggatgatggggagagcctggaggtgctggagagaggggcctacacccaggtgatctttctggccaggaacaacaccattgtgaatgagctggtgagggtgacttctgagggggctggcctgcagctgcagaaggtgactgtgctgggggtggccactgccccccagcaggtgctgagcaatggggtgcctgtgtctaacttcacctacagccctgatactaaggtgctggatatctgtgtgagcctgctgatgggggagcagtttctggtgagctggtgctga

SEQ ID NO: 5 - DNA, 2808 bp, synthetic construct
  source 1..2808: mol_type = other DNA
atggcctttctgtggctgctgtcctgctgggccctgctggggaccacctttggcctgctggtgcccagggagctgtctgggagcagcccagtgctggaggagacccaccctgcccaccagcagggggccagcaggcctggccctagggatgcccaggcccaccctggcaggcccagggctgtgcctacccagtgtgatgtgccacccaattctaggtttgactgtgctcctgacaaggccatcactcaggagcagtgtgaagctagggggtgctgctacatcccagccaagcagggcctgcagggggcccagatgggccagccctggtgcttcttcccccccagctaccctagctacaagctggagaatctgagcagctctgagatgggctacactgctaccctgaccaggaccactcctaccttcttccccaaggacatcctgactctgaggctggatgtcatgatggagactgaaaataggctgcacttcaccatcaaggaccctgccaataggaggtatgaggtgcctctggagaccccccatgtgcatagcagggctcccagccccctgtattctgtggagttctctgaggagccctttggggtcattgtgaggagacagctggatgggagggtgctgctgaacactactgtggctcccctgttctttgctgaccagttcctgcagctgtctaccagcctgcccagccagtacatcactgggctggctgagcatctgagccccctgatgctgagcaccagctggaccaggatcactctgtggaacagggatctggcccccactcctggggccaacctgtatgggagccatcccttctacctggccctggaggatgggggctctgcccatggggtgttcctgctgaacagcaatgccatggatgtggtgctgcagcctagccctgccctgagctggaggagcactgggggcatcctggatgtctacatcttcctggggcctgagcccaagtctgtggtgcagcagtatctggatgtggtggggtatcccttcatgcccccctactggggcctgggctttcacctgtgcaggtggggctacagcagcactgccatcaccaggcaggtggtggagaacatgaccagggcccacttccctctggatgtgcagtggaatgacctggactatatggattctaggagagactttacttttaacaaggatggcttcagggatttccctgccatggtgcaggagctgcaccaggggggcaggaggtacatgatgattgtggaccctgctattagcagctctggccctgctgggtcttacaggccttatgatgagggcctgaggaggggggtgttcatcaccaatgagactggccagcccctgattggcaaggtgtggcctggcagcactgccttccctgacttcaccaaccccactgccctggcctggtgggaggacatggtggctgagttccatgaccaggtgccctttgatgggatgtggattgacatgaatgagccctctaacttcatcagggggtctgaggatggctgccccaacaatgagctggagaaccccccctatgtgcctggggtggtggggggcactctgcaggctgccactatctgtgcttcttctcaccagtttctgagcacccactataatctgcacaacctgtatggcctgactgaggccattgccagccatagggccctggtgaaggccaggggcaccaggccctttgtgatcagcaggtctacctttgctggccatggcaggtatgctggccactggactggggatgtgtggtcttcttgggagcagctggccagctctgtgcctgagatcctgcagttcaacctgctgggggtgcctctggtgggggctgatgtgtgtggctttctgggcaacacctctgaggagctgtgtgtgaggtggacccagctgggggccttttaccccttcatgaggaaccacaatagcctgctgagcctgccccaggagccttactctttctctgagcctgcccagcaggccatgaggaaggccctgactctgaggtatgccctgctgccccatctgtataccctgtttcaccaggcccatgtggctggggagactgtggctaggcctctgtttctggagttccctaaggactctagcacctggactgtggaccaccagctgctgtggggggaggccctgctgatcacccctgtgctgcaggctggcaaggctgaggtgactggctacttccccctgggcacctggtatgacctgcagactgtgcctgtggaggccctggggagcctgcctcccccccctgctgcccccagggagcctgccattcattctgagggccagtgggtgaccctgcctgcccctctggacaccatcaatgtgcacctgagggctgggtacatcatccccctgcagggccctggcctgaccaccactgagagcaggcagcagcccatggccctggctgtggctctgaccaaggggggggaggccaggggggagctgttctgggatgatggggagtctctggaggtgctggagaggggggcctacacccaggtgatctttctggccaggaacaatactattgtgaatgagctggtgagggtgacctctgagggggctggcctgcagctgcagaaggtgactgtgctgggggtggccactgccccccagcaggtcctgagcaatggggtgcctgtgagcaacttcacctactctcctgacaccaaggtgctggacatttgtgtgtctctgctgatgggggagcagttcctggtgagctggtgctga

SEQ ID NO: 6 - DNA, 3016 bp, synthetic construct
  source 1..3016: mol_type = other DNA
atggctttcctgtggctgctgagctgctgggctctgctgggcaccacctttgggctgctggtgcctagggagctgtctgggtctagccctgtgctggaggagactcaccctgcccatcagcagggggctagcaggcctggccccagggatgctcaggcccaccctggcaggcccagggctgtgcccacccagtgtgatgtgccccccaacagcaggtttgactgtgcccctgacaaggccattacccaggagcagtgtgaggccaggggctgctgctacattccagctaagcagggcctgcagggggcccagatgggccagccctggtgcttcttcccccccagctatcctagctataaactggagaacctgagcagctctgagatgggctatactgccaccctgactaggactactcccaccttttttcctaaggatatcctgaccctgaggctggatgtgatgatggagactgagaacaggctgcacttcactattaaggaccctgccaataggaggtatgaagtgcctctggagactcctcatgtgcactctagggcccccagccccctgtattctgtggagttctctgaggagccctttggggtgattgtgaggaggcagctggatggcagggtgctgctgaacaccactgtggcccccctgttctttgctgaccagttcctgcagctgagcaccagcctgcccagccagtacatcactgggctggctgagcatctgagccctctgatgctgagcacctcttggaccaggatcaccctgtggaatagggatctggcccccacccctggggctaatctgtatggctctcatcccttttacctggccctggaggatgggggctctgcccatggggtgtttctgctgaacagcaatgccatggatgtggtgctgcagccctctcctgccctgagctggaggagcactgggggcatcctggatgtgtacatcttcctgggccctgagcccaagtctgtggtccagcagtatctggatgtggtgggctacccctttatgcccccctattggggcctgggcttccacctgtgcaggtgggggtattcttctactgctatcaccaggcaggtggtggagaacatgaccagggctcacttccccctggatgtgcagtggaatgacctggactatatggactctaggagggatttcaccttcaacaaggatggcttcagggacttccctgctatggtccaggagctgcatcaggggggcaggaggtacatgatgattgtggaccctgccatcagcagctctggccctgctggcagctataggccctatgatgagggcctgaggaggggggtgtttatcactaatgaaactgggcagcccctgattggcaaggtgtggcctggctctactgccttccctgacttcaccaaccccactgctctggcctggtgggaggacatggtggctgagttccatgaccaggtgccttttgatggcatgtggattgacatgaatgagcccagcaacttcatcaggggctctgaggatgggtgccccaataatgagctggagaaccccccctatgtgcctggggtggtggggggcaccctgcaggctgccactatttgtgccagctctcaccagttcctgagcacccactacaacctgcacaatctgtatggcctgactgaggccattgccagccacagggccctggtgaaggccaggggcactaggccctttgtgatctctagaagcacctttgctggccatgggaggtatgctggccactggactggggatgtgtggagctcttgggagcagctggccagctctgtgcctgagatcctgcagttcaacctgctgggggtgcccctggtgggggctgatgtgtgtggcttcctgggcaacacctctgaagagctgtgtgtgaggtggacccagctgggggccttctaccctttcatgaggaaccacaacagcctgctgagcctgcctcaggagccttactctttctctgagcctgcccagcaggccatgaggaaggccctgaccctgaggtatgctctgctgccccacctgtacaccctgttccaccaggcccatgtggctggggagactgtggccaggcccctgttcctggagtttcctaaggatagcagcacctggactgtggaccaccagctgctgtggggggaggccctgctgattacccctgtgctgcaggctggcaaggctgaggtgactggctacttccccctgggcacttggtatgacctgcagactgtgcctgtggaagccctgggcagcctgcctcccccccctgctgcccccagggagcctgccatccactctgagggccagtgggtgaccctgcctgcccccctggacaccattaatgtgcatctgagggctgggtatattatccccctgcaggggcctgggctgactaccactgagagcaggcagcagcctatggccctggctgtggctctgactaaggggggggaggccaggggggagctgttctgggatgatggggagagcctggaggtgctggagaggggggcctacacccaggtgattttcctggccaggaacaacaccattgtgaatgagctggtgagggtgacctctgagggggctggcctgcagctgcagaaagtgactgtgctgggggtggccactgccccccagcaggtgctgagcaatggggtgcctgtgagcaacttcacctacagccctgacaccaaggtgctggatatttgtgtgagcctgctgatgggggagcagttcctggtgagctggtgctgactcgagagatctaccggtgaattcaccgcgggtttaaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtgggggctagctctaga

SEQ ID NO: 7 - DNA, 3016 bp, synthetic construct
  source 1..3016: mol_type = other DNA
atggctttcctgtggctgctgtcttgctgggccctgctggggactacctttggcctgctggtgcccagggaactgtctggctctagcccagtgctggaggagacccaccctgcccaccagcagggggcttctaggcctggccccagggatgcccaggcccaccctggcaggccaagggctgtgcccacccagtgtgatgtgccccccaactctagatttgattgtgcccctgataaggccatcacccaggagcagtgtgaggctaggggctgctgctacatccctgctaagcagggcctgcagggggctcagatgggccagccctggtgcttcttcccccccagctatccctcttacaagctggagaatctgagcagctctgagatgggctacactgccaccctgaccaggactactcccaccttcttccccaaggacatcctgaccctgaggctggatgtgatgatggagactgagaacaggctgcatttcaccatcaaggatcctgccaacaggaggtatgaggtgcctctggagaccccccatgtgcacagcagggctccttctcccctgtactctgtggagttctctgaggaaccctttggggtgattgtgaggaggcagctggatggcagggtcctgctgaacaccactgtggcccccctgttctttgctgatcagttcctgcagctgtccacttctctgcctagccagtacatcactgggctggctgagcacctgagccctctgatgctgagcacctcttggactaggatcaccctgtggaacagggacctggcccccacccctggggccaacctgtatggcagccaccccttctatctggccctggaggatgggggctctgcccatggggtgttcctgctgaatagcaatgctatggatgtggtgctgcagcccagccctgccctgtcttggaggagcactgggggcatcctggatgtgtacattttcctggggcctgagcccaagtctgtggtgcagcagtacctggatgtggtgggctaccccttcatgcctccctactggggcctgggcttccacctgtgcaggtggggctacagctctactgccatcaccaggcaggtggtggagaatatgaccagggcccacttccccctggatgtgcagtggaatgacctggactacatggactctaggagggacttcaccttcaataaggatggcttcagagacttccctgccatggtgcaggagctgcatcaggggggcaggaggtacatgatgattgtggaccctgccatcagctcttctggccctgctggctcttacaggccctatgatgagggcctgaggaggggggtgttcatcaccaatgagactgggcagcccctgattgggaaggtgtggcctggctctactgccttccctgacttcaccaatcctactgccctggcctggtgggaggacatggtggctgagttccatgaccaggtgccctttgatggcatgtggattgacatgaatgagccctctaatttcatcaggggctctgaggatggctgccccaacaatgagctggagaaccccccctatgtgcctggggtggtggggggcaccctgcaggctgccaccatctgtgctagctctcaccagttcctgagcacccactacaatctgcataacctgtatggcctgactgaggccattgccagccacagggccctggtgaaggctaggggcaccaggccctttgtgatttctaggagcacttttgctggccatggcaggtatgctgggcactggactggggatgtgtggtctagctgggagcagctggcttcttctgtgcctgagatcctgcagttcaacctgctgggggtgcctctggtgggggctgatgtgtgtgggttcctgggcaacacttctgaggagctgtgtgtgaggtggacccagctgggggccttctaccctttcatgaggaaccacaacagcctgctgagcctgccccaggagccctacagcttctctgagcctgcccagcaggccatgaggaaggccctgaccctgaggtatgccctgctgccccacctgtacaccctgttccaccaggcccatgtggctggggagactgtggctaggcctctgttcctggagttccccaaggactctagcacctggactgtggaccaccagctgctgtggggggaggccctgctgatcactcctgtgctgcaggctgggaaggctgaggtgactggctatttccccctgggcacctggtatgacctgcagactgtgcctgtggaggccctggggagcctgccccccccccctgctgcccccagggagcctgccatccactctgagggccagtgggtgaccctgcctgcccctctggataccatcaatgtgcacctgagggctggctacatcattcccctgcagggccctggcctgaccactactgagtctaggcagcagcccatggccctggctgtggccctgaccaaggggggggaggctaggggggagctgttttgggatgatggggagagcctggaggtgctggagaggggggcctacactcaggtgatcttcctggccaggaacaataccattgtgaatgagctggtgagggtgacctctgagggggctggcctgcagctgcagaaggtgactgtgctgggggtggccactgccccccagcaggtgctgagcaatggggtgcctgtgagcaacttcacctatagccctgataccaaggtgctggatatttgtgtgagcctgctgatgggggagcagttcctggtgagctggtgctgactcgagagatctaccggtgaattcaccgcgggtttaaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtgggggctagctctaga

SEQ ID NO: 8 - DNA, 3016 bp, synthetic construct
  source 1..3016: mol_type = other DNA
atggctttcctgtggctgctgtcttgttgggctctgctgggcaccacctttggcctgctggtgcccagggagctgtctggcagcagccctgtgctggaggagacccaccctgctcatcagcagggggctagcaggcctggccccagggatgcccaggctcaccctgggagacccagggctgtgcccactcagtgtgatgtgccccccaacagcaggtttgactgtgctcctgacaaggctatcacccaggagcagtgtgaggccagggggtgctgctacattcctgctaagcagggcctgcagggggcccagatgggccagccctggtgcttcttccccccctcttatcccagctataagctggagaacctgagcagctctgagatgggctacactgccaccctgaccaggaccactcccaccttctttcccaaggatattctgactctgaggctggatgtgatgatggagactgagaacaggctgcacttcactatcaaggaccctgccaataggaggtatgaggtgcccctggagactcctcatgtgcatagcagggccccttctcctctgtattctgtggagttctctgaggagccctttggggtgattgtgaggaggcagctggatggcagggtgctgctgaacaccactgtggcccccctgttctttgctgaccagttcctgcagctgagcacttctctgcccagccagtacattactgggctggctgagcatctgagccccctgatgctgagcacctcttggaccaggatcaccctgtggaacagggacctggcccccactcctggggctaacctgtatggctctcaccccttttacctggccctggaggatgggggctctgcccatggggtgtttctgctgaacagcaatgctatggatgtggtgctgcagccctctccagccctgtcttggaggagcactgggggcattctggatgtgtacattttcctggggcctgaacccaagtctgtggtgcagcagtacctggatgtggtgggctaccccttcatgcccccctattgggggctggggtttcacctgtgcaggtggggctacagcagcactgccatcaccaggcaggtggtggagaacatgaccagggcccatttccccctggatgtgcagtggaatgacctggactacatggatagcaggagggatttcaccttcaacaaggatggcttcagggactttcctgccatggtgcaggagctgcaccaggggggcaggaggtatatgatgattgtggaccctgctatcagcagctctggccctgctggctcttacaggccctatgatgagggcctgaggaggggggtgtttatcactaatgaaactggccagcctctgattggcaaggtctggcctggctctactgccttccctgattttactaaccccactgccctggcctggtgggaggacatggtggctgagttccatgatcaggtgccttttgatggcatgtggattgatatgaatgaaccaagcaacttcatcagaggctctgaggatggctgccccaacaatgagctggagaaccccccctatgtgcctggggtggtggggggcactctgcaggctgccaccatttgtgctagcagccaccagttcctgagcacccactacaatctgcacaacctgtatggcctgactgaagccattgccagccatagggccctggtgaaggccaggggcactaggccttttgtgatcagcaggagcacttttgctggccatggcaggtatgctggccactggactggggatgtgtggagcagctgggagcagctggccagctctgtgcctgagattctgcagtttaacctgctgggggtgcccctggtgggggctgatgtgtgtggcttcctgggcaacacctctgaggagctgtgtgtgaggtggacccagctgggggccttttatcccttcatgaggaaccacaacagcctgctgagcctgcctcaggagccctactctttctctgagcctgcccagcaggccatgaggaaggccctgaccctgaggtatgccctgctgccccacctgtataccctgttccaccaggcccatgtggctggggagactgtggccaggcccctgttcctggagttccccaaggacagcagcacctggactgtggatcatcagctgctgtggggggaggccctgctgatcacccctgtgctgcaggctggcaaggctgaggtcactggctacttccctctgggcacctggtatgacctgcagactgtgcctgtggaggctctgggcagcctgccccccccccctgctgctcccagggagcctgccatccactctgagggccagtgggtgaccctgcctgctcccctggacaccatcaatgtgcacctgagggctggctacattatccccctgcagggcccagggctgactaccactgagagcagacagcagcccatggctctggctgtggccctgaccaaggggggggaagctaggggggagctgttctgggatgatggggagagcctggaggtgctggagaggggggcctatacccaggtgatcttcctggctaggaacaacaccattgtcaatgagctggtgagggtgacttctgagggggctgggctgcagctgcagaaggtgactgtgctgggggtggccactgctccccagcaggtgctgagcaatggggtgcctgtgagcaacttcacctacagccctgacaccaaggtgctggacatctgtgtgtctctgctgatgggggagcagttcctggtgagctggtgctgactcgagagatctaccggtgaattcaccgcgggtttaaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtgggggctagctctaga

SEQ ID NO: 9 - DNA, 3016 bp, synthetic construct
  source 1..3016: mol_type = other DNA
atggccttcctgtggctgctgtcttgctgggctctgctggggaccacctttggcctgctggtccccagggagctgtctggctcttctcctgtcctggaggagacccaccctgcccaccagcagggggctagcaggcctggccccagggatgcccaggcccaccctggcaggcccagggctgtgcccacccagtgtgatgtgcctcccaacagcaggtttgactgtgcccctgacaaggccatcacccaggagcagtgtgaggccaggggctgctgctatatccctgccaagcagggcctgcagggggctcagatgggccagccctggtgcttctttcccccctcttatcctagctataagctggagaacctgagcagctctgagatggggtacactgccaccctgaccaggaccacccccactttcttccctaaggacatcctgaccctgaggctggatgtgatgatggagactgagaataggctgcactttactatcaaggaccctgccaacaggaggtatgaggtgcctctggagaccccccatgtgcattctagggcccccagccccctgtactctgtggagttctctgaggagccctttggggtgattgtgaggagacagctggatggcagggtcctgctgaacaccactgtggctcccctgttttttgctgaccagttcctgcagctgagcaccagcctgcccagccagtacatcactgggctggctgagcacctgagccccctgatgctgagcaccagctggaccaggatcaccctgtggaacagggatctggctcctacccctggggccaacctgtatggctctcaccccttttacctggccctggaggatgggggctctgcccatggggtgttcctgctgaacagcaatgctatggatgtggtgctgcagcccagccctgccctgagctggaggtctactgggggcatcctggatgtgtacatctttctggggcctgagcccaagtctgtggtgcagcagtacctggatgtggtgggctatccttttatgcccccctattggggcctgggcttccacctgtgcaggtggggctacagcagcactgccatcaccagacaggtggtggagaacatgaccagggcccacttccccctggatgtgcagtggaatgacctggactacatggacagcaggagggacttcacctttaacaaggatggctttagggacttccctgccatggtgcaggagctgcatcaggggggcaggaggtacatgatgattgtggacccagccatcagcagctctgggcctgctgggtcttacaggccctatgatgagggcctgaggaggggggtgttcatcaccaatgagactggccagcccctgattggcaaggtgtggcctgggagcactgccttccctgattttaccaaccccactgccctggcctggtgggaggatatggtggctgagtttcatgaccaggtgccctttgatggcatgtggattgacatgaatgagcccagcaatttcatcaggggctctgaggatggctgccccaacaatgagctggagaatcctccctatgtgcctggggtggtggggggcaccctgcaggctgccaccatctgtgcctctagccaccagttcctgagcacccactataacctgcataacctgtatggcctgactgaggccattgccagccatagagccctggtgaaggccagagggaccaggccctttgtgatctctaggagcacctttgctggccatggcaggtatgctggccactggactggggatgtgtggagctcttgggagcagctggccagctctgtgccagagatcctgcagttcaacctgctgggggtgcctctggtgggggctgatgtgtgtggcttcctgggcaatacctctgaagagctgtgtgtgaggtggactcagctgggggccttctatcccttcatgaggaaccacaacagcctgctgtctctgccccaggagccctacagcttctctgagcctgctcagcaggctatgaggaaggccctgaccctgaggtatgccctgctgccccatctgtacaccctgttccaccaggcccatgtggctggggagactgtggccaggcccctgtttctggagtttcccaaggacagcagcacctggactgtggaccatcagctgctgtggggggaggctctgctgattacccctgtgctgcaggctggcaaggctgaggtgactgggtacttccccctggggacttggtatgacctgcagactgtgcctgtggaagctctgggcagcctgcccccaccccctgctgcccctagggagcctgccatccactctgagggccagtgggtgaccctgcctgcccctctggacaccatcaatgtgcacctgagggctggctatatcatccccctgcagggccctgggctgaccaccactgagagcaggcagcagcccatggccctggctgtggccctgactaaggggggggaggccaggggggagctgttctgggatgatggggagagcctggaggtgctggagagaggggcctacacccaggtgatctttctggccaggaacaacaccattgtgaatgagctggtgagggtgacttctgagggggctggcctgcagctgcagaaggtgactgtgctgggggtggccactgccccccagcaggtgctgagcaatggggtgcctgtgtctaacttcacctacagccctgatactaaggtgctggatatctgtgtgagcctgctgatgggggagcagtttctggtgagctggtgctgactcgagagatctaccggtgaattcaccgcgggtttaaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtgggggctagctctaga

SEQ ID NO: 10 - DNA, 3016 bp, synthetic construct
  source 1..3016: mol_type = other DNA
atggcctttctgtggctgctgtcctgctgggccctgctggggaccacctttggcctgctggtgcccagggagctgtctgggagcagcccagtgctggaggagacccaccctgcccaccagcagggggccagcaggcctggccctagggatgcccaggcccaccctggcaggcccagggctgtgcctacccagtgtgatgtgccacccaattctaggtttgactgtgctcctgacaaggccatcactcaggagcagtgtgaagctagggggtgctgctacatcccagccaagcagggcctgcagggggcccagatgggccagccctggtgcttcttcccccccagctaccctagctacaagctggagaatctgagcagctctgagatgggctacactgctaccctgaccaggaccactcctaccttcttccccaaggacatcctgactctgaggctggatgtcatgatggagactgaaaataggctgcacttcaccatcaaggaccctgccaataggaggtatgaggtgcctctggagaccccccatgtgcatagcagggctcccagccccctgtattctgtggagttctctgaggagccctttggggtcattgtgaggagacagctggatgggagggtgctgctgaacactactgtggctcccctgttctttgctgaccagttcctgcagctgtctaccagcctgcccagccagtacatcactgggctggctgagcatctgagccccctgatgctgagcaccagctggaccaggatcactctgtggaacagggatctggcccccactcctggggccaacctgtatgggagccatcccttctacctggccctggaggatgggggctctgcccatggggtgttcctgctgaacagcaatgccatggatgtggtgctgcagcctagccctgccctgagctggaggagcactgggggcatcctggatgtctacatcttcctggggcctgagcccaagtctgtggtgcagcagtatctggatgtggtggggtatcccttcatgcccccctactggggcctgggctttcacctgtgcaggtggggctacagcagcactgccatcaccaggcaggtggtggagaacatgaccagggcccacttccctctggatgtgcagtggaatgacctggactatatggattctaggagagactttacttttaacaaggatggcttcagggatttccctgccatggtgcaggagctgcaccaggggggcaggaggtacatgatgattgtggaccctgctattagcagctctggccctgctgggtcttacaggccttatgatgagggcctgaggaggggggtgttcatcaccaatgagactggccagcccctgattggcaaggtgtggcctggcagcactgccttccctgacttcaccaaccccactgccctggcctggtgggaggacatggtggctgagttccatgaccaggtgccctttgatgggatgtggattgacatgaatgagccctctaacttcatcagggggtctgaggatggctgccccaacaatgagctggagaaccccccctatgtgcctggggtggtggggggcactctgcaggctgccactatctgtgcttcttctcaccagtttctgagcacccactataatctgcacaacctgtatggcctgactgaggccattgccagccatagggccctggtgaaggccaggggcaccaggccctttgtgatcagcaggtctacctttgctggccatggcaggtatgctggccactggactggggatgtgtggtcttcttgggagcagctggccagctctgtgcctgagatcctgcagttcaacctgctgggggtgcctctggtgggggctgatgtgtgtggctttctgggcaacacctctgaggagctgtgtgtgaggtggacccagctgggggccttttaccccttcatgaggaaccacaatagcctgctgagcctgccccaggagccttactctttctctgagcctgcccagcaggccatgaggaaggccctgactctgaggtatgccctgctgccccatctgtataccctgtttcaccaggcccatgtggctggggagactgtggctaggcctctgtttctggagttccctaaggactctagcacctggactgtggaccaccagctgctgtggggggaggccctgctgatcacccctgtgctgcaggctggcaaggctgaggtgactggctacttccccctgggcacctggtatgacctgcagactgtgcctgtggaggccctggggagcctgcctcccccccctgctgcccccagggagcctgccattcattctgagggccagtgggtgaccctgcctgcccctctggacaccatcaatgtgcacctgagggctgggtacatcatccccctgcagggccctggcctgaccaccactgagagcaggcagcagcccatggccctggctgtggctctgaccaaggggggggaggccaggggggagctgttctgggatgatggggagtctctggaggtgctggagaggggggcctacacccaggtgatctttctggccaggaacaatactattgtgaatgagctggtgagggtgacctctgagggggctggcctgcagctgcagaaggtgactgtgctgggggtggccactgccccccagcaggtcctgagcaatggggtgcctgtgagcaacttcacctactctcctgacaccaaggtgctggacatttgtgtgtctctgctgatgggggagcagttcctggtgagctggtgctgactcgagagatctaccggtgaattcaccgcgggtttaaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtgggggctagctctaga

SEQ ID NO: 11 - DNA, 3076 bp, synthetic construct
  source 1..3076: mol_type = other DNA
atggctttcctgtggctgctgagctgctgggctctgctgggcaccacctttgggctgctggtgcctagggagctgtctgggtctagccctgtgctggaggagactcaccctgcccatcagcagggggctagcaggcctggccccagggatgctcaggcccaccctggcaggcccagggctgtgcccacccagtgtgatgtgccccccaacagcaggtttgactgtgcccctgacaaggccattacccaggagcagtgtgaggccaggggctgctgctacattccagctaagcagggcctgcagggggcccagatgggccagccctggtgcttcttcccccccagctatcctagctataaactggagaacctgagcagctctgagatgggctatactgccaccctgactaggactactcccaccttttttcctaaggatatcctgaccctgaggctggatgtgatgatggagactgagaacaggctgcacttcactattaaggaccctgccaataggaggtatgaagtgcctctggagactcctcatgtgcactctagggcccccagccccctgtattctgtggagttctctgaggagccctttggggtgattgtgaggaggcagctggatggcagggtgctgctgaacaccactgtggcccccctgttctttgctgaccagttcctgcagctgagcaccagcctgcccagccagtacatcactgggctggctgagcatctgagccctctgatgctgagcacctcttggaccaggatcaccctgtggaatagggatctggcccccacccctggggctaatctgtatggctctcatcccttttacctggccctggaggatgggggctctgcccatggggtgtttctgctgaacagcaatgccatggatgtggtgctgcagccctctcctgccctgagctggaggagcactgggggcatcctggatgtgtacatcttcctgggccctgagcccaagtctgtggtccagcagtatctggatgtggtgggctacccctttatgcccccctattggggcctgggcttccacctgtgcaggtgggggtattcttctactgctatcaccaggcaggtggtggagaacatgaccagggctcacttccccctggatgtgcagtggaatgacctggactatatggactctaggagggatttcaccttcaacaaggatggcttcagggacttccctgctatggtccaggagctgcatcaggggggcaggaggtacatgatgattgtggaccctgccatcagcagctctggccctgctggcagctataggccctatgatgagggcctgaggaggggggtgtttatcactaatgaaactgggcagcccctgattggcaaggtgtggcctggctctactgccttccctgacttcaccaaccccactgctctggcctggtgggaggacatggtggctgagttccatgaccaggtgccttttgatggcatgtggattgacatgaatgagcccagcaacttcatcaggggctctgaggatgggtgccccaataatgagctggagaaccccccctatgtgcctggggtggtggggggcaccctgcaggctgccactatttgtgccagctctcaccagttcctgagcacccactacaacctgcacaatctgtatggcctgactgaggccattgccagccacagggccctggtgaaggccaggggcactaggccctttgtgatctctagaagcacctttgctggccatgggaggtatgctggccactggactggggatgtgtggagctcttgggagcagctggccagctctgtgcctgagatcctgcagttcaacctgctgggggtgcccctggtgggggctgatgtgtgtggcttcctgggcaacacctctgaagagctgtgtgtgaggtggacccagctgggggccttctaccctttcatgaggaaccacaacagcctgctgagcctgcctcaggagccttactctttctctgagcctgcccagcaggccatgaggaaggccctgaccctgaggtatgctctgctgccccacctgtacaccctgttccaccaggcccatgtggctggggagactgtggccaggcccctgttcctggagtttcctaaggatagcagcacctggactgtggaccaccagctgctgtggggggaggccctgctgattacccctgtgctgcaggctggcaaggctgaggtgactggctacttccccctgggcacttggtatgacctgcagactgtgcctgtggaagccctgggcagcctgcctcccccccctgctgcccccagggagcctgccatccactctgagggccagtgggtgaccctgcctgcccccctggacaccattaatgtgcatctgagggctgggtatattatccccctgcaggggcctgggctgactaccactgagagcaggcagcagcctatggccctggctgtggctctgactaaggggggggaggccaggggggagctgttctgggatgatggggagagcctggaggtgctggagaggggggcctacacccaggtgattttcctggccaggaacaacaccattgtgaatgagctggtgagggtgacctctgagggggctggcctgcagctgcagaaagtgactgtgctgggggtggccactgccccccagcaggtgctgagcaatggggtgcctgtgagcaacttcacctacagccctgacaccaaggtgctggatatttgtgtgagcctgctgatgggggagcagttcctggtgagctggtgctgaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatgg

SEQ ID NO: 12 - DNA, 3076 bp, synthetic construct
  source 1..3076: mol_type = other DNA
atggctttcctgtggctgctgtcttgctgggccctgctggggactacctttggcctgctggtgcccagggaactgtctggctctagcccagtgctggaggagacccaccctgcccaccagcagggggcttctaggcctggccccagggatgcccaggcccaccctggcaggccaagggctgtgcccacccagtgtgatgtgccccccaactctagatttgattgtgcccctgataaggccatcacccaggagcagtgtgaggctaggggctgctgctacatccctgctaagcagggcctgcagggggctcagatgggccagccctggtgcttcttcccccccagctatccctcttacaagctggagaatctgagcagctctgagatgggctacactgccaccctgaccaggactactcccaccttcttccccaaggacatcctgaccctgaggctggatgtgatgatggagactgagaacaggctgcatttcaccatcaaggatcctgccaacaggaggtatgaggtgcctctggagaccccccatgtgcacagcagggctccttctcccctgtactctgtggagttctctgaggaaccctttggggtgattgtgaggaggcagctggatggcagggtcctgctgaacaccactgtggcccccctgttctttgctgatcagttcctgcagctgtccacttctctgcctagccagtacatcactgggctggctgagcacctgagccctctgatgctgagcacctcttggactaggatcaccctgtggaacagggacctggcccccacccctggggccaacctgtatggcagccaccccttctatctggccctggaggatgggggctctgcccatggggtgttcctgctgaatagcaatgctatggatgtggtgctgcagcccagccctgccctgtcttggaggagcactgggggcatcctggatgtgtacattttcctggggcctgagcccaagtctgtggtgcagcagtacctggatgtggtgggctaccccttcatgcctccctactggggcctgggcttccacctgtgcaggtggggctacagctctactgccatcaccaggcaggtggtggagaatatgaccagggcccacttccccctggatgtgcagtggaatgacctggactacatggactctaggagggacttcaccttcaataaggatggcttcagagacttccctgccatggtgcaggagctgcatcaggggggcaggaggtacatgatgattgtggaccctgccatcagctcttctggccctgctggctcttacaggccctatgatgagggcctgaggaggggggtgttcatcaccaatgagactgggcagcccctgattgggaaggtgtggcctggctctactgccttccctgacttcaccaatcctactgccctggcctggtgggaggacatggtggctgagttccatgaccaggtgccctttgatggcatgtggattgacatgaatgagccctctaatttcatcaggggctctgaggatggctgccccaacaatgagctggagaaccccccctatgtgcctggggtggtggggggcaccctgcaggctgccaccatctgtgctagctctcaccagttcctgagcacccactacaatctgcataacctgtatggcctgactgaggccattgccagccacagggccctggtgaaggctaggggcaccaggccctttgtgatttctaggagcacttttgctggccatggcaggtatgctgggcactggactggggatgtgtggtctagctgggagcagctggcttcttctgtgcctgagatcctgcagttcaacctgctgggggtgcctctggtgggggctgatgtgtgtgggttcctgggcaacacttctgaggagctgtgtgtgaggtggacccagctgggggccttctaccctttcatgaggaaccacaacagcctgctgagcctgccccaggagccctacagcttctctgagcctgcccagcaggccatgaggaaggccctgaccctgaggtatgccctgctgccccacctgtacaccctgttccaccaggcccatgtggctggggagactgtggctaggcctctgttcctggagttccccaaggactctagcacctggactgtggaccaccagctgctgtggggggaggccctgctgatcactcctgtgctgcaggctgggaaggctgaggtgactggctatttccccctgggcacctggtatgacctgcagactgtgcctgtggaggccctggggagcctgccccccccccctgctgcccccagggagcctgccatccactctgagggccagtgggtgaccctgcctgcccctctggataccatcaatgtgcacctgagggctggctacatcattcccctgcagggccctggcctgaccactactgagtctaggcagcagcccatggccctggctgtggccctgaccaaggggggggaggctaggggggagctgttttgggatgatggggagagcctggaggtgctggagaggggggcctacactcaggtgatcttcctggccaggaacaataccattgtgaatgagctggtgagggtgacctctgagggggctggcctgcagctgcagaaggtgactgtgctgggggtggccactgccccccagcaggtgctgagcaatggggtgcctgtgagcaacttcacctatagccctgataccaaggtgctggatatttgtgtgagcctgctgatgggggagcagttcctggtgagctggtgctgaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatgg

SEQ ID NO: 13 - DNA, 3076 bp, synthetic construct
  source 1..3076: mol_type = other DNA
atggctttcctgtggctgctgtcttgttgggctctgctgggcaccacctttggcctgctggtgcccagggagctgtctggcagcagccctgtgctggaggagacccaccctgctcatcagcagggggctagcaggcctggccccagggatgcccaggctcaccctgggagacccagggctgtgcccactcagtgtgatgtgccccccaacagcaggtttgactgtgctcctgacaaggctatcacccaggagcagtgtgaggccagggggtgctgctacattcctgctaagcagggcctgcagggggcccagatgggccagccctggtgcttcttccccccctcttatcccagctataagctggagaacctgagcagctctgagatgggctacactgccaccctgaccaggaccactcccaccttctttcccaaggatattctgactctgaggctggatgtgatgatggagactgagaacaggctgcacttcactatcaaggaccctgccaataggaggtatgaggtgcccctggagactcctcatgtgcatagcagggccccttctcctctgtattctgtggagttctctgaggagccctttggggtgattgtgaggaggcagctggatggcagggtgctgctgaacaccactgtggcccccctgttctttgctgaccagttcctgcagctgagcacttctctgcccagccagtacattactgggctggctgagcatctgagccccctgatgctgagcacctcttggaccaggatcaccctgtggaacagggacctggcccccactcctggggctaacctgtatggctctcaccccttttacctggccctggaggatgggggctctgcccatggggtgtttctgctgaacagcaatgctatggatgtggtgctgcagccctctccagccctgtcttggaggagcactgggggcattctggatgtgtacattttcctggggcctgaacccaagtctgtggtgcagcagtacctggatgtggtgggctaccccttcatgcccccctattgggggctggggtttcacctgtgcaggtggggctacagcagcactgccatcaccaggcaggtggtggagaacatgaccagggcccatttccccctggatgtgcagtggaatgacctggactacatggatagcaggagggatttcaccttcaacaaggatggcttcagggactttcctgccatggtgcaggagctgcaccaggggggcaggaggtatatgatgattgtggaccctgctatcagcagctctggccctgctggctcttacaggccctatgatgagggcctgaggaggggggtgtttatcactaatgaaactggccagcctctgattggcaaggtctggcctggctctactgccttccctgattttactaaccccactgccctggcctggtgggaggacatggtggctgagttccatgatcaggtgccttttgatggcatgtggattgatatgaatgaaccaagcaacttcatcagaggctctgaggatggctgccccaacaatgagctggagaaccccccctatgtgcctggggtggtggggggcactctgcaggctgccaccatttgtgctagcagccaccagttcctgagcacccactacaatctgcacaacctgtatggcctgactgaagccattgccagccatagggccctggtgaaggccaggggcactaggccttttgtgatcagcaggagcacttttgctggccatggcaggtatgctggccactggactggggatgtgtggagcagctgggagcagctggccagctctgtgcctgagattctgcagtttaacctgctgggggtgcccctggtgggggctgatgtgtgtggcttcctgggcaacacctctgaggagctgtgtgtgaggtggacccagctgggggccttttatcccttcatgaggaaccacaacagcctgctgagcctgcctcaggagccctactctttctctgagcctgcccagcaggccatgaggaaggccctgaccctgaggtatgccctgctgccccacctgtataccctgttccaccaggcccatgtggctggggagactgtggccaggcccctgttcctggagttccccaaggacagcagcacctggactgtggatcatcagctgctgtggggggaggccctgctgatcacccctgtgctgcaggctggcaaggctgaggtcactggctacttccctctgggcacctggtatgacctgcagactgtgcctgtggaggctctgggcagcctgccccccccccctgctgctcccagggagcctgccatccactctgagggccagtgggtgaccctgcctgctcccctggacaccatcaatgtgcacctgagggctggctacattatccccctgcagggcccagggctgactaccactgagagcagacagcagcccatggctctggctgtggccctgaccaaggggggggaagctaggggggagctgttctgggatgatggggagagcctggaggtgctggagaggggggcctatacccaggtgatcttcctggctaggaacaacaccattgtcaatgagctggtgagggtgacttctgagggggctgggctgcagctgcagaaggtgactgtgctgggggtggccactgctccccagcaggtgctgagcaatggggtgcctgtgagcaacttcacctacagccctgacaccaaggtgctggacatctgtgtgtctctgctgatgggggagcagttcctggtgagctggtgctgaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatgg

SEQ ID NO: 14 - DNA, 3076 bp, synthetic construct
  source 1..3076: mol_type = other DNA
atggccttcctgtggctgctgtcttgctgggctctgctggggaccacctttggcctgctggtccccagggagctgtctggctcttctcctgtcctggaggagacccaccctgcccaccagcagggggctagcaggcctggccccagggatgcccaggcccaccctggcaggcccagggctgtgcccacccagtgtgatgtgcctcccaacagcaggtttgactgtgcccctgacaaggccatcacccaggagcagtgtgaggccaggggctgctgctatatccctgccaagcagggcctgcagggggctcagatgggccagccctggtgcttctttcccccctcttatcctagctataagctggagaacctgagcagctctgagatggggtacactgccaccctgaccaggaccacccccactttcttccctaaggacatcctgaccctgaggctggatgtgatgatggagactgagaataggctgcactttactatcaaggaccctgccaacaggaggtatgaggtgcctctggagaccccccatgtgcattctagggcccccagccccctgtactctgtggagttctctgaggagccctttggggtgattgtgaggagacagctggatggcagggtcctgctgaacaccactgtggctcccctgttttttgctgaccagttcctgcagctgagcaccagcctgcccagccagtacatcactgggctggctgagcacctgagccccctgatgctgagcaccagctggaccaggatcaccctgtggaacagggatctggctcctacccctggggccaacctgtatggctctcaccccttttacctggccctggaggatgggggctctgcccatggggtgttcctgctgaacagcaatgctatggatgtggtgctgcagcccagccctgccctgagctggaggtctactgggggcatcctggatgtgtacatctttctggggcctgagcccaagtctgtggtgcagcagtacctggatgtggtgggctatccttttatgcccccctattggggcctgggcttccacctgtgcaggtggggctacagcagcactgccatcaccagacaggtggtggagaacatgaccagggcccacttccccctggatgtgcagtggaatgacctggactacatggacagcaggagggacttcacctttaacaaggatggctttagggacttccctgccatggtgcaggagctgcatcaggggggcaggaggtacatgatgattgtggacccagccatcagcagctctgggcctgctgggtcttacaggccctatgatgagggcctgaggaggggggtgttcatcaccaatgagactggccagcccctgattggcaaggtgtggcctgggagcactgccttccctgattttaccaaccccactgccctggcctggtgggaggatatggtggctgagtttcatgaccaggtgccctttgatggcatgtggattgacatgaatgagcccagcaatttcatcaggggctctgaggatggctgccccaacaatgagctggagaatcctccctatgtgcctggggtggtggggggcaccctgcaggctgccaccatctgtgcctctagccaccagttcctgagcacccactataacctgcataacctgtatggcctgactgaggccattgccagccatagagccctggtgaaggccagagggaccaggccctttgtgatctctaggagcacctttgctggccatggcaggtatgctggccactggactggggatgtgtggagctcttgggagcagctggccagctctgtgccagagatcctgcagttcaacctgctgggggtgcctctggtgggggctgatgtgtgtggcttcctgggcaatacctctgaagagctgtgtgtgaggtggactcagctgggggccttctatcccttcatgaggaaccacaacagcctgctgtctctgccccaggagccctacagcttctctgagcctgctcagcaggctatgaggaaggccctgaccctgaggtatgccctgctgccccatctgtacaccctgttccaccaggcccatgtggctggggagactgtggccaggcccctgtttctggagtttcccaaggacagcagcacctggactgtggaccatcagctgctgtggggggaggctctgctgattacccctgtgctgcaggctggcaaggctgaggtgactgggtacttccccctggggacttggtatgacctgcagactgtgcctgtggaagctctgggcagcctgcccccaccccctgctgcccctagggagcctgccatccactctgagggccagtgggtgaccctgcctgcccctctggacaccatcaatgtgcacctgagggctggctatatcatccccctgcagggccctgggctgaccaccactgagagcaggcagcagcccatggccctggctgtggccctgactaaggggggggaggccaggggggagctgttctgggatgatggggagagcctggaggtgctggagagaggggcctacacccaggtgatctttctggccaggaacaacaccattgtgaatgagctggtgagggtgacttctgagggggctggcctgcagctgcagaaggtgactgtgctgggggtggccactgccccccagcaggtgctgagcaatggggtgcctgtgtctaacttcacctacagccctgatactaaggtgctggatatctgtgtgagcctgctgatgggggagcagtttctggtgagctggtgctgaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatgg

SEQ ID NO: 15 - DNA, 3076 bp, synthetic construct
  source 1..3076: mol_type = other DNA
atggcctttctgtggctgctgtcctgctgggccctgctggggaccacctttggcctgctggtgcccagggagctgtctgggagcagcccagtgctggaggagacccaccctgcccaccagcagggggccagcaggcctggccctagggatgcccaggcccaccctggcaggcccagggctgtgcctacccagtgtgatgtgccacccaattctaggtttgactgtgctcctgacaaggccatcactcaggagcagtgtgaagctagggggtgctgctacatcccagccaagcagggcctgcagggggcccagatgggccagccctggtgcttcttcccccccagctaccctagctacaagctggagaatctgagcagctctgagatgggctacactgctaccctgaccaggaccactcctaccttcttccccaaggacatcctgactctgaggctggatgtcatgatggagactgaaaataggctgcacttcaccatcaaggaccctgccaataggaggtatgaggtgcctctggagaccccccatgtgcatagcagggctcccagccccctgtattctgtggagttctctgaggagccctttggggtcattgtgaggagacagctggatgggagggtgctgctgaacactactgtggctcccctgttctttgctgaccagttcctgcagctgtctaccagcctgcccagccagtacatcactgggctggctgagcatctgagccccctgatgctgagcaccagctggaccaggatcactctgtggaacagggatctggcccccactcctggggccaacctgtatgggagccatcccttctacctggccctggaggatgggggctctgcccatggggtgttcctgctgaacagcaatgccatggatgtggtgctgcagcctagccctgccctgagctggaggagcactgggggcatcctggatgtctacatcttcctggggcctgagcccaagtctgtggtgcagcagtatctggatgtggtggggtatcccttcatgcccccctactggggcctgggctttcacctgtgcaggtggggctacagcagcactgccatcaccaggcaggtggtggagaacatgaccagggcccacttccctctggatgtgcagtggaatgacctggactatatggattctaggagagactttacttttaacaaggatggcttcagggatttccctgccatggtgcaggagctgcaccaggggggcaggaggtacatgatgattgtggaccctgctattagcagctctggccctgctgggtcttacaggccttatgatgagggcctgaggaggggggtgttcatcaccaatgagactggccagcccctgattggcaaggtgtggcctggcagcactgccttccctgacttcaccaaccccactgccctggcctggtgggaggacatggtggctgagttccatgaccaggtgccctttgatgggatgtggattgacatgaatgagccctctaacttcatcagggggtctgaggatggctgccccaacaatgagctggagaaccccccctatgtgcctggggtggtggggggcactctgcaggctgccactatctgtgcttcttctcaccagtttctgagcacccactataatctgcacaacctgtatggcctgactgaggccattgccagccatagggccctggtgaaggccaggggcaccaggccctttgtgatcagcaggtctacctttgctggccatggcaggtatgctggccactggactggggatgtgtggtcttcttgggagcagctggccagctctgtgcctgagatcctgcagttcaacctgctgggggtgcctctggtgggggctgatgtgtgtggctttctgggcaacacctctgaggagctgtgtgtgaggtggacccagctgggggccttttaccccttcatgaggaaccacaatagcctgctgagcctgccccaggagccttactctttctctgagcctgcccagcaggccatgaggaaggccctgactctgaggtatgccctgctgccccatctgtataccctgtttcaccaggcccatgtggctggggagactgtggctaggcctctgtttctggagttccctaaggactctagcacctggactgtggaccaccagctgctgtggggggaggccctgctgatcacccctgtgctgcaggctggcaaggctgaggtgactggctacttccccctgggcacctggtatgacctgcagactgtgcctgtggaggccctggggagcctgcctcccccccctgctgcccccagggagcctgccattcattctgagggccagtgggtgaccctgcctgcccctctggacaccatcaatgtgcacctgagggctgggtacatcatccccctgcagggccctggcctgaccaccactgagagcaggcagcagcccatggccctggctgtggctctgaccaaggggggggaggccaggggggagctgttctgggatgatggggagtctctggaggtgctggagaggggggcctacacccaggtgatctttctggccaggaacaatactattgtgaatgagctggtgagggtgacctctgagggggctggcctgcagctgcagaaggtgactgtgctgggggtggccactgccccccagcaggtcctgagcaatggggtgcctgtgagcaacttcacctactctcctgacaccaaggtgctggacatttgtgtgtctctgctgatgggggagcagttcctggtgagctggtgctgaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatgg

SEQ ID NO: 16 - DNA, 4730 bp, synthetic construct
  source 1..4730: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaataccactttcacaatctgctagcgtttaaacgatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatggcctttctgtggctgctgtcctgctgggccctgctggggaccacctttggcctgctggtgcccagggagctgtctgggagcagcccagtgctggaggagacccaccctgcccaccagcagggggccagcaggcctggccctagggatgcccaggcccaccctggcaggcccagggctgtgcctacccagtgtgatgtgccacccaattctaggtttgactgtgctcctgacaaggccatcactcaggagcagtgtgaagctagggggtgctgctacatcccagccaagcagggcctgcagggggcccagatgggccagccctggtgcttcttcccccccagctaccctagctacaagctggagaatctgagcagctctgagatgggctacactgctaccctgaccaggaccactcctaccttcttccccaaggacatcctgactctgaggctggatgtcatgatggagactgaaaataggctgcacttcaccatcaaggaccctgccaataggaggtatgaggtgcctctggagaccccccatgtgcatagcagggctcccagccccctgtattctgtggagttctctgaggagccctttggggtcattgtgaggagacagctggatgggagggtgctgctgaacactactgtggctcccctgttctttgctgaccagttcctgcagctgtctaccagcctgcccagccagtacatcactgggctggctgagcatctgagccccctgatgctgagcaccagctggaccaggatcactctgtggaacagggatctggcccccactcctggggccaacctgtatgggagccatcccttctacctggccctggaggatgggggctctgcccatggggtgttcctgctgaacagcaatgccatggatgtggtgctgcagcctagccctgccctgagctggaggagcactgggggcatcctggatgtctacatcttcctggggcctgagcccaagtctgtggtgcagcagtatctggatgtggtggggtatcccttcatgcccccctactggggcctgggctttcacctgtgcaggtggggctacagcagcactgccatcaccaggcaggtggtggagaacatgaccagggcccacttccctctggatgtgcagtggaatgacctggactatatggattctaggagagactttacttttaacaaggatggcttcagggatttccctgccatggtgcaggagctgcaccaggggggcaggaggtacatgatgattgtggaccctgctattagcagctctggccctgctgggtcttacaggccttatgatgagggcctgaggaggggggtgttcatcaccaatgagactggccagcccctgattggcaaggtgtggcctggcagcactgccttccctgacttcaccaaccccactgccctggcctggtgggaggacatggtggctgagttccatgaccaggtgccctttgatgggatgtggattgacatgaatgagccctctaacttcatcagggggtctgaggatggctgccccaacaatgagctggagaaccccccctatgtgcctggggtggtggggggcactctgcaggctgccactatctgtgcttcttctcaccagtttctgagcacccactataatctgcacaacctgtatggcctgactgaggccattgccagccatagggccctggtgaaggccaggggcaccaggccctttgtgatcagcaggtctacctttgctggccatggcaggtatgctggccactggactggggatgtgtggtcttcttgggagcagctggccagctctgtgcctgagatcctgcagttcaacctgctgggggtgcctctggtgggggctgatgtgtgtggctttctgggcaacacctctgaggagctgtgtgtgaggtggacccagctgggggccttttaccccttcatgaggaaccacaatagcctgctgagcctgccccaggagccttactctttctctgagcctgcccagcaggccatgaggaaggccctgactctgaggtatgccctgctgccccatctgtataccctgtttcaccaggcccatgtggctggggagactgtggctaggcctctgtttctggagttccctaaggactctagcacctggactgtggaccaccagctgctgtggggggaggccctgctgatcacccctgtgctgcaggctggcaaggctgaggtgactggctacttccccctgggcacctggtatgacctgcagactgtgcctgtggaggccctggggagcctgcctcccccccctgctgcccccagggagcctgccattcattctgagggccagtgggtgaccctgcctgcccctctggacaccatcaatgtgcacctgagggctgggtacatcatccccctgcagggccctggcctgaccaccactgagagcaggcagcagcccatggccctggctgtggctctgaccaaggggggggaggccaggggggagctgttctgggatgatggggagtctctggaggtgctggagaggggggcctacacccaggtgatctttctggccaggaacaatactattgtgaatgagctggtgagggtgacctctgagggggctggcctgcagctgcagaaggtgactgtgctgggggtggccactgccccccagcaggtcctgagcaatggggtgcctgtgagcaacttcacctactctcctgacaccaaggtgctggacatttgtgtgtctctgctgatgggggagcagttcctggtgagctggtgctgaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 17 - DNA, 4701 bp, synthetic construct
  source 1..4701: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatggcctttctgtggctgctgtcctgctgggccctgctggggaccacctttggcctgctggtgcccagggagctgtctgggagcagcccagtgctggaggagacccaccctgcccaccagcagggggccagcaggcctggccctagggatgcccaggcccaccctggcaggcccagggctgtgcctacccagtgtgatgtgccacccaattctaggtttgactgtgctcctgacaaggccatcactcaggagcagtgtgaagctagggggtgctgctacatcccagccaagcagggcctgcagggggcccagatgggccagccctggtgcttcttcccccccagctaccctagctacaagctggagaatctgagcagctctgagatgggctacactgctaccctgaccaggaccactcctaccttcttccccaaggacatcctgactctgaggctggatgtcatgatggagactgaaaataggctgcacttcaccatcaaggaccctgccaataggaggtatgaggtgcctctggagaccccccatgtgcatagcagggctcccagccccctgtattctgtggagttctctgaggagccctttggggtcattgtgaggagacagctggatgggagggtgctgctgaacactactgtggctcccctgttctttgctgaccagttcctgcagctgtctaccagcctgcccagccagtacatcactgggctggctgagcatctgagccccctgatgctgagcaccagctggaccaggatcactctgtggaacagggatctggcccccactcctggggccaacctgtatgggagccatcccttctacctggccctggaggatgggggctctgcccatggggtgttcctgctgaacagcaatgccatggatgtggtgctgcagcctagccctgccctgagctggaggagcactgggggcatcctggatgtctacatcttcctggggcctgagcccaagtctgtggtgcagcagtatctggatgtggtggggtatcccttcatgcccccctactggggcctgggctttcacctgtgcaggtggggctacagcagcactgccatcaccaggcaggtggtggagaacatgaccagggcccacttccctctggatgtgcagtggaatgacctggactatatggattctaggagagactttacttttaacaaggatggcttcagggatttccctgccatggtgcaggagctgcaccaggggggcaggaggtacatgatgattgtggaccctgctattagcagctctggccctgctgggtcttacaggccttatgatgagggcctgaggaggggggtgttcatcaccaatgagactggccagcccctgattggcaaggtgtggcctggcagcactgccttccctgacttcaccaaccccactgccctggcctggtgggaggacatggtggctgagttccatgaccaggtgccctttgatgggatgtggattgacatgaatgagccctctaacttcatcagggggtctgaggatggctgccccaacaatgagctggagaaccccccctatgtgcctggggtggtggggggcactctgcaggctgccactatctgtgcttcttctcaccagtttctgagcacccactataatctgcacaacctgtatggcctgactgaggccattgccagccatagggccctggtgaaggccaggggcaccaggccctttgtgatcagcaggtctacctttgctggccatggcaggtatgctggccactggactggggatgtgtggtcttcttgggagcagctggccagctctgtgcctgagatcctgcagttcaacctgctgggggtgcctctggtgggggctgatgtgtgtggctttctgggcaacacctctgaggagctgtgtgtgaggtggacccagctgggggccttttaccccttcatgaggaaccacaatagcctgctgagcctgccccaggagccttactctttctctgagcctgcccagcaggccatgaggaaggccctgactctgaggtatgccctgctgccccatctgtataccctgtttcaccaggcccatgtggctggggagactgtggctaggcctctgtttctggagttccctaaggactctagcacctggactgtggaccaccagctgctgtggggggaggccctgctgatcacccctgtgctgcaggctggcaaggctgaggtgactggctacttccccctgggcacctggtatgacctgcagactgtgcctgtggaggccctggggagcctgcctcccccccctgctgcccccagggagcctgccattcattctgagggccagtgggtgaccctgcctgcccctctggacaccatcaatgtgcacctgagggctgggtacatcatccccctgcagggccctggcctgaccaccactgagagcaggcagcagcccatggccctggctgtggctctgaccaaggggggggaggccaggggggagctgttctgggatgatggggagtctctggaggtgctggagaggggggcctacacccaggtgatctttctggccaggaacaatactattgtgaatgagctggtgagggtgacctctgagggggctggcctgcagctgcagaaggtgactgtgctgggggtggccactgccccccagcaggtcctgagcaatggggtgcctgtgagcaacttcacctactctcctgacaccaaggtgctggacatttgtgtgtctctgctgatgggggagcagttcctggtgagctggtgctgaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 18 - DNA, 4701 bp, synthetic construct
  source 1..4701: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatggctttcctgtggctgctgtcttgttgggctctgctgggcaccacctttggcctgctggtgcccagggagctgtctggcagcagccctgtgctggaggagacccaccctgctcatcagcagggggctagcaggcctggccccagggatgcccaggctcaccctgggagacccagggctgtgcccactcagtgtgatgtgccccccaacagcaggtttgactgtgctcctgacaaggctatcacccaggagcagtgtgaggccagggggtgctgctacattcctgctaagcagggcctgcagggggcccagatgggccagccctggtgcttcttccccccctcttatcccagctataagctggagaacctgagcagctctgagatgggctacactgccaccctgaccaggaccactcccaccttctttcccaaggatattctgactctgaggctggatgtgatgatggagactgagaacaggctgcacttcactatcaaggaccctgccaataggaggtatgaggtgcccctggagactcctcatgtgcatagcagggccccttctcctctgtattctgtggagttctctgaggagccctttggggtgattgtgaggaggcagctggatggcagggtgctgctgaacaccactgtggcccccctgttctttgctgaccagttcctgcagctgagcacttctctgcccagccagtacattactgggctggctgagcatctgagccccctgatgctgagcacctcttggaccaggatcaccctgtggaacagggacctggcccccactcctggggctaacctgtatggctctcaccccttttacctggccctggaggatgggggctctgcccatggggtgtttctgctgaacagcaatgctatggatgtggtgctgcagccctctccagccctgtcttggaggagcactgggggcattctggatgtgtacattttcctggggcctgaacccaagtctgtggtgcagcagtacctggatgtggtgggctaccccttcatgcccccctattgggggctggggtttcacctgtgcaggtggggctacagcagcactgccatcaccaggcaggtggtggagaacatgaccagggcccatttccccctggatgtgcagtggaatgacctggactacatggatagcaggagggatttcaccttcaacaaggatggcttcagggactttcctgccatggtgcaggagctgcaccaggggggcaggaggtatatgatgattgtggaccctgctatcagcagctctggccctgctggctcttacaggccctatgatgagggcctgaggaggggggtgtttatcactaatgaaactggccagcctctgattggcaaggtctggcctggctctactgccttccctgattttactaaccccactgccctggcctggtgggaggacatggtggctgagttccatgatcaggtgccttttgatggcatgtggattgatatgaatgaaccaagcaacttcatcagaggctctgaggatggctgccccaacaatgagctggagaaccccccctatgtgcctggggtggtggggggcactctgcaggctgccaccatttgtgctagcagccaccagttcctgagcacccactacaatctgcacaacctgtatggcctgactgaagccattgccagccatagggccctggtgaaggccaggggcactaggccttttgtgatcagcaggagcacttttgctggccatggcaggtatgctggccactggactggggatgtgtggagcagctgggagcagctggccagctctgtgcctgagattctgcagtttaacctgctgggggtgcccctggtgggggctgatgtgtgtggcttcctgggcaacacctctgaggagctgtgtgtgaggtggacccagctgggggccttttatcccttcatgaggaaccacaacagcctgctgagcctgcctcaggagccctactctttctctgagcctgcccagcaggccatgaggaaggccctgaccctgaggtatgccctgctgccccacctgtataccctgttccaccaggcccatgtggctggggagactgtggccaggcccctgttcctggagttccccaaggacagcagcacctggactgtggatcatcagctgctgtggggggaggccctgctgatcacccctgtgctgcaggctggcaaggctgaggtcactggctacttccctctgggcacctggtatgacctgcagactgtgcctgtggaggctctgggcagcctgccccccccccctgctgctcccagggagcctgccatccactctgagggccagtgggtgaccctgcctgctcccctggacaccatcaatgtgcacctgagggctggctacattatccccctgcagggcccagggctgactaccactgagagcagacagcagcccatggctctggctgtggccctgaccaaggggggggaagctaggggggagctgttctgggatgatggggagagcctggaggtgctggagaggggggcctatacccaggtgatcttcctggctaggaacaacaccattgtcaatgagctggtgagggtgacttctgagggggctgggctgcagctgcagaaggtgactgtgctgggggtggccactgctccccagcaggtgctgagcaatggggtgcctgtgagcaacttcacctacagccctgacaccaaggtgctggacatctgtgtgtctctgctgatgggggagcagttcctggtgagctggtgctgaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 19 - DNA, 4701 bp, synthetic construct
  source 1..4701: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatggccttcctgtggctgctgtcttgctgggctctgctggggaccacctttggcctgctggtccccagggagctgtctggctcttctcctgtcctggaggagacccaccctgcccaccagcagggggctagcaggcctggccccagggatgcccaggcccaccctggcaggcccagggctgtgcccacccagtgtgatgtgcctcccaacagcaggtttgactgtgcccctgacaaggccatcacccaggagcagtgtgaggccaggggctgctgctatatccctgccaagcagggcctgcagggggctcagatgggccagccctggtgcttctttcccccctcttatcctagctataagctggagaacctgagcagctctgagatggggtacactgccaccctgaccaggaccacccccactttcttccctaaggacatcctgaccctgaggctggatgtgatgatggagactgagaataggctgcactttactatcaaggaccctgccaacaggaggtatgaggtgcctctggagaccccccatgtgcattctagggcccccagccccctgtactctgtggagttctctgaggagccctttggggtgattgtgaggagacagctggatggcagggtcctgctgaacaccactgtggctcccctgttttttgctgaccagttcctgcagctgagcaccagcctgcccagccagtacatcactgggctggctgagcacctgagccccctgatgctgagcaccagctggaccaggatcaccctgtggaacagggatctggctcctacccctggggccaacctgtatggctctcaccccttttacctggccctggaggatgggggctctgcccatggggtgttcctgctgaacagcaatgctatggatgtggtgctgcagcccagccctgccctgagctggaggtctactgggggcatcctggatgtgtacatctttctggggcctgagcccaagtctgtggtgcagcagtacctggatgtggtgggctatccttttatgcccccctattggggcctgggcttccacctgtgcaggtggggctacagcagcactgccatcaccagacaggtggtggagaacatgaccagggcccacttccccctggatgtgcagtggaatgacctggactacatggacagcaggagggacttcacctttaacaaggatggctttagggacttccctgccatggtgcaggagctgcatcaggggggcaggaggtacatgatgattgtggacccagccatcagcagctctgggcctgctgggtcttacaggccctatgatgagggcctgaggaggggggtgttcatcaccaatgagactggccagcccctgattggcaaggtgtggcctgggagcactgccttccctgattttaccaaccccactgccctggcctggtgggaggatatggtggctgagtttcatgaccaggtgccctttgatggcatgtggattgacatgaatgagcccagcaatttcatcaggggctctgaggatggctgccccaacaatgagctggagaatcctccctatgtgcctggggtggtggggggcaccctgcaggctgccaccatctgtgcctctagccaccagttcctgagcacccactataacctgcataacctgtatggcctgactgaggccattgccagccatagagccctggtgaaggccagagggaccaggccctttgtgatctctaggagcacctttgctggccatggcaggtatgctggccactggactggggatgtgtggagctcttgggagcagctggccagctctgtgccagagatcctgcagttcaacctgctgggggtgcctctggtgggggctgatgtgtgtggcttcctgggcaatacctctgaagagctgtgtgtgaggtggactcagctgggggccttctatcccttcatgaggaaccacaacagcctgctgtctctgccccaggagccctacagcttctctgagcctgctcagcaggctatgaggaaggccctgaccctgaggtatgccctgctgccccatctgtacaccctgttccaccaggcccatgtggctggggagactgtggccaggcccctgtttctggagtttcccaaggacagcagcacctggactgtggaccatcagctgctgtggggggaggctctgctgattacccctgtgctgcaggctggcaaggctgaggtgactgggtacttccccctggggacttggtatgacctgcagactgtgcctgtggaagctctgggcagcctgcccccaccccctgctgcccctagggagcctgccatccactctgagggccagtgggtgaccctgcctgcccctctggacaccatcaatgtgcacctgagggctggctatatcatccccctgcagggccctgggctgaccaccactgagagcaggcagcagcccatggccctggctgtggccctgactaaggggggggaggccaggggggagctgttctgggatgatggggagagcctggaggtgctggagagaggggcctacacccaggtgatctttctggccaggaacaacaccattgtgaatgagctggtgagggtgacttctgagggggctggcctgcagctgcagaaggtgactgtgctgggggtggccactgccccccagcaggtgctgagcaatggggtgcctgtgtctaacttcacctacagccctgatactaaggtgctggatatctgtgtgagcctgctgatgggggagcagtttctggtgagctggtgctgaagatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggcagaaagaaccagctggggctcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 20 - DNA, 4611 bp, synthetic construct
  source 1..4611: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatggctttcctgtggctgctgagctgctgggctctgctgggcaccacctttgggctgctggtgcctagggagctgtctgggtctagccctgtgctggaggagactcaccctgcccatcagcagggggctagcaggcctggccccagggatgctcaggcccaccctggcaggcccagggctgtgcccacccagtgtgatgtgccccccaacagcaggtttgactgtgcccctgacaaggccattacccaggagcagtgtgaggccaggggctgctgctacattccagctaagcagggcctgcagggggcccagatgggccagccctggtgcttcttcccccccagctatcctagctataaactggagaacctgagcagctctgagatgggctatactgccaccctgactaggactactcccaccttttttcctaaggatatcctgaccctgaggctggatgtgatgatggagactgagaacaggctgcacttcactattaaggaccctgccaataggaggtatgaagtgcctctggagactcctcatgtgcactctagggcccccagccccctgtattctgtggagttctctgaggagccctttggggtgattgtgaggaggcagctggatggcagggtgctgctgaacaccactgtggcccccctgttctttgctgaccagttcctgcagctgagcaccagcctgcccagccagtacatcactgggctggctgagcatctgagccctctgatgctgagcacctcttggaccaggatcaccctgtggaatagggatctggcccccacccctggggctaatctgtatggctctcatcccttttacctggccctggaggatgggggctctgcccatggggtgtttctgctgaacagcaatgccatggatgtggtgctgcagccctctcctgccctgagctggaggagcactgggggcatcctggatgtgtacatcttcctgggccctgagcccaagtctgtggtccagcagtatctggatgtggtgggctacccctttatgcccccctattggggcctgggcttccacctgtgcaggtgggggtattcttctactgctatcaccaggcaggtggtggagaacatgaccagggctcacttccccctggatgtgcagtggaatgacctggactatatggactctaggagggatttcaccttcaacaaggatggcttcagggacttccctgctatggtccaggagctgcatcaggggggcaggaggtacatgatgattgtggaccctgccatcagcagctctggccctgctggcagctataggccctatgatgagggcctgaggaggggggtgtttatcactaatgaaactgggcagcccctgattggcaaggtgtggcctggctctactgccttccctgacttcaccaaccccactgctctggcctggtgggaggacatggtggctgagttccatgaccaggtgccttttgatggcatgtggattgacatgaatgagcccagcaacttcatcaggggctctgaggatgggtgccccaataatgagctggagaaccccccctatgtgcctggggtggtggggggcaccctgcaggctgccactatttgtgccagctctcaccagttcctgagcacccactacaacctgcacaatctgtatggcctgactgaggccattgccagccacagggccctggtgaaggccaggggcactaggccctttgtgatctctagaagcacctttgctggccatgggaggtatgctggccactggactggggatgtgtggagctcttgggagcagctggccagctctgtgcctgagatcctgcagttcaacctgctgggggtgcccctggtgggggctgatgtgtgtggcttcctgggcaacacctctgaagagctgtgtgtgaggtggacccagctgggggccttctaccctttcatgaggaaccacaacagcctgctgagcctgcctcaggagccttactctttctctgagcctgcccagcaggccatgaggaaggccctgaccctgaggtatgctctgctgccccacctgtacaccctgttccaccaggcccatgtggctggggagactgtggccaggcccctgttcctggagtttcctaaggatagcagcacctggactgtggaccaccagctgctgtggggggaggccctgctgattacccctgtgctgcaggctggcaaggctgaggtgactggctacttccccctgggcacttggtatgacctgcagactgtgcctgtggaagccctgggcagcctgcctcccccccctgctgcccccagggagcctgccatccactctgagggccagtgggtgaccctgcctgcccccctggacaccattaatgtgcatctgagggctgggtatattatccccctgcaggggcctgggctgactaccactgagagcaggcagcagcctatggccctggctgtggctctgactaaggggggggaggccaggggggagctgttctgggatgatggggagagcctggaggtgctggagaggggggcctacacccaggtgattttcctggccaggaacaacaccattgtgaatgagctggtgagggtgacctctgagggggctggcctgcagctgcagaaagtgactgtgctgggggtggccactgccccccagcaggtgctgagcaatggggtgcctgtgagcaacttcacctacagccctgacaccaaggtgctggatatttgtgtgagcctgctgatgggggagcagttcctggtgagctggtgctgactcgagagatctaccggtgaattcaccgcgggtttaaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtgggggctagctctagactcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 21 - DNA, 4611 bp, synthetic construct
  source 1..4611: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatggctttcctgtggctgctgtcttgctgggccctgctggggactacctttggcctgctggtgcccagggaactgtctggctctagcccagtgctggaggagacccaccctgcccaccagcagggggcttctaggcctggccccagggatgcccaggcccaccctggcaggccaagggctgtgcccacccagtgtgatgtgccccccaactctagatttgattgtgcccctgataaggccatcacccaggagcagtgtgaggctaggggctgctgctacatccctgctaagcagggcctgcagggggctcagatgggccagccctggtgcttcttcccccccagctatccctcttacaagctggagaatctgagcagctctgagatgggctacactgccaccctgaccaggactactcccaccttcttccccaaggacatcctgaccctgaggctggatgtgatgatggagactgagaacaggctgcatttcaccatcaaggatcctgccaacaggaggtatgaggtgcctctggagaccccccatgtgcacagcagggctccttctcccctgtactctgtggagttctctgaggaaccctttggggtgattgtgaggaggcagctggatggcagggtcctgctgaacaccactgtggcccccctgttctttgctgatcagttcctgcagctgtccacttctctgcctagccagtacatcactgggctggctgagcacctgagccctctgatgctgagcacctcttggactaggatcaccctgtggaacagggacctggcccccacccctggggccaacctgtatggcagccaccccttctatctggccctggaggatgggggctctgcccatggggtgttcctgctgaatagcaatgctatggatgtggtgctgcagcccagccctgccctgtcttggaggagcactgggggcatcctggatgtgtacattttcctggggcctgagcccaagtctgtggtgcagcagtacctggatgtggtgggctaccccttcatgcctccctactggggcctgggcttccacctgtgcaggtggggctacagctctactgccatcaccaggcaggtggtggagaatatgaccagggcccacttccccctggatgtgcagtggaatgacctggactacatggactctaggagggacttcaccttcaataaggatggcttcagagacttccctgccatggtgcaggagctgcatcaggggggcaggaggtacatgatgattgtggaccctgccatcagctcttctggccctgctggctcttacaggccctatgatgagggcctgaggaggggggtgttcatcaccaatgagactgggcagcccctgattgggaaggtgtggcctggctctactgccttccctgacttcaccaatcctactgccctggcctggtgggaggacatggtggctgagttccatgaccaggtgccctttgatggcatgtggattgacatgaatgagccctctaatttcatcaggggctctgaggatggctgccccaacaatgagctggagaaccccccctatgtgcctggggtggtggggggcaccctgcaggctgccaccatctgtgctagctctcaccagttcctgagcacccactacaatctgcataacctgtatggcctgactgaggccattgccagccacagggccctggtgaaggctaggggcaccaggccctttgtgatttctaggagcacttttgctggccatggcaggtatgctgggcactggactggggatgtgtggtctagctgggagcagctggcttcttctgtgcctgagatcctgcagttcaacctgctgggggtgcctctggtgggggctgatgtgtgtgggttcctgggcaacacttctgaggagctgtgtgtgaggtggacccagctgggggccttctaccctttcatgaggaaccacaacagcctgctgagcctgccccaggagccctacagcttctctgagcctgcccagcaggccatgaggaaggccctgaccctgaggtatgccctgctgccccacctgtacaccctgttccaccaggcccatgtggctggggagactgtggctaggcctctgttcctggagttccccaaggactctagcacctggactgtggaccaccagctgctgtggggggaggccctgctgatcactcctgtgctgcaggctgggaaggctgaggtgactggctatttccccctgggcacctggtatgacctgcagactgtgcctgtggaggccctggggagcctgccccccccccctgctgcccccagggagcctgccatccactctgagggccagtgggtgaccctgcctgcccctctggataccatcaatgtgcacctgagggctggctacatcattcccctgcagggccctggcctgaccactactgagtctaggcagcagcccatggccctggctgtggccctgaccaaggggggggaggctaggggggagctgttttgggatgatggggagagcctggaggtgctggagaggggggcctacactcaggtgatcttcctggccaggaacaataccattgtgaatgagctggtgagggtgacctctgagggggctggcctgcagctgcagaaggtgactgtgctgggggtggccactgccccccagcaggtgctgagcaatggggtgcctgtgagcaacttcacctatagccctgataccaaggtgctggatatttgtgtgagcctgctgatgggggagcagttcctggtgagctggtgctgactcgagagatctaccggtgaattcaccgcgggtttaaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtgggggctagctctagactcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 22 - DNA, 4611 bp, synthetic construct
  source 1..4611: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatggctttcctgtggctgctgtcttgttgggctctgctgggcaccacctttggcctgctggtgcccagggagctgtctggcagcagccctgtgctggaggagacccaccctgctcatcagcagggggctagcaggcctggccccagggatgcccaggctcaccctgggagacccagggctgtgcccactcagtgtgatgtgccccccaacagcaggtttgactgtgctcctgacaaggctatcacccaggagcagtgtgaggccagggggtgctgctacattcctgctaagcagggcctgcagggggcccagatgggccagccctggtgcttcttccccccctcttatcccagctataagctggagaacctgagcagctctgagatgggctacactgccaccctgaccaggaccactcccaccttctttcccaaggatattctgactctgaggctggatgtgatgatggagactgagaacaggctgcacttcactatcaaggaccctgccaataggaggtatgaggtgcccctggagactcctcatgtgcatagcagggccccttctcctctgtattctgtggagttctctgaggagccctttggggtgattgtgaggaggcagctggatggcagggtgctgctgaacaccactgtggcccccctgttctttgctgaccagttcctgcagctgagcacttctctgcccagccagtacattactgggctggctgagcatctgagccccctgatgctgagcacctcttggaccaggatcaccctgtggaacagggacctggcccccactcctggggctaacctgtatggctctcaccccttttacctggccctggaggatgggggctctgcccatggggtgtttctgctgaacagcaatgctatggatgtggtgctgcagccctctccagccctgtcttggaggagcactgggggcattctggatgtgtacattttcctggggcctgaacccaagtctgtggtgcagcagtacctggatgtggtgggctaccccttcatgcccccctattgggggctggggtttcacctgtgcaggtggggctacagcagcactgccatcaccaggcaggtggtggagaacatgaccagggcccatttccccctggatgtgcagtggaatgacctggactacatggatagcaggagggatttcaccttcaacaaggatggcttcagggactttcctgccatggtgcaggagctgcaccaggggggcaggaggtatatgatgattgtggaccctgctatcagcagctctggccctgctggctcttacaggccctatgatgagggcctgaggaggggggtgtttatcactaatgaaactggccagcctctgattggcaaggtctggcctggctctactgccttccctgattttactaaccccactgccctggcctggtgggaggacatggtggctgagttccatgatcaggtgccttttgatggcatgtggattgatatgaatgaaccaagcaacttcatcagaggctctgaggatggctgccccaacaatgagctggagaaccccccctatgtgcctggggtggtggggggcactctgcaggctgccaccatttgtgctagcagccaccagttcctgagcacccactacaatctgcacaacctgtatggcctgactgaagccattgccagccatagggccctggtgaaggccaggggcactaggccttttgtgatcagcaggagcacttttgctggccatggcaggtatgctggccactggactggggatgtgtggagcagctgggagcagctggccagctctgtgcctgagattctgcagtttaacctgctgggggtgcccctggtgggggctgatgtgtgtggcttcctgggcaacacctctgaggagctgtgtgtgaggtggacccagctgggggccttttatcccttcatgaggaaccacaacagcctgctgagcctgcctcaggagccctactctttctctgagcctgcccagcaggccatgaggaaggccctgaccctgaggtatgccctgctgccccacctgtataccctgttccaccaggcccatgtggctggggagactgtggccaggcccctgttcctggagttccccaaggacagcagcacctggactgtggatcatcagctgctgtggggggaggccctgctgatcacccctgtgctgcaggctggcaaggctgaggtcactggctacttccctctgggcacctggtatgacctgcagactgtgcctgtggaggctctgggcagcctgccccccccccctgctgctcccagggagcctgccatccactctgagggccagtgggtgaccctgcctgctcccctggacaccatcaatgtgcacctgagggctggctacattatccccctgcagggcccagggctgactaccactgagagcagacagcagcccatggctctggctgtggccctgaccaaggggggggaagctaggggggagctgttctgggatgatggggagagcctggaggtgctggagaggggggcctatacccaggtgatcttcctggctaggaacaacaccattgtcaatgagctggtgagggtgacttctgagggggctgggctgcagctgcagaaggtgactgtgctgggggtggccactgctccccagcaggtgctgagcaatggggtgcctgtgagcaacttcacctacagccctgacaccaaggtgctggacatctgtgtgtctctgctgatgggggagcagttcctggtgagctggtgctgactcgagagatctaccggtgaattcaccgcgggtttaaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtgggggctagctctagactcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 23 - DNA, 4611 bp, synthetic construct
  source 1..4611: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatggccttcctgtggctgctgtcttgctgggctctgctggggaccacctttggcctgctggtccccagggagctgtctggctcttctcctgtcctggaggagacccaccctgcccaccagcagggggctagcaggcctggccccagggatgcccaggcccaccctggcaggcccagggctgtgcccacccagtgtgatgtgcctcccaacagcaggtttgactgtgcccctgacaaggccatcacccaggagcagtgtgaggccaggggctgctgctatatccctgccaagcagggcctgcagggggctcagatgggccagccctggtgcttctttcccccctcttatcctagctataagctggagaacctgagcagctctgagatggggtacactgccaccctgaccaggaccacccccactttcttccctaaggacatcctgaccctgaggctggatgtgatgatggagactgagaataggctgcactttactatcaaggaccctgccaacaggaggtatgaggtgcctctggagaccccccatgtgcattctagggcccccagccccctgtactctgtggagttctctgaggagccctttggggtgattgtgaggagacagctggatggcagggtcctgctgaacaccactgtggctcccctgttttttgctgaccagttcctgcagctgagcaccagcctgcccagccagtacatcactgggctggctgagcacctgagccccctgatgctgagcaccagctggaccaggatcaccctgtggaacagggatctggctcctacccctggggccaacctgtatggctctcaccccttttacctggccctggaggatgggggctctgcccatggggtgttcctgctgaacagcaatgctatggatgtggtgctgcagcccagccctgccctgagctggaggtctactgggggcatcctggatgtgtacatctttctggggcctgagcccaagtctgtggtgcagcagtacctggatgtggtgggctatccttttatgcccccctattggggcctgggcttccacctgtgcaggtggggctacagcagcactgccatcaccagacaggtggtggagaacatgaccagggcccacttccccctggatgtgcagtggaatgacctggactacatggacagcaggagggacttcacctttaacaaggatggctttagggacttccctgccatggtgcaggagctgcatcaggggggcaggaggtacatgatgattgtggacccagccatcagcagctctgggcctgctgggtcttacaggccctatgatgagggcctgaggaggggggtgttcatcaccaatgagactggccagcccctgattggcaaggtgtggcctgggagcactgccttccctgattttaccaaccccactgccctggcctggtgggaggatatggtggctgagtttcatgaccaggtgccctttgatggcatgtggattgacatgaatgagcccagcaatttcatcaggggctctgaggatggctgccccaacaatgagctggagaatcctccctatgtgcctggggtggtggggggcaccctgcaggctgccaccatctgtgcctctagccaccagttcctgagcacccactataacctgcataacctgtatggcctgactgaggccattgccagccatagagccctggtgaaggccagagggaccaggccctttgtgatctctaggagcacctttgctggccatggcaggtatgctggccactggactggggatgtgtggagctcttgggagcagctggccagctctgtgccagagatcctgcagttcaacctgctgggggtgcctctggtgggggctgatgtgtgtggcttcctgggcaatacctctgaagagctgtgtgtgaggtggactcagctgggggccttctatcccttcatgaggaaccacaacagcctgctgtctctgccccaggagccctacagcttctctgagcctgctcagcaggctatgaggaaggccctgaccctgaggtatgccctgctgccccatctgtacaccctgttccaccaggcccatgtggctggggagactgtggccaggcccctgtttctggagtttcccaaggacagcagcacctggactgtggaccatcagctgctgtggggggaggctctgctgattacccctgtgctgcaggctggcaaggctgaggtgactgggtacttccccctggggacttggtatgacctgcagactgtgcctgtggaagctctgggcagcctgcccccaccccctgctgcccctagggagcctgccatccactctgagggccagtgggtgaccctgcctgcccctctggacaccatcaatgtgcacctgagggctggctatatcatccccctgcagggccctgggctgaccaccactgagagcaggcagcagcccatggccctggctgtggccctgactaaggggggggaggccaggggggagctgttctgggatgatggggagagcctggaggtgctggagagaggggcctacacccaggtgatctttctggccaggaacaacaccattgtgaatgagctggtgagggtgacttctgagggggctggcctgcagctgcagaaggtgactgtgctgggggtggccactgccccccagcaggtgctgagcaatggggtgcctgtgtctaacttcacctacagccctgatactaaggtgctggatatctgtgtgagcctgctgatgggggagcagtttctggtgagctggtgctgactcgagagatctaccggtgaattcaccgcgggtttaaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtgggggctagctctagactcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 24 - DNA, 4611 bp, synthetic construct
  source 1..4611: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatagatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaagcacgcgtgccaccatggcctttctgtggctgctgtcctgctgggccctgctggggaccacctttggcctgctggtgcccagggagctgtctgggagcagcccagtgctggaggagacccaccctgcccaccagcagggggccagcaggcctggccctagggatgcccaggcccaccctggcaggcccagggctgtgcctacccagtgtgatgtgccacccaattctaggtttgactgtgctcctgacaaggccatcactcaggagcagtgtgaagctagggggtgctgctacatcccagccaagcagggcctgcagggggcccagatgggccagccctggtgcttcttcccccccagctaccctagctacaagctggagaatctgagcagctctgagatgggctacactgctaccctgaccaggaccactcctaccttcttccccaaggacatcctgactctgaggctggatgtcatgatggagactgaaaataggctgcacttcaccatcaaggaccctgccaataggaggtatgaggtgcctctggagaccccccatgtgcatagcagggctcccagccccctgtattctgtggagttctctgaggagccctttggggtcattgtgaggagacagctggatgggagggtgctgctgaacactactgtggctcccctgttctttgctgaccagttcctgcagctgtctaccagcctgcccagccagtacatcactgggctggctgagcatctgagccccctgatgctgagcaccagctggaccaggatcactctgtggaacagggatctggcccccactcctggggccaacctgtatgggagccatcccttctacctggccctggaggatgggggctctgcccatggggtgttcctgctgaacagcaatgccatggatgtggtgctgcagcctagccctgccctgagctggaggagcactgggggcatcctggatgtctacatcttcctggggcctgagcccaagtctgtggtgcagcagtatctggatgtggtggggtatcccttcatgcccccctactggggcctgggctttcacctgtgcaggtggggctacagcagcactgccatcaccaggcaggtggtggagaacatgaccagggcccacttccctctggatgtgcagtggaatgacctggactatatggattctaggagagactttacttttaacaaggatggcttcagggatttccctgccatggtgcaggagctgcaccaggggggcaggaggtacatgatgattgtggaccctgctattagcagctctggccctgctgggtcttacaggccttatgatgagggcctgaggaggggggtgttcatcaccaatgagactggccagcccctgattggcaaggtgtggcctggcagcactgccttccctgacttcaccaaccccactgccctggcctggtgggaggacatggtggctgagttccatgaccaggtgccctttgatgggatgtggattgacatgaatgagccctctaacttcatcagggggtctgaggatggctgccccaacaatgagctggagaaccccccctatgtgcctggggtggtggggggcactctgcaggctgccactatctgtgcttcttctcaccagtttctgagcacccactataatctgcacaacctgtatggcctgactgaggccattgccagccatagggccctggtgaaggccaggggcaccaggccctttgtgatcagcaggtctacctttgctggccatggcaggtatgctggccactggactggggatgtgtggtcttcttgggagcagctggccagctctgtgcctgagatcctgcagttcaacctgctgggggtgcctctggtgggggctgatgtgtgtggctttctgggcaacacctctgaggagctgtgtgtgaggtggacccagctgggggccttttaccccttcatgaggaaccacaatagcctgctgagcctgccccaggagccttactctttctctgagcctgcccagcaggccatgaggaaggccctgactctgaggtatgccctgctgccccatctgtataccctgtttcaccaggcccatgtggctggggagactgtggctaggcctctgtttctggagttccctaaggactctagcacctggactgtggaccaccagctgctgtggggggaggccctgctgatcacccctgtgctgcaggctggcaaggctgaggtgactggctacttccccctgggcacctggtatgacctgcagactgtgcctgtggaggccctggggagcctgcctcccccccctgctgcccccagggagcctgccattcattctgagggccagtgggtgaccctgcctgcccctctggacaccatcaatgtgcacctgagggctgggtacatcatccccctgcagggccctggcctgaccaccactgagagcaggcagcagcccatggccctggctgtggctctgaccaaggggggggaggccaggggggagctgttctgggatgatggggagtctctggaggtgctggagaggggggcctacacccaggtgatctttctggccaggaacaatactattgtgaatgagctggtgagggtgacctctgagggggctggcctgcagctgcagaaggtgactgtgctgggggtggccactgccccccagcaggtcctgagcaatggggtgcctgtgagcaacttcacctactctcctgacaccaaggtgctggacatttgtgtgtctctgctgatgggggagcagttcctggtgagctggtgctgactcgagagatctaccggtgaattcaccgcgggtttaaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtgggggctagctctagactcgagatccactagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 25 - protein, 935 aa, synthetic construct
MAFLWLLSCWALLGTTFGLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 26 - DNA, 268 bp, synthetic construct
  source 1..268: mol_type = other DNA
agatctagagctgaattcctgcagccagggggatcagcctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatgg

SEQ ID NO: 27 - DNA, 202 bp, Bos taurus
  source 1..202: mol_type = other DNA
agatctaccggtgaattcaccgcgggtttaaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtgggggctagctctaga

SEQ ID NO: 28 - DNA, 456 bp, Homo sapiens
  source 1..456: mol_type = other DNA
gggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccc

SEQ ID NO: 29 - DNA, 444 bp, Homo sapiens
  source 1..444: mol_type = other DNA
atgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggaca

SEQ ID NO: 30 - protein, 738 aa, synthetic construct
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAAPSGVGPNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 31 - protein, 601 aa, synthetic construct
TAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 32 - protein, 535 aa, synthetic construct
MAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 33 - protein, 736 aa, synthetic construct
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

(19) United States
(12) Patent Application Publication
(10) Pub. No.: US 2025/0101396 A1
(43) Pub. Date: Mar. 27, 2025

(54) CODON-OPTIMIZED ACID ALPHA-GLUCOSIDASE EXPRESSION CASSETTES AND METHODS OF USING SAME

(71) Applicant: Spark Therapeutics, Inc., Philadelphia, PA (US)

(72) Inventors: Xavier Anguela, Philadelphia, PA (US); Sean Armour, Holland, PA (US); Jayme Nordin, Philadelphia, PA (US)

(21) Appl. No.: 18/828,493

(22) Filed: Sep. 9, 2024

Related U.S. Application Data
(63) Continuation of application No. 17/055,523, filed on Nov. 13, 2020, now Pat. No. 12,084,693.
(60) Provisional application No. 62/672,419, filed on May 16, 2018.
(60) Provisional application No. 62/734,454, filed on Sep. 21, 2018.

(51) Int. Cl. C12N 9/26; A61K 48/00; C12N 15/86

(52) U.S. Cl. CPC C12N 9/2408; A61K 48/005; C12N 15/86; C12Y 302/0102; C12N 2750/14143; C12N 2750/14152; C12N 2800/22; C12N 2830/001; C12N 2830/50

(57) ABSTRACT

The invention provides nucleic acids encoding acid α-glucosidase (GAA). In certain embodiments, nucleic acids have greater than about 86% sequence identity to a sequence selected from the group consisting of any of the sequences set forth as SEQ ID NOs: 1-5. In certain embodiments, nucleic acids encoding acid α-glucosidase (GAA) contain less than 127 CpG dinucleotides. Expression cassettes, vectors, cells and cell lines and methods of using such nucleic acids encoding acid α-glucosidase (GAA) are also provided.

### RELATED APPLICATIONS

This application claims priority to U.S. Provisional Patent Application No. 62/672,419, filed May 16, 2018, and U.S. Provisional Patent Application No. 62/734,454, filed Sep. 21, 2018. The entire contents of the foregoing applications are incorporated herein by reference, including all text, tables, sequence listing and drawings.

### INTRODUCTION

Glycogen storage disease type II, also called Pompe disease, is an autosomal recessive disorder caused by mutations in the gene encoding the lysosomal enzyme acid α-glucosidase (GAA), which catalyzes the degradation of glycogen. The resulting enzyme deficiency leads to pathological accumulation of glycogen and lysosomal alterations in all tissues of the body, resulting in cardiac, respiratory, and skeletal muscle dysfunction (van der Ploeg & Reuser, 2008 Lancet, 372:1342-1353). Traditionally, Pompe disease has been separated into two major phenotypes-infantile-onset Pompe disease (IOPD) (also referred to as infantile Pompe disease (IPD) or early-onset Pompe disease) and late-onset Pompe disease (LOPD)-based on residual GAA enzyme activity, age of onset, organ involvement (i.e., presence of cardiomyopathy), severity, and rate of progression. Enzyme replacement therapy (ERT) is available for Pompe disease; however, it has several limitations (i.e., limited biodistribution and high immunogenicity) leading to treatment failures and limited long-term efficacy (van der Ploeg & Reuser, 2008).

### SUMMARY

Disclosed herein are optimized cassettes for liver-directed expression of a secretable version of human GAA. These optimizations to the cassettes lead to an increase in GAA secretion from liver and enable hepatic gene transfer to achieve circulating levels of GAA sufficient to cross-correct GAA deficiency systemically in subjects. These cassettes achieve increased transgene expression, improved safety characteristics and potentially reduced immunogenicity. These cassettes will be useful as a gene therapy treatment of subjects with Pompe disease and other diseases and disorders treatable with GAA.

Codon optimization of GAA expression cassette was undertaken to improve expression of GAA. In one embodiment, nucleic acid sequences encoding GAA were modified to eliminate CpG dinucleotides. In total, 20 new codon optimized transgene sequences were created (GAA1-GAA20). Based on in vitro comparison of GAA activity, 5 codon-optimized sequences were selected (GAA 2, 5, 7, 8 and 13) for further optimization. The GAA13 sequence was used to analyze the differences in expression of GAA after addition of a 29-base pair polynucleotide sequence to the 5′ untranslated region (UTR). Two different polyadenylation sequences derived from bovine growth hormone (bGH or BGH) (wild-type and CpG-reduced) were also evaluated for GAA7, 8 and 13. The resulting 9 expression cassettes (Table 1) were packaged within the SEQ ID NOs: 30-32 capsid; one was also packaged into AAV6 capsid.

In accordance with the invention, there are provided nucleic acids encoding an acid α-glucosidase (GAA), expression cassettes comprising the nucleic acids encoding an acid α-glucosidase (GAA) and viral vectors comprising the nucleic acids encoding an acid α-glucosidase (GAA).

In one embodiment, a nucleic acid encoding a GAA has greater than 86% sequence identity to any of the sequences set forth as SEQ ID NOs: 1-5. In additional aspects, a nucleic acid encoding a GAA has greater than 87% sequence identity to any of the sequences set forth as SEQ ID NOs: 1-5.

In another embodiment, a nucleic acid encoding a GAA has greater than 87% sequence identity to any of the sequences set forth as SEQ ID NOs: 6-15. In particular aspects, a nucleic acid encoding a GAA has greater than 88% sequence identity to any of the sequences set forth as SEQ ID NOs: 6-15. In additional particular aspects, a nucleic acid encoding a GAA has greater than 89% sequence identity to any of the sequences set forth as SEQ ID NOs: 6-15. In further particular aspects, a nucleic acid encoding a GAA has greater than 90% sequence identity to any of the sequences set forth as SEQ ID NOs: 6-15. In yet additional particular aspects, a nucleic acid encoding a GAA has greater than 91% sequence identity to any of the sequences set forth as SEQ ID NOs: 6-15.

In another embodiment, a nucleic acid encoding a GAA has greater than 91% sequence identity to any of the sequences set forth as SEQ ID NOs: 16-24.

In another embodiment, a nucleic acid encoding a GAA has greater than 92% sequence identity to any of the sequences set forth as SEQ ID NOs: 1-24.

In another embodiment, a nucleic acid encoding a GAA has greater than 93% sequence identity to any of the sequences set forth as SEQ ID NOs: 1-24.

In another embodiment, a nucleic acid encoding a GAA has greater than 94% sequence identity to any of the sequences set forth as SEQ ID NOs: 1-24.

In another embodiment, a nucleic acid encoding a GAA has greater than 95% sequence identity to any of the sequences set forth as SEQ ID NOs: 1-24.

In another embodiment, a nucleic acid encoding a GAA has greater than 96% sequence identity to any of the sequences set forth as SEQ ID NOs: 1-24.

In another embodiment, a nucleic acid encoding a GAA has greater than 97% sequence identity to any of the sequences set forth as SEQ ID NOs: 1-24.

In another embodiment, a nucleic acid encoding a GAA has greater than 98% sequence identity to any of the sequences set forth as SEQ ID NOs: 1-24.

In another embodiment, a nucleic acid encoding a GAA has greater than 99% sequence identity to any of the sequences set forth as SEQ ID NOs: 1-24.

In another embodiment, a nucleic acid encoding a GAA has greater than 99.5% sequence identity to any of the sequences set forth as SEQ ID NOs: 1-24.

In another embodiment, a nucleic acid encoding a GAA has 100% sequence identity to any of the sequences set forth as SEQ ID NOs: 1-24.

In another embodiment, a nucleic acid encoding a GAA contains less than 127 CpG dinucleotides.

In another embodiment, a nucleic acid encoding a GAA contains less than 126 CpG dinucleotides.

In another embodiment, a nucleic acid encoding a GAA contains from about 126-120 CpG dinucleotides.

In another embodiment, a nucleic acid encoding a GAA contains from about 120-110 CpG dinucleotides.

In another embodiment, a nucleic acid encoding a GAA contains from about 110-100 CpG dinucleotides.

In another embodiment, a nucleic acid encoding a GAA contains from about 100-90 CpG dinucleotides.

In another embodiment, a nucleic acid encoding a GAA contains from about 90-80 CpG dinucleotides.

In another embodiment, a nucleic acid encoding a GAA contains from about 80-70 CpG dinucleotides.

In another embodiment, a nucleic acid encoding a GAA contains from about 70-60 CpG dinucleotides.

In another embodiment, a nucleic acid encoding a GAA contains from about 60-50 CpG dinucleotides.

In another embodiment, a nucleic acid encoding a GAA contains from about 50-40 CpG dinucleotides.

In another embodiment, a nucleic acid encoding a GAA contains from about 40-30 CpG dinucleotides.

In another embodiment, a nucleic acid encoding a GAA contains from about 30-20 CpG dinucleotides.

In another embodiment, a nucleic acid encoding a GAA contains less than 20 CpG dinucleotides.

In another embodiment, a nucleic acid encoding a GAA contains from about 20-10 CpG dinucleotides.

In another embodiment, a nucleic acid encoding a GAA contains less than 10 CpG dinucleotides.

In another embodiment, a nucleic acid encoding a GAA contains from about 10-5 CpG dinucleotides.

In another embodiment, a nucleic acid encoding a GAA contains 5 CpG dinucleotides.

In another embodiment, a nucleic acid encoding a GAA contains 4 CpG dinucleotides.

In another embodiment, a nucleic acid encoding a GAA contains 3 CpG dinucleotides.

In another embodiment, a nucleic acid encoding a GAA contains 2 CpG dinucleotides.

In another embodiment, a nucleic acid encoding a GAA contains 1 CpG dinucleotide.

In another embodiment, a nucleic acid encoding a GAA contains 0 CpG dinucleotides.

The invention also provides expression cassettes comprising the nucleic acids encoding GAA as set forth herein, operably linked to an expression control element.

In one embodiment, an expression cassette comprises a nucleic acid encoding a GAA with greater than 86% sequence identity (e.g., 87%-100% identity) to any of the sequences set forth as SEQ ID NOs: 1-5.

In another embodiment, an expression cassette comprises a nucleic acid encoding a GAA with greater than 87% sequence identity (e.g., 88%-100% identity) to any of the sequences set forth as SEQ ID NOs: 6-15.

In another embodiment, an expression cassette comprises a nucleic acid encoding a GAA with less than 127 CpG dinucleotides, less than 127 CpG dinucleotides, less than 127 CpG dinucleotides, less than 126 CpG dinucleotides, less than 125 CpG dinucleotides, less than 124 CpG dinucleotides, less than 123 CpG dinucleotides, less than 122 CpG dinucleotides, less than 121 CpG dinucleotides, less than 120 CpG dinucleotides, less than 119 CpG dinucleotides, less than 118 CpG dinucleotides, less than 117 CpG dinucleotides, less than 116 CpG dinucleotides, less than 115 CpG dinucleotides, less than 114 CpG dinucleotides, less than 113 CpG dinucleotides, less than 112 CpG dinucleotides, less than 111 CpG dinucleotides, less than 110 CpG dinucleotides, less than 109 CpG dinucleotides, less than 108 CpG dinucleotides, less than 107 CpG dinucleotides, less than 106 CpG dinucleotides, less than 105 CpG dinucleotides, less than 104 CpG dinucleotides, less than 103 CpG dinucleotides, less than 102 CpG dinucleotides, less than 101 CpG dinucleotides, less than 101 CpG dinucleotides, and so forth all the way down to zero (0) CpG dinucleotides.

In one embodiment, an expression control element is positioned 5′ of a nucleic acid encoding a GAA.

In another embodiment, an expression cassette includes a poly-adenylation (polyA) sequence positioned 3′ of a nucleic acid encoding a GAA.

In another embodiment, an expression control element or poly-adenylation sequence is CpG reduced compared to wild-type expression control element or poly-adenylation sequence.

In another embodiment, an expression control element comprises an ApoE/hAAT enhancer/promoter sequence.

In another embodiment, a poly-adenylation sequence comprises a bovine growth hormone (bGH) polyadenylation sequence.

In another embodiment, an ApoE/hAAT enhancer/promoter sequence or bGH polyadenylation sequence is CpG reduced compared to wild-type ApoE/hAAT enhancer/promoter sequence or bGH polyadenylation sequence.

In one aspect, a wild-type bGH polyadenylation sequence comprises the sequence of SEQ ID NO:27.

In another aspect, a wild-type ApoE/hAAT enhancer/promoter sequence comprises the sequence of SEQ ID NO:28 or 29.

In another aspect, a CpG reduced bGH polyadenylation sequence comprises the sequence of SEQ ID NO:26.

In another embodiment, an expression cassette further comprises an intron positioned between the 3′ end of the expression control element and the 5′ end of the nucleic acid encoding a GAA.

In another embodiment, a GAA comprises or consists of the sequence set forth as SEQ ID NO: 25.

The invention further provides viral vectors such as adenovirus-associated virus (AAV) vectors comprising the nucleic acids encoding GAA as set forth herein.

In one embodiment, a viral vector such as an adenovirus-associated virus (AAV) vector comprises any of the nucleic acids encoding GAA as set forth herein operably linked to an expression control element.

In another embodiment, a viral vector such as an adenovirus-associated virus (AAV) vector comprises any of the expression cassettes comprising the nucleic acids encoding GAA as set forth herein.

In another embodiment, an AAV vector comprises: one or more of an AAV capsid; and one or more AAV inverted terminal repeats (ITRs), wherein the AAV ITR(s) flanks the 5′ or 3′ terminus of the nucleic acid or the expression cassette.

In additional embodiments, an AAV vector further comprises an intron positioned 5′ or 3′ of one or more ITRs.

In additional embodiments, an AAV vector comprising at least one or more ITRs or an intron has the one or more ITRs or intron modified to have reduced CpGs.

In additional embodiments, an AAV vector has a capsid serotype comprising a modified or variant AAV VP1, VP2 and/or VP3 capsid having 90% or more sequence identity to AAV1, AAV2, AAV3, AAV4, AAV5, AAV6, AAV7, AAV8, AAV9, AAV10, AAV11, Rh10, Rh74, AAV3B, AAV-218 or SEQ ID NO:30, 31 or 32 VP1, VP2 and/or VP3 sequences, or a capsid having 95% or more sequence identity to AAV1, AAV2, AAV3, AAV4, AAV5, AAV6, AAV7, AAV8, AAV9, AAV10, AAV11, AAV12, Rh10, Rh74, AAV3B, AAV-218, or SEQ ID NO:30, 31 or 32 VP1, VP2 and/or VP3 sequences, or a capsid having 100% sequence identity to AAV1, AAV2, AAV3, AAV4, AAV5, AAV6, AAV7, AAV8, AAV9, AAV10, AAV11, Rh10, Rh74, AAV3B, AAV-218, or SEQ ID NO:30, 31 or 32 VP1, VP2 and/or VP3 sequences.

In additional embodiments, an AAV vector comprising one or more ITRs as one or more ITRs of any of: AAV1, AAV2, AAV3, AAV4, AAV5, AAV6, AAV7, AAV8, AAV9, AAV10, AAV11, Rh10, Rh74, or AAV3B AAV serotypes, or a combination thereof.

The invention additionally provides pharmaceutical compositions comprising any of the nucleic acids encoding GAA, expression cassettes comprising nucleic acids encoding GAA or viral vectors such as AAV vectors comprising nucleic acids encoding GAA or expression cassettes comprising nucleic acids encoding GAA as set forth herein.

In one embodiment, a pharmaceutical composition comprises a plurality of AAV vectors as set forth herein in a biologically compatible carrier or excipient.

In another embodiment, a pharmaceutical composition comprising any of the AAV vectors as set forth herein, further comprises empty AAV capsids.

In particular embodiments, in a pharmaceutical composition comprising AAV vectors and empty AAV capsids, the ratio of the empty AAV capsids to the AAV vector is within or between about 100:1-50:1, from about 50:1-25:1, from about 25:1-10:1, from about 10:1-1:1, from about 1:1-1:10, from about 1:10-1:25, from about 1:25-1:50, or from about 1:50-1:100.

In particular aspects, in a pharmaceutical composition comprising AAV vectors and empty AAV capsids, the ratio of the of the empty AAV capsids to the AAV vector is about 2:1, 3:1, 4:1, 5:1, 6:1, 7:1, 8:1, 9:1, or 10:1.

In another embodiment, a pharmaceutical composition includes a surfactant.

The invention still further provides methods of treating a human in need of acid α-glucosidase (GAA) by administering or delivering any of the nucleic acids encoding GAA, expression cassettes comprising nucleic acids encoding GAA, or viral vectors such as AAV vectors comprising the nucleic acids encoding GAA or expression cassettes comprising the nucleic acids encoding GAA to the human.

In one embodiment, a method of treating a human in need of acid α-glucosidase (GAA), includes: (a) providing a nucleic acid, expression cassette or viral vector such as AAV vector as set forth herein, or any pharmaceutical composition as set forth herein; and (b) administering an amount of the nucleic acid, expression cassette, viral (e.g., AAV) vector, or pharmaceutical composition to redo human, where the GAA is expressed in the human.

In particular embodiments, a human has Pompe disease, such as infantile onset Pompe disease or late onset Pompe disease.

In another embodiment, a human has a glycogen storage disease (GSD).

In particular aspects, a GSD is any of: GSD type I (von Gierke's disease), GSD type III (Forbes-Cori disease), GSD type IV (Anderson disease, amylopectinosis), GSD type V (McArdle disease), GSD type VI (Hers disease), GSD type VII (Tarui disease), or a congenital GSD of the heart (e.g., lethal congenital GSD of the heart).

In particular embodiments, a nucleic acid encoding a GAA, an expression cassette comprising a nucleic acid encoding a GAA, or an AAV vector is administered to a human intravenously, intraarterially, intra-cavity, intracavitary, intramucosally, or via catheter.

In particular embodiments, in a method GAA is expressed at increased levels, optionally greater than 1% of the levels of GAA found in a human not in need of GAA.

In particular embodiments, an AAV vector is administered in a range from about 1×10⁸ to about 1×10¹⁴ vector genomes per kilogram (vg/kg) of the weight of the human.

In particular embodiments, a method reduces, decreases or inhibits one or more symptoms of the need of GAA or the disease; or prevents or reduces progression or worsening of one or more symptoms of the need of GAA or the disease; or stabilizes one or more symptoms of the need of GAA or the disease; or improves one or more symptoms of the need of GAA or the disease.

In particular aspects, a symptom treatable in accordance with the invention can be one or more of the following: difficulty eating and/or not gaining weight; poor head and/or neck control; breathing problems and/or lung infections; enlarged and/or thickening heart; heart defects; enlarged tongue; difficulty swallowing; enlarged liver; poor muscle strength; weak muscle tone; weakness in the legs, waist and/or arms; shortness of breath; difficulty exercising; difficulty breathing while sleeping; curvature of the spine; and/or stiff joints.

The invention yet additionally provides cells comprising nucleic acids encoding GAA, cells comprising expression cassettes comprising the nucleic acids encoding GAA and cells comprising viral vectors such as AAV vectors comprising nucleic acids encoding GAA or expression cassettes comprising nucleic acids encoding GAA.

In one embodiment, a cell produces a viral vector.

In another embodiment, a cell produces an AAV vector as set forth herein.

Still further, the invention also provides methods of producing viral vectors such as AAV vectors as set forth herein.

In one embodiment, a method of producing AAV vectors include: introducing an AAV vector genome comprising a nucleic acid encoding GAA or expression cassette comprising a nucleic acid encoding GAA as set forth herein into a packaging helper cell; and culturing the helper cell under conditions to produce the AAV vectors.

In another embodiment, a method of producing AAV vector errors includes: introducing a nucleic acid encoding GAA or expression cassette comprising a nucleic acid encoding GAA as set forth herein into a packaging helper cell; and culturing the helper cells under conditions to produce the AAV vector.

In additional embodiments, cells are mammalian cells.

In additional embodiments, cells for vector production provide helper functions, such as AAV helper functions, that package the vector into a viral particle. In a particular aspect, the helper functions are Rep and/or Cap proteins for AAV vector packaging.

In additional embodiments, cells for vector production may be stably or transiently transfected with polynucleotide(s) encoding Rep and/or Cap protein sequence(s).

In additional embodiments, cells for vector production provide Rep78 and/or Rep68 proteins. In such cells, the cells may be stably or transiently transfected with Rep78 and/or Rep68 proteins polynucleotide encoding sequence(s).

In particular embodiments, cells for vector production are human embryonic kidney cells. In a particular aspect, cells for vector production are HEK-293 cells.

### DESCRIPTION OF DRAWINGS

FIG. 1 shows a schematic of pAAV-ApoE/hAAT.fixUTR.GAA13.BGH (SEQ ID NO: 16).

FIG. 2 shows a schematic of pAAV-ApoE/hAAT.GAA13.BGH (SEQ ID NO:17).

FIG. 3 shows a schematic of pAAV-ApoE/hAAT.GAA7.BGH (SEQ ID NO:18).

FIG. 4 shows a schematic of pAAV-ApoE/hAAT.GAA8.BGH (SEQ ID NO:19).

FIG. 5 shows a schematic of pAAV-ApoE/hAAT.GAA2.wtBGH (SEQ ID NO:20).

FIG. 6 shows a schematic of pAAV-ApoE/hAAT.GAA5.wtBGH (SEQ ID NO:21).

FIG. 7 shows a schematic of pAAV-ApoE/hAAT.GAA7.wtBGH (SEQ ID NO:22).

FIG. 8 shows a schematic of pAAV-ApoE/hAAT.GAA8.wtBGH (SEQ ID NO:23).

FIG. 9 shows a schematic of pAAV-ApoE/hAAT.GAA13.wtBGH. (SEQ ID NO:24)

FIG. 10 shows evaluation of GAA activity levels in Huh7 cell supernatants by GAA activity assay.

FIG. 11 shows evaluation of GAA activity levels in mouse plasma by GAA activity assay.

FIG. 12 shows evaluation of GAA levels in mouse plasma by GAA western blot.

FIG. 13 shows evaluation of GAA activity levels in mouse plasma by GAA activity assay.

FIG. 14 shows evaluation of GAA levels in mouse plasma by GAA western blot.

FIG. 15 shows evaluation of GAA activity levels in mouse plasma by GAA activity assay.

FIG. 16 shows evaluation of GAA activity levels in rat and mouse plasma by GAA activity assay.

FIG. 17 shows evaluation of GAA activity levels in plasma of non-human primates (male rhesus macaque) by GAA activity assay.

FIG. 18 shows evaluation of GAA activity levels in plasma of non-human primates (African green monkey) by GAA activity assay. The X-axis is days post injection.

FIG. 19 shows evaluation of GAA levels in plasma of non-human primates (African green monkey) by western blot, with graphical presentation of certain data.

FIG. 20 shows evaluation of GAA activity in supernatants of Huh7 cells transduced with SPK-AAV-01, SPK-AAV-02 or a negative control (rAAV with non-GAA transgene packaged into AAV SEQ ID NOs: 30-32 vector).

FIG. 21 shows evaluation of GAA activity in plasma from rhesus macaques following a single intravenous administration of SPK-AAV-02 at doses of 2×10¹², 6×10¹², 2×10¹³ vg/kg.

### DETAILED DESCRIPTION

The invention provides modified nucleic acids encoding GAA, expression cassettes comprising modified nucleic acids encoding GAA, AAV vector genomes comprising modified nucleic acids encoding GAA, and recombinant AAV vectors and particles comprising the modified nucleic acids encoding GAA. The invention modified nucleic acids encoding GAA, expression cassettes comprising modified nucleic acids encoding GAA, AAV vector genomes comprising modified nucleic acids encoding GAA, and recombinant AAV vectors and particles are useful for treating Pompe disease as well as other glycogen storage diseases (GSDs).

As used herein, the terms “modify” and grammatical variations thereof, mean that a nucleic acid or protein deviates from a reference or parental sequence. A modified nucleic acid encoding GAA has been altered compared to reference (e.g., wild-type) or parental nucleic acid. Modified nucleic acids may therefore have substantially the same, greater or less activity or function than a reference or parental nucleic acid, but at least retain partial activity, function and or sequence identity to the reference or parental nucleic acid. The modified nucleic acid may be genetically modified to encode a modified or variant GAA.

A “modified nucleic acid encoding GAA” means that the GAA nucleic acid has alteration compared the parental unmodified nucleic acid encoding GAA. A particular example of a modification is a nucleotide substitution. The terms “modification” herein need not appear in each instance of a reference made to a nucleic acid encoding GAA.

In particular embodiments, for a modified nucleic acid encoding GAA, the GAA protein retains at least part of a function or activity of wild type GAA protein. The function or activity of GAA protein includes acid alpha glucosidase activity, a lysosomal hydrolase which degrades glycogen, maltose and isomaltose. Accordingly, the modified nucleic acids encoding GAA include modified forms so long as the encoded GAA retains some degree or aspect of lysosomal hydrolase activity of GAA.

As set forth herein, modified nucleic acids encoding GAA can exhibit different features or characteristics compared to a reference or parental nucleic acid. For example, modified nucleic acids include sequences with 100% identity to a reference nucleic acid encoding GAA as set forth herein, as well as sequences with less than 100% identity to a reference nucleic acid encoding GAA.

The term “identity,” “homology” and grammatical variations thereof, mean that two or more referenced entities are the same, when they are “aligned” sequences. Thus, by way of example, when two nucleic acids are identical, they have the same sequence, at least within the referenced region or portion. The identity can be over a defined area (region or domain) of the sequence.

An “area” or “region” of identity refers to a portion of two or more referenced entities that are the same. Thus, where two protein or nucleic acid sequences are identical over one or more sequence areas or regions they share identity within that region. An “aligned” sequence refers to multiple protein (amino acid) or nucleic acid sequences, often containing corrections for missing or additional bases or amino acids (gaps) as compared to a reference sequence.

The identity can extend over the entire length or a portion of the sequence. In certain embodiments, the length of the sequence sharing the percent identity is 2, 3, 4, 5 or more contiguous amino acids or nucleic acids, e.g., 6, 7, 8, 9, 10, 11, 12, 13, 14, 15, 16, 17, 18, 19, 20, etc. contiguous nucleic acids or amino acids. In additional embodiments, the length of the sequence sharing identity is 21 or more contiguous amino acids or nucleic acids, e.g., 21, 22, 23, 24, 25, 26, 27, 28, 29, 30, 31, 32, 33, 34, 35, 36, 37, 38, 39, 40, etc. contiguous amino acids or nucleic acids. In further embodiments, the length of the sequence sharing identity is 41 or more contiguous amino acids or nucleic acids, e.g., 42, 43, 44, 45, 45, 47, 48, 49, 50, etc., contiguous amino acids or nucleic acids. In yet further embodiments, the length of the sequence sharing identity is 50 or more contiguous amino acids or nucleic acids, e.g., 50-55, 55-60, 60-65, 65-70, 70-75, 75-80, 80-85, 85-90, 90-95, 95-100, 100-150, 150-200, 200-250, 250-300, 300-500, 500-1,000, etc. contiguous amino acids or nucleic acids.

As set forth herein, modified nucleic acids encoding GAA can be distinct from or exhibit 100% identity or less than 100% identity to a reference nucleic acid encoding GAA.

In particular embodiments, nucleic acids of the invention encoding GAA can be more than 86% identical to any nucleic acid set forth in SEQ ID NOs: 1-5.

In particular embodiments, nucleic acids of the invention encoding GAA can be more than 87% identical to any nucleic acid set forth in SEQ ID NOs: 6-14.

In particular embodiments, nucleic acids of the invention encoding GAA can be more than 91% identical to any nucleic acid set forth in SEQ ID NOs: 16-24.

Such modified nucleic acids encoding GAA can even exhibits greater identity, for example, more than 87% identical; more than 88% identical, more than 89% identical, more than 90% identical, more than 91% identical, more than 92% identical, more than 93% identical, more than 94% identical, more than 95% identical, more than 96% identical, more than 97%, more than 98% identical, more than 99% identical or 100% identical to any of SEQ ID NOs: 1-24.

The extent of identity (homology) or “percent identity” between two sequences can be ascertained using a computer program and/or mathematical algorithm. For purposes of this invention comparisons of nucleic acid sequences are performed using the GCG Wisconsin Package version 9.1, available from the Genetics Computer Group in Madison, Wisconsin. For convenience, the default parameters (gap creation penalty=12, gap extension penalty=4) specified by that program are intended for use herein to compare sequence identity. Alternately, the Blastn 2.0 program provided by the National Center for Biotechnology Information (found on the world wide web at ncbi.nlm.nih.gov/blast/; Altschul et al., 1990, J Mol Biol 215:403-410) using a gapped alignment with default parameters, may be used to determine the level of identity and similarity between nucleic acid sequences and amino acid sequences. For polypeptide sequence comparisons, a BLASTP algorithm is typically used in combination with a scoring matrix, such as PAM100, PAM 250, BLOSUM 62 or BLOSUM 50. FASTA (e.g., FASTA2 and FASTA3) and SSEARCH sequence comparison programs are also used to quantitate extent of identity (Pearson et al., Proc. Natl. Acad. Sci. USA 85:2444 (1988); Pearson, Methods Mol Biol. 132:185 (2000); and Smith et al., J. Mol. Biol. 147:195 (1981)). Programs for quantitating protein structural similarity using Delaunay-based topological mapping have also been developed (Bostick et al., Biochem Biophys Res Commun. 304:320 (2003)).

Modified nucleic acids encoding GAA that exhibit different features or characteristics compared to a reference or parental nucleic acid include substitutions of nucleotides. For example, modified nucleic acids encoding GAA include nucleic acids with a reduced number of CpG dinucleotides compared to a reference nucleic acid encoding GAA.

In certain embodiments, a nucleic acid encoding GAA contains less than 127 CpG dinucleotides.

In certain embodiments, a nucleic acid encoding GAA contains less than 126 CpG dinucleotides.

In certain embodiments, a nucleic acid encoding GAA contains less than 125 CpG dinucleotides.

In certain embodiments, a nucleic acid encoding GAA contains less than 124 CpG dinucleotides.

In certain embodiments, a nucleic acid encoding GAA contains less than 123 CpG dinucleotides.

In certain embodiments, a nucleic acid encoding GAA contains less than 122 CpG dinucleotides.

In certain embodiments, a nucleic acid encoding GAA contains less than 121 CpG dinucleotides.

In certain embodiments, a nucleic acid encoding GAA contains less than 120 CpG dinucleotides.

In certain embodiments, a nucleic acid encoding GAA contains less than 119 CpG dinucleotides.

In certain embodiments, a nucleic acid encoding GAA contains less than 118 CpG dinucleotides.

In certain embodiments, a nucleic acid encoding GAA contains less than 117 CpG dinucleotides.

In certain embodiments, a nucleic acid encoding GAA contains less than 116 CpG dinucleotides.

In certain embodiments, a nucleic acid encoding GAA contains less than 115 CpG dinucleotides.

In certain embodiments, a nucleic acid encoding GAA contains less than 114 CpG dinucleotides.

In certain embodiments, a nucleic acid encoding GAA contains less than 113 CpG dinucleotides.

In certain embodiments, a nucleic acid encoding GAA contains less than 112 CpG dinucleotides.

In certain embodiments, a nucleic acid encoding GAA contains less than 111 CpG dinucleotides.

In certain embodiments, a nucleic acid encoding GAA contains less than 110 CpG dinucleotides.

In certain embodiments, a nucleic acid encoding GAA contains less than 109 CpG dinucleotides.

In certain embodiments, a nucleic acid encoding GAA contains less than 108 CpG dinucleotides.

In certain embodiments, a nucleic acid encoding GAA contains less than 107 CpG dinucleotides.

In certain embodiments, a nucleic acid encoding GAA contains less than 106 CpG dinucleotides.

In certain embodiments, a nucleic acid encoding GAA contains less than 105 CpG dinucleotides.

In certain embodiments, a nucleic acid encoding GAA contains less than 104 CpG dinucleotides.

In certain embodiments, a nucleic acid encoding GAA contains less than 103 CpG dinucleotides.

In certain embodiments, a nucleic acid encoding GAA contains less than 102 CpG dinucleotides.

In certain embodiments, a nucleic acid encoding GAA contains less than 101 CpG dinucleotides.

In certain embodiments, a nucleic acid encoding GAA contains less than 100 CpG dinucleotides.

In certain embodiments, a nucleic acid encoding GAA contains less than 99 CpG dinucleotides.

In certain embodiments, a nucleic acid encoding GAA contains less than 98 CpG dinucleotides.

In certain embodiments, a nucleic acid encoding GAA contains less than 97 CpG dinucleotides.

In certain embodiments, a nucleic acid encoding GAA contains less than 96 CpG dinucleotides.

In certain embodiments, a nucleic acid encoding GAA contains less than 95 CpG dinucleotides.

In certain embodiments, a nucleic acid encoding GAA contains less than 94 CpG dinucleotides.

In certain embodiments, a nucleic acid encoding GAA contains less than 93 CpG dinucleotides.

In certain embodiments, a nucleic acid encoding GAA contains less than 92 CpG dinucleotides.

In certain embodiments, a nucleic acid encoding GAA contains less than 91 CpG dinucleotides.

In certain embodiments, a nucleic acid encoding GAA contains less than 90 CpG dinucleotides.

In certain embodiments, a nucleic acid encoding GAA contains less than 89 CpG dinucleotides.

In certain embodiments, a nucleic acid encoding GAA contains less than 88 CpG dinucleotides.

In certain embodiments, a nucleic acid encoding GAA contains less than 87 CpG dinucleotides.

In certain embodiments, a nucleic acid encoding GAA contains less than 86 CpG dinucleotides.

In certain embodiments, a nucleic acid encoding GAA contains less than 85 CpG dinucleotides.

In certain embodiments, a nucleic acid encoding GAA contains less than 84 CpG dinucleotides.

In certain embodiments, a nucleic acid encoding GAA contains less than 83 CpG dinucleotides.

In certain embodiments, a nucleic acid encoding GAA contains less than 82 CpG dinucleotides.

In certain embodiments, a nucleic acid encoding GAA contains less than 81 CpG dinucleotides.

In certain embodiments, a nucleic acid encoding GAA contains less than 80 CpG dinucleotides.

In certain embodiments, a nucleic acid encoding GAA contains less than 79 CpG dinucleotides.

In certain embodiments, a nucleic acid encoding GAA contains less than 78 CpG dinucleotides.

In certain embodiments, a nucleic acid encoding GAA contains less than 77 CpG dinucleotides.

In certain embodiments, a nucleic acid encoding GAA contains less than 76 CpG dinucleotides.

In certain embodiments, a nucleic acid encoding GAA contains less than 75 CpG dinucleotides.

In certain embodiments, a nucleic acid encoding GAA contains less than 74 CpG dinucleotides.

In certain embodiments, a nucleic acid encoding GAA contains less than 73 CpG dinucleotides.

In certain embodiments, a nucleic acid encoding GAA contains less than 72 CpG dinucleotides.

In certain embodiments, a nucleic acid encoding GAA contains less than 71 CpG dinucleotides.

In certain embodiments, a nucleic acid encoding GAA contains less than 70 CpG dinucleotides.

In certain embodiments, a nucleic acid encoding GAA contains less than 69 CpG dinucleotides.

In certain embodiments, a nucleic acid encoding GAA contains less than 68 CpG dinucleotides.

In certain embodiments, a nucleic acid encoding GAA contains less than 67 CpG dinucleotides.

In certain embodiments, a nucleic acid encoding GAA contains less than 66 CpG dinucleotides.

In certain embodiments, a nucleic acid encoding GAA contains less than 65 CpG dinucleotides.

In certain embodiments, a nucleic acid encoding GAA contains less than 64 CpG dinucleotides.

In certain embodiments, a nucleic acid encoding GAA contains less than 63 CpG dinucleotides.

In certain embodiments, a nucleic acid encoding GAA contains less than 62 CpG dinucleotides.

In certain embodiments, a nucleic acid encoding GAA contains less than 61 CpG dinucleotides.

In certain embodiments, a nucleic acid encoding GAA contains less than 60 CpG dinucleotides.

In certain embodiments, a nucleic acid encoding GAA contains less than 59 CpG dinucleotides.

In certain embodiments, a nucleic acid encoding GAA contains less than 58 CpG dinucleotides.

In certain embodiments, a nucleic acid encoding GAA contains less than 57 CpG dinucleotides.

In certain embodiments, a nucleic acid encoding GAA contains less than 56 CpG dinucleotides.

In certain embodiments, a nucleic acid encoding GAA contains less than 55 CpG dinucleotides.

In certain embodiments, a nucleic acid encoding GAA contains less than 54 CpG dinucleotides.

In certain embodiments, a nucleic acid encoding GAA contains less than 53 CpG dinucleotides.

In certain embodiments, a nucleic acid encoding GAA contains less than 52 CpG dinucleotides.

In certain embodiments, a nucleic acid encoding GAA contains less than 51 CpG dinucleotides.

In certain embodiments, a nucleic acid encoding GAA contains less than 50 CpG dinucleotides.

In certain embodiments, a nucleic acid encoding GAA contains less than 49 CpG dinucleotides.

In certain embodiments, a nucleic acid encoding GAA contains less than 48 CpG dinucleotides.

In certain embodiments, a nucleic acid encoding GAA contains less than 47 CpG dinucleotides.

In certain embodiments, a nucleic acid encoding GAA contains less than 46 CpG dinucleotides.

In certain embodiments, a nucleic acid encoding GAA contains less than 45 CpG dinucleotides.

In certain embodiments, a nucleic acid encoding GAA contains less than 44 CpG dinucleotides.

In certain embodiments, a nucleic acid encoding GAA contains less than 43 CpG dinucleotides.

In certain embodiments, a nucleic acid encoding GAA contains less than 42 CpG dinucleotides.

In certain embodiments, a nucleic acid encoding GAA contains less than 41 CpG dinucleotides.

In certain embodiments, a nucleic acid encoding GAA contains less than 40 CpG dinucleotides.

In certain embodiments, a nucleic acid encoding GAA contains less than 39 CpG dinucleotides.

In certain embodiments, a nucleic acid encoding GAA contains less than 38 CpG dinucleotides.

In certain embodiments, a nucleic acid encoding GAA contains less than 37 CpG dinucleotides.

In certain embodiments, a nucleic acid encoding GAA contains less than 36 CpG dinucleotides.

In certain embodiments, a nucleic acid encoding GAA contains less than 35 CpG dinucleotides.

In certain embodiments, a nucleic acid encoding GAA contains less than 34 CpG dinucleotides.

In certain embodiments, a nucleic acid encoding GAA contains less than 33 CpG dinucleotides.

In certain embodiments, a nucleic acid encoding GAA contains less than 32 CpG dinucleotides.

In certain embodiments, a nucleic acid encoding GAA contains less than 31 CpG dinucleotides.

In certain embodiments, a nucleic acid encoding GAA contains less than 30 CpG dinucleotides.

In certain embodiments, a nucleic acid encoding GAA contains less than 29 CpG dinucleotides.

In certain embodiments, a nucleic acid encoding GAA contains less than 28 CpG dinucleotides.

In certain embodiments, a nucleic acid encoding GAA contains less than 27 CpG dinucleotides.

In certain embodiments, a nucleic acid encoding GAA contains less than 26 CpG dinucleotides.

In certain embodiments, a nucleic acid encoding GAA contains less than 25 CpG dinucleotides.

In certain embodiments, a nucleic acid encoding GAA contains less than 24 CpG dinucleotides.

In certain embodiments, a nucleic acid encoding GAA contains less than 23 CpG dinucleotides.

In certain embodiments, a nucleic acid encoding GAA contains less than 22 CpG dinucleotides.

In certain embodiments, a nucleic acid encoding GAA contains less than 21 CpG dinucleotides.

In certain embodiments, a nucleic acid encoding GAA contains less than 20 CpG dinucleotides.

In certain embodiments, a nucleic acid encoding GAA contains less than 19 CpG dinucleotides.

In certain embodiments, a nucleic acid encoding GAA contains less than 18 CpG dinucleotides.

In certain embodiments, a nucleic acid encoding GAA contains less than 17 CpG dinucleotides.

In certain embodiments, a nucleic acid encoding GAA contains less than 16 CpG dinucleotides.

In certain embodiments, a nucleic acid encoding GAA contains less than 15 CpG dinucleotides.

In certain embodiments, a nucleic acid encoding GAA contains less than 14 CpG dinucleotides.

In certain embodiments, a nucleic acid encoding GAA contains less than 13 CpG dinucleotides.

In certain embodiments, a nucleic acid encoding GAA contains less than 12 CpG dinucleotides.

In certain embodiments, a nucleic acid encoding GAA contains less than 11 CpG dinucleotides.

In certain embodiments, a nucleic acid encoding GAA contains less than 10 CpG dinucleotides.

In certain embodiments, a nucleic acid encoding GAA contains less than 9 CpG dinucleotides.

In certain embodiments, a nucleic acid encoding GAA contains less than 8 CpG dinucleotides.

In certain embodiments, a nucleic acid encoding GAA contains less than 7 CpG dinucleotides.

In certain embodiments, a nucleic acid encoding GAA contains less than 6 CpG dinucleotides.

In certain embodiments, a nucleic acid encoding GAA contains less than 5 CpG dinucleotides.

In certain embodiments, a nucleic acid encoding GAA contains less than 4 CpG dinucleotides.

In certain embodiments, a nucleic acid encoding GAA contains less than 3 CpG dinucleotides.

In certain embodiments, a nucleic acid encoding GAA contains less than 2 CpG dinucleotides.

In certain embodiments, a nucleic acid encoding GAA contains less than 1 CpG dinucleotides.

The term “vector” refers to small carrier nucleic acid molecule, a plasmid, virus (e.g., AAV vector), or other vehicle that can be manipulated by insertion or incorporation of a nucleic acid. Such vectors can be used for genetic manipulation (i.e., “cloning vectors”), to introduce/transfer polynucleotides into cells, and to transcribe or translate the inserted polynucleotide in cells. An “expression vector” is a specialized vector that contains a gene or nucleic acid sequence with the necessary regulatory regions needed for expression in a host cell.

A vector nucleic acid sequence generally contains at least an origin of replication for propagation in a cell and optionally additional elements, such as a heterologous polynucleotide sequence, expression control element (e.g., a promoter, enhancer), intron, an inverted terminal repeat (ITR), selectable marker (e.g., antibiotic resistance), polyadenylation signal.

A viral vector is derived from or based upon one or more nucleic acid elements that comprise a viral genome. Particular viral vectors include lentiviral and adeno-associated virus (AAV) vectors.

The term “recombinant,” as a modifier of vector, such as recombinant AAV (rAAV) vector, as well as a modifier of sequences such as recombinant polynucleotides and polypeptides, means that the compositions have been manipulated (i.e., engineered) in a fashion that generally does not occur in nature. A particular example of a recombinant AAV vector would be where a click acid sequence that is not normally present in the wild-type AAV genome is inserted within the AAV genome. Although the term “recombinant” is not always used herein in reference to AAV vectors, as well as sequences such as polynucleotides, recombinant forms including polynucleotides, are expressly included in spite of any such omission.

A “recombinant AAV vector” or “rAAV” is derived from the wild type genome of AAV by using molecular methods to remove the wild type genome from the AAV genome, and replacing with a non-native nucleic acid sequence, referred to as a heterologous nucleic acid. Typically, for AAV one or both inverted terminal repeat (ITR) sequences of AAV genome are retained in the AAV vector. rAAV is distinguished from an AAV genome, since all or a part of the AAV genome has been replaced with a non-native sequence with respect to the AAV genomic nucleic acid. Incorporation of a non-native sequence therefore defines the AAV vector as a “recombinant” vector, which can be referred to as a “rAAV vector.”

A rAAV sequence can be packaged-referred to herein as a “particle”—for subsequent infection (transduction) of a cell, ex vivo, in vitro or in vivo. Where a recombinant AAV vector sequence is encapsidated or packaged into an AAV particle, the particle can also be referred to as a “rAAV vector” or “rAAV particle.” Such rAAV particles include proteins that encapsidate or package the vector genome and in the case of AAV, they are referred to as capsid proteins.

A vector “genome” refers to the portion of the recombinant plasmid sequence that is ultimately packaged or encapsidated to form a viral (e.g., rAAV) particle. In cases where recombinant plasmids are used to construct or manufacture recombinant vectors, the vector genome does not include the portion of the “plasmid” that does not correspond to the vector genome sequence of the recombinant plasmid. This non vector genome portion of the recombinant plasmid can be referred to as the “plasmid backbone,” which is important for cloning and amplification of the plasmid, a process that is needed for propagation and recombinant virus production, but is not itself packaged or encapsidated into virus (e.g., AAV) particles. Thus, a vector “genome” refers to the nucleic acid that is packaged or encapsidated by virus (e.g., AAV).

Host cells for producing recombinant AAV particles include but are not limited to microorganisms, yeast cells, insect cells, and mammalian cells that can be, or have been, used as recipients of a heterologous rAAV vectors. Cells from the stable human cell line, HEK293 (readily available through, e.g., the American Type Culture Collection under Accession Number ATCC CRL1573) can be used. In certain embodiments a modified human embryonic kidney cell line (e.g., HEK293), which is transformed with adenovirus type-5 DNA fragments, and expresses the adenoviral Ela and Elb genes is used to generate recombinant AAV particles. The modified HEK293 cell line is readily transfected, and provides a particularly convenient platform in which to produce rAAV particles. Other host cell lines appropriate for recombinant AAV production are described in International Application PCT/2017/024951.

In certain embodiments, AAV helper functions are introduced into the host cell by transfecting the host cell with an AAV helper construct either prior to, or concurrently with, the transfection of an AAV expression vector. A host cell having AAV helper functions can be referred to as a “helper cell” or “packaging helper cell.” AAV helper constructs are thus sometimes used to provide at least transient expression of AAV rep and/or cap genes to complement missing AAV functions necessary for productive AAV transduction. AAV helper constructs often lack AAV ITRs and can neither replicate nor package themselves. These constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. A number of other vectors are known which encode Rep and/or Cap expression products.

Methods of generating recombinant AAV particles capable of transducing mammalian cells are known in the art. For example, recombinant AAV particles can be produced as described in U.S. Pat. No. 9,408,904; and International Applications PCT/US2017/025396 and PCT/US2016/064414.

The terms “nucleic acid” and “polynucleotide” are used interchangeably herein to refer to all forms of nucleic acid, oligonucleotides, including deoxyribonucleic acid (DNA) and ribonucleic acid (RNA). Nucleic acids include genomic DNA, cDNA and antisense DNA, and spliced or unspliced mRNA, rRNA tRNA and inhibitory DNA or RNA (RNAi, e.g., small or short hairpin (sh) RNA, microRNA (miRNA), small or short interfering (si) RNA, trans-splicing RNA, or antisense RNA). Nucleic acids include naturally occurring, synthetic, and intentionally modified or altered polynucleotides (e.g., variant nucleic acid). The nucleic acids such as cDNA, genomic DNA, RNA, and fragments thereof which may be single- or double-stranded.

Polynucleotides can be single, double, or triplex, linear or circular, and can be of any length. In discussing polynucleotides, a sequence or structure of a particular polynucleotide may be described herein according to the convention of providing the sequence in the 5′ to 3′ direction.

A “transgene” is used herein to conveniently refer to a heterologous nucleic acid that is intended or has been introduced into a cell or organism. Transgenes include any heterologous nucleic acid, such as a modified nucleic acid encoding GAA.

The term “transduce” and grammatical variations thereof refer to introduction of a molecule such as an rAAV vector into a cell or host organism. The heterologous nucleic acid/transgene may or may not be integrated into genomic nucleic acid of the recipient cell. The introduced heterologous nucleic acid may also exist in the recipient cell or host organism extrachromosomally, or only transiently.

A “transduced cell” is a cell into which the transgene has been introduced. Accordingly, a “transduced” cell (e.g., in a mammal, such as a cell or tissue or organ cell), means a genetic change in a cell following incorporation, for example, of a nucleic acid (e.g., a transgene) into the cell. Thus, a “transduced” cell is a cell into which, or a progeny thereof in which an exogenous nucleic acid has been introduced. The cell(s) can be propagated and the introduced protein expressed. For gene therapy uses and methods, a transduced cell can be in a subject.

An “expression control element” refers to nucleic acid sequence(s) that influence expression of an operably linked nucleic acid. Expression control elements as set forth herein include promoters and enhancers. Vector sequences including AAV vectors can include one or more “expression control elements.” Typically, such elements are included to facilitate proper heterologous polynucleotide transcription and as appropriate translation (e.g., a promoter, enhancer, splicing signal for introns, maintenance of the correct reading frame of the gene to permit in-frame translation of mRNA and, stop codons etc.). Such elements typically act in cis, referred to as a “cis acting” element, but may also act in trans.

Expression control can be effected at the level of transcription, translation, splicing, message stability, etc. Typically, an expression control element that modulates transcription is juxtaposed near the 5′ end (i.e., “upstream”) of a transcribed nucleic acid. Expression control elements can also be located at the 3′ end (i.e., “downstream”) of the transcribed sequence or within the transcript (e.g., in an intron). Expression control elements can be located adjacent to or at a distance away from the transcribed sequence (e.g., 1-10, 10-25, 25-50, 50-100, 100 to 500, or more nucleotides from the polynucleotide), even at considerable distances. Nevertheless, owing to the length limitations of AAV vectors, expression control elements will typically be within 1 to 1000 nucleotides from the transcription start site of the heterologous nucleic acid.

Functionally, expression of operably linked nucleic acid is at least in part controllable by the element (e.g., promoter) such that the element modulates transcription of the nucleic acid and, as appropriate, translation of the transcript. A specific example of an expression control element is a promoter, which is usually located 5′ of the transcribed nucleic acid sequence. A promoter typically increases an amount expressed from operably linked nucleic acid as compared to an amount expressed when no promoter exists.

An “enhancer” as used herein can refer to a sequence that is located adjacent to the heterologous nucleic acid. Enhancer elements are typically located upstream of a promoter element but also function and can be located downstream of or within a sequence. Hence, an enhancer element can be located 10-50 base pairs, 50-100 base pairs, 100-200 base pairs, or 200-300 base pairs, or more base pairs upstream or downstream of a heterologous nucleic acid sequence. Enhancer elements typically increase expressed of an operably linked nucleic acid above expression afforded by a promoter element.

An expression construct may comprise regulatory elements which serve to drive expression in a particular cell or tissue type. Expression control elements (e.g., promoters) include those active in a particular tissue or cell type, referred to herein as a “tissue-specific expression control elements/promoters.” Tissue-specific expression control elements are typically active in specific cell or tissue (e.g., liver). Expression control elements are typically active in particular cells, tissues or organs because they are recognized by transcriptional activator proteins, or other regulators of transcription, that are unique to a specific cell, tissue or organ type. Such regulatory elements are known to those of skill in the art (see, e.g., Sambrook et al. (1989) and Ausubel et al. (1992)).

The incorporation of tissue specific regulatory elements in the expression constructs provides for at least partial tissue tropism for the expression of a heterologous nucleic acid encoding a protein or inhibitory RNA. Examples of promoters that are active in liver are the transthyretin (TTR) gene promoter; human alpha 1-antitrypsin (hAAT) promoter; albumin, Miyatake, et al., J. Virol., 71:5124-32 (1997); hepatitis B virus core promoter, Sandig, et al., Gene Ther. 3:1002-9 (1996); alpha-fetoprotein (AFP), Arbuthnot, et al., Hum. Gene. Ther., 7:1503-14 (1996), among others. An example of an enhancer active in liver is apolipoprotein E (apoE) HCR-1 and HCR-2 (Allan et al., J. Biol. Chem., 272:29113-19 (1997)).

Expression control elements also include ubiquitous or promiscuous promoters/enhancers which are capable of driving expression of a polynucleotide in many different cell types. Such elements include, but are not limited to the cytomegalovirus (CMV) immediate early promoter/enhancer sequences, the Rous sarcoma virus (RSV) promoter/enhancer sequences and the other viral promoters/enhancers active in a variety of mammalian cell types, or synthetic elements that are not present in nature (see, e.g., Boshart et al., Cell, 41:521-530 (1985)), the SV40 promoter, the dihydrofolate reductase promoter, the cytoplasmic β-actin promoter and the phosphoglycerol kinase (PGK) promoter.

Expression control elements also can confer expression in a manner that is regulatable, that is, a signal or stimuli increases or decreases expression of the operably linked heterologous polynucleotide. A regulatable element that increases expression of the operably linked polynucleotide in response to a signal or stimuli is also referred to as an “inducible element” (i.e., is induced by a signal). Particular examples include, but are not limited to, a hormone (e.g., steroid) inducible promoter. Typically, the amount of increase or decrease conferred by such elements is proportional to the amount of signal or stimuli present; the greater the amount of signal or stimuli, the greater the increase or decrease in expression. Particular non-limiting examples include zinc-inducible sheep metallothionine (MT) promoter; the steroid hormone-inducible mouse mammary tumor virus (MMTV) promoter; the T7 polymerase promoter system (WO 98/10088); the tetracycline-repressible system (Gossen, et al., Proc. Natl. Acad. Sci. USA, 89:5547-5551 (1992)); the tetracycline-inducible system (Gossen, et al., Science. 268:1766-1769 (1995); see also Harvey, et al., Curr. Opin. Chem. Biol. 2:512-518 (1998)); the RU486-inducible system (Wang, et al., Nat. Biotech. 15:239-243 (1997) and Wang, et al., Gene Ther. 4:432-441 (1997)]; and the rapamycin-inducible system (Magari, et al., J. Clin. Invest. 100:2865-2872 (1997); Rivera, et al., Nat. Medicine. 2:1028-1032 (1996)). Other regulatable control elements which may be useful in this context are those which are regulated by a specific physiological state, e.g., temperature, acute phase, development.

Expression control elements also include the native elements(s) for the heterologous polynucleotide. A native control element (e.g., promoter) may be used when it is desired that expression of the heterologous polynucleotide should mimic the native expression. The native element may be used when expression of the heterologous polynucleotide is to be regulated temporally or developmentally, or in a tissue-specific manner, or in response to specific transcriptional stimuli. Other native expression control elements, such as introns, polyadenylation sites or Kozak consensus sequences may also be used.

The term “operably linked” means that the regulatory sequences necessary for expression of a nucleic acid sequence are placed in the appropriate positions relative to the sequence so as to effect expression of the nucleic acid sequence. This same definition is sometimes applied to the arrangement of nucleic acid sequences and transcription control elements (e.g., promoters, enhancers, and termination elements) in an expression vector, e.g., rAAV vector.

In the example of an expression control element in operable linkage with a nucleic acid, the relationship is such that the control element modulates expression of the nucleic acid. More specifically, for example, two DNA sequences operably linked means that the two DNAs are arranged (cis or trans) in such a relationship that at least one of the DNA sequences is able to exert a physiological effect upon the other sequence.

Accordingly, additional elements for vectors include, without limitation, an expression control (e.g., promoter/enhancer) element, a transcription termination signal or stop codon, 5′ or 3′ untranslated regions (e.g., polyadenylation (polyA) sequences) which flank a sequence, such as one or more copies of an AAV ITR sequence, or an intron.

Further elements include, for example, filler or stuffer polynucleotide sequences, for example to improve packaging and reduce the presence of contaminating nucleic acid. AAV vectors typically accept inserts of DNA having a size range which is generally about 4 kb to about 5.2 kb, or slightly more. Thus, for shorter sequences, inclusion of a stuffer or filler in order to adjust the length to near or at the normal size of the virus genomic sequence acceptable for AAV vector packaging into virus particle. In various embodiments, a filler/stuffer nucleic acid sequence is an untranslated (non-protein encoding) segment of nucleic acid. For a nucleic acid sequence less than 4.7 kb, the filler or stuffer polynucleotide sequence has a length that when combined (e.g., inserted into a vector) with the sequence has a total length between about 3.0-5.5 kb, or between about 4.0-5.0 kb, or between about 4.3-4.8 kb.

The term “isolated,” when used as a modifier of a composition, means that the compositions are made by the hand of man or are separated, completely or at least in part, from their naturally occurring in vivo environment. Generally, isolated compositions are substantially free of one or more materials with which they normally associate with in nature, for example, one or more protein, nucleic acid, lipid, carbohydrate, cell membrane.

The term “isolated” does not exclude combinations produced by the hand of man, for example, a rAAV sequence, or rAAV particle that packages or encapsidates an AAV vector genome and a pharmaceutical formulation. The term “isolated” also does not exclude alternative physical forms of the composition, such as hybrids/chimeras, multimers/oligomers, modifications (e.g., phosphorylation, glycosylation, lipidation) or derivatized forms, or forms expressed in host cells produced by the hand of man.

The term “substantially pure” refers to a preparation comprising at least 50-60% by weight the compound of interest (e.g., nucleic acid, oligonucleotide, protein, etc.). The preparation can comprise at least 75% by weight, or at least 85% by weight, or about 90-99% by weight, of the compound of interest. Purity is measured by methods appropriate for the compound of interest (e.g., chromatographic methods, agarose or polyacrylamide gel electrophoresis, HPLC analysis, and the like).

The phrase “consisting essentially of” when referring to a particular nucleotide sequence or amino acid sequence means a sequence having the properties of a given SEQ ID NO. For example, when used in reference to an amino acid sequence, the phrase includes the sequence per se and molecular modifications that would not affect the basic and novel characteristics of the sequence.

Nucleic acids, expression vectors (e.g., AAV vector genomes), plasmids, including modified nucleic acids encoding GAA of the invention may be prepared by using recombinant DNA technology methods. The availability of nucleotide sequence information enables preparation of isolated nucleic acid molecules of the invention by a variety of means. Nucleic acids encoding GAA can be made using various standard cloning, recombinant DNA technology, via cell expression or in vitro translation and chemical synthesis techniques. Purity of polynucleotides can be determined through sequencing, gel electrophoresis and the like. For example, nucleic acids can be isolated using hybridization or computer-based database screening techniques. Such techniques include, but are not limited to: (1) hybridization of genomic DNA or cDNA libraries with probes to detect homologous nucleotide sequences; (2) antibody screening to detect polypeptides having shared structural features, for example, using an expression library; (3) polymerase chain reaction (PCR) on genomic DNA or cDNA using primers capable of annealing to a nucleic acid sequence of interest; (4) computer searches of sequence databases for related sequences; and (5) differential screening of a subtracted nucleic acid library.

Nucleic acids may be maintained as DNA in any convenient cloning vector. In one embodiment, clones are maintained in a plasmid cloning/expression vector, such as pBluescript (Stratagene, La Jolla, CA), which is propagated in a suitable E. coli host cell. Alternatively, nucleic acids may be maintained in vector suitable for expression in mammalian cells, for example, an AAV vector. In cases where post-translational modification affects protein function, nucleic acid molecule can be expressed in mammalian cells.

As disclosed herein, rAAV vectors may optionally comprise regulatory elements necessary for expression of the heterologous nucleic acid in a cell positioned in such a manner as to permit expression of the encoded protein in the host cell. Such regulatory elements required for expression include, but are not limited to, promoter sequences, enhancer sequences and transcription initiation sequences as set forth herein and known to the skilled artisan.

Methods and uses of the invention include delivering (transducing) nucleic acid (transgene) into host cells, including dividing and/or non-dividing cells. The nucleic acids, rAAV vector, methods, uses and pharmaceutical formulations of the invention are additionally useful in a method of delivering, administering or providing sequence encoded by heterologous nucleic acid to a subject in need thereof, as a method of treatment. In this manner, the nucleic acid is transcribed and a protein produced in vivo in a subject. The subject may benefit from or be in need of the protein because the subject has a deficiency of the protein, or because production of the protein in the subject may impart some therapeutic effect, as a method of treatment or otherwise.

The invention is useful in animals including human and veterinary medical applications. Suitable subjects therefore include mammals, such as humans, as well as non-human mammals. The term “subject” refers to an animal, typically a mammal, such as humans, non-human primates (apes, gibbons, gorillas, chimpanzees, orangutans, macaques), a domestic animal (dogs and cats), a farm animal (poultry such as chickens and ducks, horses, cows, goats, sheep, pigs), and experimental animals (mouse, rat, rabbit, guinea pig). Human subjects include fetal, neonatal, infant, juvenile and adult subjects. Subjects include animal disease models, for example, mouse and other animal models of protein/enzyme deficiencies such as Pompe disease, and glycogen storage diseases (GSDs) and others known to those of skill in the art.

Subjects appropriate for treatment in accordance with the invention include those having or at risk of producing an insufficient amount of GAA, or produce an aberrant, partially functional or non-functional GAA. Subjects can be tested for GAA activity to determine if such subjects are appropriate for treatment according to a method of the invention. Subjects appropriate for treatment in accordance with the invention also include those subjects that would benefit from GAA. Such subjects that may benefit from GAA include those having a glycogen storage disease (GSD). Treated subjects can be monitored after treatment periodically, e.g., every 1-4 weeks, 1-6 months, 6-12 months, or 1, 2, 3, 4, 5 or more years.

Subjects can be tested for an immune response, e.g., antibodies against AAV. Candidate subjects can therefore be screened prior to treatment according to a method of the invention. Subjects also can be tested for antibodies against AAV after treatment, and optionally monitored for a period of time after treatment. Subjects having pre-existing or developing AAV antibodies can be treated with an immunosuppressive agent, or other regimen as set forth herein.

Subjects appropriate for treatment in accordance with the invention also include those having or at risk of producing antibodies against AAV. rAAV vectors can be administered or delivered to such subjects using several techniques. For example, AAV empty capsid (i.e., AAV lacking a modified nucleic acid encoding GAA) can be delivered to bind to the AAV antibodies in the subject thereby allowing the rAAV vector comprising the heterologous nucleic acid to transduce cells of the subject.

The modified nucleic acids, expression cassettes and rAAV vectors of the invention can be used for treatment of a GAA deficiency. Accordingly, in various embodiments, modified nucleic acids encoding GAA, expression cassettes comprising modified nucleic acids encoding GAA and rAAV vectors of the invention can be used as a therapeutic and/or prophylactic agent.

In particular embodiments, the modified nucleic acids encoding GAA, expression cassettes comprising modified nucleic acids encoding GAA and rAAV vectors of the invention can be used for treatment of Pompe disease as well as other glycogen storage diseases. Administration of modified nucleic acids encoding GAA, expression cassettes comprising modified nucleic acids encoding GAA and rAAV vectors of the invention to a patient with Pompe or another glycogen storage disease leads to the expression of the GAA protein which serves to suppress, inhibit or reduce the accumulation of glycogen, prevent the accumulation of glycogen or degrade glycogen, which in turn can reduce or decrease one or more adverse effects or symptoms of Pompe disease.

Subjects, animals or patients administered the modified nucleic acids encoding GAA, expression cassettes comprising modified nucleic acids encoding GAA and rAAV vectors of the invention can be evaluated by a variety of tests, assays and functional assessments to demonstrate, measure and/or assess efficacy of the modified nucleic acids encoding GAA, expression cassettes comprising modified nucleic acids encoding GAA and rAAV vectors of the invention as therapeutic and/or prophylactic agents. Such tests and assays include, but are not limited to, measurement of GAA activity (such as by use of standard GAA activity assays) and or GAA amount (such as by western blot with anti-GAA antibody) in a biological sample such as blood or plasma; measurement of glycogen content in tissues, such as muscle samples; histological evaluation of muscle (such as muscle from triceps brachii, quadriceps femoris, diaphragm and heart), spinal cord (including examination and counting of ChAT-positive motor neurons in the ventral horn of the cervical thoracic and lumbar spinal cord segments, and evaluation of astroglial reaction and microglial activation); assessment of respiratory function during quiet breathing; forelimbs wire-hanging test; measurement of grip strength; rotarod testing of motor coordination (such as by rotarod protocol); chest x-ray which can show cardiomegaly; electrocardiogram (ECG) to examine heart function; electromyogram (EMG) which can show myopathy; measurement of GAA activity in skin fibroblasts; assays of GAA activity in dried blood samples; blood/serum tests for serum creatine kinase, which when elevated is a nonspecific marker of Pompe disease; blood/serum tests for or aminotransferase, alanine aminotransferase, or lactate dehydrogenase, which may be elevated as indicator of release from muscle in Pompe disease; test for glucose tetrasaccharide in urine (a sensive, nonspecific marker for Pompe disease; analysis of peak and steady-state vector-derived GAA enzyme levels assessed by total GAA protein and activity in plasma; testing of pulmonary function; testing of muscle function; muscle biopsy and staining for presence of glycogen in cell vacuoles; biomarkers of liver health; examination of lysosomal health; testing for immune responses against AAV capsid; testing for immune responses against the GAA transgene protein product; six-minute walk test (6MWT); forced vital capacity test; peak and steady-state AAV vector-derived GAA enzyme levels (assessed by total GAA protein and activity measured in plasma; gait, stairs, gower, and chair (GSGC) testing; muscle strength testing using Rasch-built Medical Research Council (MRC) grading scale; patient-reported life activity/social participation; quantitative sleep and sleep breathing measures from polysomnography (PSG); patient-reported measures of fatigue, daytime sleepiness, and sleep quality; Walton and Gardner-Medwin (WGM) score; respiratory function tests, including, but not limited to sniff nasal inspiratory pressure (SNIP) and maximum inspiratory and expiratory pressures (MIP and MEP, respectively); liver biomarkers; Bhexosaminidase (BHexo) testing; health outcome measurements, including but not limited to short form-36 health survey (SF-36); Rasch-built Pompe-specific activity (R-PAct) scale; fatigue severity score (FSS); and patient reported outcomes measurement information system (PROMIS) item banks.

Additionally, the modified nucleic acids encoding GAA, expression cassettes comprising modified nucleic acids encoding GAA and rAAV vectors of the invention can be used for treatment of a glycogen storage disease (GSD). Glycogen storage diseases include, for example, GSD type I (von Gierke's disease), GSD type II (Pompe disease), GSD type III (Forbes-Cori disease), GSD type IV (Anderson disease, amylopectinosis), GSD type V (McArdle disease), GSD type VI (Hers disease), GSD type VII (Tarui disease), or a lethal congenital glycogen storage disease of the heart.

As set forth herein, rAAV are useful as gene therapy vectors as they can penetrate cells and introduce nucleic acid/genetic material into the cells. Because AAV are not associated with pathogenic disease in humans, rAAV vectors are able to deliver heterologous polynucleotide sequences (e.g., therapeutic proteins and agents) to human patients without causing substantial AAV pathogenesis or disease.

rAAV vectors possess a number of desirable features for such applications, including tropism for dividing and non-dividing cells. Early clinical experience with these vectors also demonstrated no sustained toxicity and immune responses are typically minimal or undetectable. AAV are known to infect a wide variety of cell types in vivo by receptor-mediated endocytosis or by transcytosis. These vector systems have been tested in humans targeting many tissues, such as, retinal epithelium, liver, skeletal muscle, airways, brain, joints and hematopoietic stem cells.

It may be desirable to introduce a rAAV vector that can provide, for example, multiple copies of GAA and hence greater amounts of GAA protein. Improved rAAV vectors and methods for producing these vectors have been described in detail in a number of references, patents, and patent applications, including: Wright J. F. (Hum. Gene Ther., 20:698-706, 2009).

Direct delivery of rAAV vectors or exvivo transduction of human cells followed by infusion into the body will result in expression of the heterologous nucleic acid thereby exerting a beneficial therapeutic effect on hemostasis. In the context of modified nucleic acids encoding GAA, administration suppresses, inhibits or reduces the amount or accumulation of glycogen, prevents accumulation of glycogen or degrades glycogen. This, in turn, can suppress, inhibit, reduce or decrease one or more adverse effects of Pompe disease such as promoting or improving muscle tone and/or muscle strength and/or reducing or decreasing enlarged liver.

Recombinant AAV vector, as well as methods and uses thereof, include any viral strain or serotype. As a non-limiting example, a recombinant AAV vector can be based upon any AAV genome, such as AAV (SEQ ID NOs: 30-32), LK03 (SEQ ID NO:33), AAV1, AAV2, AAV3, AAV4, AAV5, AAV6, AAV7, AAV8, AAV9, AAV10, AAV11, AAV12, Rh10, Rh74, AAV3B or AAV-218, for example. Such vectors can be based on the same strain or serotype (or subgroup or variant), or be different from each other. As a non-limiting example, a recombinant AAV vector based upon a particular serotype genome can be identical to the serotype of the capsid proteins that package the vector. In addition, a recombinant AAV vector genome can be based upon an AAV serotype genome distinct from the serotype of the AAV capsid proteins that package the vector. For example, the AAV vector genome can be based upon AAV2, whereas at least one of the three capsid proteins could be an AAV (SEQ ID NOs: 30-32), LK03 (SEQ ID NO:33), AAV1, AAV3, AAV4, AAV5, AAV6, AAV7, AAV8, AAV9, AAV10, AAV11, AAV12, Rh10, Rh74, AAV3B or AAV-218 or variant thereof, for example.

In particular embodiments, adeno-associated virus (AAV) vectors include AAV (SEQ ID NOs: 30-32), LK03 (SEQ ID NO:33), AAV1, AAV2, AAV3, AAV4, AAV5, AAV6, AAV7, AAV8, AAV9, AAV10, AAV11, AAV12, Rh10, Rh74, AAV3B and AAV-2i8, as well as variants (e.g., capsid variants, such as amino acid insertions, additions, substitutions and deletions) thereof, for example, as set forth in WO 2013/158879 (International Application PCT/US2013/037170), WO 2015/013313 (International Application PCT/US2014/047670) and US 2013/0059732 (U.S. Pat. No. 9,169,299, discloses LK01, LK02, LK03, etc.).

As used herein, the term “serotype” is a distinction used to refer to an AAV having a capsid that is serologically distinct from other AAV serotypes. Serologic distinctiveness is determined on the basis of the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes). Despite the possibility that AAV variants including capsid variants may not be serologically distinct from a reference AAV or other AAV serotype, they differ by at least one nucleotide or amino acid residue compared to the reference or other AAV serotype.

Under the traditional definition, a serotype means that the virus of interest has been tested against serum specific for all existing and characterized serotypes for neutralizing activity and no antibodies have been found that neutralize the virus of interest. As more naturally occurring virus isolates of are discovered and/or capsid mutants generated, there may or may not be serological differences with any of the currently existing serotypes. Thus, in cases where the new virus (e.g., AAV) has no serological difference, this new virus (e.g., AAV) would be a subgroup or variant of the corresponding serotype. In many cases, serology testing for neutralizing activity has yet to be performed on mutant viruses with capsid sequence modifications to determine if they are of another serotype according to the traditional definition of serotype. Accordingly, for the sake of convenience and to avoid repetition, the term “serotype” broadly refers to both serologically distinct viruses (e.g., AAV) as well as viruses (e.g., AAV) that are not serologically distinct that may be within a subgroup or a variant of a given serotype.

As set forth herein, AAV capsid proteins can exhibit less than 100% sequence identity to a reference or parental AAV serotype such as AAV (SEQ ID NOs: 30-32), LK03 (SEQ ID NO: 33), AAV1, AAV2, AAV3, AAV4, AAV5, AAV6, AAV7, AAV8, AAV9, AAV10, AAV11, AAV12, Rh10, Rh74, AAV3B or AAV-218, but are distinct from and not identical to known AAV genes or proteins, such as AAV1, AAV2, AAV3, AAV4, AAV5, AAV6, AAV7, AAV8, AAV9, AAV10, AAV11, AAV12, Rh10, Rh74, AAV3B or AAV-218. In one embodiment, a modified/variant AAV capsid protein includes or consists of a sequence at least 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 99.9% identical to a reference or parental AAV capsid protein, such as AAV (SEQ ID NOs: 30-32), LK03 (SEQ ID NO:33), AAV1, AAV2, AAV3, AAV4, AAV5, AAV6, AAV7, AAV8, AAV9, AAV10, AAV11, AAV12, Rh10, Rh74, AAV3B or AAV-2i8.

rAAV vectors may be administered to a patient via infusion in a biologically compatible carrier, for example, via intravenous injection. rAAV vectors may be administered alone or in combination with other molecules. Accordingly, rAAV vectors and other compositions, agents, drugs, biologics (proteins) can be incorporated into pharmaceutical compositions. Such pharmaceutical compositions are useful for, among other things, administration and delivery to a subject in vivo or ex vivo.

In particular embodiments, pharmaceutical compositions also contain a pharmaceutically acceptable carrier or excipient. Such excipients include any pharmaceutical agent that does not itself induce an immune response harmful to the individual receiving the composition, and which may be administered without undue toxicity.

As used herein the term “pharmaceutically acceptable” and “physiologically acceptable” mean a biologically acceptable formulation, gaseous, liquid or solid, or mixture thereof, which is suitable for one or more routes of administration, in vivo delivery or contact. A “pharmaceutically acceptable” or “physiologically acceptable” composition is a material that is not biologically or otherwise undesirable, e.g., the material may be administered to a subject without causing substantial undesirable biological effects. Thus, such a pharmaceutical composition may be used, for example in administering a nucleic acid, vector, viral particle or protein to a subject.

Pharmaceutically acceptable excipients include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Excipients also include proteins such as albumin. Additionally, auxiliary substances, such as wetting or emulsifying agents, pH buffering substances, and the like, may be present in such vehicles.

The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding, free base forms. In other cases, a preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

Pharmaceutical compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration or delivery, as set forth herein or known to one of skill in the art. Thus, pharmaceutical compositions include carriers, diluents, or excipients suitable for administration by various routes.

Compositions suitable for parenteral administration comprise aqueous and non-aqueous solutions, suspensions or emulsions of the active compound, which preparations are typically sterile and can be isotonic with the blood of the intended recipient. Non-limiting illustrative examples include water, buffered saline, Hanks' solution, Ringer's solution, dextrose, fructose, ethanol, animal, vegetable or synthetic oils. Aqueous injection suspensions may contain substances which increase the viscosity of the suspension, such as sodium carboxymethyl cellulose, sorbitol, or dextran.

Additionally, suspensions of the active compounds may be prepared as appropriate oil injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

After pharmaceutical compositions have been prepared, they may be placed in an appropriate container and labeled for treatment. Such labeling could include amount, frequency, and method of administration.

Pharmaceutical compositions and delivery systems appropriate for the compositions, methods and uses of the invention are known in the art (see, e.g., Remington: The Science and Practice of Pharmacy (2003) 20th ed., Mack Publishing Co., Easton, PA; Remington's Pharmaceutical Sciences (1990) 18th ed., Mack Publishing Co., Easton, PA; The Merck Index (1996) 12th ed., Merck Publishing Group, Whitehouse, NJ; Pharmaceutical Principles of Solid Dosage Forms (1993), Technomic Publishing Co., Inc., Lancaster, Pa.; Ansel and Stoklosa, Pharmaceutical Calculations (2001) 11th ed., Lippincott Williams & Wilkins, Baltimore, MD; and Poznansky et al., Drug Delivery Systems (1980), R. L. Juliano, ed., Oxford, N.Y., pp. 253-315).

An “effective amount” or “sufficient amount” refers to an amount that provides, in single or multiple doses, alone or in combination, with one or more other compositions (therapeutic or immunosuppressive agents such as a drug), treatments, protocols, or therapeutic regimens agents, a detectable response of any duration of time (long or short term), an expected or desired outcome in or a benefit to a subject of any measurable or detectable degree or for any duration of time (e.g., for minutes, hours, days, months, years, or cured).

Doses can vary and depend upon the type, onset, progression, severity, frequency, duration, or probability of the disease to which treatment is directed, the clinical endpoint desired, previous or simultaneous treatments, the general health, age, gender, race or immunological competency of the subject and other factors that will be appreciated by the skilled artisan. The dose amount, number, frequency or duration may be proportionally increased or reduced, as indicated by any adverse side effects, complications or other risk factors of the treatment or therapy and the status of the subject. The skilled artisan will appreciate the factors that may influence the dosage and timing required to provide an amount sufficient for providing a therapeutic or prophylactic benefit.

The dose to achieve a therapeutic effect, e.g., the dose in vector genomes/per kilogram of body weight (vg/kg), will vary based on several factors including, but not limited to: route of administration, the level of heterologous polynucleotide expression required to achieve a therapeutic effect, the specific disease treated, any host immune response to the viral vector, a host immune response to the heterologous polynucleotide or expression product (protein), and the stability of the protein expressed. One skilled in the art can determine a rAAV/vector genome dose range to treat a patient having a particular disease or disorder based on the aforementioned factors, as well as other factors.

Generally, doses will range from at least 1×10⁸ vector genomes per kilogram (vg/kg) of the weight of the subject, or more, for example, 1×10⁹, 1×10¹⁰, 1×10¹¹, 1×10¹², 1×10¹³ or 1×10¹⁴, or more, vector genomes per kilogram (vg/kg) of the weight of the subject, to achieve a therapeutic effect. An rAAV dose in the range of 1×10¹⁰-1×10¹¹ vg/kg in mice, and 1×10¹². 1×10¹³ vg/kg in dogs have been effective. Doses can be less, for example, a dose of less than 6×10¹² vg/kg. More particularly, a dose of 5×10¹¹ vg/kg or 1×10¹² vg/kg.

rAAV vector doses can be at a level, typically at the lower end of the dose spectrum, such that there is not a substantial immune response against the heterologous nucleic acid sequence, the encoded protein or inhibitory nucleic acid, or rAAV vector. More particularly, a dose of up to but less than 6×10¹² vg/kg, such as about 5×10¹¹ to about 5×10¹² vg/kg, or more particularly, about 5×10¹¹ vg/kg or about 1×10¹² vg/kg.

A “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, is calculated to produce a desired effect (e.g., prophylactic or therapeutic effect). Unit dosage forms may be within, for example, ampules and vials, which may include a liquid composition, or a composition in a freeze-dried or lyophilized state; a sterile liquid carrier, for example, can be added prior to administration or delivery in vivo. Individual unit dosage forms can be included in multi-dose kits or containers. rAAV particles, and pharmaceutical compositions thereof can be packaged in single or multiple unit dosage form for ease of administration and uniformity of dosage.

The doses of an “effective amount” or “sufficient amount” for treatment (e.g., to ameliorate or to provide a therapeutic benefit or improvement) typically are effective to provide a response to one, multiple or all adverse symptoms, consequences or complications of the disease, one or more adverse symptoms, disorders, illnesses, pathologies, or complications, for example, caused by or associated with the disease, to a measurable extent, although decreasing, reducing, inhibiting, suppressing, limiting or controlling progression or worsening of the disease is a satisfactory outcome.

An effective amount or a sufficient amount can but need not be provided in a single administration, may require multiple administrations, and, can but need not be, administered alone or in combination with another composition (e.g., agent), treatment, protocol or therapeutic regimen. For example, the amount may be proportionally increased as indicated by the need of the subject, type, status and severity of the disease treated or side effects (if any) of treatment. In addition, an effective amount or a sufficient amount need not be effective or sufficient if given in single or multiple doses without a second composition (e.g., another drug or agent), treatment, protocol or therapeutic regimen, since additional doses, amounts or duration above and beyond such doses, or additional compositions (e.g., drugs or agents), treatments, protocols or therapeutic regimens may be included in order to be considered effective or sufficient in a given subject. Amounts considered effective also include amounts that result in a reduction of the use of another treatment, therapeutic regimen or protocol, such as administration of modified nucleic acid encoding GAA for treatment of a GAA deficiency (e.g., Pompe disease) or another glycogen storage disease.

Accordingly, methods and uses of the invention also include, among other things, methods and uses that result in a reduced need or use of another compound, agent, drug, therapeutic regimen, treatment protocol, process, or remedy. For example, for GAA deficiency, a method or use of the invention has a therapeutic benefit if in a given subject a less frequent or reduced dose or elimination of administration of a recombinant GAA to supplement for the deficient or defective GAA in the subject. Thus, in accordance with the invention, methods and uses of reducing need or use of another treatment or therapy are provided.

An effective amount or a sufficient amount need not be effective in each and every subject treated, nor a majority of treated subjects in a given group or population. An effective amount or a sufficient amount means effectiveness or sufficiency in a particular subject, not a group or the general population. As is typical for such methods, some subjects will exhibit a greater response, or less or no response to a given treatment method or use.

Administration or in vivo delivery to a subject can be performed prior to development of an adverse symptom, condition, complication, etc. caused by or associated with the disease. For example, a screen (e.g., genetic) can be used to identify such subjects as candidates for invention compositions, methods and uses. Such subjects therefore include those screened positive for an insufficient amount or a deficiency in a functional gene product (e.g., GAA or a protein deficiency that leads to a GSD), or that produce an aberrant, partially functional or non-functional gene product (e.g., GAA or a protein implicated in a GSD).

Administration or in vivo delivery to a subject in accordance with the methods and uses of the invention as disclosed herein can be practiced within 1-2, 2-4, 4-12, 12-24 or 24-72 hours after a subject has been identified as having the disease targeted for treatment, has one or more symptoms of the disease, or has been screened and is identified as positive as set forth herein even though the subject does not have one or more symptoms of the disease. Of course, methods and uses of the invention can be practiced 1-7, 7-14, 14-24, 24-48, 48-64 or more days, months or years after a subject has been identified as having the disease targeted for treatment, has one or more symptoms of the disease, or has been screened and is identified as positive as set forth herein.

The term “ameliorate” means a detectable or measurable improvement in a subject's disease or symptom thereof, or an underlying cellular response. A detectable or measurable improvement includes a subjective or objective decrease, reduction, inhibition, suppression, limit or control in the occurrence, frequency, severity, progression, or duration of the disease, or complication caused by or associated with the disease, or an improvement in a symptom or an underlying cause or a consequence of the disease, or a reversal of the disease.

For Pompe disease, an effective amount would be an amount that inhibits or reduces glycogen production or accumulation, enhances or increases glycogen degradation or removal, for example. An effective amount would also be an amount that improves or ameliorates difficulty eating and/or not gaining weight; poor head and/or neck control; breathing problems and/or lung infections; enlarged and/or thickening heart; heart defects; enlarged tongue; difficulty swallowing; enlarged liver; poor muscle strength; week muscle tone; weakness in the legs, waist and/or arms; shortness of breath; difficulty exercising; difficulty breathing while sleeping; curvature of the spine; and/or stiff joints; week muscle tone and/or lack of muscle strength. An effective amount would also be an amount that decreases or inhibits one or more symptoms, or prevents or reduces the progression or worsening of one or more symptoms, or stabilizes one or more symptoms, or improves one or more symptoms in a patient or subject in need of GAA or having Pompe disease.

Therapeutic doses will depend on, among other factors, the age and general condition of the subject, the severity of the disease or disorder. A therapeutically effective amount in humans will fall in a relatively broad range that may be determined by a medical practitioner based on the response of an individual patient.

Compositions such as pharmaceutical compositions may be delivered to a subject, so as to allow production of the encoded protein. In a particular embodiment, pharmaceutical compositions comprise sufficient genetic material to enable a recipient to produce a therapeutically effective amount of a protein in the subject.

Compositions may be formulated and/or administered in any sterile, biocompatible pharmaceutical carrier, including, but not limited to, saline, buffered saline, dextrose, and water. The compositions may be formulated and/or administered to a patient alone, or in combination with other agents (e.g., co-factors) which influence hemostasis.

Methods and uses of the invention include delivery and administration systemically, regionally or locally, or by any route, for example, by injection or infusion. Delivery of the pharmaceutical compositions in vivo may generally be accomplished via injection using a conventional syringe, although other delivery methods such as convection-enhanced delivery are envisioned (See e.g., U.S. Pat. No. 5,720,720). For example, compositions may be delivered subcutaneously, epidermally, intradermally, intrathecally, intraorbitally, intramucosally, intranasally, intraperitoneally, intravenously, intra-pleurally, intraarterially, intracavitary, orally, intrahepatically, via the portal vein, or intramuscularly. Other modes of administration include oral and pulmonary administration, suppositories, and transdermal applications. A clinician specializing in the treatment of patients with Pompe or other glycogen storage diseases may determine the optimal route for administration of the adenoviral-associated vectors based on a number of criteria, including, but not limited to: the condition of the patient and the purpose of the treatment (e.g., enhanced or reduced GAA levels).

The compositions may be administered alone. In certain embodiments, a rAAV particle provides a therapeutic effect without an immunosuppressive agent. The therapeutic effect optionally is sustained for a period of time, e.g., 2-4, 4-6, 6-8, 8-10, 10-14, 14-20, 20-25, 25-30, or 30-50 days or more, for example, 50-75, 75-100, 100-150, 150-200 days or more without administering an immunosuppressive agent. Accordingly, a therapeutic effect is provided for a period of time.

Invention rAAV vectors, methods and uses can be combined with any compound, agent, drug, treatment or other therapeutic regimen or protocol having a desired therapeutic, beneficial, additive, synergistic or complementary activity or effect. Exemplary combination compositions and treatments include second actives, such as, biologics (proteins), agents (e.g., immunosuppressive agents) and drugs. Such biologics (proteins), agents, drugs, treatments and therapies can be administered or performed prior to, substantially contemporaneously with or following any other method or use of the invention.

The compound, agent, drug, treatment or other therapeutic regimen or protocol can be administered as a combination composition, or administered separately, such as concurrently or in series or sequentially (prior to or following) delivery or administration of a nucleic acid, vector, or rAAV particle. The invention therefore provides combinations in which a method or use of the invention is in a combination with any compound, agent, drug, therapeutic regimen, treatment protocol, process, remedy or composition, set forth herein or known to one of skill in the art. The compound, agent, drug, therapeutic regimen, treatment protocol, process, remedy or composition can be administered or performed prior to, substantially contemporaneously with or following administration of a nucleic acid, vector or rAAV particle of the invention, to a subject.

In certain embodiments, nucleic acid, vector, or rAAV particle of the invention is administered to a patient in combination with an immunosuppressive agent or regimen where the patient has or is at risk of developing an immune response against the rAAV particle and/or the GAA protein. Such immunosuppressive agent or regimen can be administered prior to, substantially at the same time or after administering a nucleic acid, vector, or rAAV vector of the invention

In some embodiments, a subject or patient, such as a human patient, with Pompe disease has developed inhibitors to the GAA protein (including anti-GAA antibodies and/or anti-GAA T-cells), which can occur following treatment with traditional enzyme replacement therapy (e.g., following administration of recombinantly produced GAA protein). The development of such GAA inhibitors can occur in patients that receive enzyme replacement therapy, particularly where the patient has undetectable GAA levels (as can be the case in infantile Pompe disease), leading the patient's immune system to see the replacement GAA protein as “foreign.” In certain embodiments, a Pompe patient having GAA inhibitors is administered one or more regimen intended to achieve immune tolerance or mitigate the immune response to the GAA protein in the patient, prior to, substantially at the same time or after administering an rAAV vector of the invention. Such regimens to achieve immune tolerance or mitigate the immune response to the GAA protein can include administration of one or more immunosuppressive agent, including but not limited to methotrexate, rituximab, intravenous gamma globulin (IVIG), omalizumab, and synthetic vaccine particle (SVP™)-rapamycin (rapamycin encapsulated in a biodegradable nanoparticle) and/or administration of one or more immunosuppressive protocol or procedure, such as B-cell depletion, immunoadsorption, and plasmapheresis.

In certain embodiments, rAAV vector is administered in conjunction with one or more immunosuppressive agents prior to, substantially at the same time or after administering a rAAV vector. In certain embodiments, e.g., 1-12, 12-24 or 24-48 hours, or 2-4, 4-6, 6-8, 8-10, 10-14, 14-20, 20-25, 25-30, 30-50, or more than 50 days following administering rAAV vector. Such administration of immunosuppressive agents after a period of time following administering rAAV vector if there is a decrease in the encoded protein or inhibitory nucleic acid after the initial expression levels for a period of time, e.g., 20-25, 25-30, 30-50, 50-75, 75-100, 100-150, 150-200 or more than 200 days following rAAV vector.

In certain embodiments, an immunosuppressive agent is an anti-inflammatory agent. In certain embodiments, an immunosuppressive agent is a steroid, e.g., a corticosteroid. In certain embodiments, an immunosuppressive agent is prednisone, prednisolone, cyclosporine (e.g., cyclosporine A), mycophenolate, a B cell targeting antibody, e.g., rituximab; a proteasome inhibitor, e.g., bortezomib; a mammalian target of rapamycin (mTOR) inhibitor, e.g., rapamycin; a tyrosine kinase inhibitor, e.g., ibrutinib; an inhibitor of B-cell activating factor (BAFF); or an inhibitor of a proliferation-inducing ligand (APRIL) or a derivative thereof. In certain embodiments, the immunosuppressive agent is an anti-IL-1 β agent (e.g., anti-IL-1ß monoclonal antibody canakinumab (Ilaris®)) or an anti-IL-6 agent (e.g., anti-IL-6 antibody sirukumab or anti-IL-6 receptor antibody tocilizumab (Actemra®)), or a combination thereof.

Immune-suppression protocols, including the use of rapamycin, alone or in combination with IL-10, can be used to decrease, reduce, inhibit, prevent or block humoral and cellular immune responses to the GAA protein. Hepatic gene transfer with AAV vectors of the invention can be used to induce immune tolerance to the GAA protein through induction of regulatory T cells (Tregs) and other mechanisms. Strategies to reduce (overcome) or avoid humoral immunity to AAV in systemic gene transfer include, administering high vector doses, use of AAV empty capsids as decoys to adsorb anti-AAV antibodies, administration of immunosuppressive drugs to decrease, reduce, inhibit, prevent or eradicate the humoral immune response to AAV, changing the AAV capsid serotype or engineering the AAV capsid to be less susceptible to neutralizing antibodies, use of plasma exchange cycles to adsorb anti-AAV immunoglobulins, thereby reducing anti-AAV antibody titer, and use of delivery techniques such as balloon catheters followed by saline flushing. Such strategies are described in Mingozzi et al., 2013, Blood, 122:23-36. Procedures and approaches to induce tolerance to GAA in Pompe patients in order to improve therapeutic treatment are reviewed in Doerfler et al., 2016, Mol. Ther., 3:15053.

Ratio of AAV empty capsids to the rAAV vector can be within or between about 100:1-50:1, from about 50:1-25:1, from about 25:1-10:1, from about 10:1-1:1, from about 1:1-1:10, from about 1:10-1:25, from about 1:25-1:50, or from about 1:50-1:100. Ratios can also be about 2:1, 3:1, 4:1, 5:1, 6:1, 7:1, 8:1, 9:1, or 10:1.

Amounts of AAV empty capsids to administer can be calibrated based upon the amount (titer) of AAV antibodies produced in a particular subject. AAV empty capsids can be of any serotype, for example, AAV (SEQ ID NOs: 30-32), LK03 (SEQ ID NO:33), AAV1, AAV2, AAV3, AAV4, AAV5, AAV6, AAV7, AAV8, AAV9, AAV10, AAV11, AAV12, Rh10, Rh74, AAV3B or AAV-2i8.

Alternatively, or in addition to, rAAV vector can be delivered by direct intramuscular injection (e.g., one or more slow-twitch fibers of a muscle). In another alternative, a catheter introduced into the femoral artery can be used to delivery rAAV vectors to liver via the hepatic artery. Non-surgical means can also be employed, such as endoscopic retrograde cholangiopancreatography (ERCP), to deliver rAAV vectors directly to the liver, thereby bypassing the bloodstream and AAV antibodies. Other ductal systems, such as the ducts of the submandibular gland, can also be used as portals for delivering rAAV vectors into a subject that develops or has preexisting anti-AAV antibodies.

Additional strategies to reduce humoral immunity to AAV include methods to remove, deplete, capture, and/or inactivate AAV antibodies, commonly referred to as apheresis and more particularly, plasmapheresis where blood products are involved. Apheresis or plasmapheresis, is a process in which a human subject's plasma is circulated ex vivo (extracorporal) through a device that modifies the plasma through addition, removal and/or replacement of components before its return to the patient. Plasmapheresis can be used to remove human immunoglobulins (e.g., IgG, IgE, IgA, IgD) from a blood product (e.g., plasma). This procedure depletes, captures, inactivates, reduces or removes immunoglobulins (antibodies) that bind AAV thereby reducing the titer of AAV antibodies in the treated subject that may contribute to AAV vector neutralization. An example is a device composed of an AAV capsid affinity matrix column. Passing blood product (e.g., plasma) through an AAV capsid affinity matrix would result in binding only of AAV antibodies, and of all isotypes (including IgG, IgM, etc.).

A sufficient amount of plasmapheresis using an AAV capsid affinity matrix is predicted to substantially remove AAV capsid antibodies, and reduce the AAV capsid antibody titer (load) in the human. In certain embodiments, titer in a treated subject is reduced substantially to low levels (to <1:5, or less, such as <1:4, or <1:3, or <1:2, or <1:1). A reduction in antibody titer will be temporary because the B lymphocytes that produce the AAV capsid antibodies would be expected to gradually cause the AAV capsid antibody titer to rebound to the steady state level prior to plasmapheresis.

In the case where a pre-existing AAV antibody titer was reduced from 1:100 to 1:1, AAV antibody titer rebounds of approximately 0.15% (corresponding to a titer of 1:1.2) 0.43% (1:1.4), 0.9% (1:1.9), 1.7% (1:2.7), and 3.4% (1:4.4), occur at 1 hour, 3 hours, 6 hours, 12 hours and 24 hours, respectively, after completion of the plasmapheresis method. Temporary removal of AAV antibodies from such a subject would correspond to a window of time (for example, of about 24 hours or less, such as 12 hours or less, or 6 hours or less, or 3 hours or less, or 2 hours or less, or 1 hour or less) during which an AAV vector could be administered to the subject and predicted to efficiently transduce target tissues without substantial neutralization of the AAV vector with the AAV antibodies.

In the case where a pre-existing AAV antibody titer was reduced from 1:1000 to 1:1, AAV antibody titer rebounds of approximately 0.15% (corresponding to a titer of 1:2.5) 0.4% (1:5.3), 0.9% (1:9.7), 1.7% (1:18), and 3.4% (1:35), occur at 1 hour, 3 hours, 6 hours, 12 hours and 24 hours, respectively, after completion of the plasmapheresis method. Thus, a window for administration of AAV vector will be comparatively shorter.

AAV antibodies may be preexisting and may be present at levels that reduce or block therapeutic GAA gene transfer vector transduction of target cells. Alternatively, AAV antibodies may develop after exposure to AAV or administration of an AAV vector. If such antibodies develop after administration of an AAV vector, these subjects can also be treated via apheresis, more particularly, plasmapheresis.

In some embodiments, the nucleic acids, expression cassettes and AAV vectors of the invention can be used in combination with symptomatic and support therapies, including, for example, respiratory support (including mechanical ventilation), physical therapy to strengthen muscles, physiotherapy to improve strength and physical ability, occupational therapy, including use of canes, walkers and wheelchairs, speech therapy to improve articulation and speech, use of orthopedic devices, including braces, and dietary therapy and feeding tubes to ensure proper nutrition and weight gain.

In some embodiments, the nucleic acids, expression cassettes and AAV vectors of the invention can be used in combination with pharmacological chaperone therapy (also known as enzyme enhancement therapy), where one or more pharmacological chaperones is administered before, concomitant with, or after administration of the nucleic acid, expression cassette or AAV vectors of the invention, for the treatment of a GSD, such as Pompe disease.

In some embodiments, the nucleic acids, expression cassettes and AAV vectors of the invention can be used in combination with one or more pharmacological chaperone, which may stabilize GAA protein. Pharmacological chaperones that can be used in combination with the nucleic acids, expression cassettes and AAV vectors of the invention include 1-deoxynojirimycin (1-DNJ, also known as duvoglustat), N-butyl-1-deoxynojirimycin (also known as miglustat), N-methyl-DNJ, N-ethyl-DNJ, N-propyl-DNJ, N-pentyl-DNJ, N-hexyl-DNJ, N-heptyl-DNJ, N-octyl-DNJ, N-nonyl-DNJ, N-methylcyclopropyl-DNJ, N-methylcyclopentyl-DNJ, N-2-hydroxyethyl-DNJ, 5-N-carboxypentyl DNJ, and pharmacological chaperones described in U.S. Pat. Nos. 6,599,919 and 9,181,184, and in International Patent Application publication WO/2013/182652.

In some embodiments, the nucleic acids, expression cassettes and AAV vectors of the invention can be used in combination with adjunctive therapy, with one or more β2 agonist, including, for example, clenbuterol, albuterol, formoterol and salmeterol, and as described in International Patent Application publication WO/2017/049161.

In particular embodiments, the nucleic acids, and expression cassettes of the invention are delivered or administered via AAV vector particles. In other embodiments, the nucleic acids and expression cassettes of the invention can be delivered or administered via other types of viral particles, including retroviral, adenoviral, helper-dependent adenoviral, hybrid adenoviral, herpes simplex virus, lentiviral, poxvirus, Epstein-Barr virus, vaccinia virus, and human cytomegalovirus particles.

In other embodiments, the nucleic acids and expression cassettes of the invention are delivered or administered with a non-viral delivery system. Non-viral delivery systems include for example, chemical methods, such as liposomes, nanoparticles, lipid nanoparticles, polymers, microparticles, microcapsules, micelles, or extracellular vesicles and physical methods, such as gene gun, electroporation, particle bombardment, ultrasound utilization and magnetofection.

In some embodiments, the nucleic acids and expression cassettes of the invention are delivered as naked DNA, minicircles, transposons, of closed-ended linear duplex DNA.

In other embodiments, the nucleic acids, and expression cassettes of the invention are delivered or administered in AAV vector particles, or other viral particles, that are further encapsulated or complexed with liposomes, nanoparticles, lipid nanoparticles, polymers, microparticles, microcapsules, micelles, or extracellular vesicles.

A “lipid nanoparticle” or “LNP” refers to a lipid-based vesicle useful for delivery of AAV and having dimensions on the nanoscale, i.e., from about 10 nm to about 1000 nm, or from about 50 to about 500 nm, or from about 75 to about 127 nm. Without being bound by theory, the LNP is believed to provide the nucleic acid, expression cassette, or AAV vector with partial or complete shielding from the immune system. Shielding allows delivery of the nucleic acid, expression cassette, or AAV vector to a tissue or cell while avoiding inducing a substantial immune response against the nucleic acid, expression cassette, or AAV vector in vivo. Shielding may also allow repeated administration without inducing a substantial immune response against the nucleic acid, expression vector or AAV vector in vivo (e.g., in a subject such as a human). Shielding may also improve or increase nucleic acid, expression cassette, or AAV vector delivery efficiency in vivo.

The pI (isoelectric point) of AAV is in a range from about 6 to about 6.5. Thus, the AAV surface carries a slight negative charge. As such it may be beneficial for the LNP to comprise a cationic lipid such as, for example, an amino lipid. Exemplary amino lipids have been described in U.S. Pat. Nos. 9,352,042, 9,220,683, 9,186,325, 9,139,554, 9,126,966 9,018,187, 8,999,351, 8,722,082, 8,642,076, 8,569,256, 8,466,122, and 7,745,651 and U.S. Patent Publication Nos. 2016/0213785, 2016/0199485, 2015/0265708, 2014/0288146, 2013/0123338, 2013/0116307, 2013/0064894, 2012/0172411, and 2010/0117125.

The terms “cationic lipid” and “amino lipid” are used interchangeably herein to include those lipids and salts thereof having one, two, three, or more fatty acid or fatty alkyl chains and a pH-titratable amino group (e.g., an alkylamino or dialkylamino group). The cationic lipid is typically protonated (i.e., positively charged) at a pH below the pKa of the cationic lipid and is substantially neutral at a pH above the pKa. The cationic lipids may also be titratable cationic lipids. In some embodiments, the cationic lipids comprise: a protonatable tertiary amine (e.g., pH-titratable) group; C18 alkyl chains, wherein each alkyl chain independently has 0 to 3 (e.g., 0, 1, 2, or 3) double bonds; and ether, ester, or ketal linkages between the head group and alkyl chains.

Cationic lipids may include, without limitation, 1,2-dilinoleyloxy-N,N-dimethylaminopropane (DLinDMA), 1,2-dilinolenyloxy-N,N-dimethylaminopropane (DLenDMA), 1,2-di-γ-linolenyloxy-N,N-dimethylaminopropane (γ-DLenDMA), 2,2-dilinoleyl-4-(2-dimethylaminoethyl)-[1,3]-dioxolane (DLin-K-C2-DMA, also known as DLin-C2K-DMA, XTC2, and C2K), 2,2-dilinoleyl-4-dimethylaminomethyl-[1,3]-dioxolane (DLin-K-DMA), dilinoleylmethyl-3-dimethylaminopropionate (DLin-M-C2-DMA, also known as MC2), (6Z,9Z,28Z,31 Z)-heptatriaconta-6,9,28,31-tetraen-19-yl 4-(dimethylamino) butanoate (DLin-M-C3-DMA, also known as MC3), salts thereof, and mixtures thereof. Other cationic lipids also include, but are not limited to, 1,2-distearyloxy-N,N-dimethyl-3-aminopropane (DSDMA), 1,2-dioleyloxy-N,N-dimethyl-3-aminopropane (DODMA), 2,2-dilinoleyl-4-(3-dimethylaminopropyl)-[1,3]-dioxolane (DLin-K-C3-DMA), 2,2-dilinoleyl-4-(3-dimethylaminobutyl)-[1,3]-dioxolane (DLin-K-C4-DMA), DLen-C2K-DMA, Y-DLen-C2K-DMA, and (DLin-MP-DMA) (also known as 1-B11).

Still further cationic lipids may include, without limitation, 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 1,2-dilinoleylcarbamoyloxy-3-dimethylaminopropane (DLin-C-DAP), 1,2-dilinoleyoxy-3-(dimethylamino) acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino) propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino) ethoxypropane (DLin-EG-DMA), N,N-dioleyl-N,N-dimethylammonium chloride (DODAC), N-(1-(2,3-dioleyloxy) propyl)-N,N,N-trimethylammonium chloride (DOTMA), N,N-distearyl-N,N-dimethylammonium bromide (DDAB), N-(1-(2,3-dioleoyloxy) propyl)-N,N,N-trimethylammonium chloride (DOTAP), 3-(N—(N′,N′-dimethylaminoethane)-carbamoyl) cholesterol (DC-Chol), N-(1,2-dimyristyloxyprop-3-yl)-N,N-dimethyl-N-hydroxyethyl ammonium bromide (DMRIE), 2,3-dioleyloxy-N-[2 (spermine-carboxamido)ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy) propane (CLinDMA), 2-[5′-(cholest-5-en-3-beta-oxy)-3′-oxapentoxy)-3-dimethyl-1-(cis,cis-9′,1-2′-octadecadienoxy) propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-dilinoleylcarbamyl-3-dimethylaminopropane (DLincarbDAP), dexamethasone-sperimine (DS) and disubstituted spermine (D2S) or mixtures thereof.

A number of commercial preparations of cationic lipids can be used, such as, LIPOFECTIN® (including DOTMA and DOPE, available from GIBCO/BRL), and LIPOFECTAMINE® (comprising DOSPA and DOPE, available from GIBCO/BRL).

In certain embodiments, cationic lipid may be present in an amount from about 10% by weight of the LNP to about 85% by weight of the lipid nanoparticle, or from about 50% by weight of the LNP to about 75% by weight of the LNP.

Sterols may confer fluidity to the LNP. As used herein, “sterol” refers to any naturally occurring sterol of plant (phytosterols) or animal (zoosterols) origin as well as non-naturally occurring synthetic sterols, all of which are characterized by the presence of a hydroxyl group at the 3-position of the steroid A-ring. The sterol can be any sterol conventionally used in the field of liposome, lipid vesicle or lipid particle preparation, most commonly cholesterol. Phytosterols may include campesterol, sitosterol, and stigmasterol. Sterols also includes sterol-modified lipids, such as those described in U.S. Patent Application Publication 2011/0177156. In some embodiments, a sterol may be present in an amount from about 5% by weight of the LNP to about 50% by weight of the lipid nanoparticle or from about 10% by weight of the LNP to about 25% by weight of the LNP.

LNP can comprise a neutral lipid. Neutral lipids may comprise any lipid species which exists either in an uncharged or neutral zwitterionic form at physiological pH. Such lipids include, without limitation, diacylphosphatidylcholine, diacylphosphatidylethanolamine, ceramide, sphingomyelin, dihydrosphingomyelin, cephalin, and cerebrosides. The selection of neutral lipids is generally guided by consideration of, inter alia, particle size and the requisite stability. In some embodiments, the neutral lipid component may be a lipid having two acyl groups (e.g., diacylphosphatidylcholine and diacylphosphatidylethanolamine).

Lipids having a variety of acyl chain groups of varying chain length and degree of saturation are available or may be isolated or synthesized by well-known techniques. In some embodiments, lipids containing saturated fatty acids with carbon chain lengths in the range of C14 to C22 may be used. In another group of embodiments, lipids with mono or diunsaturated fatty acids with carbon chain lengths in the range of C14 to C22 are used. Additionally, lipids having mixtures of saturated and unsaturated fatty acid chains can be used. Exemplary neutral lipids include, without limitation, 1,2-dioleoyl-sn-glycero-3-phosphatidyl-ethanolamine (DOPE), 1,2-distearoyl-sn-glycero-3-phosphocholine (DSPC), 1-palmitoyl-2-oleoyl-sn-glycero-3-phosphocholine (POPC), or any related phosphatidylcholine. The neutral lipids may also be composed of sphingomyelin, dihydrosphingomyelin, or phospholipids with other head groups, such as serine and inositol.

In some embodiments, the neutral lipid may be present in an amount from about 0.1% by weight of the lipid nanoparticle to about 75% by weight of the LNP, or from about 5% by weight of the LNP to about 15% by weight of the LNP.

LNP encapsulated nucleic acids, expression cassettes and AAV vector can be incorporated into pharmaceutical compositions, e.g., a pharmaceutically acceptable carrier or excipient. Such pharmaceutical compositions are useful for, among other things, administration and delivery of LNP encapsulated acids, expression cassettes and AAV vector to a subject in vivo or ex vivo.

Preparations of LNP can be combined with additional components. Non-limiting examples include polyethylene glycol (PEG) and sterols.

The term “PEG” refers to a polyethylene glycol, a linear, water-soluble polymer of ethylene PEG repeating units with two terminal hydroxyl groups. PEGs are classified by their molecular weights; for example, PEG 2000 has an average molecular weight of about 2,000 daltons, and PEG 5000 has an average molecular weight of about 5,000 daltons. PEGs are commercially available from Sigma Chemical Co. and other companies and include, for example, the following functional PEGs: monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH2), monomethoxypolyethylene glycol-tresylate (MePEG-TRES), and monomethoxypolyethylene glycol-imidazolyl-carbonyl (MePEG-IM).

In some embodiments, PEG may be a polyethylene glycol with an average molecular weight of about 550 to about 10,000 daltons and is optionally substituted by alkyl, alkoxy, acyl or aryl. In some embodiments, the PEG may be substituted with methyl at the terminal hydroxyl position. In another preferred embodiment, the PEG may have an average molecular weight from about 750 to about 5,000 daltons, or from about 1,000 to about 5,000 daltons, or from about 1,500 to about 3,000 daltons or from about 2,000 daltons or of about 750 daltons. The PEG can be optionally substituted with alkyl, alkoxy, acyl or aryl. In some embodiments, the terminal hydroxyl group may be substituted with a methoxy or methyl group.

PEG-modified lipids include the PEG-dialkyloxypropyl conjugates (PEG-DAA) described in U.S. Pat. Nos. 8,936,942 and 7,803,397. PEG-modified lipids (or lipid-polyoxyethylene conjugates) that are useful may have a variety of “anchoring” lipid portions to secure the PEG portion to the surface of the lipid vesicle. Examples of suitable PEG-modified lipids include PEG-modified phosphatidylethanolamine and phosphatidic acid, PEG-ceramide conjugates (e.g., PEG-CerC14 or PEG-CerC20) which are described in U.S. Pat. No. 5,820,873, PEG-modified dialkylamines and PEG-modified 1,2-diacyloxypropan-3-amines. In some embodiments, the PEG-modified lipid may be PEG-modified diacylglycerols and dialkylglycerols. In some embodiments, the PEG may be in an amount from about 0.5% by weight of the LNP to about 20% by weight of the LNP, or from about 5% by weight of the LNP to about 15% by weight of the LNP.

Furthermore, LNP can be a PEG-modified and a sterol-modified LNP. The LNPs, combined with additional components, can be the same or separate LNPs. In other words, the same LNP can be PEG modified and sterol modified or, alternatively, a first LNP can be PEG modified and a second LNP can be sterol modified. Optionally, the first and second modified LNPs can be combined.

In some embodiments, prior to encapsulating LNPs may have a size in a range from about 10 nm to 500 nm, or from about 50 nm to about 200 nm, or from 75 nm to about 125 nm. In some embodiments, LNP encapsulated nucleic acid, expression vector or AAV vector may have a size in a range from about 10 nm to 500 nm.

Recombinant cells capable of expressing the GAA sequences of the invention can be used for delivery or administration.

Naked DNA such as minicircles and transposons can be used for administration or delivery or lentiviral vectors. Additionally, gene editing technologies such as zinc finger nucleases, meganucleases, TALENs, and CRISPR can also be used to deliver the coding sequence of the invention.

A glycogen storage disease (GSD) results from absence of an enzyme that ultimately converts glycogen compounds to glucose. Enzyme deficiency results in glycogen accumulation in tissues. In many cases, the defect has systemic consequences, but, in some cases, the defect is limited to specific tissues. Most patients experience muscle symptoms, such as weakness and cramps, although certain GSDs manifest as specific syndromes, such as hypoglycemic seizures or cardiomegaly.

The following are non-limiting examples of GSDs:
0—Glycogen synthase deficiency; Ia—Glucose—6-phosphatase deficiency (von Gierke disease); II—Acid maltase deficiency (Pompe disease); III—Debranching enzyme deficiency (Forbes—Cori disease); IV—Transglucosidase deficiency (Andersen disease, amylopectinosis); V—Myophosphorylase deficiency (McArdle disease); VI—Phosphorylase deficiency (Hers disease); and VII—Phosphofructokinase deficiency (Tarui disease).

Various forms of GSD affect metabolic carbohydrate pathways. Although at least 14 unique GSDs are discussed in the literature, the 4 that cause clinically significant muscle weakness are Pompe disease (GSD type II, acid maltase deficiency), Cori disease (GSD type IIIa, debranching enzyme deficiency), McArdle disease (GSD type V, myophosphorylase deficiency), and Tarui disease (GSD type VII, phosphofructokinase deficiency). One form, Von Gierke disease (GSD type Ia, glucose-6-phosphatase deficiency), causes clinically significant end-organ disease with significant morbidity.

In general, GSDs are inherited as autosomal recessive conditions. These inherited enzyme defects usually present in childhood, although some, such as McArdle disease and Pompe disease, have separate adult-onset forms.

GSDs can be treated by enzyme replacement therapy (ERT), for example, with recombinantly produced GAA. Enzyme replacement therapy is an approved treatment for all patients with Pompe disease. It involves the intravenous administration of recombinant human acid α-glucosidase. This treatment, manufactured by Genzyme, a Sanofi Corporation, is Lumizyme (marketed as Myozyme outside the United States), and was first approved by the U.S. Food and Drug Administration (FDA) in 2006. It has been approved for all patients with Pompe disease. The benefits of ERT may be attenuated by antibody formation, so ERT can also be combined with immune expression.

GSDs can be treated by diet therapy, involving meticulous adherence to a dietary regimen, may reduce liver size, prevent hypoglycemia, allow for reduction in symptoms, and allow for growth and development.

Additional treatment of Pompe disease is symptomatic and supportive. Respiratory support may be required, as most patients have some degree of respiratory compromise and/or respiratory failure. Physical therapy may be helpful to strengthen respiratory muscles. Some patients may need respiratory assistance through mechanical ventilation (i.e., bipap or volume ventilators) during the night and/or periods of the day. In addition, it may be necessary for additional support during respiratory tract infections. Mechanical ventilation support can be through noninvasive or invasive techniques. The decision about the duration of respiratory support is best made by the family in careful consultation with the patient's physicians and other members of the healthcare team based upon the specifics of the patient. A high-protein diet may be beneficial in the noninfantile form of Pompe.

Physiotherapy is recommended to improve strength and physical ability. Occupational therapy, including the use of canes or walkers, may be necessary. Eventually, some individuals may require the use of a wheelchair. Speech therapy can be beneficial to improve articulation and speech for some patients.

Orthopedic devices including braces may be recommended for some patients. Surgery may be required for certain orthopedic symptoms such as contractures or spinal deformity.

Since Pompe disease can weaken muscles used for chewing and swallowing, measures may be required to ensure proper nutrition and weight gain. Some patients may need specialized, high-calorie diets and may need to learn techniques to change the size and texture of food to lower the risk of aspiration. Some infants may require the insertion of a feeding tube that is run through the nose, down the esophagus and into the stomach (nasogastric tube). In some children, a feeding tube may need to be inserted directly into the stomach through a small surgical opening in the abdominal wall. Some individuals with late onset Pompe disease may require a soft diet, but few require feeding tubes.

Subjects can be tested for one or more liver enzymes for an adverse response to treatment or to determine if such subjects, pretreatment, are appropriate for treatment according to a method of the invention. Candidate subjects can therefore be screened for amounts of one or more liver enzymes prior to or after treatment according to a method of the invention. Treated subjects can be monitored after treatment for elevated liver enzymes, periodically, e.g., every 1-4 weeks, 1-6 months, 6-12 months, or 1, 2, 3, 4, 5 or more years.

Exemplary liver enzymes include alanine aminotransferase (ALT), aspartate aminotransferase (AST), and lactate dehydrogenase (LDH), but other enzymes indicative of liver damage can also be monitored. A normal level of these enzymes in the circulation is typically defined as a range that has an upper level, above which the enzyme level is considered elevated, and therefore indicative of liver damage. A normal range depends in part on the standards used by the clinical laboratory conducting the assay.

The invention provides kits with packaging material and one or more components therein. A kit typically includes a label or packaging insert including a description of the components or instructions for use in vitro, in vivo, or ex vivo, of the components therein. A kit can contain a collection of such components, e.g., a rAAV particle and optionally a second active, such as another compound, agent, drug or composition.

A kit refers to a physical structure housing one or more components of the kit. Packaging material can maintain the components sterilely, and can be made of material commonly used for such purposes (e.g., paper, corrugated fiber, glass, plastic, foil, ampules, vials, tubes, etc.).

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer, lot numbers, manufacture location and date, expiration dates. Labels or inserts can include information identifying manufacturer information, lot numbers, manufacturer location and date. Labels or inserts can include information on a disease for which a kit component may be used. Labels or inserts can include instructions for the clinician or subject for using one or more of the kit components in a method, use, or treatment protocol or therapeutic regimen. Instructions can include dosage amounts, frequency or duration, and instructions for practicing any of the methods, uses, treatment protocols or prophylactic or therapeutic regimes described herein.

Labels or inserts can include information on any benefit that a component may provide, such as a prophylactic or therapeutic benefit. Labels or inserts can include information on potential adverse side effects, complications or reactions, such as warnings to the subject or clinician regarding situations where it would not be appropriate to use a particular composition. Adverse side effects or complications could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a bar-coded printed label, a disk, optical disk such as CD- or DVD-ROM/RAM, DVD, MP3, magnetic tape, or an electrical storage media such as RAM and ROM or hybrids of these such as magnetic/optical storage media, FLASH media or memory type cards.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All patents, patent applications, publications, and other references, GenBank citations and ATCC citations cited herein are incorporated by reference in their entirety. In case of conflict, the specification, including definitions, will control.

All of the features disclosed herein may be combined in any combination. Each feature disclosed in the specification may be replaced by an alternative feature serving a same, equivalent, or similar purpose. Thus, unless expressly stated otherwise, disclosed features (e.g., modified nucleic acids encoding GAA, expression cassettes comprising modified nucleic acids encoding GAA, and rAAV particles comprising the modified nucleic acids encoding GAA) are an example of a genus of equivalent or similar features.

As used herein, the singular forms “a”, “and,” and “the” include plural referents unless the context clearly indicates otherwise. Thus, for example, reference to “a nucleic acid” includes a plurality of such nucleic acids, reference to “a vector” includes a plurality of such vectors, and reference to “a virus” or “particle” includes a plurality of such viruses/particles.

As used herein, all numerical values or numerical ranges include integers within such ranges and fractions of the values or the integers within ranges unless the context clearly indicates otherwise. Thus, to illustrate, reference to 86% or more identity, includes 86%, 87%, 88%, 89%, 90%, 91%, 92%, 93%, 94%, 95%, 96%, 97%, 98%, 99%, 100% etc., as well as 86.1%, 86.2%, 86.3%, 86.4%, 86.5%, etc., 87.1%, 88.2%, 88.3%, 88.4%, 88.5%, etc., and so forth.

Reference to an integer with more (greater) or less than includes any number greater or less than the reference number, respectively. Thus, for example, a reference to less than 127, includes 126, 125, 124, 123, 122, 121, 120, 119, 118, 117, 116, 115, 114, 113, 112, 111, 110, etc. all the way down to zero (0); and less than 10, includes 9, 8, 7, etc. all the way down to zero (0).

As used herein, all numerical values or ranges include sub ranges and fractions of the values and integers within such ranges and sub ranges and the wrong 1 as well as the file okay thanks fractions of the integers within such ranges unless the context clearly indicates otherwise. Thus, to illustrate, reference to a numerical range, such as 1-10 includes 1-2, 1-3, 1-4, 1-5, 1-6, 1-7, 1-8, 1-9, 2-3, 2-4, 2-5, 2-6, 2-7, 2-8, 2-9, 2-10, 3
4, 3-5, 3-6, 3-7, 3-8, 3-9, 3-10, etc.; and 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-850, includes ranges of 1-20, 1-30, 1-40, 1-50, 1-60, 10-30, 10-40, 10-50, 10-60, 10-70, 10-80, 20-40, 20-50, 20-60, 20-70, 20-80, 20-90, 50-75, 50-100, 50-150, 50-200, 50-250, 100-200, 100-250, 100-300, 100-350, 100-400, 100-500, 150-250, 150-300, 150-350, 150-400, 150-450, 150-500, etc.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in certain embodiments or aspects of the invention, materials and/or method steps are excluded. Thus, even though the invention is generally not expressed herein in terms of what the invention does not include aspects that are not expressly excluded in the invention are nevertheless disclosed herein.

A number of embodiments of the invention have been described. Nevertheless, one skilled in the art, without departing from the spirit and scope of the invention, can make various changes and modifications of the invention to adapt it to various usages and conditions. Accordingly, the following examples are intended to illustrate but not limit the scope of the invention claimed in any way.

### Example 1

TABLE 1
GAA Expression Cassette Overview

| | SEQ | Codon | BGH | Packaged into |
|---|---|---|---|---|
| Expression Cassette | ID | Optimized | PolyA | (SEQ ID NOs: |
| (SEQ ID NOs: 16-24) | NO | Variant | Signal | 30-32) Vector |
| pAAV-ApoE/ | 16 | GAA13 | CpG- | SPK-AAV-01 |
| hAAT.fixUTR.GAA13.BGH | | | reduced | |
| pAAV-ApoE/ | 17 | GAA13 | CpG- | SPK-AAV-02 |
| hAAT.GAA13.BGH | | | reduced | |
| pAAV-ApoE/ | 18 | GAA7 | CpG- | SPK-AAV-03 |
| hAAT.GAA7.BGH | | | reduced | |
| pAAV-ApoE/ | 19 | GAA8 | CpG- | SPK-AAV-04 |
| hAAT.GAA8.BGH | | | reduced | |
| pAAV-ApoE/ | 20 | GAA2 | wtBGH | SPK-AAV-05 |
| hAAT.GAA2.wtBGH | | | | |
| pAAV-ApoE/ | 21 | GAA5 | wtBGH | SPK-AAV-06 |
| hAAT.GAA5.wtBGH | | | | |
| pAAV-ApoE/ | 22 | GAA7 | wtBGH | SPK-AAV-07 |
| hAAT.GAA7.wtBGH | | | | |
| pAAV-ApoE/ | 23 | GAA8 | wtBGH | SPK-AAV-08 |
| hAAT.GAA8.wtBGH | | | | |
| pAAV-ApoE/ | 24 | GAA13 | wtBGH | SPK-AAV-09 |
| hAAT.GAA13.wtBGH | | | | |
| pAAV-ApoE/ | 17 | GAA13 | CpG- | SPK-AAV-10* |
| hAAT.GAA13.BGH | | | reduced | |

*SPK-AAV-10 is packaged in AAV6 capsid and all the others are packaged in AAV-4-1 capsid variant, described in International Patent Application publication WO 2016/210170.

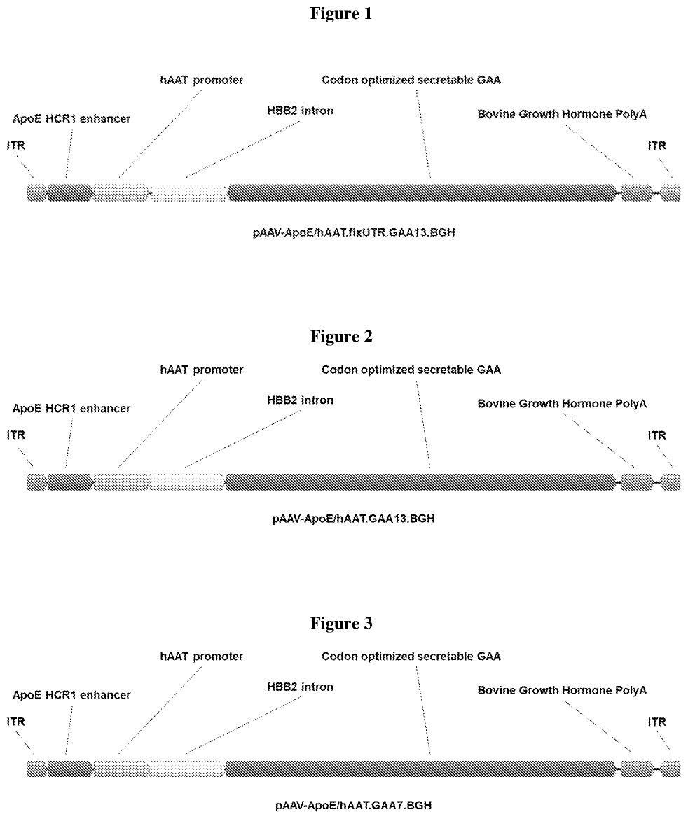

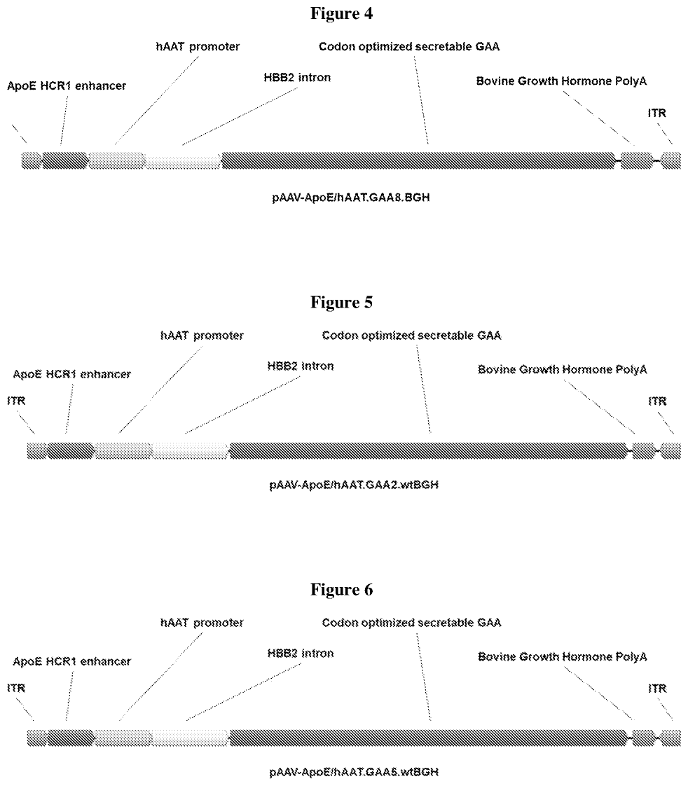

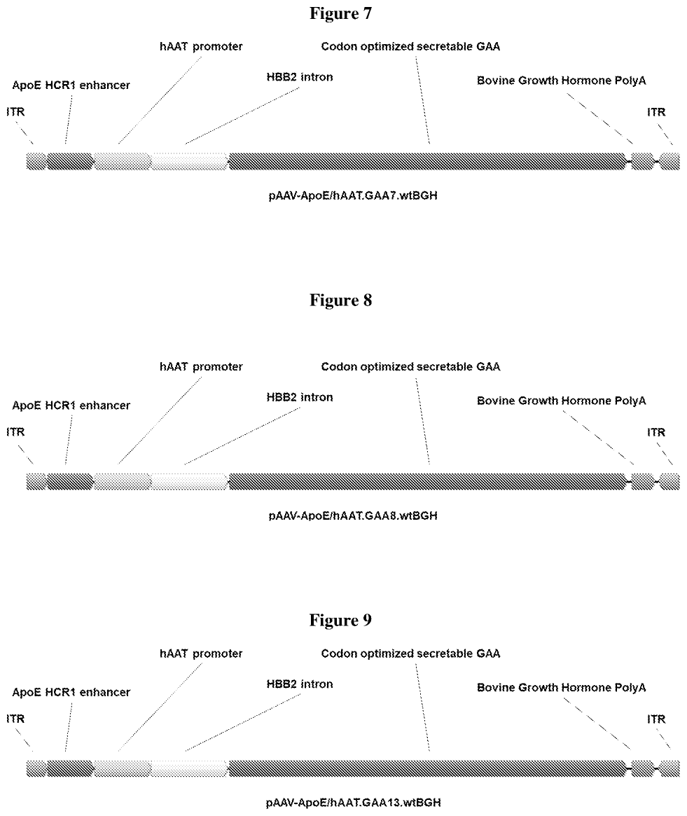

GAA expression cassettes are shown in FIGS. 1 through 9. All contain 5′ and 3′ flanking AAV inverted terminal repeats (ITRs), a liver-specific ApoE/hAAT enhancer/promoter sequence operably linked to an optimized GAA coding sequence, including a human hemoglobin subunit beta (HBB2) intron, followed by a wild type or CpG reduced bovine growth hormone (bGH) polyadenylation (poly A) sequence.

SEQ ID NO: 1: nucleic acid sequence for GAA2
ATGGCTTTCCTGTGGCTGCTGAGCTGCTGGGCTCTGCTGGGCACCACCTTTGGGCTGCTGGTGCCTAGGGAGCTG
TCTGGGTCTAGCCCTGTGCTGGAGGAGACTCACCCTGCCCATCAGCAGGGGGCTAGCAGGCCTGGCCCCAGGGAT
GCTCAGGCCCACCCTGGCAGGCCCAGGGCTGTGCCCACCCAGTGTGATGTGCCCCCCAACAGCAGGTTTGACTGT
GCCCCTGACAAGGCCATTACCCAGGAGCAGTGTGAGGCCAGGGGCTGCTGCTACATTCCAGCTAAGCAGGGCCTG
CAGGGGGCCCAGATGGGCCAGCCCTGGTGCTTCTTCCCCCCCAGCTATCCTAGCTATAAACTGGAGAACCTGAGC
AGCTCTGAGATGGGCTATACTGCCACCCTGACTAGGACTACTCCCACCTTTTTTCCTAAGGATATCCTGACCCTG
AGGCTGGATGTGATGATGGAGACTGAGAACAGGCTGCACTTCACTATTAAGGACCCTGCCAATAGGAGGTATGAA
GTGCCTCTGGAGACTCCTCATGTGCACTCTAGGGCCCCCAGCCCCCTGTATTCTGTGGAGTTCTCTGAGGAGCCC
TTTGGGGTGATTGTGAGGAGGCAGCTGGATGGCAGGGTGCTGCTGAACACCACTGTGGCCCCCCTGTTCTTTGCT
GACCAGTTCCTGCAGCTGAGCACCAGCCTGCCCAGCCAGTACATCACTGGGCTGGCTGAGCATCTGAGCCCTCTG
ATGCTGAGCACCTCTTGGACCAGGATCACCCTGTGGAATAGGGATCTGGCCCCCACCCCTGGGGCTAATCTGTAT
GGCTCTCATCCCTTTTACCTGGCCCTGGAGGATGGGGGCTCTGCCCATGGGGTGTTTCTGCTGAACAGCAATGCC
ATGGATGTGGTGCTGCAGCCCTCTCCTGCCCTGAGCTGGAGGAGCACTGGGGGCATCCTGGATGTGTACATCTTC
CTGGGCCCTGAGCCCAAGTCTGTGGTCCAGCAGTATCTGGATGTGGTGGGCTACCCCTTTATGCCCCCCTATTGG
GGCCTGGGCTTCCACCTGTGCAGGTGGGGGTATTCTTCTACTGCTATCACCAGGCAGGTGGTGGAGAACATGACC
AGGGCTCACTTCCCCCTGGATGTGCAGTGGAATGACCTGGACTATATGGACTCTAGGAGGGATTTCACCTTCAAC
AAGGATGGCTTCAGGGACTTCCCTGCTATGGTCCAGGAGCTGCATCAGGGGGGCAGGAGGTACATGATGATTGTG
GACCCTGCCATCAGCAGCTCTGGCCCTGCTGGCAGCTATAGGCCCTATGATGAGGGCCTGAGGAGGGGGGTGTTT
ATCACTAATGAAACTGGGCAGCCCCTGATTGGCAAGGTGTGGCCTGGCTCTACTGCCTTCCCTGACTTCACCAAC
CCCACTGCTCTGGCCTGGTGGGAGGACATGGTGGCTGAGTTCCATGACCAGGTGCCTTTTGATGGCATGTGGATT
GACATGAATGAGCCCAGCAACTTCATCAGGGGCTCTGAGGATGGGTGCCCCAATAATGAGCTGGAGAACCCCCCC
TATGTGCCTGGGGTGGTGGGGGGCACCCTGCAGGCTGCCACTATTTGTGCCAGCTCTCACCAGTTCCTGAGCACC
CACTACAACCTGCACAATCTGTATGGCCTGACTGAGGCCATTGCCAGCCACAGGGCCCTGGTGAAGGCCAGGGGC
ACTAGGCCCTTTGTGATCTCTAGAAGCACCTTTGCTGGCCATGGGAGGTATGCTGGCCACTGGACTGGGGATGTG
TGGAGCTCTTGGGAGCAGCTGGCCAGCTCTGTGCCTGAGATCCTGCAGTTCAACCTGCTGGGGGTGCCCCTGGTG
GGGGCTGATGTGTGTGGCTTCCTGGGCAACACCTCTGAAGAGCTGTGTGTGAGGTGGACCCAGCTGGGGGCCTTC
TACCCTTTCATGAGGAACCACAACAGCCTGCTGAGCCTGCCTCAGGAGCCTTACTCTTTCTCTGAGCCTGCCCAG
CAGGCCATGAGGAAGGCCCTGACCCTGAGGTATGCTCTGCTGCCCCACCTGTACACCCTGTTCCACCAGGCCCAT
GTGGCTGGGGAGACTGTGGCCAGGCCCCTGTTCCTGGAGTTTCCTAAGGATAGCAGCACCTGGACTGTGGACCAC
CAGCTGCTGTGGGGGGAGGCCCTGCTGATTACCCCTGTGCTGCAGGCTGGCAAGGCTGAGGTGACTGGCTACTTC
CCCCTGGGCACTTGGTATGACCTGCAGACTGTGCCTGTGGAAGCCCTGGGCAGCCTGCCTCCCCCCCCTGCTGCC
CCCAGGGAGCCTGCCATCCACTCTGAGGGCCAGTGGGTGACCCTGCCTGCCCCCCTGGACACCATTAATGTGCAT
CTGAGGGCTGGGTATATTATCCCCCTGCAGGGGCCTGGGCTGACTACCACTGAGAGCAGGCAGCAGCCTATGGCC
CTGGCTGTGGCTCTGACTAAGGGGGGGGAGGCCAGGGGGGAGCTGTTCTGGGATGATGGGGAGAGCCTGGAGGTG
CTGGAGAGGGGGGCCTACACCCAGGTGATTTTCCTGGCCAGGAACAACACCATTGTGAATGAGCTGGTGAGGGTG
ACCTCTGAGGGGGCTGGCCTGCAGCTGCAGAAAGTGACTGTGCTGGGGGTGGCCACTGCCCCCCAGCAGGTGCTG
AGCAATGGGGTGCCTGTGAGCAACTTCACCTACAGCCCTGACACCAAGGTGCTGGATATTTGTGTGAGCCTGCTG
ATGGGGGAGCAGTTCCTGGTGAGCTGGTGCTGA
SEQ ID NO: 2: nucleic acid sequence for GAA5
ATGGCTTTCCTGTGGCTGCTGTCTTGCTGGGCCCTGCTGGGGACTACCTTTGGCCTGCTGGTGCCCAGGGAACTG
TCTGGCTCTAGCCCAGTGCTGGAGGAGACCCACCCTGCCCACCAGCAGGGGGCTTCTAGGCCTGGCCCCAGGGAT
GCCCAGGCCCACCCTGGCAGGCCAAGGGCTGTGCCCACCCAGTGTGATGTGCCCCCCAACTCTAGATTTGATTGT
GCCCCTGATAAGGCCATCACCCAGGAGCAGTGTGAGGCTAGGGGCTGCTGCTACATCCCTGCTAAGCAGGGCCTG
CAGGGGGCTCAGATGGGCCAGCCCTGGTGCTTCTTCCCCCCCAGCTATCCCTCTTACAAGCTGGAGAATCTGAGC
AGCTCTGAGATGGGCTACACTGCCACCCTGACCAGGACTACTCCCACCTTCTTCCCCAAGGACATCCTGACCCTG
AGGCTGGATGTGATGATGGAGACTGAGAACAGGCTGCATTTCACCATCAAGGATCCTGCCAACAGGAGGTATGAG
GTGCCTCTGGAGACCCCCCATGTGCACAGCAGGGCTCCTTCTCCCCTGTACTCTGTGGAGTTCTCTGAGGAACCC
TTTGGGGTGATTGTGAGGAGGCAGCTGGATGGCAGGGTCCTGCTGAACACCACTGTGGCCCCCCTGTTCTTTGCT
GATCAGTTCCTGCAGCTGTCCACTTCTCTGCCTAGCCAGTACATCACTGGGCTGGCTGAGCACCTGAGCCCTCTG
ATGCTGAGCACCTCTTGGACTAGGATCACCCTGTGGAACAGGGACCTGGCCCCCACCCCTGGGGCCAACCTGTAT
GGCAGCCACCCCTTCTATCTGGCCCTGGAGGATGGGGGCTCTGCCCATGGGGTGTTCCTGCTGAATAGCAATGCT
ATGGATGTGGTGCTGCAGCCCAGCCCTGCCCTGTCTTGGAGGAGCACTGGGGGCATCCTGGATGTGTACATTTTC
CTGGGGCCTGAGCCCAAGTCTGTGGTGCAGCAGTACCTGGATGTGGTGGGCTACCCCTTCATGCCTCCCTACTGG
GGCCTGGGCTTCCACCTGTGCAGGTGGGGCTACAGCTCTACTGCCATCACCAGGCAGGTGGTGGAGAATATGACC
AGGGCCCACTTCCCCCTGGATGTGCAGTGGAATGACCTGGACTACATGGACTCTAGGAGGGACTTCACCTTCAAT
AAGGATGGCTTCAGAGACTTCCCTGCCATGGTGCAGGAGCTGCATCAGGGGGGCAGGAGGTACATGATGATTGTG
GACCCTGCCATCAGCTCTTCTGGCCCTGCTGGCTCTTACAGGCCCTATGATGAGGGCCTGAGGAGGGGGGTGTTC
ATCACCAATGAGACTGGGCAGCCCCTGATTGGGAAGGTGTGGCCTGGCTCTACTGCCTTCCCTGACTTCACCAAT
CCTACTGCCCTGGCCTGGTGGGAGGACATGGTGGCTGAGTTCCATGACCAGGTGCCCTTTGATGGCATGTGGATT
GACATGAATGAGCCCTCTAATTTCATCAGGGGCTCTGAGGATGGCTGCCCCAACAATGAGCTGGAGAACCCCCCC
TATGTGCCTGGGGTGGTGGGGGGCACCCTGCAGGCTGCCACCATCTGTGCTAGCTCTCACCAGTTCCTGAGCACC
CACTACAATCTGCATAACCTGTATGGCCTGACTGAGGCCATTGCCAGCCACAGGGCCCTGGTGAAGGCTAGGGGC
ACCAGGCCCTTTGTGATTTCTAGGAGCACTTTTGCTGGCCATGGCAGGTATGCTGGGCACTGGACTGGGGATGTG
TGGTCTAGCTGGGAGCAGCTGGCTTCTTCTGTGCCTGAGATCCTGCAGTTCAACCTGCTGGGGGTGCCTCTGGTG
GGGGCTGATGTGTGTGGGTTCCTGGGCAACACTTCTGAGGAGCTGTGTGTGAGGTGGACCCAGCTGGGGGCCTTC
TACCCTTTCATGAGGAACCACAACAGCCTGCTGAGCCTGCCCCAGGAGCCCTACAGCTTCTCTGAGCCTGCCCAG
CAGGCCATGAGGAAGGCCCTGACCCTGAGGTATGCCCTGCTGCCCCACCTGTACACCCTGTTCCACCAGGCCCAT
GTGGCTGGGGAGACTGTGGCTAGGCCTCTGTTCCTGGAGTTCCCCAAGGACTCTAGCACCTGGACTGTGGACCAC
CAGCTGCTGTGGGGGGAGGCCCTGCTGATCACTCCTGTGCTGCAGGCTGGGAAGGCTGAGGTGACTGGCTATTTC
CCCCTGGGCACCTGGTATGACCTGCAGACTGTGCCTGTGGAGGCCCTGGGGAGCCTGCCCCCCCCCCCTGCTGCC
CCCAGGGAGCCTGCCATCCACTCTGAGGGCCAGTGGGTGACCCTGCCTGCCCCTCTGGATACCATCAATGTGCAC
CTGAGGGCTGGCTACATCATTCCCCTGCAGGGCCCTGGCCTGACCACTACTGAGTCTAGGCAGCAGCCCATGGCC
CTGGCTGTGGCCCTGACCAAGGGGGGGGAGGCTAGGGGGGAGCTGTTTTGGGATGATGGGGAGAGCCTGGAGGTG
CTGGAGAGGGGGGCCTACACTCAGGTGATCTTCCTGGCCAGGAACAATACCATTGTGAATGAGCTGGTGAGGGTG
ACCTCTGAGGGGGCTGGCCTGCAGCTGCAGAAGGTGACTGTGCTGGGGGTGGCCACTGCCCCCCAGCAGGTGCTG
AGCAATGGGGTGCCTGTGAGCAACTTCACCTATAGCCCTGATACCAAGGTGCTGGATATTTGTGTGAGCCTGCTG
ATGGGGGAGCAGTTCCTGGTGAGCTGGTGCTGA
SEQ ID NO: 3: nucleic acid sequence for GAA7
ATGGCTTTCCTGTGGCTGCTGTCTTGTTGGGCTCTGCTGGGCACCACCTTTGGCCTGCTGGTGCCCAGGGAGCTG
TCTGGCAGCAGCCCTGTGCTGGAGGAGACCCACCCTGCTCATCAGCAGGGGGCTAGCAGGCCTGGCCCCAGGGAT
GCCCAGGCTCACCCTGGGAGACCCAGGGCTGTGCCCACTCAGTGTGATGTGCCCCCCAACAGCAGGTTTGACTGT
GCTCCTGACAAGGCTATCACCCAGGAGCAGTGTGAGGCCAGGGGGTGCTGCTACATTCCTGCTAAGCAGGGCCTG
CAGGGGGCCCAGATGGGCCAGCCCTGGTGCTTCTTCCCCCCCTCTTATCCCAGCTATAAGCTGGAGAACCTGAGC
AGCTCTGAGATGGGCTACACTGCCACCCTGACCAGGACCACTCCCACCTTCTTTCCCAAGGATATTCTGACTCTG
AGGCTGGATGTGATGATGGAGACTGAGAACAGGCTGCACTTCACTATCAAGGACCCTGCCAATAGGAGGTATGAG
GTGCCCCTGGAGACTCCTCATGTGCATAGCAGGGCCCCTTCTCCTCTGTATTCTGTGGAGTTCTCTGAGGAGCCC
TTTGGGGTGATTGTGAGGAGGCAGCTGGATGGCAGGGTGCTGCTGAACACCACTGTGGCCCCCCTGTTCTTTGCT
GACCAGTTCCTGCAGCTGAGCACTTCTCTGCCCAGCCAGTACATTACTGGGCTGGCTGAGCATCTGAGCCCCCTG
ATGCTGAGCACCTCTTGGACCAGGATCACCCTGTGGAACAGGGACCTGGCCCCCACTCCTGGGGCTAACCTGTAT
GGCTCTCACCCCTTTTACCTGGCCCTGGAGGATGGGGGCTCTGCCCATGGGGTGTTTCTGCTGAACAGCAATGCT
ATGGATGTGGTGCTGCAGCCCTCTCCAGCCCTGTCTTGGAGGAGCACTGGGGGCATTCTGGATGTGTACATTTTC
CTGGGGCCTGAACCCAAGTCTGTGGTGCAGCAGTACCTGGATGTGGTGGGCTACCCCTTCATGCCCCCCTATTGG
GGGCTGGGGTTTCACCTGTGCAGGTGGGGCTACAGCAGCACTGCCATCACCAGGCAGGTGGTGGAGAACATGACC
AGGGCCCATTTCCCCCTGGATGTGCAGTGGAATGACCTGGACTACATGGATAGCAGGAGGGATTTCACCTTCAAC
AAGGATGGCTTCAGGGACTTTCCTGCCATGGTGCAGGAGCTGCACCAGGGGGGCAGGAGGTATATGATGATTGTG
GACCCTGCTATCAGCAGCTCTGGCCCTGCTGGCTCTTACAGGCCCTATGATGAGGGCCTGAGGAGGGGGGTGTTT
ATCACTAATGAAACTGGCCAGCCTCTGATTGGCAAGGTCTGGCCTGGCTCTACTGCCTTCCCTGATTTTACTAAC
CCCACTGCCCTGGCCTGGTGGGAGGACATGGTGGCTGAGTTCCATGATCAGGTGCCTTTTGATGGCATGTGGATT
GATATGAATGAACCAAGCAACTTCATCAGAGGCTCTGAGGATGGCTGCCCCAACAATGAGCTGGAGAACCCCCCC
TATGTGCCTGGGGTGGTGGGGGGCACTCTGCAGGCTGCCACCATTTGTGCTAGCAGCCACCAGTTCCTGAGCACC
CACTACAATCTGCACAACCTGTATGGCCTGACTGAAGCCATTGCCAGCCATAGGGCCCTGGTGAAGGCCAGGGGC
ACTAGGCCTTTTGTGATCAGCAGGAGCACTTTTGCTGGCCATGGCAGGTATGCTGGCCACTGGACTGGGGATGTG
TGGAGCAGCTGGGAGCAGCTGGCCAGCTCTGTGCCTGAGATTCTGCAGTTTAACCTGCTGGGGGTGCCCCTGGTG
GGGGCTGATGTGTGTGGCTTCCTGGGCAACACCTCTGAGGAGCTGTGTGTGAGGTGGACCCAGCTGGGGGCCTTT
TATCCCTTCATGAGGAACCACAACAGCCTGCTGAGCCTGCCTCAGGAGCCCTACTCTTTCTCTGAGCCTGCCCAG
CAGGCCATGAGGAAGGCCCTGACCCTGAGGTATGCCCTGCTGCCCCACCTGTATACCCTGTTCCACCAGGCCCAT
GTGGCTGGGGAGACTGTGGCCAGGCCCCTGTTCCTGGAGTTCCCCAAGGACAGCAGCACCTGGACTGTGGATCAT
CAGCTGCTGTGGGGGGAGGCCCTGCTGATCACCCCTGTGCTGCAGGCTGGCAAGGCTGAGGTCACTGGCTACTTC
CCTCTGGGCACCTGGTATGACCTGCAGACTGTGCCTGTGGAGGCTCTGGGCAGCCTGCCCCCCCCCCCTGCTGCT
CCCAGGGAGCCTGCCATCCACTCTGAGGGCCAGTGGGTGACCCTGCCTGCTCCCCTGGACACCATCAATGTGCAC
CTGAGGGCTGGCTACATTATCCCCCTGCAGGGCCCAGGGCTGACTACCACTGAGAGCAGACAGCAGCCCATGGCT
CTGGCTGTGGCCCTGACCAAGGGGGGGGAAGCTAGGGGGGAGCTGTTCTGGGATGATGGGGAGAGCCTGGAGGTG
CTGGAGAGGGGGGCCTATACCCAGGTGATCTTCCTGGCTAGGAACAACACCATTGTCAATGAGCTGGTGAGGGTG
ACTTCTGAGGGGGCTGGGCTGCAGCTGCAGAAGGTGACTGTGCTGGGGGTGGCCACTGCTCCCCAGCAGGTGCTG
AGCAATGGGGTGCCTGTGAGCAACTTCACCTACAGCCCTGACACCAAGGTGCTGGACATCTGTGTGTCTCTGCTG
ATGGGGGAGCAGTTCCTGGTGAGCTGGTGCTGA
SEQ ID NO: 4: nucleic acid sequence for GAA8
ATGGCCTTCCTGTGGCTGCTGTCTTGCTGGGCTCTGCTGGGGACCACCTTTGGCCTGCTGGTCCCCAGGGAGCTG
TCTGGCTCTTCTCCTGTCCTGGAGGAGACCCACCCTGCCCACCAGCAGGGGGCTAGCAGGCCTGGCCCCAGGGAT
GCCCAGGCCCACCCTGGCAGGCCCAGGGCTGTGCCCACCCAGTGTGATGTGCCTCCCAACAGCAGGTTTGACTGT
GCCCCTGACAAGGCCATCACCCAGGAGCAGTGTGAGGCCAGGGGCTGCTGCTATATCCCTGCCAAGCAGGGCCTG
CAGGGGGCTCAGATGGGCCAGCCCTGGTGCTTCTTTCCCCCCTCTTATCCTAGCTATAAGCTGGAGAACCTGAGC
AGCTCTGAGATGGGGTACACTGCCACCCTGACCAGGACCACCCCCACTTTCTTCCCTAAGGACATCCTGACCCTG
AGGCTGGATGTGATGATGGAGACTGAGAATAGGCTGCACTTTACTATCAAGGACCCTGCCAACAGGAGGTATGAG
GTGCCTCTGGAGACCCCCCATGTGCATTCTAGGGCCCCCAGCCCCCTGTACTCTGTGGAGTTCTCTGAGGAGCCC
TTTGGGGTGATTGTGAGGAGACAGCTGGATGGCAGGGTCCTGCTGAACACCACTGTGGCTCCCCTGTTTTTTGCT
GACCAGTTCCTGCAGCTGAGCACCAGCCTGCCCAGCCAGTACATCACTGGGCTGGCTGAGCACCTGAGCCCCCTG
ATGCTGAGCACCAGCTGGACCAGGATCACCCTGTGGAACAGGGATCTGGCTCCTACCCCTGGGGCCAACCTGTAT
GGCTCTCACCCCTTTTACCTGGCCCTGGAGGATGGGGGCTCTGCCCATGGGGTGTTCCTGCTGAACAGCAATGCT
ATGGATGTGGTGCTGCAGCCCAGCCCTGCCCTGAGCTGGAGGTCTACTGGGGGCATCCTGGATGTGTACATCTTT
CTGGGGCCTGAGCCCAAGTCTGTGGTGCAGCAGTACCTGGATGTGGTGGGCTATCCTTTTATGCCCCCCTATTGG
GGCCTGGGCTTCCACCTGTGCAGGTGGGGCTACAGCAGCACTGCCATCACCAGACAGGTGGTGGAGAACATGACC
AGGGCCCACTTCCCCCTGGATGTGCAGTGGAATGACCTGGACTACATGGACAGCAGGAGGGACTTCACCTTTAAC
AAGGATGGCTTTAGGGACTTCCCTGCCATGGTGCAGGAGCTGCATCAGGGGGGCAGGAGGTACATGATGATTGTG
GACCCAGCCATCAGCAGCTCTGGGCCTGCTGGGTCTTACAGGCCCTATGATGAGGGCCTGAGGAGGGGGGTGTTC
ATCACCAATGAGACTGGCCAGCCCCTGATTGGCAAGGTGTGGCCTGGGAGCACTGCCTTCCCTGATTTTACCAAC
CCCACTGCCCTGGCCTGGTGGGAGGATATGGTGGCTGAGTTTCATGACCAGGTGCCCTTTGATGGCATGTGGATT
GACATGAATGAGCCCAGCAATTTCATCAGGGGCTCTGAGGATGGCTGCCCCAACAATGAGCTGGAGAATCCTCCC
TATGTGCCTGGGGTGGTGGGGGGCACCCTGCAGGCTGCCACCATCTGTGCCTCTAGCCACCAGTTCCTGAGCACC
CACTATAACCTGCATAACCTGTATGGCCTGACTGAGGCCATTGCCAGCCATAGAGCCCTGGTGAAGGCCAGAGGG
ACCAGGCCCTTTGTGATCTCTAGGAGCACCTTTGCTGGCCATGGCAGGTATGCTGGCCACTGGACTGGGGATGTG
TGGAGCTCTTGGGAGCAGCTGGCCAGCTCTGTGCCAGAGATCCTGCAGTTCAACCTGCTGGGGGTGCCTCTGGTG
GGGGCTGATGTGTGTGGCTTCCTGGGCAATACCTCTGAAGAGCTGTGTGTGAGGTGGACTCAGCTGGGGGCCTTC
TATCCCTTCATGAGGAACCACAACAGCCTGCTGTCTCTGCCCCAGGAGCCCTACAGCTTCTCTGAGCCTGCTCAG
CAGGCTATGAGGAAGGCCCTGACCCTGAGGTATGCCCTGCTGCCCCATCTGTACACCCTGTTCCACCAGGCCCAT
GTGGCTGGGGAGACTGTGGCCAGGCCCCTGTTTCTGGAGTTTCCCAAGGACAGCAGCACCTGGACTGTGGACCAT
CAGCTGCTGTGGGGGGAGGCTCTGCTGATTACCCCTGTGCTGCAGGCTGGCAAGGCTGAGGTGACTGGGTACTTC
CCCCTGGGGACTTGGTATGACCTGCAGACTGTGCCTGTGGAAGCTCTGGGCAGCCTGCCCCCACCCCCTGCTGCC
CCTAGGGAGCCTGCCATCCACTCTGAGGGCCAGTGGGTGACCCTGCCTGCCCCTCTGGACACCATCAATGTGCAC
CTGAGGGCTGGCTATATCATCCCCCTGCAGGGCCCTGGGCTGACCACCACTGAGAGCAGGCAGCAGCCCATGGCC
CTGGCTGTGGCCCTGACTAAGGGGGGGGAGGCCAGGGGGGAGCTGTTCTGGGATGATGGGGAGAGCCTGGAGGTG
CTGGAGAGAGGGGCCTACACCCAGGTGATCTTTCTGGCCAGGAACAACACCATTGTGAATGAGCTGGTGAGGGTG
ACTTCTGAGGGGGCTGGCCTGCAGCTGCAGAAGGTGACTGTGCTGGGGGTGGCCACTGCCCCCCAGCAGGTGCTG
AGCAATGGGGTGCCTGTGTCTAACTTCACCTACAGCCCTGATACTAAGGTGCTGGATATCTGTGTGAGCCTGCTG
ATGGGGGAGCAGTTTCTGGTGAGCTGGTGCTGA
SEQ ID NO: 5: nucleic acid sequence for GAA13
ATGGCCTTTCTGTGGCTGCTGTCCTGCTGGGCCCTGCTGGGGACCACCTTTGGCCTGCTGGTGCCCAGGGAGCTG
TCTGGGAGCAGCCCAGTGCTGGAGGAGACCCACCCTGCCCACCAGCAGGGGGCCAGCAGGCCTGGCCCTAGGGAT
GCCCAGGCCCACCCTGGCAGGCCCAGGGCTGTGCCTACCCAGTGTGATGTGCCACCCAATTCTAGGTTTGACTGT
GCTCCTGACAAGGCCATCACTCAGGAGCAGTGTGAAGCTAGGGGGTGCTGCTACATCCCAGCCAAGCAGGGCCTG
CAGGGGGCCCAGATGGGCCAGCCCTGGTGCTTCTTCCCCCCCAGCTACCCTAGCTACAAGCTGGAGAATCTGAGC
AGCTCTGAGATGGGCTACACTGCTACCCTGACCAGGACCACTCCTACCTTCTTCCCCAAGGACATCCTGACTCTG
AGGCTGGATGTCATGATGGAGACTGAAAATAGGCTGCACTTCACCATCAAGGACCCTGCCAATAGGAGGTATGAG
GTGCCTCTGGAGACCCCCCATGTGCATAGCAGGGCTCCCAGCCCCCTGTATTCTGTGGAGTTCTCTGAGGAGCCC
TTTGGGGTCATTGTGAGGAGACAGCTGGATGGGAGGGTGCTGCTGAACACTACTGTGGCTCCCCTGTTCTTTGCT
GACCAGTTCCTGCAGCTGTCTACCAGCCTGCCCAGCCAGTACATCACTGGGCTGGCTGAGCATCTGAGCCCCCTG
ATGCTGAGCACCAGCTGGACCAGGATCACTCTGTGGAACAGGGATCTGGCCCCCACTCCTGGGGCCAACCTGTAT
GGGAGCCATCCCTTCTACCTGGCCCTGGAGGATGGGGGCTCTGCCCATGGGGTGTTCCTGCTGAACAGCAATGCC
ATGGATGTGGTGCTGCAGCCTAGCCCTGCCCTGAGCTGGAGGAGCACTGGGGGCATCCTGGATGTCTACATCTTC
CTGGGGCCTGAGCCCAAGTCTGTGGTGCAGCAGTATCTGGATGTGGTGGGGTATCCCTTCATGCCCCCCTACTGG
GGCCTGGGCTTTCACCTGTGCAGGTGGGGCTACAGCAGCACTGCCATCACCAGGCAGGTGGTGGAGAACATGACC
AGGGCCCACTTCCCTCTGGATGTGCAGTGGAATGACCTGGACTATATGGATTCTAGGAGAGACTTTACTTTTAAC
AAGGATGGCTTCAGGGATTTCCCTGCCATGGTGCAGGAGCTGCACCAGGGGGGCAGGAGGTACATGATGATTGTG
GACCCTGCTATTAGCAGCTCTGGCCCTGCTGGGTCTTACAGGCCTTATGATGAGGGCCTGAGGAGGGGGGTGTTC
ATCACCAATGAGACTGGCCAGCCCCTGATTGGCAAGGTGTGGCCTGGCAGCACTGCCTTCCCTGACTTCACCAAC
CCCACTGCCCTGGCCTGGTGGGAGGACATGGTGGCTGAGTTCCATGACCAGGTGCCCTTTGATGGGATGTGGATT
GACATGAATGAGCCCTCTAACTTCATCAGGGGGTCTGAGGATGGCTGCCCCAACAATGAGCTGGAGAACCCCCCC
TATGTGCCTGGGGTGGTGGGGGGCACTCTGCAGGCTGCCACTATCTGTGCTTCTTCTCACCAGTTTCTGAGCACC
CACTATAATCTGCACAACCTGTATGGCCTGACTGAGGCCATTGCCAGCCATAGGGCCCTGGTGAAGGCCAGGGGC
ACCAGGCCCTTTGTGATCAGCAGGTCTACCTTTGCTGGCCATGGCAGGTATGCTGGCCACTGGACTGGGGATGTG
TGGTCTTCTTGGGAGCAGCTGGCCAGCTCTGTGCCTGAGATCCTGCAGTTCAACCTGCTGGGGGTGCCTCTGGTG
GGGGCTGATGTGTGTGGCTTTCTGGGCAACACCTCTGAGGAGCTGTGTGTGAGGTGGACCCAGCTGGGGGCCTTT
TACCCCTTCATGAGGAACCACAATAGCCTGCTGAGCCTGCCCCAGGAGCCTTACTCTTTCTCTGAGCCTGCCCAG
CAGGCCATGAGGAAGGCCCTGACTCTGAGGTATGCCCTGCTGCCCCATCTGTATACCCTGTTTCACCAGGCCCAT
GTGGCTGGGGAGACTGTGGCTAGGCCTCTGTTTCTGGAGTTCCCTAAGGACTCTAGCACCTGGACTGTGGACCAC
CAGCTGCTGTGGGGGGAGGCCCTGCTGATCACCCCTGTGCTGCAGGCTGGCAAGGCTGAGGTGACTGGCTACTTC
CCCCTGGGCACCTGGTATGACCTGCAGACTGTGCCTGTGGAGGCCCTGGGGAGCCTGCCTCCCCCCCCTGCTGCC
CCCAGGGAGCCTGCCATTCATTCTGAGGGCCAGTGGGTGACCCTGCCTGCCCCTCTGGACACCATCAATGTGCAC
CTGAGGGCTGGGTACATCATCCCCCTGCAGGGCCCTGGCCTGACCACCACTGAGAGCAGGCAGCAGCCCATGGCC
CTGGCTGTGGCTCTGACCAAGGGGGGGGAGGCCAGGGGGGAGCTGTTCTGGGATGATGGGGAGTCTCTGGAGGTG
CTGGAGAGGGGGGCCTACACCCAGGTGATCTTTCTGGCCAGGAACAATACTATTGTGAATGAGCTGGTGAGGGTG
ACCTCTGAGGGGGCTGGCCTGCAGCTGCAGAAGGTGACTGTGCTGGGGGTGGCCACTGCCCCCCAGCAGGTCCTG
AGCAATGGGGTGCCTGTGAGCAACTTCACCTACTCTCCTGACACCAAGGTGCTGGACATTTGTGTGTCTCTGCTG
ATGGGGGAGCAGTTCCTGGTGAGCTGGTGCTGA
SEQ ID NO: 6: nucleic acid sequence for GAA2 plus WT BGH poly A
ATGGCTTTCCTGTGGCTGCTGAGCTGCTGGGCTCTGCTGGGCACCACCTTTGGGCTGCTGGTGCCTAGGGAGCTG
TCTGGGTCTAGCCCTGTGCTGGAGGAGACTCACCCTGCCCATCAGCAGGGGGCTAGCAGGCCTGGCCCCAGGGAT
GCTCAGGCCCACCCTGGCAGGCCCAGGGCTGTGCCCACCCAGTGTGATGTGCCCCCCAACAGCAGGTTTGACTGT
GCCCCTGACAAGGCCATTACCCAGGAGCAGTGTGAGGCCAGGGGCTGCTGCTACATTCCAGCTAAGCAGGGCCTG
CAGGGGGCCCAGATGGGCCAGCCCTGGTGCTTCTTCCCCCCCAGCTATCCTAGCTATAAACTGGAGAACCTGAGC
AGCTCTGAGATGGGCTATACTGCCACCCTGACTAGGACTACTCCCACCTTTTTTCCTAAGGATATCCTGACCCTG
AGGCTGGATGTGATGATGGAGACTGAGAACAGGCTGCACTTCACTATTAAGGACCCTGCCAATAGGAGGTATGAA
GTGCCTCTGGAGACTCCTCATGTGCACTCTAGGGCCCCCAGCCCCCTGTATTCTGTGGAGTTCTCTGAGGAGCCC
TTTGGGGTGATTGTGAGGAGGCAGCTGGATGGCAGGGTGCTGCTGAACACCACTGTGGCCCCCCTGTTCTTTGCT
GACCAGTTCCTGCAGCTGAGCACCAGCCTGCCCAGCCAGTACATCACTGGGCTGGCTGAGCATCTGAGCCCTCTG
ATGCTGAGCACCTCTTGGACCAGGATCACCCTGTGGAATAGGGATCTGGCCCCCACCCCTGGGGCTAATCTGTAT
GGCTCTCATCCCTTTTACCTGGCCCTGGAGGATGGGGGCTCTGCCCATGGGGTGTTTCTGCTGAACAGCAATGCC
ATGGATGTGGTGCTGCAGCCCTCTCCTGCCCTGAGCTGGAGGAGCACTGGGGGCATCCTGGATGTGTACATCTTC
CTGGGCCCTGAGCCCAAGTCTGTGGTCCAGCAGTATCTGGATGTGGTGGGCTACCCCTTTATGCCCCCCTATTGG
GGCCTGGGCTTCCACCTGTGCAGGTGGGGGTATTCTTCTACTGCTATCACCAGGCAGGTGGTGGAGAACATGACC
AGGGCTCACTTCCCCCTGGATGTGCAGTGGAATGACCTGGACTATATGGACTCTAGGAGGGATTTCACCTTCAAC
AAGGATGGCTTCAGGGACTTCCCTGCTATGGTCCAGGAGCTGCATCAGGGGGGCAGGAGGTACATGATGATTGTG
GACCCTGCCATCAGCAGCTCTGGCCCTGCTGGCAGCTATAGGCCCTATGATGAGGGCCTGAGGAGGGGGGTGTTT
ATCACTAATGAAACTGGGCAGCCCCTGATTGGCAAGGTGTGGCCTGGCTCTACTGCCTTCCCTGACTTCACCAAC
CCCACTGCTCTGGCCTGGTGGGAGGACATGGTGGCTGAGTTCCATGACCAGGTGCCTTTTGATGGCATGTGGATT
GACATGAATGAGCCCAGCAACTTCATCAGGGGCTCTGAGGATGGGTGCCCCAATAATGAGCTGGAGAACCCCCCC
TATGTGCCTGGGGTGGTGGGGGGCACCCTGCAGGCTGCCACTATTTGTGCCAGCTCTCACCAGTTCCTGAGCACC
CACTACAACCTGCACAATCTGTATGGCCTGACTGAGGCCATTGCCAGCCACAGGGCCCTGGTGAAGGCCAGGGGC
ACTAGGCCCTTTGTGATCTCTAGAAGCACCTTTGCTGGCCATGGGAGGTATGCTGGCCACTGGACTGGGGATGTG
TGGAGCTCTTGGGAGCAGCTGGCCAGCTCTGTGCCTGAGATCCTGCAGTTCAACCTGCTGGGGGTGCCCCTGGTG
GGGGCTGATGTGTGTGGCTTCCTGGGCAACACCTCTGAAGAGCTGTGTGTGAGGTGGACCCAGCTGGGGGCCTTC
TACCCTTTCATGAGGAACCACAACAGCCTGCTGAGCCTGCCTCAGGAGCCTTACTCTTTCTCTGAGCCTGCCCAG
CAGGCCATGAGGAAGGCCCTGACCCTGAGGTATGCTCTGCTGCCCCACCTGTACACCCTGTTCCACCAGGCCCAT
GTGGCTGGGGAGACTGTGGCCAGGCCCCTGTTCCTGGAGTTTCCTAAGGATAGCAGCACCTGGACTGTGGACCAC
CAGCTGCTGTGGGGGGAGGCCCTGCTGATTACCCCTGTGCTGCAGGCTGGCAAGGCTGAGGTGACTGGCTACTTC
CCCCTGGGCACTTGGTATGACCTGCAGACTGTGCCTGTGGAAGCCCTGGGCAGCCTGCCTCCCCCCCCTGCTGCC
CCCAGGGAGCCTGCCATCCACTCTGAGGGCCAGTGGGTGACCCTGCCTGCCCCCCTGGACACCATTAATGTGCAT
CTGAGGGCTGGGTATATTATCCCCCTGCAGGGGCCTGGGCTGACTACCACTGAGAGCAGGCAGCAGCCTATGGCC
CTGGCTGTGGCTCTGACTAAGGGGGGGGAGGCCAGGGGGGAGCTGTTCTGGGATGATGGGGAGAGCCTGGAGGTG
CTGGAGAGGGGGGCCTACACCCAGGTGATTTTCCTGGCCAGGAACAACACCATTGTGAATGAGCTGGTGAGGGTG
ACCTCTGAGGGGGCTGGCCTGCAGCTGCAGAAAGTGACTGTGCTGGGGGTGGCCACTGCCCCCCAGCAGGTGCTG
AGCAATGGGGTGCCTGTGAGCAACTTCACCTACAGCCCTGACACCAAGGTGCTGGATATTTGTGTGAGCCTGCTG
ATGGGGGAGCAGTTCCTGGTGAGCTGGTGCTGACTCGAGAGATCTACCGGTGAATTCACCGCGGGTTTAAACTGT
GCCTTCTAGTTGCCAGCCATCTGTTGTTTGCCCCTCCCCCGTGCCTTCCTTGACCCTGGAAGGTGCCACTCCCAC
TGTCCTTTCCTAATAAAATGAGGAAATTGCATCGCATTGTCTGAGTAGGTGTCATTCTATTCTGGGGGGTGGGGT
GGGGGCTAGCTCTAGA
SEQ ID NO: 7: nucleic acid sequence for GAA5 plus WT BGH poly A
ATGGCTTTCCTGTGGCTGCTGTCTTGCTGGGCCCTGCTGGGGACTACCTTTGGCCTGCTGGTGCCCAGGGAACTG
TCTGGCTCTAGCCCAGTGCTGGAGGAGACCCACCCTGCCCACCAGCAGGGGGCTTCTAGGCCTGGCCCCAGGGAT
GCCCAGGCCCACCCTGGCAGGCCAAGGGCTGTGCCCACCCAGTGTGATGTGCCCCCCAACTCTAGATTTGATTGT
GCCCCTGATAAGGCCATCACCCAGGAGCAGTGTGAGGCTAGGGGCTGCTGCTACATCCCTGCTAAGCAGGGCCTG
CAGGGGGCTCAGATGGGCCAGCCCTGGTGCTTCTTCCCCCCCAGCTATCCCTCTTACAAGCTGGAGAATCTGAGC
AGCTCTGAGATGGGCTACACTGCCACCCTGACCAGGACTACTCCCACCTTCTTCCCCAAGGACATCCTGACCCTG
AGGCTGGATGTGATGATGGAGACTGAGAACAGGCTGCATTTCACCATCAAGGATCCTGCCAACAGGAGGTATGAG
GTGCCTCTGGAGACCCCCCATGTGCACAGCAGGGCTCCTTCTCCCCTGTACTCTGTGGAGTTCTCTGAGGAACCC
TTTGGGGTGATTGTGAGGAGGCAGCTGGATGGCAGGGTCCTGCTGAACACCACTGTGGCCCCCCTGTTCTTTGCT
GATCAGTTCCTGCAGCTGTCCACTTCTCTGCCTAGCCAGTACATCACTGGGCTGGCTGAGCACCTGAGCCCTCTG
ATGCTGAGCACCTCTTGGACTAGGATCACCCTGTGGAACAGGGACCTGGCCCCCACCCCTGGGGCCAACCTGTAT
GGCAGCCACCCCTTCTATCTGGCCCTGGAGGATGGGGGCTCTGCCCATGGGGTGTTCCTGCTGAATAGCAATGCT
ATGGATGTGGTGCTGCAGCCCAGCCCTGCCCTGTCTTGGAGGAGCACTGGGGGCATCCTGGATGTGTACATTTTC
CTGGGGCCTGAGCCCAAGTCTGTGGTGCAGCAGTACCTGGATGTGGTGGGCTACCCCTTCATGCCTCCCTACTGG
GGCCTGGGCTTCCACCTGTGCAGGTGGGGCTACAGCTCTACTGCCATCACCAGGCAGGTGGTGGAGAATATGACC
AGGGCCCACTTCCCCCTGGATGTGCAGTGGAATGACCTGGACTACATGGACTCTAGGAGGGACTTCACCTTCAAT
AAGGATGGCTTCAGAGACTTCCCTGCCATGGTGCAGGAGCTGCATCAGGGGGGCAGGAGGTACATGATGATTGTG
GACCCTGCCATCAGCTCTTCTGGCCCTGCTGGCTCTTACAGGCCCTATGATGAGGGCCTGAGGAGGGGGGTGTTC
ATCACCAATGAGACTGGGCAGCCCCTGATTGGGAAGGTGTGGCCTGGCTCTACTGCCTTCCCTGACTTCACCAAT
CCTACTGCCCTGGCCTGGTGGGAGGACATGGTGGCTGAGTTCCATGACCAGGTGCCCTTTGATGGCATGTGGATT
GACATGAATGAGCCCTCTAATTTCATCAGGGGCTCTGAGGATGGCTGCCCCAACAATGAGCTGGAGAACCCCCCC
TATGTGCCTGGGGTGGTGGGGGGCACCCTGCAGGCTGCCACCATCTGTGCTAGCTCTCACCAGTTCCTGAGCACC
CACTACAATCTGCATAACCTGTATGGCCTGACTGAGGCCATTGCCAGCCACAGGGCCCTGGTGAAGGCTAGGGGC
ACCAGGCCCTTTGTGATTTCTAGGAGCACTTTTGCTGGCCATGGCAGGTATGCTGGGCACTGGACTGGGGATGTG
TGGTCTAGCTGGGAGCAGCTGGCTTCTTCTGTGCCTGAGATCCTGCAGTTCAACCTGCTGGGGGTGCCTCTGGTG
GGGGCTGATGTGTGTGGGTTCCTGGGCAACACTTCTGAGGAGCTGTGTGTGAGGTGGACCCAGCTGGGGGCCTTC
TACCCTTTCATGAGGAACCACAACAGCCTGCTGAGCCTGCCCCAGGAGCCCTACAGCTTCTCTGAGCCTGCCCAG
CAGGCCATGAGGAAGGCCCTGACCCTGAGGTATGCCCTGCTGCCCCACCTGTACACCCTGTTCCACCAGGCCCAT
GTGGCTGGGGAGACTGTGGCTAGGCCTCTGTTCCTGGAGTTCCCCAAGGACTCTAGCACCTGGACTGTGGACCAC
CAGCTGCTGTGGGGGGAGGCCCTGCTGATCACTCCTGTGCTGCAGGCTGGGAAGGCTGAGGTGACTGGCTATTTC
CCCCTGGGCACCTGGTATGACCTGCAGACTGTGCCTGTGGAGGCCCTGGGGAGCCTGCCCCCCCCCCCTGCTGCC
CCCAGGGAGCCTGCCATCCACTCTGAGGGCCAGTGGGTGACCCTGCCTGCCCCTCTGGATACCATCAATGTGCAC
CTGAGGGCTGGCTACATCATTCCCCTGCAGGGCCCTGGCCTGACCACTACTGAGTCTAGGCAGCAGCCCATGGCC
CTGGCTGTGGCCCTGACCAAGGGGGGGGAGGCTAGGGGGGAGCTGTTTTGGGATGATGGGGAGAGCCTGGAGGTG
CTGGAGAGGGGGGCCTACACTCAGGTGATCTTCCTGGCCAGGAACAATACCATTGTGAATGAGCTGGTGAGGGTG
ACCTCTGAGGGGGCTGGCCTGCAGCTGCAGAAGGTGACTGTGCTGGGGGTGGCCACTGCCCCCCAGCAGGTGCTG
AGCAATGGGGTGCCTGTGAGCAACTTCACCTATAGCCCTGATACCAAGGTGCTGGATATTTGTGTGAGCCTGCTG
ATGGGGGAGCAGTTCCTGGTGAGCTGGTGCTGACTCGAGAGATCTACCGGTGAATTCACCGCGGGTTTAAACTGT
GCCTTCTAGTTGCCAGCCATCTGTTGTTTGCCCCTCCCCCGTGCCTTCCTTGACCCTGGAAGGTGCCACTCCCAC
TGTCCTTTCCTAATAAAATGAGGAAATTGCATCGCATTGTCTGAGTAGGTGTCATTCTATTCTGGGGGGTGGGGT
GGGGGCTAGCTCTAGA
SEQ ID NO: 8: nucleic acid sequence for GAA7 plus WT BGH poly A
ATGGCTTTCCTGTGGCTGCTGTCTTGTTGGGCTCTGCTGGGCACCACCTTTGGCCTGCTGGTGCCCAGGGAGCTG
TCTGGCAGCAGCCCTGTGCTGGAGGAGACCCACCCTGCTCATCAGCAGGGGGCTAGCAGGCCTGGCCCCAGGGAT
GCCCAGGCTCACCCTGGGAGACCCAGGGCTGTGCCCACTCAGTGTGATGTGCCCCCCAACAGCAGGTTTGACTGT
GCTCCTGACAAGGCTATCACCCAGGAGCAGTGTGAGGCCAGGGGGTGCTGCTACATTCCTGCTAAGCAGGGCCTG
CAGGGGGCCCAGATGGGCCAGCCCTGGTGCTTCTTCCCCCCCTCTTATCCCAGCTATAAGCTGGAGAACCTGAGC
AGCTCTGAGATGGGCTACACTGCCACCCTGACCAGGACCACTCCCACCTTCTTTCCCAAGGATATTCTGACTCTG
AGGCTGGATGTGATGATGGAGACTGAGAACAGGCTGCACTTCACTATCAAGGACCCTGCCAATAGGAGGTATGAG
GTGCCCCTGGAGACTCCTCATGTGCATAGCAGGGCCCCTTCTCCTCTGTATTCTGTGGAGTTCTCTGAGGAGCCC
TTTGGGGTGATTGTGAGGAGGCAGCTGGATGGCAGGGTGCTGCTGAACACCACTGTGGCCCCCCTGTTCTTTGCT
GACCAGTTCCTGCAGCTGAGCACTTCTCTGCCCAGCCAGTACATTACTGGGCTGGCTGAGCATCTGAGCCCCCTG
ATGCTGAGCACCTCTTGGACCAGGATCACCCTGTGGAACAGGGACCTGGCCCCCACTCCTGGGGCTAACCTGTAT
GGCTCTCACCCCTTTTACCTGGCCCTGGAGGATGGGGGCTCTGCCCATGGGGTGTTTCTGCTGAACAGCAATGCT
ATGGATGTGGTGCTGCAGCCCTCTCCAGCCCTGTCTTGGAGGAGCACTGGGGGCATTCTGGATGTGTACATTTTC
CTGGGGCCTGAACCCAAGTCTGTGGTGCAGCAGTACCTGGATGTGGTGGGCTACCCCTTCATGCCCCCCTATTGG
GGGCTGGGGTTTCACCTGTGCAGGTGGGGCTACAGCAGCACTGCCATCACCAGGCAGGTGGTGGAGAACATGACC
AGGGCCCATTTCCCCCTGGATGTGCAGTGGAATGACCTGGACTACATGGATAGCAGGAGGGATTTCACCTTCAAC
AAGGATGGCTTCAGGGACTTTCCTGCCATGGTGCAGGAGCTGCACCAGGGGGGCAGGAGGTATATGATGATTGTG
GACCCTGCTATCAGCAGCTCTGGCCCTGCTGGCTCTTACAGGCCCTATGATGAGGGCCTGAGGAGGGGGGTGTTT
ATCACTAATGAAACTGGCCAGCCTCTGATTGGCAAGGTCTGGCCTGGCTCTACTGCCTTCCCTGATTTTACTAAC
CCCACTGCCCTGGCCTGGTGGGAGGACATGGTGGCTGAGTTCCATGATCAGGTGCCTTTTGATGGCATGTGGATT
GATATGAATGAACCAAGCAACTTCATCAGAGGCTCTGAGGATGGCTGCCCCAACAATGAGCTGGAGAACCCCCCC
TATGTGCCTGGGGTGGTGGGGGGCACTCTGCAGGCTGCCACCATTIGTGCTAGCAGCCACCAGTTCCTGAGCACC
CACTACAATCTGCACAACCTGTATGGCCTGACTGAAGCCATTGCCAGCCATAGGGCCCTGGTGAAGGCCAGGGGC
ACTAGGCCTTTTGTGATCAGCAGGAGCACTTTTGCTGGCCATGGCAGGTATGCTGGCCACTGGACTGGGGATGTG
TGGAGCAGCTGGGAGCAGCTGGCCAGCTCTGTGCCTGAGATTCTGCAGTTTAACCTGCTGGGGGTGCCCCTGGTG
GGGGCTGATGTGTGTGGCTTCCTGGGCAACACCTCTGAGGAGCTGTGTGTGAGGTGGACCCAGCTGGGGGCCTTT
TATCCCTTCATGAGGAACCACAACAGCCTGCTGAGCCTGCCTCAGGAGCCCTACTCTTTCTCTGAGCCTGCCCAG
CAGGCCATGAGGAAGGCCCTGACCCTGAGGTATGCCCTGCTGCCCCACCTGTATACCCTGTTCCACCAGGCCCAT
GTGGCTGGGGAGACTGTGGCCAGGCCCCTGTTCCTGGAGTTCCCCAAGGACAGCAGCACCTGGACTGTGGATCAT
CAGCTGCTGTGGGGGGAGGCCCTGCTGATCACCCCTGTGCTGCAGGCTGGCAAGGCTGAGGTCACTGGCTACTTC
CCTCTGGGCACCTGGTATGACCTGCAGACTGTGCCTGTGGAGGCTCTGGGCAGCCTGCCCCCCCCCCCTGCTGCT
CCCAGGGAGCCTGCCATCCACTCTGAGGGCCAGTGGGTGACCCTGCCTGCTCCCCTGGACACCATCAATGTGCAC
CTGAGGGCTGGCTACATTATCCCCCTGCAGGGCCCAGGGCTGACTACCACTGAGAGCAGACAGCAGCCCATGGCT
CTGGCTGTGGCCCTGACCAAGGGGGGGGAAGCTAGGGGGGAGCTGTTCTGGGATGATGGGGAGAGCCTGGAGGTG
CTGGAGAGGGGGGCCTATACCCAGGTGATCTTCCTGGCTAGGAACAACACCATTGTCAATGAGCTGGTGAGGGTG
ACTTCTGAGGGGGCTGGGCTGCAGCTGCAGAAGGTGACTGTGCTGGGGGTGGCCACTGCTCCCCAGCAGGTGCTG
AGCAATGGGGTGCCTGTGAGCAACTTCACCTACAGCCCTGACACCAAGGTGCTGGACATCTGTGTGTCTCTGCTG
ATGGGGGAGCAGTTCCTGGTGAGCTGGTGCTGACTCGAGAGATCTACCGGTGAATTCACCGCGGGTTTAAACTGT
GCCTTCTAGTTGCCAGCCATCTGTTGTTTGCCCCTCCCCCGTGCCTTCCTTGACCCTGGAAGGTGCCACTCCCAC
TGTCCTTTCCTAATAAAATGAGGAAATTGCATCGCATTGTCTGAGTAGGTGTCATTCTATTCTGGGGGGTGGGGT
GGGGGCTAGCTCTAGA
SEQ ID NO: 9: nucleic acid sequence for GAA8 plus WT BGH poly A
ATGGCCTTCCTGTGGCTGCTGTCTTGCTGGGCTCTGCTGGGGACCACCTTTGGCCTGCTGGTCCCCAGGGAGCTG
TCTGGCTCTTCTCCTGTCCTGGAGGAGACCCACCCTGCCCACCAGCAGGGGGCTAGCAGGCCTGGCCCCAGGGAT
GCCCAGGCCCACCCTGGCAGGCCCAGGGCTGTGCCCACCCAGTGTGATGTGCCTCCCAACAGCAGGTTTGACTGT
GCCCCTGACAAGGCCATCACCCAGGAGCAGTGTGAGGCCAGGGGCTGCTGCTATATCCCTGCCAAGCAGGGCCTG
CAGGGGGCTCAGATGGGCCAGCCCTGGTGCTTCTTTCCCCCCTCTTATCCTAGCTATAAGCTGGAGAACCTGAGC
AGCTCTGAGATGGGGTACACTGCCACCCTGACCAGGACCACCCCCACTTTCTTCCCTAAGGACATCCTGACCCTG
AGGCTGGATGTGATGATGGAGACTGAGAATAGGCTGCACTTTACTATCAAGGACCCTGCCAACAGGAGGTATGAG
GTGCCTCTGGAGACCCCCCATGTGCATTCTAGGGCCCCCAGCCCCCTGTACTCTGTGGAGTTCTCTGAGGAGCCC
TTTGGGGTGATTGTGAGGAGACAGCTGGATGGCAGGGTCCTGCTGAACACCACTGTGGCTCCCCTGTTTTTTGCT
GACCAGTTCCTGCAGCTGAGCACCAGCCTGCCCAGCCAGTACATCACTGGGCTGGCTGAGCACCTGAGCCCCCTG
ATGCTGAGCACCAGCTGGACCAGGATCACCCTGTGGAACAGGGATCTGGCTCCTACCCCTGGGGCCAACCTGTAT
GGCTCTCACCCCTTTTACCTGGCCCTGGAGGATGGGGGCTCTGCCCATGGGGTGTTCCTGCTGAACAGCAATGCT
ATGGATGTGGTGCTGCAGCCCAGCCCTGCCCTGAGCTGGAGGTCTACTGGGGGCATCCTGGATGTGTACATCTTT
CTGGGGCCTGAGCCCAAGTCTGTGGTGCAGCAGTACCTGGATGTGGTGGGCTATCCTTTTATGCCCCCCTATTGG
GGCCTGGGCTTCCACCTGTGCAGGTGGGGCTACAGCAGCACTGCCATCACCAGACAGGTGGTGGAGAACATGACC
AGGGCCCACTTCCCCCTGGATGTGCAGTGGAATGACCTGGACTACATGGACAGCAGGAGGGACTTCACCTTTAAC
AAGGATGGCTTTAGGGACTTCCCTGCCATGGTGCAGGAGCTGCATCAGGGGGGCAGGAGGTACATGATGATTGTG
GACCCAGCCATCAGCAGCTCTGGGCCTGCTGGGTCTTACAGGCCCTATGATGAGGGCCTGAGGAGGGGGGTGTTC
ATCACCAATGAGACTGGCCAGCCCCTGATTGGCAAGGTGTGGCCTGGGAGCACTGCCTTCCCTGATTTTACCAAC
CCCACTGCCCTGGCCTGGTGGGAGGATATGGTGGCTGAGTTTCATGACCAGGTGCCCTTTGATGGCATGTGGATT
GACATGAATGAGCCCAGCAATTTCATCAGGGGCTCTGAGGATGGCTGCCCCAACAATGAGCTGGAGAATCCTCCC
TATGTGCCTGGGGTGGTGGGGGGCACCCTGCAGGCTGCCACCATCTGTGCCTCTAGCCACCAGTTCCTGAGCACC
CACTATAACCTGCATAACCTGTATGGCCTGACTGAGGCCATTGCCAGCCATAGAGCCCTGGTGAAGGCCAGAGGG
ACCAGGCCCTTTGTGATCTCTAGGAGCACCTTTGCTGGCCATGGCAGGTATGCTGGCCACTGGACTGGGGATGTG
TGGAGCTCTTGGGAGCAGCTGGCCAGCTCTGTGCCAGAGATCCTGCAGTTCAACCTGCTGGGGGTGCCTCTGGTG
GGGGCTGATGTGTGTGGCTTCCTGGGCAATACCTCTGAAGAGCTGTGTGTGAGGTGGACTCAGCTGGGGGCCTTC
TATCCCTTCATGAGGAACCACAACAGCCTGCTGTCTCTGCCCCAGGAGCCCTACAGCTTCTCTGAGCCTGCTCAG
CAGGCTATGAGGAAGGCCCTGACCCTGAGGTATGCCCTGCTGCCCCATCTGTACACCCTGTTCCACCAGGCCCAT
GTGGCTGGGGAGACTGTGGCCAGGCCCCTGTTTCTGGAGTTTCCCAAGGACAGCAGCACCTGGACTGTGGACCAT
CAGCTGCTGTGGGGGGAGGCTCTGCTGATTACCCCTGTGCTGCAGGCTGGCAAGGCTGAGGTGACTGGGTACTTC
CCCCTGGGGACTTGGTATGACCTGCAGACTGTGCCTGTGGAAGCTCTGGGCAGCCTGCCCCCACCCCCTGCTGCC
CCTAGGGAGCCTGCCATCCACTCTGAGGGCCAGTGGGTGACCCTGCCTGCCCCTCTGGACACCATCAATGTGCAC
CTGAGGGCTGGCTATATCATCCCCCTGCAGGGCCCTGGGCTGACCACCACTGAGAGCAGGCAGCAGCCCATGGCC
CTGGCTGTGGCCCTGACTAAGGGGGGGGAGGCCAGGGGGGAGCTGTTCTGGGATGATGGGGAGAGCCTGGAGGTG
CTGGAGAGAGGGGCCTACACCCAGGTGATCTTTCTGGCCAGGAACAACACCATTGTGAATGAGCTGGTGAGGGTG
ACTTCTGAGGGGGCTGGCCTGCAGCTGCAGAAGGTGACTGTGCTGGGGGTGGCCACTGCCCCCCAGCAGGTGCTG
AGCAATGGGGTGCCTGTGTCTAACTTCACCTACAGCCCTGATACTAAGGTGCTGGATATCTGTGTGAGCCTGCTG
ATGGGGGAGCAGTTTCTGGTGAGCTGGTGCTGACTCGAGAGATCTACCGGTGAATTCACCGCGGGTTTAAACTGT
GCCTTCTAGTTGCCAGCCATCTGTTGTTTGCCCCTCCCCCGTGCCTTCCTTGACCCTGGAAGGTGCCACTCCCAC
TGTCCTTTCCTAATAAAATGAGGAAATTGCATCGCATTGTCTGAGTAGGTGTCATTCTATTCTGGGGGGTGGGGT
GGGGGCTAGCTCTAGA
SEQ ID NO: 10: nucleic acid sequence for GAA13 plus WT BGH poly A
ATGGCCTTTCTGTGGCTGCTGTCCTGCTGGGCCCTGCTGGGGACCACCTTTGGCCTGCTGGTGCCCAGGGAGCTG
TCTGGGAGCAGCCCAGTGCTGGAGGAGACCCACCCTGCCCACCAGCAGGGGGCCAGCAGGCCTGGCCCTAGGGAT
GCCCAGGCCCACCCTGGCAGGCCCAGGGCTGTGCCTACCCAGTGTGATGTGCCACCCAATTCTAGGTTTGACTGT
GCTCCTGACAAGGCCATCACTCAGGAGCAGTGTGAAGCTAGGGGGTGCTGCTACATCCCAGCCAAGCAGGGCCTG
CAGGGGGCCCAGATGGGCCAGCCCTGGTGCTTCTTCCCCCCCAGCTACCCTAGCTACAAGCTGGAGAATCTGAGC
AGCTCTGAGATGGGCTACACTGCTACCCTGACCAGGACCACTCCTACCTTCTTCCCCAAGGACATCCTGACTCTG
AGGCTGGATGTCATGATGGAGACTGAAAATAGGCTGCACTTCACCATCAAGGACCCTGCCAATAGGAGGTATGAG
GTGCCTCTGGAGACCCCCCATGTGCATAGCAGGGCTCCCAGCCCCCTGTATTCTGTGGAGTTCTCTGAGGAGCCC
TTTGGGGTCATTGTGAGGAGACAGCTGGATGGGAGGGTGCTGCTGAACACTACTGTGGCTCCCCTGTTCTTTGCT
GACCAGTTCCTGCAGCTGTCTACCAGCCTGCCCAGCCAGTACATCACTGGGCTGGCTGAGCATCTGAGCCCCCTG
ATGCTGAGCACCAGCTGGACCAGGATCACTCTGTGGAACAGGGATCTGGCCCCCACTCCTGGGGCCAACCTGTAT
GGGAGCCATCCCTTCTACCTGGCCCTGGAGGATGGGGGCTCTGCCCATGGGGTGTTCCTGCTGAACAGCAATGCC
ATGGATGTGGTGCTGCAGCCTAGCCCTGCCCTGAGCTGGAGGAGCACTGGGGGCATCCTGGATGTCTACATCTTC
CTGGGGCCTGAGCCCAAGTCTGTGGTGCAGCAGTATCTGGATGTGGTGGGGTATCCCTTCATGCCCCCCTACTGG
GGCCTGGGCTTTCACCTGTGCAGGTGGGGCTACAGCAGCACTGCCATCACCAGGCAGGTGGTGGAGAACATGACC
AGGGCCCACTTCCCTCTGGATGTGCAGTGGAATGACCTGGACTATATGGATTCTAGGAGAGACTTTACTTTTAAC
AAGGATGGCTTCAGGGATTTCCCTGCCATGGTGCAGGAGCTGCACCAGGGGGGCAGGAGGTACATGATGATTGTG
GACCCTGCTATTAGCAGCTCTGGCCCTGCTGGGTCTTACAGGCCTTATGATGAGGGCCTGAGGAGGGGGGTGTTC
ATCACCAATGAGACTGGCCAGCCCCTGATTGGCAAGGTGTGGCCTGGCAGCACTGCCTTCCCTGACTTCACCAAC
CCCACTGCCCTGGCCTGGTGGGAGGACATGGTGGCTGAGTTCCATGACCAGGTGCCCTTTGATGGGATGTGGATT
GACATGAATGAGCCCTCTAACTTCATCAGGGGGTCTGAGGATGGCTGCCCCAACAATGAGCTGGAGAACCCCCCC
TATGTGCCTGGGGTGGTGGGGGGCACTCTGCAGGCTGCCACTATCTGTGCTTCTTCTCACCAGTTTCTGAGCACC
CACTATAATCTGCACAACCTGTATGGCCTGACTGAGGCCATTGCCAGCCATAGGGCCCTGGTGAAGGCCAGGGGC
ACCAGGCCCTTTGTGATCAGCAGGTCTACCTTTGCTGGCCATGGCAGGTATGCTGGCCACTGGACTGGGGATGTG
TGGTCTTCTTGGGAGCAGCTGGCCAGCTCTGTGCCTGAGATCCTGCAGTTCAACCTGCTGGGGGTGCCTCTGGTG
GGGGCTGATGTGTGTGGCTTTCTGGGCAACACCTCTGAGGAGCTGTGTGTGAGGTGGACCCAGCTGGGGGCCTTT
TACCCCTTCATGAGGAACCACAATAGCCTGCTGAGCCTGCCCCAGGAGCCTTACTCTTTCTCTGAGCCTGCCCAG
CAGGCCATGAGGAAGGCCCTGACTCTGAGGTATGCCCTGCTGCCCCATCTGTATACCCTGTTTCACCAGGCCCAT
GTGGCTGGGGAGACTGTGGCTAGGCCTCTGTTTCTGGAGTTCCCTAAGGACTCTAGCACCTGGACTGTGGACCAC
CAGCTGCTGTGGGGGGAGGCCCTGCTGATCACCCCTGTGCTGCAGGCTGGCAAGGCTGAGGTGACTGGCTACTTC
CCCCTGGGCACCTGGTATGACCTGCAGACTGTGCCTGTGGAGGCCCTGGGGAGCCTGCCTCCCCCCCCTGCTGCC
CCCAGGGAGCCTGCCATTCATTCTGAGGGCCAGTGGGTGACCCTGCCTGCCCCTCTGGACACCATCAATGTGCAC
CTGAGGGCTGGGTACATCATCCCCCTGCAGGGCCCTGGCCTGACCACCACTGAGAGCAGGCAGCAGCCCATGGCC
CTGGCTGTGGCTCTGACCAAGGGGGGGGAGGCCAGGGGGGAGCTGTTCTGGGATGATGGGGAGTCTCTGGAGGTG
CTGGAGAGGGGGGCCTACACCCAGGTGATCTTTCTGGCCAGGAACAATACTATTGTGAATGAGCTGGTGAGGGTG
ACCTCTGAGGGGGCTGGCCTGCAGCTGCAGAAGGTGACTGTGCTGGGGGTGGCCACTGCCCCCCAGCAGGTCCTG
AGCAATGGGGTGCCTGTGAGCAACTTCACCTACTCTCCTGACACCAAGGTGCTGGACATTTGTGTGTCTCTGCTG
ATGGGGGAGCAGTTCCTGGTGAGCTGGTGCTGACTCGAGAGATCTACCGGTGAATTCACCGCGGGTTTAAACTGT
GCCTTCTAGTTGCCAGCCATCTGTTGTTTGCCCCTCCCCCGTGCCTTCCTTGACCCTGGAAGGTGCCACTCCCAC
TGTCCTTTCCTAATAAAATGAGGAAATTGCATCGCATTGTCTGAGTAGGTGTCATTCTATTCTGGGGGGTGGGGT
GGGGGCTAGCTCTAGA
SEQ ID NO: 11: nucleic acid sequence for GAA2 plus CpG-reduced BGH poly A
ATGGCTTTCCTGTGGCTGCTGAGCTGCTGGGCTCTGCTGGGCACCACCTTTGGGCTGCTGGTGCCTAGGGAGCTG
TCTGGGTCTAGCCCTGTGCTGGAGGAGACTCACCCTGCCCATCAGCAGGGGGCTAGCAGGCCTGGCCCCAGGGAT
GCTCAGGCCCACCCTGGCAGGCCCAGGGCTGTGCCCACCCAGTGTGATGTGCCCCCCAACAGCAGGTTTGACTGT
GCCCCTGACAAGGCCATTACCCAGGAGCAGTGTGAGGCCAGGGGCTGCTGCTACATTCCAGCTAAGCAGGGCCTG
CAGGGGGCCCAGATGGGCCAGCCCTGGTGCTTCTTCCCCCCCAGCTATCCTAGCTATAAACTGGAGAACCTGAGC
AGCTCTGAGATGGGCTATACTGCCACCCTGACTAGGACTACTCCCACCTTTTTTCCTAAGGATATCCTGACCCTG
AGGCTGGATGTGATGATGGAGACTGAGAACAGGCTGCACTTCACTATTAAGGACCCTGCCAATAGGAGGTATGAA
GTGCCTCTGGAGACTCCTCATGTGCACTCTAGGGCCCCCAGCCCCCTGTATTCTGTGGAGTTCTCTGAGGAGCCC
TTTGGGGTGATTGTGAGGAGGCAGCTGGATGGCAGGGTGCTGCTGAACACCACTGTGGCCCCCCTGTTCTTTGCT
GACCAGTTCCTGCAGCTGAGCACCAGCCTGCCCAGCCAGTACATCACTGGGCTGGCTGAGCATCTGAGCCCTCTG
ATGCTGAGCACCTCTTGGACCAGGATCACCCTGTGGAATAGGGATCTGGCCCCCACCCCTGGGGCTAATCTGTAT
GGCTCTCATCCCTTTTACCTGGCCCTGGAGGATGGGGGCTCTGCCCATGGGGTGTTTCTGCTGAACAGCAATGCC
ATGGATGTGGTGCTGCAGCCCTCTCCTGCCCTGAGCTGGAGGAGCACTGGGGGCATCCTGGATGTGTACATCTTC
CTGGGCCCTGAGCCCAAGTCTGTGGTCCAGCAGTATCTGGATGTGGTGGGCTACCCCTTTATGCCCCCCTATTGG
GGCCTGGGCTTCCACCTGTGCAGGTGGGGGTATTCTTCTACTGCTATCACCAGGCAGGTGGTGGAGAACATGACC
AGGGCTCACTTCCCCCTGGATGTGCAGTGGAATGACCTGGACTATATGGACTCTAGGAGGGATTTCACCTTCAAC
AAGGATGGCTTCAGGGACTTCCCTGCTATGGTCCAGGAGCTGCATCAGGGGGGCAGGAGGTACATGATGATTGTG
GACCCTGCCATCAGCAGCTCTGGCCCTGCTGGCAGCTATAGGCCCTATGATGAGGGCCTGAGGAGGGGGGTGTTT
ATCACTAATGAAACTGGGCAGCCCCTGATTGGCAAGGTGTGGCCTGGCTCTACTGCCTTCCCTGACTTCACCAAC
CCCACTGCTCTGGCCTGGTGGGAGGACATGGTGGCTGAGTTCCATGACCAGGTGCCTTTTGATGGCATGTGGATT
GACATGAATGAGCCCAGCAACTTCATCAGGGGCTCTGAGGATGGGTGCCCCAATAATGAGCTGGAGAACCCCCCC
TATGTGCCTGGGGTGGTGGGGGGCACCCTGCAGGCTGCCACTATTTGTGCCAGCTCTCACCAGTTCCTGAGCACC
CACTACAACCTGCACAATCTGTATGGCCTGACTGAGGCCATTGCCAGCCACAGGGCCCTGGTGAAGGCCAGGGGC
ACTAGGCCCTTTGTGATCTCTAGAAGCACCTTTGCTGGCCATGGGAGGTATGCTGGCCACTGGACTGGGGATGTG
TGGAGCTCTTGGGAGCAGCTGGCCAGCTCTGTGCCTGAGATCCTGCAGTTCAACCTGCTGGGGGTGCCCCTGGTG
GGGGCTGATGTGTGTGGCTTCCTGGGCAACACCTCTGAAGAGCTGTGTGTGAGGTGGACCCAGCTGGGGGCCTTC
TACCCTTTCATGAGGAACCACAACAGCCTGCTGAGCCTGCCTCAGGAGCCTTACTCTTTCTCTGAGCCTGCCCAG
CAGGCCATGAGGAAGGCCCTGACCCTGAGGTATGCTCTGCTGCCCCACCTGTACACCCTGTTCCACCAGGCCCAT
GTGGCTGGGGAGACTGTGGCCAGGCCCCTGTTCCTGGAGTTTCCTAAGGATAGCAGCACCTGGACTGTGGACCAC
CAGCTGCTGTGGGGGGAGGCCCTGCTGATTACCCCTGTGCTGCAGGCTGGCAAGGCTGAGGTGACTGGCTACTTC
CCCCTGGGCACTTGGTATGACCTGCAGACTGTGCCTGTGGAAGCCCTGGGCAGCCTGCCTCCCCCCCCTGCTGCC
CCCAGGGAGCCTGCCATCCACTCTGAGGGCCAGTGGGTGACCCTGCCTGCCCCCCTGGACACCATTAATGTGCAT
CTGAGGGCTGGGTATATTATCCCCCTGCAGGGGCCTGGGCTGACTACCACTGAGAGCAGGCAGCAGCCTATGGCC
CTGGCTGTGGCTCTGACTAAGGGGGGGGAGGCCAGGGGGGAGCTGTTCTGGGATGATGGGGAGAGCCTGGAGGTG
CTGGAGAGGGGGGCCTACACCCAGGTGATTTTCCTGGCCAGGAACAACACCATTGTGAATGAGCTGGTGAGGGTG
ACCTCTGAGGGGGCTGGCCTGCAGCTGCAGAAAGTGACTGTGCTGGGGGTGGCCACTGCCCCCCAGCAGGTGCTG
AGCAATGGGGTGCCTGTGAGCAACTTCACCTACAGCCCTGACACCAAGGTGCTGGATATTTGTGTGAGCCTGCTG
ATGGGGGAGCAGTTCCTGGTGAGCTGGTGCTGAAGATCTAGAGCTGAATTCCTGCAGCCAGGGGGATCAGCCTCT
ACTGTGCCTTCTAGTTGCCAGCCATCTGTTGTTTGCCCCTCCCCCTTGCCTTCCTTGACCCTGGAAGGTGCCACT
CCCACTGTCCTTTCCTAATAAAATGAGGAAATTGCATCACATTGTCTGAGTAGGTGTCATTCTATTCTGGGGGGT
GGGGTGGGGCAGGACAGCAAGGGGGAGGATTGGGAAGACAATAGCAGGCATGCTGGGGATGCAGTGGGCTCTATG
G
SEQ ID NO: 12: nucleic acid sequence for GAA5 plus CpG-reduced BGH poly A
ATGGCTTTCCTGTGGCTGCTGTCTTGCTGGGCCCTGCTGGGGACTACCTTTGGCCTGCTGGTGCCCAGGGAACTG
TCTGGCTCTAGCCCAGTGCTGGAGGAGACCCACCCTGCCCACCAGCAGGGGGCTTCTAGGCCTGGCCCCAGGGAT
GCCCAGGCCCACCCTGGCAGGCCAAGGGCTGTGCCCACCCAGTGTGATGTGCCCCCCAACTCTAGATTTGATTGT
GCCCCTGATAAGGCCATCACCCAGGAGCAGTGTGAGGCTAGGGGCTGCTGCTACATCCCTGCTAAGCAGGGCCTG
CAGGGGGCTCAGATGGGCCAGCCCTGGTGCTTCTTCCCCCCCAGCTATCCCTCTTACAAGCTGGAGAATCTGAGC
AGCTCTGAGATGGGCTACACTGCCACCCTGACCAGGACTACTCCCACCTTCTTCCCCAAGGACATCCTGACCCTG
AGGCTGGATGTGATGATGGAGACTGAGAACAGGCTGCATTTCACCATCAAGGATCCTGCCAACAGGAGGTATGAG
GTGCCTCTGGAGACCCCCCATGTGCACAGCAGGGCTCCTTCTCCCCTGTACTCTGTGGAGTTCTCTGAGGAACCC
TTTGGGGTGATTGTGAGGAGGCAGCTGGATGGCAGGGTCCTGCTGAACACCACTGTGGCCCCCCTGTTCTTTGCT
GATCAGTTCCTGCAGCTGTCCACTTCTCTGCCTAGCCAGTACATCACTGGGCTGGCTGAGCACCTGAGCCCTCTG
ATGCTGAGCACCTCTTGGACTAGGATCACCCTGTGGAACAGGGACCTGGCCCCCACCCCTGGGGCCAACCTGTAT
GGCAGCCACCCCTTCTATCTGGCCCTGGAGGATGGGGGCTCTGCCCATGGGGTGTTCCTGCTGAATAGCAATGCT
ATGGATGTGGTGCTGCAGCCCAGCCCTGCCCTGTCTTGGAGGAGCACTGGGGGCATCCTGGATGTGTACATTTTC
CTGGGGCCTGAGCCCAAGTCTGTGGTGCAGCAGTACCTGGATGTGGTGGGCTACCCCTTCATGCCTCCCTACTGG
GGCCTGGGCTTCCACCTGTGCAGGTGGGGCTACAGCTCTACTGCCATCACCAGGCAGGTGGTGGAGAATATGACC
AGGGCCCACTTCCCCCTGGATGTGCAGTGGAATGACCTGGACTACATGGACTCTAGGAGGGACTTCACCTTCAAT
AAGGATGGCTTCAGAGACTTCCCTGCCATGGTGCAGGAGCTGCATCAGGGGGGCAGGAGGTACATGATGATTGTG
GACCCTGCCATCAGCTCTTCTGGCCCTGCTGGCTCTTACAGGCCCTATGATGAGGGCCTGAGGAGGGGGGTGTTC
ATCACCAATGAGACTGGGCAGCCCCTGATTGGGAAGGTGTGGCCTGGCTCTACTGCCTTCCCTGACTTCACCAAT
CCTACTGCCCTGGCCTGGTGGGAGGACATGGTGGCTGAGTTCCATGACCAGGTGCCCTTTGATGGCATGTGGATT
GACATGAATGAGCCCTCTAATTTCATCAGGGGCTCTGAGGATGGCTGCCCCAACAATGAGCTGGAGAACCCCCCC
TATGTGCCTGGGGTGGTGGGGGGCACCCTGCAGGCTGCCACCATCTGTGCTAGCTCTCACCAGTTCCTGAGCACC
CACTACAATCTGCATAACCTGTATGGCCTGACTGAGGCCATTGCCAGCCACAGGGCCCTGGTGAAGGCTAGGGGC
ACCAGGCCCTTTGTGATTTCTAGGAGCACTTTTGCTGGCCATGGCAGGTATGCTGGGCACTGGACTGGGGATGTG
TGGTCTAGCTGGGAGCAGCTGGCTTCTTCTGTGCCTGAGATCCTGCAGTTCAACCTGCTGGGGGTGCCTCTGGTG
GGGGCTGATGTGTGTGGGTTCCTGGGCAACACTTCTGAGGAGCTGTGTGTGAGGTGGACCCAGCTGGGGGCCTTC
TACCCTTTCATGAGGAACCACAACAGCCTGCTGAGCCTGCCCCAGGAGCCCTACAGCTTCTCTGAGCCTGCCCAG
CAGGCCATGAGGAAGGCCCTGACCCTGAGGTATGCCCTGCTGCCCCACCTGTACACCCTGTTCCACCAGGCCCAT
GTGGCTGGGGAGACTGTGGCTAGGCCTCTGTTCCTGGAGTTCCCCAAGGACTCTAGCACCTGGACTGTGGACCAC
CAGCTGCTGTGGGGGGAGGCCCTGCTGATCACTCCTGTGCTGCAGGCTGGGAAGGCTGAGGTGACTGGCTATTTC
CCCCTGGGCACCTGGTATGACCTGCAGACTGTGCCTGTGGAGGCCCTGGGGAGCCTGCCCCCCCCCCCTGCTGCC
CCCAGGGAGCCTGCCATCCACTCTGAGGGCCAGTGGGTGACCCTGCCTGCCCCTCTGGATACCATCAATGTGCAC
CTGAGGGCTGGCTACATCATTCCCCTGCAGGGCCCTGGCCTGACCACTACTGAGTCTAGGCAGCAGCCCATGGCC
CTGGCTGTGGCCCTGACCAAGGGGGGGGAGGCTAGGGGGGAGCTGTTTTGGGATGATGGGGAGAGCCTGGAGGTG
CTGGAGAGGGGGGCCTACACTCAGGTGATCTTCCTGGCCAGGAACAATACCATTGTGAATGAGCTGGTGAGGGTG
ACCTCTGAGGGGGCTGGCCTGCAGCTGCAGAAGGTGACTGTGCTGGGGGTGGCCACTGCCCCCCAGCAGGTGCTG
AGCAATGGGGTGCCTGTGAGCAACTTCACCTATAGCCCTGATACCAAGGTGCTGGATATTTGTGTGAGCCTGCTG
ATGGGGGAGCAGTTCCTGGTGAGCTGGTGCTGAAGATCTAGAGCTGAATTCCTGCAGCCAGGGGGATCAGCCTCT
ACTGTGCCTTCTAGTTGCCAGCCATCTGTTGTTTGCCCCTCCCCCTTGCCTTCCTTGACCCTGGAAGGTGCCACT
CCCACTGTCCTTTCCTAATAAAATGAGGAAATTGCATCACATTGTCTGAGTAGGTGTCATTCTATTCTGGGGGGT
GGGGTGGGGCAGGACAGCAAGGGGGAGGATTGGGAAGACAATAGCAGGCATGCTGGGGATGCAGTGGGCTCTATG
SEQ ID NO: 13: nucleic acid sequence for GAA7 plus CpG-reduced BGH poly A
ATGGCTTTCCTGTGGCTGCTGTCTTGTTGGGCTCTGCTGGGCACCACCTTTGGCCTGCTGGTGCCCAGGGAGCTG
TCTGGCAGCAGCCCTGTGCTGGAGGAGACCCACCCTGCTCATCAGCAGGGGGCTAGCAGGCCTGGCCCCAGGGAT
GCCCAGGCTCACCCTGGGAGACCCAGGGCTGTGCCCACTCAGTGTGATGTGCCCCCCAACAGCAGGTTTGACTGT
GCTCCTGACAAGGCTATCACCCAGGAGCAGTGTGAGGCCAGGGGGTGCTGCTACATTCCTGCTAAGCAGGGCCTG
CAGGGGGCCCAGATGGGCCAGCCCTGGTGCTTCTTCCCCCCCTCTTATCCCAGCTATAAGCTGGAGAACCTGAGC
AGCTCTGAGATGGGCTACACTGCCACCCTGACCAGGACCACTCCCACCTTCTTTCCCAAGGATATTCTGACTCTG
AGGCTGGATGTGATGATGGAGACTGAGAACAGGCTGCACTTCACTATCAAGGACCCTGCCAATAGGAGGTATGAG
GTGCCCCTGGAGACTCCTCATGTGCATAGCAGGGCCCCTTCTCCTCTGTATTCTGTGGAGTTCTCTGAGGAGCCC
TTTGGGGTGATTGTGAGGAGGCAGCTGGATGGCAGGGTGCTGCTGAACACCACTGTGGCCCCCCTGTTCTTTGCT
GACCAGTTCCTGCAGCTGAGCACTTCTCTGCCCAGCCAGTACATTACTGGGCTGGCTGAGCATCTGAGCCCCCTG
ATGCTGAGCACCTCTTGGACCAGGATCACCCTGTGGAACAGGGACCTGGCCCCCACTCCTGGGGCTAACCTGTAT
GGCTCTCACCCCTTTTACCTGGCCCTGGAGGATGGGGGCTCTGCCCATGGGGTGTTTCTGCTGAACAGCAATGCT
ATGGATGTGGTGCTGCAGCCCTCTCCAGCCCTGTCTTGGAGGAGCACTGGGGGCATTCTGGATGTGTACATTTTC
CTGGGGCCTGAACCCAAGTCTGTGGTGCAGCAGTACCTGGATGTGGTGGGCTACCCCTTCATGCCCCCCTATTGG
GGGCTGGGGTTTCACCTGTGCAGGTGGGGCTACAGCAGCACTGCCATCACCAGGCAGGTGGTGGAGAACATGACC
AGGGCCCATTTCCCCCTGGATGTGCAGTGGAATGACCTGGACTACATGGATAGCAGGAGGGATTTCACCTTCAAC
AAGGATGGCTTCAGGGACTTTCCTGCCATGGTGCAGGAGCTGCACCAGGGGGGCAGGAGGTATATGATGATTGTG
GACCCTGCTATCAGCAGCTCTGGCCCTGCTGGCTCTTACAGGCCCTATGATGAGGGCCTGAGGAGGGGGGTGTTT
ATCACTAATGAAACTGGCCAGCCTCTGATTGGCAAGGTCTGGCCTGGCTCTACTGCCTTCCCTGATTTTACTAAC
CCCACTGCCCTGGCCTGGTGGGAGGACATGGTGGCTGAGTTCCATGATCAGGTGCCTTTTGATGGCATGTGGATT
GATATGAATGAACCAAGCAACTTCATCAGAGGCTCTGAGGATGGCTGCCCCAACAATGAGCTGGAGAACCCCCCC
TATGTGCCTGGGGTGGTGGGGGGCACTCTGCAGGCTGCCACCATTIGTGCTAGCAGCCACCAGTTCCTGAGCACC
CACTACAATCTGCACAACCTGTATGGCCTGACTGAAGCCATTGCCAGCCATAGGGCCCTGGTGAAGGCCAGGGGC
ACTAGGCCTTTTGTGATCAGCAGGAGCACTTTTGCTGGCCATGGCAGGTATGCTGGCCACTGGACTGGGGATGTG
TGGAGCAGCTGGGAGCAGCTGGCCAGCTCTGTGCCTGAGATTCTGCAGTTTAACCTGCTGGGGGTGCCCCTGGTG
GGGGCTGATGTGTGTGGCTTCCTGGGCAACACCTCTGAGGAGCTGTGTGTGAGGTGGACCCAGCTGGGGGCCTTT
TATCCCTTCATGAGGAACCACAACAGCCTGCTGAGCCTGCCTCAGGAGCCCTACTCTTTCTCTGAGCCTGCCCAG
CAGGCCATGAGGAAGGCCCTGACCCTGAGGTATGCCCTGCTGCCCCACCTGTATACCCTGTTCCACCAGGCCCAT
GTGGCTGGGGAGACTGTGGCCAGGCCCCTGTTCCTGGAGTTCCCCAAGGACAGCAGCACCTGGACTGTGGATCAT
CAGCTGCTGTGGGGGGAGGCCCTGCTGATCACCCCTGTGCTGCAGGCTGGCAAGGCTGAGGTCACTGGCTACTTC
CCTCTGGGCACCTGGTATGACCTGCAGACTGTGCCTGTGGAGGCTCTGGGCAGCCTGCCCCCCCCCCCTGCTGCT
CCCAGGGAGCCTGCCATCCACTCTGAGGGCCAGTGGGTGACCCTGCCTGCTCCCCTGGACACCATCAATGTGCAC
CTGAGGGCTGGCTACATTATCCCCCTGCAGGGCCCAGGGCTGACTACCACTGAGAGCAGACAGCAGCCCATGGCT
CTGGCTGTGGCCCTGACCAAGGGGGGGGAAGCTAGGGGGGAGCTGTTCTGGGATGATGGGGAGAGCCTGGAGGTG
CTGGAGAGGGGGGCCTATACCCAGGTGATCTTCCTGGCTAGGAACAACACCATTGTCAATGAGCTGGTGAGGGTG
ACTTCTGAGGGGGCTGGGCTGCAGCTGCAGAAGGTGACTGTGCTGGGGGTGGCCACTGCTCCCCAGCAGGTGCTG
AGCAATGGGGTGCCTGTGAGCAACTTCACCTACAGCCCTGACACCAAGGTGCTGGACATCTGTGTGTCTCTGCTG
ATGGGGGAGCAGTTCCTGGTGAGCTGGTGCTGAAGATCTAGAGCTGAATTCCTGCAGCCAGGGGGATCAGCCTCT
ACTGTGCCTTCTAGTTGCCAGCCATCTGTTGTTTGCCCCTCCCCCTTGCCTTCCTTGACCCTGGAAGGTGCCACT
CCCACTGTCCTTTCCTAATAAAATGAGGAAATTGCATCACATTGTCTGAGTAGGTGTCATTCTATTCTGGGGGGT
GGGGTGGGGCAGGACAGCAAGGGGGAGGATTGGGAAGACAATAGCAGGCATGCTGGGGATGCAGTGGGCTCTATG
G
SEQ ID NO: 14: nucleic acid sequence for GAA8 plus CpG-reduced BGH poly A
ATGGCCTTCCTGTGGCTGCTGTCTTGCTGGGCTCTGCTGGGGACCACCTTTGGCCTGCTGGTCCCCAGGGAGCTG
TCTGGCTCTTCTCCTGTCCTGGAGGAGACCCACCCTGCCCACCAGCAGGGGGCTAGCAGGCCTGGCCCCAGGGAT
GCCCAGGCCCACCCTGGCAGGCCCAGGGCTGTGCCCACCCAGTGTGATGTGCCTCCCAACAGCAGGTTTGACTGT
GCCCCTGACAAGGCCATCACCCAGGAGCAGTGTGAGGCCAGGGGCTGCTGCTATATCCCTGCCAAGCAGGGCCTG
CAGGGGGCTCAGATGGGCCAGCCCTGGTGCTTCTTTCCCCCCTCTTATCCTAGCTATAAGCTGGAGAACCTGAGC
AGCTCTGAGATGGGGTACACTGCCACCCTGACCAGGACCACCCCCACTTTCTTCCCTAAGGACATCCTGACCCTG
AGGCTGGATGTGATGATGGAGACTGAGAATAGGCTGCACTTTACTATCAAGGACCCTGCCAACAGGAGGTATGAG
GTGCCTCTGGAGACCCCCCATGTGCATTCTAGGGCCCCCAGCCCCCTGTACTCTGTGGAGTTCTCTGAGGAGCCC
TTTGGGGTGATTGTGAGGAGACAGCTGGATGGCAGGGTCCTGCTGAACACCACTGTGGCTCCCCTGTTTTTTGCT
GACCAGTTCCTGCAGCTGAGCACCAGCCTGCCCAGCCAGTACATCACTGGGCTGGCTGAGCACCTGAGCCCCCTG
ATGCTGAGCACCAGCTGGACCAGGATCACCCTGTGGAACAGGGATCTGGCTCCTACCCCTGGGGCCAACCTGTAT
GGCTCTCACCCCTTTTACCTGGCCCTGGAGGATGGGGGCTCTGCCCATGGGGTGTTCCTGCTGAACAGCAATGCT
ATGGATGTGGTGCTGCAGCCCAGCCCTGCCCTGAGCTGGAGGTCTACTGGGGGCATCCTGGATGTGTACATCTTT
CTGGGGCCTGAGCCCAAGTCTGTGGTGCAGCAGTACCTGGATGTGGTGGGCTATCCTTTTATGCCCCCCTATTGG
GGCCTGGGCTTCCACCTGTGCAGGTGGGGCTACAGCAGCACTGCCATCACCAGACAGGTGGTGGAGAACATGACC
AGGGCCCACTTCCCCCTGGATGTGCAGTGGAATGACCTGGACTACATGGACAGCAGGAGGGACTTCACCTTTAAC
AAGGATGGCTTTAGGGACTTCCCTGCCATGGTGCAGGAGCTGCATCAGGGGGGCAGGAGGTACATGATGATTGTG
GACCCAGCCATCAGCAGCTCTGGGCCTGCTGGGTCTTACAGGCCCTATGATGAGGGCCTGAGGAGGGGGGTGTTC
ATCACCAATGAGACTGGCCAGCCCCTGATTGGCAAGGTGTGGCCTGGGAGCACTGCCTTCCCTGATTTTACCAAC
CCCACTGCCCTGGCCTGGTGGGAGGATATGGTGGCTGAGTTTCATGACCAGGTGCCCTTTGATGGCATGTGGATT
GACATGAATGAGCCCAGCAATTTCATCAGGGGCTCTGAGGATGGCTGCCCCAACAATGAGCTGGAGAATCCTCCC
TATGTGCCTGGGGTGGTGGGGGGCACCCTGCAGGCTGCCACCATCTGTGCCTCTAGCCACCAGTTCCTGAGCACC
CACTATAACCTGCATAACCTGTATGGCCTGACTGAGGCCATTGCCAGCCATAGAGCCCTGGTGAAGGCCAGAGGG
ACCAGGCCCTTTGTGATCTCTAGGAGCACCTTTGCTGGCCATGGCAGGTATGCTGGCCACTGGACTGGGGATGTG
TGGAGCTCTTGGGAGCAGCTGGCCAGCTCTGTGCCAGAGATCCTGCAGTTCAACCTGCTGGGGGTGCCTCTGGTG
GGGGCTGATGTGTGTGGCTTCCTGGGCAATACCTCTGAAGAGCTGTGTGTGAGGTGGACTCAGCTGGGGGCCTTC
TATCCCTTCATGAGGAACCACAACAGCCTGCTGTCTCTGCCCCAGGAGCCCTACAGCTTCTCTGAGCCTGCTCAG
CAGGCTATGAGGAAGGCCCTGACCCTGAGGTATGCCCTGCTGCCCCATCTGTACACCCTGTTCCACCAGGCCCAT
GTGGCTGGGGAGACTGTGGCCAGGCCCCTGTTTCTGGAGTTTCCCAAGGACAGCAGCACCTGGACTGTGGACCAT
CAGCTGCTGTGGGGGGAGGCTCTGCTGATTACCCCTGTGCTGCAGGCTGGCAAGGCTGAGGTGACTGGGTACTTC
CCCCTGGGGACTTGGTATGACCTGCAGACTGTGCCTGTGGAAGCTCTGGGCAGCCTGCCCCCACCCCCTGCTGCC
CCTAGGGAGCCTGCCATCCACTCTGAGGGCCAGTGGGTGACCCTGCCTGCCCCTCTGGACACCATCAATGTGCAC
CTGAGGGCTGGCTATATCATCCCCCTGCAGGGCCCTGGGCTGACCACCACTGAGAGCAGGCAGCAGCCCATGGCC
CTGGCTGTGGCCCTGACTAAGGGGGGGGAGGCCAGGGGGGAGCTGTTCTGGGATGATGGGGAGAGCCTGGAGGTG
CTGGAGAGAGGGGCCTACACCCAGGTGATCTTTCTGGCCAGGAACAACACCATTGTGAATGAGCTGGTGAGGGTG
ACTTCTGAGGGGGCTGGCCTGCAGCTGCAGAAGGTGACTGTGCTGGGGGTGGCCACTGCCCCCCAGCAGGTGCTG
AGCAATGGGGTGCCTGTGTCTAACTTCACCTACAGCCCTGATACTAAGGTGCTGGATATCTGTGTGAGCCTGCTG
ATGGGGGAGCAGTTTCTGGTGAGCTGGTGCTGAAGATCTAGAGCTGAATTCCTGCAGCCAGGGGGATCAGCCTCT
ACTGTGCCTTCTAGTTGCCAGCCATCTGTTGTTTGCCCCTCCCCCTTGCCTTCCTTGACCCTGGAAGGTGCCACT
CCCACTGTCCTTTCCTAATAAAATGAGGAAATTGCATCACATTGTCTGAGTAGGTGTCATTCTATTCTGGGGGGT
GGGGTGGGGCAGGACAGCAAGGGGGAGGATTGGGAAGACAATAGCAGGCATGCTGGGGATGCAGTGGGCTCTATG
G
SEQ ID NO: 15: nucleic acid sequence for GAA13 plus CpG-reduced BGH poly A
ATGGCCTTTCTGTGGCTGCTGTCCTGCTGGGCCCTGCTGGGGACCACCTTTGGCCTGCTGGTGCCCAGGGAGCTG
TCTGGGAGCAGCCCAGTGCTGGAGGAGACCCACCCTGCCCACCAGCAGGGGGCCAGCAGGCCTGGCCCTAGGGAT
GCCCAGGCCCACCCTGGCAGGCCCAGGGCTGTGCCTACCCAGTGTGATGTGCCACCCAATTCTAGGTTTGACTGT
GCTCCTGACAAGGCCATCACTCAGGAGCAGTGTGAAGCTAGGGGGTGCTGCTACATCCCAGCCAAGCAGGGCCTG
CAGGGGGCCCAGATGGGCCAGCCCTGGTGCTTCTTCCCCCCCAGCTACCCTAGCTACAAGCTGGAGAATCTGAGC
AGCTCTGAGATGGGCTACACTGCTACCCTGACCAGGACCACTCCTACCTTCTTCCCCAAGGACATCCTGACTCTG
AGGCTGGATGTCATGATGGAGACTGAAAATAGGCTGCACTTCACCATCAAGGACCCTGCCAATAGGAGGTATGAG
GTGCCTCTGGAGACCCCCCATGTGCATAGCAGGGCTCCCAGCCCCCTGTATTCTGTGGAGTTCTCTGAGGAGCCC
TTTGGGGTCATTGTGAGGAGACAGCTGGATGGGAGGGTGCTGCTGAACACTACTGTGGCTCCCCTGTTCTTTGCT
GACCAGTTCCTGCAGCTGTCTACCAGCCTGCCCAGCCAGTACATCACTGGGCTGGCTGAGCATCTGAGCCCCCTG
ATGCTGAGCACCAGCTGGACCAGGATCACTCTGTGGAACAGGGATCTGGCCCCCACTCCTGGGGCCAACCTGTAT
GGGAGCCATCCCTTCTACCTGGCCCTGGAGGATGGGGGCTCTGCCCATGGGGTGTTCCTGCTGAACAGCAATGCC
ATGGATGTGGTGCTGCAGCCTAGCCCTGCCCTGAGCTGGAGGAGCACTGGGGGCATCCTGGATGTCTACATCTTC
CTGGGGCCTGAGCCCAAGTCTGTGGTGCAGCAGTATCTGGATGTGGTGGGGTATCCCTTCATGCCCCCCTACTGG
GGCCTGGGCTTTCACCTGTGCAGGTGGGGCTACAGCAGCACTGCCATCACCAGGCAGGTGGTGGAGAACATGACC
AGGGCCCACTTCCCTCTGGATGTGCAGTGGAATGACCTGGACTATATGGATTCTAGGAGAGACTTTACTTTTAAC
AAGGATGGCTTCAGGGATTTCCCTGCCATGGTGCAGGAGCTGCACCAGGGGGGCAGGAGGTACATGATGATTGTG
GACCCTGCTATTAGCAGCTCTGGCCCTGCTGGGTCTTACAGGCCTTATGATGAGGGCCTGAGGAGGGGGGTGTTC
ATCACCAATGAGACTGGCCAGCCCCTGATTGGCAAGGTGTGGCCTGGCAGCACTGCCTTCCCTGACTTCACCAAC
CCCACTGCCCTGGCCTGGTGGGAGGACATGGTGGCTGAGTTCCATGACCAGGTGCCCTTTGATGGGATGTGGATT
GACATGAATGAGCCCTCTAACTTCATCAGGGGGTCTGAGGATGGCTGCCCCAACAATGAGCTGGAGAACCCCCCC
TATGTGCCTGGGGTGGTGGGGGGCACTCTGCAGGCTGCCACTATCTGTGCTTCTTCTCACCAGTTTCTGAGCACC
CACTATAATCTGCACAACCTGTATGGCCTGACTGAGGCCATTGCCAGCCATAGGGCCCTGGTGAAGGCCAGGGGC
ACCAGGCCCTTTGTGATCAGCAGGTCTACCTTTGCTGGCCATGGCAGGTATGCTGGCCACTGGACTGGGGATGTG
TGGTCTTCTTGGGAGCAGCTGGCCAGCTCTGTGCCTGAGATCCTGCAGTTCAACCTGCTGGGGGTGCCTCTGGTG
GGGGCTGATGTGTGTGGCTTTCTGGGCAACACCTCTGAGGAGCTGTGTGTGAGGTGGACCCAGCTGGGGGCCTTT
TACCCCTTCATGAGGAACCACAATAGCCTGCTGAGCCTGCCCCAGGAGCCTTACTCTTTCTCTGAGCCTGCCCAG
CAGGCCATGAGGAAGGCCCTGACTCTGAGGTATGCCCTGCTGCCCCATCTGTATACCCTGTTTCACCAGGCCCAT
GTGGCTGGGGAGACTGTGGCTAGGCCTCTGTTTCTGGAGTTCCCTAAGGACTCTAGCACCTGGACTGTGGACCAC
CAGCTGCTGTGGGGGGAGGCCCTGCTGATCACCCCTGTGCTGCAGGCTGGCAAGGCTGAGGTGACTGGCTACTTC
CCCCTGGGCACCTGGTATGACCTGCAGACTGTGCCTGTGGAGGCCCTGGGGAGCCTGCCTCCCCCCCCTGCTGCC
CCCAGGGAGCCTGCCATTCATTCTGAGGGCCAGTGGGTGACCCTGCCTGCCCCTCTGGACACCATCAATGTGCAC
CTGAGGGCTGGGTACATCATCCCCCTGCAGGGCCCTGGCCTGACCACCACTGAGAGCAGGCAGCAGCCCATGGCC
CTGGCTGTGGCTCTGACCAAGGGGGGGGAGGCCAGGGGGGAGCTGTTCTGGGATGATGGGGAGTCTCTGGAGGTG
CTGGAGAGGGGGGCCTACACCCAGGTGATCTTTCTGGCCAGGAACAATACTATTGTGAATGAGCTGGTGAGGGTG
ACCTCTGAGGGGGCTGGCCTGCAGCTGCAGAAGGTGACTGTGCTGGGGGTGGCCACTGCCCCCCAGCAGGTCCTG
AGCAATGGGGTGCCTGTGAGCAACTTCACCTACTCTCCTGACACCAAGGTGCTGGACATTTGTGTGTCTCTGCTG
ATGGGGGAGCAGTTCCTGGTGAGCTGGTGCTGAAGATCTAGAGCTGAATTCCTGCAGCCAGGGGGATCAGCCTCT
ACTGTGCCTTCTAGTTGCCAGCCATCTGTTGTTTGCCCCTCCCCCTTGCCTTCCTTGACCCTGGAAGGTGCCACT
CCCACTGTCCTTTCCTAATAAAATGAGGAAATTGCATCACATTGTCTGAGTAGGTGTCATTCTATTCTGGGGGGT
GGGGTGGGGCAGGACAGCAAGGGGGAGGATTGGGAAGACAATAGCAGGCATGCTGGGGATGCAGTGGGCTCTATG
G
>pAAV-ApoE/hAAT.fixUTR.GAA13.BGH
(SEQ ID NO: 16)
CCTGCAGGCAGCTGCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAACTCCATCACTAGGGGTTCCTGCGGCCTAG
TAGGCTCAGAGGCACACAGGAGTTTCTGGGCTCACCCTGCCCCCTTCCAACCCCTCAGTTCCCATCCTCCAGCAG
CTGTTTGTGTGCTGCCTCTGAAGTCCACACTGAACAAACTTCAGCCTACTCATGTCCCTAAAATGGGCAAACATT
GCAAGCAGCAAACAGCAAACACACAGCCCTCCCTGCCTGCTGACCTTGGAGCTGGGGCAGAGGTCAGAGACCTCT
CTGGGCCCATGCCACCTCCAACATCCACTCGACCCCTTGGAATTTCGGTGGAGAGGAGCAGAGGTTGTCCTGGCG
TGGTTTAGGTAGTGTGAGAGGGGTACCCGGGGATCTTGCTACCAGTGGAACAGCCACTAAGGATTCTGCAGTGAG
AGCAGAGGGCCAGCTAAGTGGTACTCTCCCAGAGACTGTCTGACTCACGCCACCCCCTCCACCTTGGACACAGGA
CGCTGTGGTTTCTGAGCCAGGTACAATGACTCCTTTCGGTAAGTGCAGTGGAAGCTGTACACTGCCCAGGCAAAG
CGTCCGGGCAGCGTAGGCGGGCGACTCAGATCCCAGCCAGTGGACTTAGCCCCTGTTTGCTCCTCCGATAACTGG
GGTGACCTTGGTTAATATTCACCAGCAGCCTCCCCCGTTGCCCCTCTGGATCCACTGCTTAAATACGGACGAGGA
CAGGGCCCTGTCTCCTCAGCTTCAGGCACCACCACTGACCTGGGACAGTGAATACCACTTTCACAATCTGCTAGC
GTTTAAACGATCCTGAGAACTTCAGGGTGAGTCTATGGGACCCTTGATGTTTTCTTTCCCCTTCTTTTCTATGGT
TAAGTTCATGTCATAGGAAGGGGAGAAGTAACAGGGTACACATATTGACCAAATCAGGGTAATTTTGCATTTGTA
ATTTTAAAAAATGCTTTCTTCTTTTAATATACTTTTTTGTTTATCTTATTTCTAATACTTTCCCTAATCTCTTTC
TTTCAGGGCAATAATGATACAATGTATCATGCCTCTTTGCACCATTCTAAAGAATAACAGTGATAATTTCTGGGT
TAAGGCAATAGCAATATTTCTGCATATAAATATTTCTGCATATAAATTGTAACTGATGTAAGAGGTTTCATATTG
CTAATAGCAGCTACAATCCAGCTACCATTCTGCTTTTATTTTCTGGTTGGGATAAGGCTGGATTATTCTGAGTCC
AAGCTAGGCCCTTTTGCTAATCTTGTTCATACCTCTTATCTTCCTCCCACAGCTCCTGGGCAACCTGCTGGTCTC
TCTGCTGGCCCATCACTTTGGCAAAGCACGCGTGCCACCATGGCCTTTCTGTGGCTGCTGTCCTGCTGGGCCCTG
CTGGGGACCACCTTTGGCCTGCTGGTGCCCAGGGAGCTGTCTGGGAGCAGCCCAGTGCTGGAGGAGACCCACCCT
GCCCACCAGCAGGGGGCCAGCAGGCCTGGCCCTAGGGATGCCCAGGCCCACCCTGGCAGGCCCAGGGCTGTGCCT
ACCCAGTGTGATGTGCCACCCAATTCTAGGTTTGACTGTGCTCCTGACAAGGCCATCACTCAGGAGCAGTGTGAA
GCTAGGGGGTGCTGCTACATCCCAGCCAAGCAGGGCCTGCAGGGGGCCCAGATGGGCCAGCCCTGGTGCTTCTTC
CCCCCCAGCTACCCTAGCTACAAGCTGGAGAATCTGAGCAGCTCTGAGATGGGCTACACTGCTACCCTGACCAGG
ACCACTCCTACCTTCTTCCCCAAGGACATCCTGACTCTGAGGCTGGATGTCATGATGGAGACTGAAAATAGGCTG
CACTTCACCATCAAGGACCCTGCCAATAGGAGGTATGAGGTGCCTCTGGAGACCCCCCATGTGCATAGCAGGGCT
CCCAGCCCCCTGTATTCTGTGGAGTTCTCTGAGGAGCCCTTTGGGGTCATTGTGAGGAGACAGCTGGATGGGAGG
GTGCTGCTGAACACTACTGTGGCTCCCCTGTTCTTTGCTGACCAGTTCCTGCAGCTGTCTACCAGCCTGCCCAGC
CAGTACATCACTGGGCTGGCTGAGCATCTGAGCCCCCTGATGCTGAGCACCAGCTGGACCAGGATCACTCTGTGG
AACAGGGATCTGGCCCCCACTCCTGGGGCCAACCTGTATGGGAGCCATCCCTTCTACCTGGCCCTGGAGGATGGG
GGCTCTGCCCATGGGGTGTTCCTGCTGAACAGCAATGCCATGGATGTGGTGCTGCAGCCTAGCCCTGCCCTGAGC
TGGAGGAGCACTGGGGGCATCCTGGATGTCTACATCTTCCTGGGGCCTGAGCCCAAGTCTGTGGTGCAGCAGTAT
CTGGATGTGGTGGGGTATCCCTTCATGCCCCCCTACTGGGGCCTGGGCTTTCACCTGTGCAGGTGGGGCTACAGC
AGCACTGCCATCACCAGGCAGGTGGTGGAGAACATGACCAGGGCCCACTTCCCTCTGGATGTGCAGTGGAATGAC
CTGGACTATATGGATTCTAGGAGAGACTTTACTTTTAACAAGGATGGCTTCAGGGATTTCCCTGCCATGGTGCAG
GAGCTGCACCAGGGGGGCAGGAGGTACATGATGATTGTGGACCCTGCTATTAGCAGCTCTGGCCCTGCTGGGTCT
TACAGGCCTTATGATGAGGGCCTGAGGAGGGGGGTGTTCATCACCAATGAGACTGGCCAGCCCCTGATTGGCAAG
GTGTGGCCTGGCAGCACTGCCTTCCCTGACTTCACCAACCCCACTGCCCTGGCCTGGTGGGAGGACATGGTGGCT
GAGTTCCATGACCAGGTGCCCTTTGATGGGATGTGGATTGACATGAATGAGCCCTCTAACTTCATCAGGGGGTCT
GAGGATGGCTGCCCCAACAATGAGCTGGAGAACCCCCCCTATGTGCCTGGGGTGGTGGGGGGCACTCTGCAGGCT
GCCACTATCTGTGCTTCTTCTCACCAGTTTCTGAGCACCCACTATAATCTGCACAACCTGTATGGCCTGACTGAG
GCCATTGCCAGCCATAGGGCCCTGGTGAAGGCCAGGGGCACCAGGCCCTTTGTGATCAGCAGGTCTACCTTTGCT
GGCCATGGCAGGTATGCTGGCCACTGGACTGGGGATGTGTGGTCTTCTTGGGAGCAGCTGGCCAGCTCTGTGCCT
GAGATCCTGCAGTTCAACCTGCTGGGGGTGCCTCTGGTGGGGGCTGATGTGTGTGGCTTTCTGGGCAACACCTCT
GAGGAGCTGTGTGTGAGGTGGACCCAGCTGGGGGCCTTTTACCCCTTCATGAGGAACCACAATAGCCTGCTGAGC
CTGCCCCAGGAGCCTTACTCTTTCTCTGAGCCTGCCCAGCAGGCCATGAGGAAGGCCCTGACTCTGAGGTATGCC
CTGCTGCCCCATCTGTATACCCTGTTTCACCAGGCCCATGTGGCTGGGGAGACTGTGGCTAGGCCTCTGTTTCTG
GAGTTCCCTAAGGACTCTAGCACCTGGACTGTGGACCACCAGCTGCTGTGGGGGGAGGCCCTGCTGATCACCCCT
GTGCTGCAGGCTGGCAAGGCTGAGGTGACTGGCTACTTCCCCCTGGGCACCTGGTATGACCTGCAGACTGTGCCT
GTGGAGGCCCTGGGGAGCCTGCCTCCCCCCCCTGCTGCCCCCAGGGAGCCTGCCATTCATTCTGAGGGCCAGTGG
GTGACCCTGCCTGCCCCTCTGGACACCATCAATGTGCACCTGAGGGCTGGGTACATCATCCCCCTGCAGGGCCCT
GGCCTGACCACCACTGAGAGCAGGCAGCAGCCCATGGCCCTGGCTGTGGCTCTGACCAAGGGGGGGGAGGCCAGG
GGGGAGCTGTTCTGGGATGATGGGGAGTCTCTGGAGGTGCTGGAGAGGGGGGCCTACACCCAGGTGATCTTTCTG
GCCAGGAACAATACTATTGTGAATGAGCTGGTGAGGGTGACCTCTGAGGGGGCTGGCCTGCAGCTGCAGAAGGTG
ACTGTGCTGGGGGTGGCCACTGCCCCCCAGCAGGTCCTGAGCAATGGGGTGCCTGTGAGCAACTTCACCTACTCT
CCTGACACCAAGGTGCTGGACATTTGTGTGTCTCTGCTGATGGGGGAGCAGTTCCTGGTGAGCTGGTGCTGAAGA
TCTAGAGCTGAATTCCTGCAGCCAGGGGGATCAGCCTCTACTGTGCCTTCTAGTTGCCAGCCATCTGTTGTTTGC
CCCTCCCCCTTGCCTTCCTTGACCCTGGAAGGTGCCACTCCCACTGTCCTTTCCTAATAAAATGAGGAAATTGCA
TCACATTGTCTGAGTAGGTGTCATTCTATTCTGGGGGGTGGGGTGGGGCAGGACAGCAAGGGGGAGGATTGGGAA
GACAATAGCAGGCATGCTGGGGATGCAGTGGGCTCTATGGCTTCTGAGGCAGAAAGAACCAGCTGGGGCTCGAGA
TCCACTAGGGCCGCAGGAACCCCTAGTGATGGAGTTGGCCACTCCCTCTCTGCGCGCTCGCTCGCTCACTGAGGC
CGGGCGACCAAAGGTCGCCCGACGCCCGGGCTTTGCCCGGGCGGCCTCAGTGAGCGAGCGAGCGCGCAGCTGCCT
GCAGG
>pAAV-ApoE/hAAT.GAA13.BGH
(SEQ ID NO: 17)
CCTGCAGGCAGCTGCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAACTCCATCACTAGGGGTTCCTGCGGCCTAG
TAGGCTCAGAGGCACACAGGAGTTTCTGGGCTCACCCTGCCCCCTTCCAACCCCTCAGTTCCCATCCTCCAGCAG
CTGTTTGTGTGCTGCCTCTGAAGTCCACACTGAACAAACTTCAGCCTACTCATGTCCCTAAAATGGGCAAACATT
GCAAGCAGCAAACAGCAAACACACAGCCCTCCCTGCCTGCTGACCTTGGAGCTGGGGCAGAGGTCAGAGACCTCT
CTGGGCCCATGCCACCTCCAACATCCACTCGACCCCTTGGAATTTCGGTGGAGAGGAGCAGAGGTTGTCCTGGCG
TGGTTTAGGTAGTGTGAGAGGGGTACCCGGGGATCTTGCTACCAGTGGAACAGCCACTAAGGATTCTGCAGTGAG
AGCAGAGGGCCAGCTAAGTGGTACTCTCCCAGAGACTGTCTGACTCACGCCACCCCCTCCACCTTGGACACAGGA
CGCTGTGGTTTCTGAGCCAGGTACAATGACTCCTTTCGGTAAGTGCAGTGGAAGCTGTACACTGCCCAGGCAAAG
CGTCCGGGCAGCGTAGGCGGGCGACTCAGATCCCAGCCAGTGGACTTAGCCCCTGTTTGCTCCTCCGATAACTGG
GGTGACCTTGGTTAATATTCACCAGCAGCCTCCCCCGTTGCCCCTCTGGATCCACTGCTTAAATACGGACGAGGA
CAGGGCCCTGTCTCCTCAGCTTCAGGCACCACCACTGACCTGGGACAGTGAATAGATCCTGAGAACTTCAGGGTG
AGTCTATGGGACCCTTGATGTTTTCTTTCCCCTTCTTTTCTATGGTTAAGTTCATGTCATAGGAAGGGGAGAAGT
AACAGGGTACACATATTGACCAAATCAGGGTAATTTTGCATTTGTAATTTTAAAAAATGCTTTCTTCTTTTAATA
TACTTTTTTGTTTATCTTATTTCTAATACTTTCCCTAATCTCTTTCTTTCAGGGCAATAATGATACAATGTATCA
TGCCTCTTTGCACCATTCTAAAGAATAACAGTGATAATTTCTGGGTTAAGGCAATAGCAATATTTCTGCATATAA
ATATTTCTGCATATAAATTGTAACTGATGTAAGAGGTTTCATATTGCTAATAGCAGCTACAATCCAGCTACCATT
CTGCTTTTATTTTCTGGTTGGGATAAGGCTGGATTATTCTGAGTCCAAGCTAGGCCCTTTTGCTAATCTTGTTCA
TACCTCTTATCTTCCTCCCACAGCTCCTGGGCAACCTGCTGGTCTCTCTGCTGGCCCATCACTTTGGCAAAGCAC
GCGTGCCACCATGGCCTTTCTGTGGCTGCTGTCCTGCTGGGCCCTGCTGGGGACCACCTTTGGCCTGCTGGTGCC
CAGGGAGCTGTCTGGGAGCAGCCCAGTGCTGGAGGAGACCCACCCTGCCCACCAGCAGGGGGCCAGCAGGCCTGG
CCCTAGGGATGCCCAGGCCCACCCTGGCAGGCCCAGGGCTGTGCCTACCCAGTGTGATGTGCCACCCAATTCTAG
GTTTGACTGTGCTCCTGACAAGGCCATCACTCAGGAGCAGTGTGAAGCTAGGGGGTGCTGCTACATCCCAGCCAA
GCAGGGCCTGCAGGGGGCCCAGATGGGCCAGCCCTGGTGCTTCTTCCCCCCCAGCTACCCTAGCTACAAGCTGGA
GAATCTGAGCAGCTCTGAGATGGGCTACACTGCTACCCTGACCAGGACCACTCCTACCTTCTTCCCCAAGGACAT
CCTGACTCTGAGGCTGGATGTCATGATGGAGACTGAAAATAGGCTGCACTTCACCATCAAGGACCCTGCCAATAG
GAGGTATGAGGTGCCTCTGGAGACCCCCCATGTGCATAGCAGGGCTCCCAGCCCCCTGTATTCTGTGGAGTTCTC
TGAGGAGCCCTTTGGGGTCATTGTGAGGAGACAGCTGGATGGGAGGGTGCTGCTGAACACTACTGTGGCTCCCCT
GTTCTTTGCTGACCAGTTCCTGCAGCTGTCTACCAGCCTGCCCAGCCAGTACATCACTGGGCTGGCTGAGCATCT
GAGCCCCCTGATGCTGAGCACCAGCTGGACCAGGATCACTCTGTGGAACAGGGATCTGGCCCCCACTCCTGGGGC
CAACCTGTATGGGAGCCATCCCTTCTACCTGGCCCTGGAGGATGGGGGCTCTGCCCATGGGGTGTTCCTGCTGAA
CAGCAATGCCATGGATGTGGTGCTGCAGCCTAGCCCTGCCCTGAGCTGGAGGAGCACTGGGGGCATCCTGGATGT
CTACATCTTCCTGGGGCCTGAGCCCAAGTCTGTGGTGCAGCAGTATCTGGATGTGGTGGGGTATCCCTTCATGCC
CCCCTACTGGGGCCTGGGCTTTCACCTGTGCAGGTGGGGCTACAGCAGCACTGCCATCACCAGGCAGGTGGTGGA
GAACATGACCAGGGCCCACTTCCCTCTGGATGTGCAGTGGAATGACCTGGACTATATGGATTCTAGGAGAGACTT
TACTTTTAACAAGGATGGCTTCAGGGATTTCCCTGCCATGGTGCAGGAGCTGCACCAGGGGGGCAGGAGGTACAT
GATGATTGTGGACCCTGCTATTAGCAGCTCTGGCCCTGCTGGGTCTTACAGGCCTTATGATGAGGGCCTGAGGAG
GGGGGTGTTCATCACCAATGAGACTGGCCAGCCCCTGATTGGCAAGGTGTGGCCTGGCAGCACTGCCTTCCCTGA
CTTCACCAACCCCACTGCCCTGGCCTGGTGGGAGGACATGGTGGCTGAGTTCCATGACCAGGTGCCCTTTGATGG
GATGTGGATTGACATGAATGAGCCCTCTAACTTCATCAGGGGGTCTGAGGATGGCTGCCCCAACAATGAGCTGGA
GAACCCCCCCTATGTGCCTGGGGTGGTGGGGGGCACTCTGCAGGCTGCCACTATCTGTGCTTCTTCTCACCAGTT
TCTGAGCACCCACTATAATCTGCACAACCTGTATGGCCTGACTGAGGCCATTGCCAGCCATAGGGCCCTGGTGAA
GGCCAGGGGCACCAGGCCCTTTGTGATCAGCAGGTCTACCTTTGCTGGCCATGGCAGGTATGCTGGCCACTGGAC
TGGGGATGTGTGGTCTTCTTGGGAGCAGCTGGCCAGCTCTGTGCCTGAGATCCTGCAGTTCAACCTGCTGGGGGT
GCCTCTGGTGGGGGCTGATGTGTGTGGCTTTCTGGGCAACACCTCTGAGGAGCTGTGTGTGAGGTGGACCCAGCT
GGGGGCCTTTTACCCCTTCATGAGGAACCACAATAGCCTGCTGAGCCTGCCCCAGGAGCCTTACTCTTTCTCTGA
GCCTGCCCAGCAGGCCATGAGGAAGGCCCTGACTCTGAGGTATGCCCTGCTGCCCCATCTGTATACCCTGTTTCA
CCAGGCCCATGTGGCTGGGGAGACTGTGGCTAGGCCTCTGTTTCTGGAGTTCCCTAAGGACTCTAGCACCTGGAC
TGTGGACCACCAGCTGCTGTGGGGGGAGGCCCTGCTGATCACCCCTGTGCTGCAGGCTGGCAAGGCTGAGGTGAC
TGGCTACTTCCCCCTGGGCACCTGGTATGACCTGCAGACTGTGCCTGTGGAGGCCCTGGGGAGCCTGCCTCCCCC
CCCTGCTGCCCCCAGGGAGCCTGCCATTCATTCTGAGGGCCAGTGGGTGACCCTGCCTGCCCCTCTGGACACCAT
CAATGTGCACCTGAGGGCTGGGTACATCATCCCCCTGCAGGGCCCTGGCCTGACCACCACTGAGAGCAGGCAGCA
GCCCATGGCCCTGGCTGTGGCTCTGACCAAGGGGGGGGAGGCCAGGGGGGAGCTGTTCTGGGATGATGGGGAGTC
TCTGGAGGTGCTGGAGAGGGGGGCCTACACCCAGGTGATCTTTCTGGCCAGGAACAATACTATTGTGAATGAGCT
GGTGAGGGTGACCTCTGAGGGGGCTGGCCTGCAGCTGCAGAAGGTGACTGTGCTGGGGGTGGCCACTGCCCCCCA
GCAGGTCCTGAGCAATGGGGTGCCTGTGAGCAACTTCACCTACTCTCCTGACACCAAGGTGCTGGACATTTGTGT
GTCTCTGCTGATGGGGGAGCAGTTCCTGGTGAGCTGGTGCTGAAGATCTAGAGCTGAATTCCTGCAGCCAGGGGG
ATCAGCCTCTACTGTGCCTTCTAGTTGCCAGCCATCTGTTGTTTGCCCCTCCCCCTTGCCTTCCTTGACCCTGGA
AGGTGCCACTCCCACTGTCCTTTCCTAATAAAATGAGGAAATTGCATCACATTGTCTGAGTAGGTGTCATTCTAT
TCTGGGGGGTGGGGTGGGGCAGGACAGCAAGGGGGAGGATTGGGAAGACAATAGCAGGCATGCTGGGGATGCAGT
GGGCTCTATGGCTTCTGAGGCAGAAAGAACCAGCTGGGGCTCGAGATCCACTAGGGCCGCAGGAACCCCTAGTGA
TGGAGTTGGCCACTCCCTCTCTGCGCGCTCGCTCGCTCACTGAGGCCGGGCGACCAAAGGTCGCCCGACGCCCGG
GCTTTGCCCGGGCGGCCTCAGTGAGCGAGCGAGCGCGCAGCTGCCTGCAGG
>pAAV-ApoE/hAAT.GAA7.BGH
(SEQ ID NO: 18)
CCTGCAGGCAGCTGCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAACTCCATCACTAGGGGTTCCTGCGGCCTAG
TAGGCTCAGAGGCACACAGGAGTTTCTGGGCTCACCCTGCCCCCTTCCAACCCCTCAGTTCCCATCCTCCAGCAG
CTGTTTGTGTGCTGCCTCTGAAGTCCACACTGAACAAACTTCAGCCTACTCATGTCCCTAAAATGGGCAAACATT
GCAAGCAGCAAACAGCAAACACACAGCCCTCCCTGCCTGCTGACCTTGGAGCTGGGGCAGAGGTCAGAGACCTCT
CTGGGCCCATGCCACCTCCAACATCCACTCGACCCCTTGGAATTTCGGTGGAGAGGAGCAGAGGTTGTCCTGGCG
TGGTTTAGGTAGTGTGAGAGGGGTACCCGGGGATCTTGCTACCAGTGGAACAGCCACTAAGGATTCTGCAGTGAG
AGCAGAGGGCCAGCTAAGTGGTACTCTCCCAGAGACTGTCTGACTCACGCCACCCCCTCCACCTTGGACACAGGA
CGCTGTGGTTTCTGAGCCAGGTACAATGACTCCTTTCGGTAAGTGCAGTGGAAGCTGTACACTGCCCAGGCAAAG
CGTCCGGGCAGCGTAGGCGGGCGACTCAGATCCCAGCCAGTGGACTTAGCCCCTGTTTGCTCCTCCGATAACTGG
GGTGACCTTGGTTAATATTCACCAGCAGCCTCCCCCGTTGCCCCTCTGGATCCACTGCTTAAATACGGACGAGGA
CAGGGCCCTGTCTCCTCAGCTTCAGGCACCACCACTGACCTGGGACAGTGAATAGATCCTGAGAACTTCAGGGTG
AGTCTATGGGACCCTTGATGTTTTCTTTCCCCTTCTTTTCTATGGTTAAGTTCATGTCATAGGAAGGGGAGAAGT
AACAGGGTACACATATTGACCAAATCAGGGTAATTTTGCATTTGTAATTTTAAAAAATGCTTTCTTCTTTTAATA
TACTTTTTTGTTTATCTTATTTCTAATACTTTCCCTAATCTCTTTCTTTCAGGGCAATAATGATACAATGTATCA
TGCCTCTTTGCACCATTCTAAAGAATAACAGTGATAATTTCTGGGTTAAGGCAATAGCAATATTTCTGCATATAA
ATATTTCTGCATATAAATTGTAACTGATGTAAGAGGTTTCATATTGCTAATAGCAGCTACAATCCAGCTACCATT
CTGCTTTTATTTTCTGGTTGGGATAAGGCTGGATTATTCTGAGTCCAAGCTAGGCCCTTTTGCTAATCTTGTTCA
TACCTCTTATCTTCCTCCCACAGCTCCTGGGCAACCTGCTGGTCTCTCTGCTGGCCCATCACTTTGGCAAAGCAC
GCGTGCCACCATGGCTTTCCTGTGGCTGCTGTCTTGTTGGGCTCTGCTGGGCACCACCTTTGGCCTGCTGGTGCC
CAGGGAGCTGTCTGGCAGCAGCCCTGTGCTGGAGGAGACCCACCCTGCTCATCAGCAGGGGGCTAGCAGGCCTGG
CCCCAGGGATGCCCAGGCTCACCCTGGGAGACCCAGGGCTGTGCCCACTCAGTGTGATGTGCCCCCCAACAGCAG
GTTTGACTGTGCTCCTGACAAGGCTATCACCCAGGAGCAGTGTGAGGCCAGGGGGTGCTGCTACATTCCTGCTAA
GCAGGGCCTGCAGGGGGCCCAGATGGGCCAGCCCTGGTGCTTCTTCCCCCCCTCTTATCCCAGCTATAAGCTGGA
GAACCTGAGCAGCTCTGAGATGGGCTACACTGCCACCCTGACCAGGACCACTCCCACCTTCTTTCCCAAGGATAT
TCTGACTCTGAGGCTGGATGTGATGATGGAGACTGAGAACAGGCTGCACTTCACTATCAAGGACCCTGCCAATAG
GAGGTATGAGGTGCCCCTGGAGACTCCTCATGTGCATAGCAGGGCCCCTTCTCCTCTGTATTCTGTGGAGTTCTC
TGAGGAGCCCTTTGGGGTGATTGTGAGGAGGCAGCTGGATGGCAGGGTGCTGCTGAACACCACTGTGGCCCCCCT
GTTCTTTGCTGACCAGTTCCTGCAGCTGAGCACTTCTCTGCCCAGCCAGTACATTACTGGGCTGGCTGAGCATCT
GAGCCCCCTGATGCTGAGCACCTCTTGGACCAGGATCACCCTGTGGAACAGGGACCTGGCCCCCACTCCTGGGGC
TAACCTGTATGGCTCTCACCCCTTTTACCTGGCCCTGGAGGATGGGGGCTCTGCCCATGGGGTGTTTCTGCTGAA
CAGCAATGCTATGGATGTGGTGCTGCAGCCCTCTCCAGCCCTGTCTTGGAGGAGCACTGGGGGCATTCTGGATGT
GTACATTTTCCTGGGGCCTGAACCCAAGTCTGTGGTGCAGCAGTACCTGGATGTGGTGGGCTACCCCTTCATGCC
CCCCTATTGGGGGCTGGGGTTTCACCTGTGCAGGTGGGGCTACAGCAGCACTGCCATCACCAGGCAGGTGGTGGA
GAACATGACCAGGGCCCATTTCCCCCTGGATGTGCAGTGGAATGACCTGGACTACATGGATAGCAGGAGGGATTT
CACCTTCAACAAGGATGGCTTCAGGGACTTTCCTGCCATGGTGCAGGAGCTGCACCAGGGGGGCAGGAGGTATAT
GATGATTGTGGACCCTGCTATCAGCAGCTCTGGCCCTGCTGGCTCTTACAGGCCCTATGATGAGGGCCTGAGGAG
GGGGGTGTTTATCACTAATGAAACTGGCCAGCCTCTGATTGGCAAGGTCTGGCCTGGCTCTACTGCCTTCCCTGA
TTTTACTAACCCCACTGCCCTGGCCTGGTGGGAGGACATGGTGGCTGAGTTCCATGATCAGGTGCCTTTTGATGG
CATGTGGATTGATATGAATGAACCAAGCAACTTCATCAGAGGCTCTGAGGATGGCTGCCCCAACAATGAGCTGGA
GAACCCCCCCTATGTGCCTGGGGTGGTGGGGGGCACTCTGCAGGCTGCCACCATTTGTGCTAGCAGCCACCAGTT
CCTGAGCACCCACTACAATCTGCACAACCTGTATGGCCTGACTGAAGCCATTGCCAGCCATAGGGCCCTGGTGAA
GGCCAGGGGCACTAGGCCTTTTGTGATCAGCAGGAGCACTTTTGCTGGCCATGGCAGGTATGCTGGCCACTGGAC
TGGGGATGTGTGGAGCAGCTGGGAGCAGCTGGCCAGCTCTGTGCCTGAGATTCTGCAGTTTAACCTGCTGGGGGT
GCCCCTGGTGGGGGCTGATGTGTGTGGCTTCCTGGGCAACACCTCTGAGGAGCTGTGTGTGAGGTGGACCCAGCT
GGGGGCCTTTTATCCCTTCATGAGGAACCACAACAGCCTGCTGAGCCTGCCTCAGGAGCCCTACTCTTTCTCTGA
GCCTGCCCAGCAGGCCATGAGGAAGGCCCTGACCCTGAGGTATGCCCTGCTGCCCCACCTGTATACCCTGTTCCA
CCAGGCCCATGTGGCTGGGGAGACTGTGGCCAGGCCCCTGTTCCTGGAGTTCCCCAAGGACAGCAGCACCTGGAC
TGTGGATCATCAGCTGCTGTGGGGGGAGGCCCTGCTGATCACCCCTGTGCTGCAGGCTGGCAAGGCTGAGGTCAC
TGGCTACTTCCCTCTGGGCACCTGGTATGACCTGCAGACTGTGCCTGTGGAGGCTCTGGGCAGCCTGCCCCCCCC
CCCTGCTGCTCCCAGGGAGCCTGCCATCCACTCTGAGGGCCAGTGGGTGACCCTGCCTGCTCCCCTGGACACCAT
CAATGTGCACCTGAGGGCTGGCTACATTATCCCCCTGCAGGGCCCAGGGCTGACTACCACTGAGAGCAGACAGCA
GCCCATGGCTCTGGCTGTGGCCCTGACCAAGGGGGGGGAAGCTAGGGGGGAGCTGTTCTGGGATGATGGGGAGAG
CCTGGAGGTGCTGGAGAGGGGGGCCTATACCCAGGTGATCTTCCTGGCTAGGAACAACACCATTGTCAATGAGCT
GGTGAGGGTGACTTCTGAGGGGGCTGGGCTGCAGCTGCAGAAGGTGACTGTGCTGGGGGTGGCCACTGCTCCCCA
GCAGGTGCTGAGCAATGGGGTGCCTGTGAGCAACTTCACCTACAGCCCTGACACCAAGGTGCTGGACATCTGTGT
GTCTCTGCTGATGGGGGAGCAGTTCCTGGTGAGCTGGTGCTGAAGATCTAGAGCTGAATTCCTGCAGCCAGGGGG
ATCAGCCTCTACTGTGCCTTCTAGTTGCCAGCCATCTGTTGTTTGCCCCTCCCCCTTGCCTTCCTTGACCCTGGA
AGGTGCCACTCCCACTGTCCTTTCCTAATAAAATGAGGAAATTGCATCACATTGTCTGAGTAGGTGTCATTCTAT
TCTGGGGGGTGGGGTGGGGCAGGACAGCAAGGGGGAGGATTGGGAAGACAATAGCAGGCATGCTGGGGATGCAGT
GGGCTCTATGGCTTCTGAGGCAGAAAGAACCAGCTGGGGCTCGAGATCCACTAGGGCCGCAGGAACCCCTAGTGA
TGGAGTTGGCCACTCCCTCTCTGCGCGCTCGCTCGCTCACTGAGGCCGGGCGACCAAAGGTCGCCCGACGCCCGG
GCTTTGCCCGGGCGGCCTCAGTGAGCGAGCGAGCGCGCAGCTGCCTGCAGG
>pAAV-ApoE/hAAT.GAA8.BGH
(SEQ ID NO: 19)
CCTGCAGGCAGCTGCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAACTCCATCACTAGGGGTTCCTGCGGCCTAG
TAGGCTCAGAGGCACACAGGAGTTTCTGGGCTCACCCTGCCCCCTTCCAACCCCTCAGTTCCCATCCTCCAGCAG
CTGTTTGTGTGCTGCCTCTGAAGTCCACACTGAACAAACTTCAGCCTACTCATGTCCCTAAAATGGGCAAACATT
GCAAGCAGCAAACAGCAAACACACAGCCCTCCCTGCCTGCTGACCTTGGAGCTGGGGCAGAGGTCAGAGACCTCT
CTGGGCCCATGCCACCTCCAACATCCACTCGACCCCTTGGAATTTCGGTGGAGAGGAGCAGAGGTTGTCCTGGCG
TGGTTTAGGTAGTGTGAGAGGGGTACCCGGGGATCTTGCTACCAGTGGAACAGCCACTAAGGATTCTGCAGTGAG
AGCAGAGGGCCAGCTAAGTGGTACTCTCCCAGAGACTGTCTGACTCACGCCACCCCCTCCACCTTGGACACAGGA
CGCTGTGGTTTCTGAGCCAGGTACAATGACTCCTTTCGGTAAGTGCAGTGGAAGCTGTACACTGCCCAGGCAAAG
CGTCCGGGCAGCGTAGGCGGGCGACTCAGATCCCAGCCAGTGGACTTAGCCCCTGTTTGCTCCTCCGATAACTGG
GGTGACCTTGGTTAATATTCACCAGCAGCCTCCCCCGTTGCCCCTCTGGATCCACTGCTTAAATACGGACGAGGA
CAGGGCCCTGTCTCCTCAGCTTCAGGCACCACCACTGACCTGGGACAGTGAATAGATCCTGAGAACTTCAGGGTG
AGTCTATGGGACCCTTGATGTTTTCTTTCCCCTTCTTTTCTATGGTTAAGTTCATGTCATAGGAAGGGGAGAAGT
AACAGGGTACACATATTGACCAAATCAGGGTAATTTTGCATTTGTAATTTTAAAAAATGCTTTCTTCTTTTAATA
TACTTTTTTGTTTATCTTATTTCTAATACTTTCCCTAATCTCTTTCTTTCAGGGCAATAATGATACAATGTATCA
TGCCTCTTTGCACCATTCTAAAGAATAACAGTGATAATTTCTGGGTTAAGGCAATAGCAATATTTCTGCATATAA
ATATTTCTGCATATAAATTGTAACTGATGTAAGAGGTTTCATATTGCTAATAGCAGCTACAATCCAGCTACCATT
CTGCTTTTATTTTCTGGTTGGGATAAGGCTGGATTATTCTGAGTCCAAGCTAGGCCCTTTTGCTAATCTTGTTCA
TACCTCTTATCTTCCTCCCACAGCTCCTGGGCAACCTGCTGGTCTCTCTGCTGGCCCATCACTTTGGCAAAGCAC
GCGTGCCACCATGGCCTTCCTGTGGCTGCTGTCTTGCTGGGCTCTGCTGGGGACCACCTTTGGCCTGCTGGTCCC
CAGGGAGCTGTCTGGCTCTTCTCCTGTCCTGGAGGAGACCCACCCTGCCCACCAGCAGGGGGCTAGCAGGCCTGG
CCCCAGGGATGCCCAGGCCCACCCTGGCAGGCCCAGGGCTGTGCCCACCCAGTGTGATGTGCCTCCCAACAGCAG
GTTTGACTGTGCCCCTGACAAGGCCATCACCCAGGAGCAGTGTGAGGCCAGGGGCTGCTGCTATATCCCTGCCAA
GCAGGGCCTGCAGGGGGCTCAGATGGGCCAGCCCTGGTGCTTCTTTCCCCCCTCTTATCCTAGCTATAAGCTGGA
GAACCTGAGCAGCTCTGAGATGGGGTACACTGCCACCCTGACCAGGACCACCCCCACTTTCTTCCCTAAGGACAT
CCTGACCCTGAGGCTGGATGTGATGATGGAGACTGAGAATAGGCTGCACTTTACTATCAAGGACCCTGCCAACAG
GAGGTATGAGGTGCCTCTGGAGACCCCCCATGTGCATTCTAGGGCCCCCAGCCCCCTGTACTCTGTGGAGTTCTC
TGAGGAGCCCTTTGGGGTGATTGTGAGGAGACAGCTGGATGGCAGGGTCCTGCTGAACACCACTGTGGCTCCCCT
GTTTTTTGCTGACCAGTTCCTGCAGCTGAGCACCAGCCTGCCCAGCCAGTACATCACTGGGCTGGCTGAGCACCT
GAGCCCCCTGATGCTGAGCACCAGCTGGACCAGGATCACCCTGTGGAACAGGGATCTGGCTCCTACCCCTGGGGC
CAACCTGTATGGCTCTCACCCCTTTTACCTGGCCCTGGAGGATGGGGGCTCTGCCCATGGGGTGTTCCTGCTGAA
CAGCAATGCTATGGATGTGGTGCTGCAGCCCAGCCCTGCCCTGAGCTGGAGGTCTACTGGGGGCATCCTGGATGT
GTACATCTTTCTGGGGCCTGAGCCCAAGTCTGTGGTGCAGCAGTACCTGGATGTGGTGGGCTATCCTTTTATGCC
CCCCTATTGGGGCCTGGGCTTCCACCTGTGCAGGTGGGGCTACAGCAGCACTGCCATCACCAGACAGGTGGTGGA
GAACATGACCAGGGCCCACTTCCCCCTGGATGTGCAGTGGAATGACCTGGACTACATGGACAGCAGGAGGGACTT
CACCTTTAACAAGGATGGCTTTAGGGACTTCCCTGCCATGGTGCAGGAGCTGCATCAGGGGGGCAGGAGGTACAT
GATGATTGTGGACCCAGCCATCAGCAGCTCTGGGCCTGCTGGGTCTTACAGGCCCTATGATGAGGGCCTGAGGAG
GGGGGTGTTCATCACCAATGAGACTGGCCAGCCCCTGATTGGCAAGGTGTGGCCTGGGAGCACTGCCTTCCCTGA
TTTTACCAACCCCACTGCCCTGGCCTGGTGGGAGGATATGGTGGCTGAGTTTCATGACCAGGTGCCCTTTGATGG
CATGTGGATTGACATGAATGAGCCCAGCAATTTCATCAGGGGCTCTGAGGATGGCTGCCCCAACAATGAGCTGGA
GAATCCTCCCTATGTGCCTGGGGTGGTGGGGGGCACCCTGCAGGCTGCCACCATCTGTGCCTCTAGCCACCAGTT
CCTGAGCACCCACTATAACCTGCATAACCTGTATGGCCTGACTGAGGCCATTGCCAGCCATAGAGCCCTGGTGAA
GGCCAGAGGGACCAGGCCCTTTGTGATCTCTAGGAGCACCTTTGCTGGCCATGGCAGGTATGCTGGCCACTGGAC
TGGGGATGTGTGGAGCTCTTGGGAGCAGCTGGCCAGCTCTGTGCCAGAGATCCTGCAGTTCAACCTGCTGGGGGT
GCCTCTGGTGGGGGCTGATGTGTGTGGCTTCCTGGGCAATACCTCTGAAGAGCTGTGTGTGAGGTGGACTCAGCT
GGGGGCCTTCTATCCCTTCATGAGGAACCACAACAGCCTGCTGTCTCTGCCCCAGGAGCCCTACAGCTTCTCTGA
GCCTGCTCAGCAGGCTATGAGGAAGGCCCTGACCCTGAGGTATGCCCTGCTGCCCCATCTGTACACCCTGTTCCA
CCAGGCCCATGTGGCTGGGGAGACTGTGGCCAGGCCCCTGTTTCTGGAGTTTCCCAAGGACAGCAGCACCTGGAC
TGTGGACCATCAGCTGCTGTGGGGGGAGGCTCTGCTGATTACCCCTGTGCTGCAGGCTGGCAAGGCTGAGGTGAC
TGGGTACTTCCCCCTGGGGACTTGGTATGACCTGCAGACTGTGCCTGTGGAAGCTCTGGGCAGCCTGCCCCCACC
CCCTGCTGCCCCTAGGGAGCCTGCCATCCACTCTGAGGGCCAGTGGGTGACCCTGCCTGCCCCTCTGGACACCAT
CAATGTGCACCTGAGGGCTGGCTATATCATCCCCCTGCAGGGCCCTGGGCTGACCACCACTGAGAGCAGGCAGCA
GCCCATGGCCCTGGCTGTGGCCCTGACTAAGGGGGGGGAGGCCAGGGGGGAGCTGTTCTGGGATGATGGGGAGAG
CCTGGAGGTGCTGGAGAGAGGGGCCTACACCCAGGTGATCTTTCTGGCCAGGAACAACACCATTGTGAATGAGCT
GGTGAGGGTGACTTCTGAGGGGGCTGGCCTGCAGCTGCAGAAGGTGACTGTGCTGGGGGTGGCCACTGCCCCCCA
GCAGGTGCTGAGCAATGGGGTGCCTGTGTCTAACTTCACCTACAGCCCTGATACTAAGGTGCTGGATATCTGTGT
GAGCCTGCTGATGGGGGAGCAGTTTCTGGTGAGCTGGTGCTGAAGATCTAGAGCTGAATTCCTGCAGCCAGGGGG
ATCAGCCTCTACTGTGCCTTCTAGTTGCCAGCCATCTGTTGTTTGCCCCTCCCCCTTGCCTTCCTTGACCCTGGA
AGGTGCCACTCCCACTGTCCTTTCCTAATAAAATGAGGAAATTGCATCACATTGTCTGAGTAGGTGTCATTCTAT
TCTGGGGGGTGGGGTGGGGCAGGACAGCAAGGGGGAGGATTGGGAAGACAATAGCAGGCATGCTGGGGATGCAGT
GGGCTCTATGGCTTCTGAGGCAGAAAGAACCAGCTGGGGCTCGAGATCCACTAGGGCCGCAGGAACCCCTAGTGA
TGGAGTTGGCCACTCCCTCTCTGCGCGCTCGCTCGCTCACTGAGGCCGGGCGACCAAAGGTCGCCCGACGCCCGG
GCTTTGCCCGGGCGGCCTCAGTGAGCGAGCGAGCGCGCAGCTGCCTGCAGG
>pAAV-ApoE/hAAT.GAA2.wtBGH
(SEQ ID NO: 20)
CCTGCAGGCAGCTGCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAACTCCATCACTAGGGGTTCCTGCGGCCGCA
GGCTCAGAGGCACACAGGAGTTTCTGGGCTCACCCTGCCCCCTTCCAACCCCTCAGTTCCCATCCTCCAGCAGCT
GTTTGTGTGCTGCCTCTGAAGTCCACACTGAACAAACTTCAGCCTACTCATGTCCCTAAAATGGGCAAACATTGC
AAGCAGCAAACAGCAAACACACAGCCCTCCCTGCCTGCTGACCTTGGAGCTGGGGCAGAGGTCAGAGACCTCTCT
GGGCCCATGCCACCTCCAACATCCACTCGACCCCTTGGAATTTCGGTGGAGAGGAGCAGAGGTTGTCCTGGCGTG
GTTTAGGTAGTGTGAGAGGGGTACCCGGGGATCTTGCTACCAGTGGAACAGCCACTAAGGATTCTGCAGTGAGAG
CAGAGGGCCAGCTAAGTGGTACTCTCCCAGAGACTGTCTGACTCACGCCACCCCCTCCACCTTGGACACAGGACG
CTGTGGTTTCTGAGCCAGGTACAATGACTCCTTTCGGTAAGTGCAGTGGAAGCTGTACACTGCCCAGGCAAAGCG
TCCGGGCAGCGTAGGCGGGCGACTCAGATCCCAGCCAGTGGACTTAGCCCCTGTTTGCTCCTCCGATAACTGGGG
TGACCTTGGTTAATATTCACCAGCAGCCTCCCCCGTTGCCCCTCTGGATCCACTGCTTAAATACGGACGAGGACA
GGGCCCTGTCTCCTCAGCTTCAGGCACCACCACTGACCTGGGACAGTGAATAGATCCTGAGAACTTCAGGGTGAG
TCTATGGGACCCTTGATGTTTTCTTTCCCCTTCTTTTCTATGGTTAAGTTCATGTCATAGGAAGGGGAGAAGTAA
CAGGGTACACATATTGACCAAATCAGGGTAATTTTGCATTTGTAATTTTAAAAAATGCTTTCTTCTTTTAATATA
CTTTTTTGTTTATCTTATTTCTAATACTTTCCCTAATCTCTTTCTTTCAGGGCAATAATGATACAATGTATCATG
CCTCTTTGCACCATTCTAAAGAATAACAGTGATAATTTCTGGGTTAAGGCAATAGCAATATTTCTGCATATAAAT
ATTTCTGCATATAAATTGTAACTGATGTAAGAGGTTTCATATTGCTAATAGCAGCTACAATCCAGCTACCATTCT
GCTTTTATTTTCTGGTTGGGATAAGGCTGGATTATTCTGAGTCCAAGCTAGGCCCTTTTGCTAATCTTGTTCATA
CCTCTTATCTTCCTCCCACAGCTCCTGGGCAACCTGCTGGTCTCTCTGCTGGCCCATCACTTTGGCAAAGCACGC
GTGCCACCATGGCTTTCCTGTGGCTGCTGAGCTGCTGGGCTCTGCTGGGCACCACCTTTGGGCTGCTGGTGCCTA
GGGAGCTGTCTGGGTCTAGCCCTGTGCTGGAGGAGACTCACCCTGCCCATCAGCAGGGGGCTAGCAGGCCTGGCC
CCAGGGATGCTCAGGCCCACCCTGGCAGGCCCAGGGCTGTGCCCACCCAGTGTGATGTGCCCCCCAACAGCAGGT
TTGACTGTGCCCCTGACAAGGCCATTACCCAGGAGCAGTGTGAGGCCAGGGGCTGCTGCTACATTCCAGCTAAGC
AGGGCCTGCAGGGGGCCCAGATGGGCCAGCCCTGGTGCTTCTTCCCCCCCAGCTATCCTAGCTATAAACTGGAGA
ACCTGAGCAGCTCTGAGATGGGCTATACTGCCACCCTGACTAGGACTACTCCCACCTTTTTTCCTAAGGATATCC
TGACCCTGAGGCTGGATGTGATGATGGAGACTGAGAACAGGCTGCACTTCACTATTAAGGACCCTGCCAATAGGA
GGTATGAAGTGCCTCTGGAGACTCCTCATGTGCACTCTAGGGCCCCCAGCCCCCTGTATTCTGTGGAGTTCTCTG
AGGAGCCCTTTGGGGTGATTGTGAGGAGGCAGCTGGATGGCAGGGTGCTGCTGAACACCACTGTGGCCCCCCTGT
TCTTTGCTGACCAGTTCCTGCAGCTGAGCACCAGCCTGCCCAGCCAGTACATCACTGGGCTGGCTGAGCATCTGA
GCCCTCTGATGCTGAGCACCTCTTGGACCAGGATCACCCTGTGGAATAGGGATCTGGCCCCCACCCCTGGGGCTA
ATCTGTATGGCTCTCATCCCTTTTACCTGGCCCTGGAGGATGGGGGCTCTGCCCATGGGGTGTTTCTGCTGAACA
GCAATGCCATGGATGTGGTGCTGCAGCCCTCTCCTGCCCTGAGCTGGAGGAGCACTGGGGGCATCCTGGATGTGT
ACATCTTCCTGGGCCCTGAGCCCAAGTCTGTGGTCCAGCAGTATCTGGATGTGGTGGGCTACCCCTTTATGCCCC
CCTATTGGGGCCTGGGCTTCCACCTGTGCAGGTGGGGGTATTCTTCTACTGCTATCACCAGGCAGGTGGTGGAGA
ACATGACCAGGGCTCACTTCCCCCTGGATGTGCAGTGGAATGACCTGGACTATATGGACTCTAGGAGGGATTTCA
CCTTCAACAAGGATGGCTTCAGGGACTTCCCTGCTATGGTCCAGGAGCTGCATCAGGGGGGCAGGAGGTACATGA
TGATTGTGGACCCTGCCATCAGCAGCTCTGGCCCTGCTGGCAGCTATAGGCCCTATGATGAGGGCCTGAGGAGGG
GGGTGTTTATCACTAATGAAACTGGGCAGCCCCTGATTGGCAAGGTGTGGCCTGGCTCTACTGCCTTCCCTGACT
TCACCAACCCCACTGCTCTGGCCTGGTGGGAGGACATGGTGGCTGAGTTCCATGACCAGGTGCCTTTTGATGGCA
TGTGGATTGACATGAATGAGCCCAGCAACTTCATCAGGGGCTCTGAGGATGGGTGCCCCAATAATGAGCTGGAGA
ACCCCCCCTATGTGCCTGGGGTGGTGGGGGGCACCCTGCAGGCTGCCACTATTTGTGCCAGCTCTCACCAGTTCC
TGAGCACCCACTACAACCTGCACAATCTGTATGGCCTGACTGAGGCCATTGCCAGCCACAGGGCCCTGGTGAAGG
CCAGGGGCACTAGGCCCTTTGTGATCTCTAGAAGCACCTTTGCTGGCCATGGGAGGTATGCTGGCCACTGGACTG
GGGATGTGTGGAGCTCTTGGGAGCAGCTGGCCAGCTCTGTGCCTGAGATCCTGCAGTTCAACCTGCTGGGGGTGC
CCCTGGTGGGGGCTGATGTGTGTGGCTTCCTGGGCAACACCTCTGAAGAGCTGTGTGTGAGGTGGACCCAGCTGG
GGGCCTTCTACCCTTTCATGAGGAACCACAACAGCCTGCTGAGCCTGCCTCAGGAGCCTTACTCTTTCTCTGAGC
CTGCCCAGCAGGCCATGAGGAAGGCCCTGACCCTGAGGTATGCTCTGCTGCCCCACCTGTACACCCTGTTCCACC
AGGCCCATGTGGCTGGGGAGACTGTGGCCAGGCCCCTGTTCCTGGAGTTTCCTAAGGATAGCAGCACCTGGACTG
TGGACCACCAGCTGCTGTGGGGGGAGGCCCTGCTGATTACCCCTGTGCTGCAGGCTGGCAAGGCTGAGGTGACTG
GCTACTTCCCCCTGGGCACTTGGTATGACCTGCAGACTGTGCCTGTGGAAGCCCTGGGCAGCCTGCCTCCCCCCC
CTGCTGCCCCCAGGGAGCCTGCCATCCACTCTGAGGGCCAGTGGGTGACCCTGCCTGCCCCCCTGGACACCATTA
ATGTGCATCTGAGGGCTGGGTATATTATCCCCCTGCAGGGGCCTGGGCTGACTACCACTGAGAGCAGGCAGCAGC
CTATGGCCCTGGCTGTGGCTCTGACTAAGGGGGGGGAGGCCAGGGGGGAGCTGTTCTGGGATGATGGGGAGAGCC
TGGAGGTGCTGGAGAGGGGGGCCTACACCCAGGTGATTTTCCTGGCCAGGAACAACACCATTGTGAATGAGCTGG
TGAGGGTGACCTCTGAGGGGGCTGGCCTGCAGCTGCAGAAAGTGACTGTGCTGGGGGTGGCCACTGCCCCCCAGC
AGGTGCTGAGCAATGGGGTGCCTGTGAGCAACTTCACCTACAGCCCTGACACCAAGGTGCTGGATATTTGTGTGA
GCCTGCTGATGGGGGAGCAGTTCCTGGTGAGCTGGTGCTGACTCGAGAGATCTACCGGTGAATTCACCGCGGGTT
TAAACTGTGCCTTCTAGTTGCCAGCCATCTGTTGTTTGCCCCTCCCCCGTGCCTTCCTTGACCCTGGAAGGTGCC
ACTCCCACTGTCCTTTCCTAATAAAATGAGGAAATTGCATCGCATTGTCTGAGTAGGTGTCATTCTATTCTGGGG
GGTGGGGTGGGGGCTAGCTCTAGACTCGAGATCCACTAGGGCCGCAGGAACCCCTAGTGATGGAGTTGGCCACTC
CCTCTCTGCGCGCTCGCTCGCTCACTGAGGCCGGGCGACCAAAGGTCGCCCGACGCCCGGGCTTTGCCCGGGCGG
CCTCAGTGAGCGAGCGAGCGCGCAGCTGCCTGCAGG
>pAAV-ApoE/hAAT.GAA5.wtBGH
(SEQ ID NO: 21)
CCTGCAGGCAGCTGCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAACTCCATCACTAGGGGTTCCTGCGGCCGCA
GGCTCAGAGGCACACAGGAGTTTCTGGGCTCACCCTGCCCCCTTCCAACCCCTCAGTTCCCATCCTCCAGCAGCT
GTTTGTGTGCTGCCTCTGAAGTCCACACTGAACAAACTTCAGCCTACTCATGTCCCTAAAATGGGCAAACATTGC
AAGCAGCAAACAGCAAACACACAGCCCTCCCTGCCTGCTGACCTTGGAGCTGGGGCAGAGGTCAGAGACCTCTCT
GGGCCCATGCCACCTCCAACATCCACTCGACCCCTTGGAATTTCGGTGGAGAGGAGCAGAGGTTGTCCTGGCGTG
GTTTAGGTAGTGTGAGAGGGGTACCCGGGGATCTTGCTACCAGTGGAACAGCCACTAAGGATTCTGCAGTGAGAG
CAGAGGGCCAGCTAAGTGGTACTCTCCCAGAGACTGTCTGACTCACGCCACCCCCTCCACCTTGGACACAGGACG
CTGTGGTTTCTGAGCCAGGTACAATGACTCCTTTCGGTAAGTGCAGTGGAAGCTGTACACTGCCCAGGCAAAGCG
TCCGGGCAGCGTAGGCGGGCGACTCAGATCCCAGCCAGTGGACTTAGCCCCTGTTTGCTCCTCCGATAACTGGGG
TGACCTTGGTTAATATTCACCAGCAGCCTCCCCCGTTGCCCCTCTGGATCCACTGCTTAAATACGGACGAGGACA
GGGCCCTGTCTCCTCAGCTTCAGGCACCACCACTGACCTGGGACAGTGAATAGATCCTGAGAACTTCAGGGTGAG
TCTATGGGACCCTTGATGTTTTCTTTCCCCTTCTTTTCTATGGTTAAGTTCATGTCATAGGAAGGGGAGAAGTAA
CAGGGTACACATATTGACCAAATCAGGGTAATTTTGCATTTGTAATTTTAAAAAATGCTTTCTTCTTTTAATATA
CTTTTTTGTTTATCTTATTTCTAATACTTTCCCTAATCTCTTTCTTTCAGGGCAATAATGATACAATGTATCATG
CCTCTTTGCACCATTCTAAAGAATAACAGTGATAATTTCTGGGTTAAGGCAATAGCAATATTTCTGCATATAAAT
ATTTCTGCATATAAATTGTAACTGATGTAAGAGGTTTCATATTGCTAATAGCAGCTACAATCCAGCTACCATTCT
GCTTTTATTTTCTGGTTGGGATAAGGCTGGATTATTCTGAGTCCAAGCTAGGCCCTTTTGCTAATCTTGTTCATA
CCTCTTATCTTCCTCCCACAGCTCCTGGGCAACCTGCTGGTCTCTCTGCTGGCCCATCACTTTGGCAAAGCACGC
GTGCCACCATGGCTTTCCTGTGGCTGCTGTCTTGCTGGGCCCTGCTGGGGACTACCTTTGGCCTGCTGGTGCCCA
GGGAACTGTCTGGCTCTAGCCCAGTGCTGGAGGAGACCCACCCTGCCCACCAGCAGGGGGCTTCTAGGCCTGGCC
CCAGGGATGCCCAGGCCCACCCTGGCAGGCCAAGGGCTGTGCCCACCCAGTGTGATGTGCCCCCCAACTCTAGAT
TTGATTGTGCCCCTGATAAGGCCATCACCCAGGAGCAGTGTGAGGCTAGGGGCTGCTGCTACATCCCTGCTAAGC
AGGGCCTGCAGGGGGCTCAGATGGGCCAGCCCTGGTGCTTCTTCCCCCCCAGCTATCCCTCTTACAAGCTGGAGA
ATCTGAGCAGCTCTGAGATGGGCTACACTGCCACCCTGACCAGGACTACTCCCACCTTCTTCCCCAAGGACATCC
TGACCCTGAGGCTGGATGTGATGATGGAGACTGAGAACAGGCTGCATTTCACCATCAAGGATCCTGCCAACAGGA
GGTATGAGGTGCCTCTGGAGACCCCCCATGTGCACAGCAGGGCTCCTTCTCCCCTGTACTCTGTGGAGTTCTCTG
AGGAACCCTTTGGGGTGATTGTGAGGAGGCAGCTGGATGGCAGGGTCCTGCTGAACACCACTGTGGCCCCCCTGT
TCTTTGCTGATCAGTTCCTGCAGCTGTCCACTTCTCTGCCTAGCCAGTACATCACTGGGCTGGCTGAGCACCTGA
GCCCTCTGATGCTGAGCACCTCTTGGACTAGGATCACCCTGTGGAACAGGGACCTGGCCCCCACCCCTGGGGCCA
ACCTGTATGGCAGCCACCCCTTCTATCTGGCCCTGGAGGATGGGGGCTCTGCCCATGGGGTGTTCCTGCTGAATA
GCAATGCTATGGATGTGGTGCTGCAGCCCAGCCCTGCCCTGTCTTGGAGGAGCACTGGGGGCATCCTGGATGTGT
ACATTTTCCTGGGGCCTGAGCCCAAGTCTGTGGTGCAGCAGTACCTGGATGTGGTGGGCTACCCCTTCATGCCTC
CCTACTGGGGCCTGGGCTTCCACCTGTGCAGGTGGGGCTACAGCTCTACTGCCATCACCAGGCAGGTGGTGGAGA
ATATGACCAGGGCCCACTTCCCCCTGGATGTGCAGTGGAATGACCTGGACTACATGGACTCTAGGAGGGACTTCA
CCTTCAATAAGGATGGCTTCAGAGACTTCCCTGCCATGGTGCAGGAGCTGCATCAGGGGGGCAGGAGGTACATGA
TGATTGTGGACCCTGCCATCAGCTCTTCTGGCCCTGCTGGCTCTTACAGGCCCTATGATGAGGGCCTGAGGAGGG
GGGTGTTCATCACCAATGAGACTGGGCAGCCCCTGATTGGGAAGGTGTGGCCTGGCTCTACTGCCTTCCCTGACT
TCACCAATCCTACTGCCCTGGCCTGGTGGGAGGACATGGTGGCTGAGTTCCATGACCAGGTGCCCTTTGATGGCA
TGTGGATTGACATGAATGAGCCCTCTAATTTCATCAGGGGCTCTGAGGATGGCTGCCCCAACAATGAGCTGGAGA
ACCCCCCCTATGTGCCTGGGGTGGTGGGGGGCACCCTGCAGGCTGCCACCATCTGTGCTAGCTCTCACCAGTTCC
TGAGCACCCACTACAATCTGCATAACCTGTATGGCCTGACTGAGGCCATTGCCAGCCACAGGGCCCTGGTGAAGG
CTAGGGGCACCAGGCCCTTTGTGATTTCTAGGAGCACTTTTGCTGGCCATGGCAGGTATGCTGGGCACTGGACTG
GGGATGTGTGGTCTAGCTGGGAGCAGCTGGCTTCTTCTGTGCCTGAGATCCTGCAGTTCAACCTGCTGGGGGTGC
CTCTGGTGGGGGCTGATGTGTGTGGGTTCCTGGGCAACACTTCTGAGGAGCTGTGTGTGAGGTGGACCCAGCTGG
GGGCCTTCTACCCTTTCATGAGGAACCACAACAGCCTGCTGAGCCTGCCCCAGGAGCCCTACAGCTTCTCTGAGC
CTGCCCAGCAGGCCATGAGGAAGGCCCTGACCCTGAGGTATGCCCTGCTGCCCCACCTGTACACCCTGTTCCACC
AGGCCCATGTGGCTGGGGAGACTGTGGCTAGGCCTCTGTTCCTGGAGTTCCCCAAGGACTCTAGCACCIGGACTG
TGGACCACCAGCTGCTGTGGGGGGAGGCCCTGCTGATCACTCCTGTGCTGCAGGCTGGGAAGGCTGAGGTGACTG
GCTATTTCCCCCTGGGCACCTGGTATGACCTGCAGACTGTGCCTGTGGAGGCCCTGGGGAGCCTGCCCCCCCCCC
CTGCTGCCCCCAGGGAGCCTGCCATCCACTCTGAGGGCCAGTGGGTGACCCTGCCTGCCCCTCTGGATACCATCA
ATGTGCACCTGAGGGCTGGCTACATCATTCCCCTGCAGGGCCCTGGCCTGACCACTACTGAGTCTAGGCAGCAGC
CCATGGCCCTGGCTGTGGCCCTGACCAAGGGGGGGGAGGCTAGGGGGGAGCTGTTTTGGGATGATGGGGAGAGCC
TGGAGGTGCTGGAGAGGGGGGCCTACACTCAGGTGATCTTCCTGGCCAGGAACAATACCATTGTGAATGAGCTGG
TGAGGGTGACCTCTGAGGGGGCTGGCCTGCAGCTGCAGAAGGTGACTGTGCTGGGGGTGGCCACTGCCCCCCAGC
AGGTGCTGAGCAATGGGGTGCCTGTGAGCAACTTCACCTATAGCCCTGATACCAAGGTGCTGGATATTTGTGTGA
GCCTGCTGATGGGGGAGCAGTTCCTGGTGAGCTGGTGCTGACTCGAGAGATCTACCGGTGAATTCACCGCGGGTT
TAAACTGTGCCTTCTAGTTGCCAGCCATCTGTTGTTTGCCCCTCCCCCGTGCCTTCCTTGACCCTGGAAGGTGCC
ACTCCCACTGTCCTTTCCTAATAAAATGAGGAAATTGCATCGCATTGTCTGAGTAGGTGTCATTCTATTCTGGGG
GGTGGGGTGGGGGCTAGCTCTAGACTCGAGATCCACTAGGGCCGCAGGAACCCCTAGTGATGGAGTTGGCCACTC
CCTCTCTGCGCGCTCGCTCGCTCACTGAGGCCGGGCGACCAAAGGTCGCCCGACGCCCGGGCTTTGCCCGGGCGG
CCTCAGTGAGCGAGCGAGCGCGCAGCTGCCTGCAGG
>pAAV-ApoE/hAAT.GAA7.wtBGH
(SEQ ID NO: 22)
CCTGCAGGCAGCTGCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAACTCCATCACTAGGGGTTCCTGCGGCCGCA
GGCTCAGAGGCACACAGGAGTTTCTGGGCTCACCCTGCCCCCTTCCAACCCCTCAGTTCCCATCCTCCAGCAGCT
GTTTGTGTGCTGCCTCTGAAGTCCACACTGAACAAACTTCAGCCTACTCATGTCCCTAAAATGGGCAAACATTGC
AAGCAGCAAACAGCAAACACACAGCCCTCCCTGCCTGCTGACCTTGGAGCTGGGGCAGAGGTCAGAGACCTCTCT
GGGCCCATGCCACCTCCAACATCCACTCGACCCCTTGGAATTTCGGTGGAGAGGAGCAGAGGTTGTCCTGGCGTG
GTTTAGGTAGTGTGAGAGGGGTACCCGGGGATCTTGCTACCAGTGGAACAGCCACTAAGGATTCTGCAGTGAGAG
CAGAGGGCCAGCTAAGTGGTACTCTCCCAGAGACTGTCTGACTCACGCCACCCCCTCCACCTTGGACACAGGACG
CTGTGGTTTCTGAGCCAGGTACAATGACTCCTTTCGGTAAGTGCAGTGGAAGCTGTACACTGCCCAGGCAAAGCG
TCCGGGCAGCGTAGGCGGGCGACTCAGATCCCAGCCAGTGGACTTAGCCCCTGTTTGCTCCTCCGATAACTGGGG
TGACCTTGGTTAATATTCACCAGCAGCCTCCCCCGTTGCCCCTCTGGATCCACTGCTTAAATACGGACGAGGACA
GGGCCCTGTCTCCTCAGCTTCAGGCACCACCACTGACCTGGGACAGTGAATAGATCCTGAGAACTTCAGGGTGAG
TCTATGGGACCCTTGATGTTTTCTTTCCCCTTCTTTTCTATGGTTAAGTTCATGTCATAGGAAGGGGAGAAGTAA
CAGGGTACACATATTGACCAAATCAGGGTAATTTTGCATTTGTAATTTTAAAAAATGCTTTCTTCTTTTAATATA
CTTTTTTGTTTATCTTATTTCTAATACTTTCCCTAATCTCTTTCTTTCAGGGCAATAATGATACAATGTATCATG
CCTCTTTGCACCATTCTAAAGAATAACAGTGATAATTTCTGGGTTAAGGCAATAGCAATATTTCTGCATATAAAT
ATTTCTGCATATAAATTGTAACTGATGTAAGAGGTTTCATATTGCTAATAGCAGCTACAATCCAGCTACCATTCT
GCTTTTATTTTCTGGTTGGGATAAGGCTGGATTATTCTGAGTCCAAGCTAGGCCCTTTTGCTAATCTTGTTCATA
CCTCTTATCTTCCTCCCACAGCTCCTGGGCAACCTGCTGGTCTCTCTGCTGGCCCATCACTTTGGCAAAGCACGC
GTGCCACCATGGCTTTCCTGTGGCTGCTGTCTTGTTGGGCTCTGCTGGGCACCACCTTTGGCCTGCTGGTGCCCA
GGGAGCTGTCTGGCAGCAGCCCTGTGCTGGAGGAGACCCACCCTGCTCATCAGCAGGGGGCTAGCAGGCCTGGCC
CCAGGGATGCCCAGGCTCACCCTGGGAGACCCAGGGCTGTGCCCACTCAGTGTGATGTGCCCCCCAACAGCAGGT
TTGACTGTGCTCCTGACAAGGCTATCACCCAGGAGCAGTGTGAGGCCAGGGGGTGCTGCTACATTCCTGCTAAGC
AGGGCCTGCAGGGGGCCCAGATGGGCCAGCCCTGGTGCTTCTTCCCCCCCTCTTATCCCAGCTATAAGCTGGAGA
ACCTGAGCAGCTCTGAGATGGGCTACACTGCCACCCTGACCAGGACCACTCCCACCTTCTTTCCCAAGGATATTC
TGACTCTGAGGCTGGATGTGATGATGGAGACTGAGAACAGGCTGCACTTCACTATCAAGGACCCTGCCAATAGGA
GGTATGAGGTGCCCCTGGAGACTCCTCATGTGCATAGCAGGGCCCCTTCTCCTCTGTATTCTGTGGAGTTCTCTG
AGGAGCCCTTTGGGGTGATTGTGAGGAGGCAGCTGGATGGCAGGGTGCTGCTGAACACCACTGTGGCCCCCCTGT
TCTTTGCTGACCAGTTCCTGCAGCTGAGCACTTCTCTGCCCAGCCAGTACATTACTGGGCTGGCTGAGCATCTGA
GCCCCCTGATGCTGAGCACCTCTTGGACCAGGATCACCCTGTGGAACAGGGACCTGGCCCCCACTCCTGGGGCTA
ACCTGTATGGCTCTCACCCCTTTTACCTGGCCCTGGAGGATGGGGGCTCTGCCCATGGGGTGTTTCTGCTGAACA
GCAATGCTATGGATGTGGTGCTGCAGCCCTCTCCAGCCCTGTCTTGGAGGAGCACTGGGGGCATTCTGGATGTGT
ACATTTTCCTGGGGCCTGAACCCAAGTCTGTGGTGCAGCAGTACCTGGATGTGGTGGGCTACCCCTTCATGCCCC
CCTATTGGGGGCTGGGGTTTCACCTGTGCAGGTGGGGCTACAGCAGCACTGCCATCACCAGGCAGGTGGTGGAGA
ACATGACCAGGGCCCATTTCCCCCTGGATGTGCAGTGGAATGACCTGGACTACATGGATAGCAGGAGGGATTTCA
CCTTCAACAAGGATGGCTTCAGGGACTTTCCTGCCATGGTGCAGGAGCTGCACCAGGGGGGCAGGAGGTATATGA
TGATTGTGGACCCTGCTATCAGCAGCTCTGGCCCTGCTGGCTCTTACAGGCCCTATGATGAGGGCCTGAGGAGGG
GGGTGTTTATCACTAATGAAACTGGCCAGCCTCTGATTGGCAAGGTCTGGCCTGGCTCTACTGCCTTCCCTGATT
TTACTAACCCCACTGCCCTGGCCTGGTGGGAGGACATGGTGGCTGAGTTCCATGATCAGGTGCCTTTTGATGGCA
TGTGGATTGATATGAATGAACCAAGCAACTTCATCAGAGGCTCTGAGGATGGCTGCCCCAACAATGAGCTGGAGA
ACCCCCCCTATGTGCCTGGGGTGGTGGGGGGCACTCTGCAGGCTGCCACCATTIGTGCTAGCAGCCACCAGTTCC
TGAGCACCCACTACAATCTGCACAACCTGTATGGCCTGACTGAAGCCATTGCCAGCCATAGGGCCCTGGTGAAGG
CCAGGGGCACTAGGCCTTTTGTGATCAGCAGGAGCACTTTTGCTGGCCATGGCAGGTATGCTGGCCACTGGACTG
GGGATGTGTGGAGCAGCTGGGAGCAGCTGGCCAGCTCTGTGCCTGAGATTCTGCAGTTTAACCTGCTGGGGGTGC
CCCTGGTGGGGGCTGATGTGTGTGGCTTCCTGGGCAACACCTCTGAGGAGCTGTGTGTGAGGTGGACCCAGCTGG
GGGCCTTTTATCCCTTCATGAGGAACCACAACAGCCTGCTGAGCCTGCCTCAGGAGCCCTACTCTTTCTCTGAGC
CTGCCCAGCAGGCCATGAGGAAGGCCCTGACCCTGAGGTATGCCCTGCTGCCCCACCTGTATACCCTGTTCCACC
AGGCCCATGTGGCTGGGGAGACTGTGGCCAGGCCCCTGTTCCTGGAGTTCCCCAAGGACAGCAGCACCTGGACTG
TGGATCATCAGCTGCTGTGGGGGGAGGCCCTGCTGATCACCCCTGTGCTGCAGGCTGGCAAGGCTGAGGTCACTG
GCTACTTCCCTCTGGGCACCTGGTATGACCTGCAGACTGTGCCTGTGGAGGCTCTGGGCAGCCTGCCCCCCCCCC
CTGCTGCTCCCAGGGAGCCTGCCATCCACTCTGAGGGCCAGTGGGTGACCCTGCCTGCTCCCCTGGACACCATCA
ATGTGCACCTGAGGGCTGGCTACATTATCCCCCTGCAGGGCCCAGGGCTGACTACCACTGAGAGCAGACAGCAGC
CCATGGCTCTGGCTGTGGCCCTGACCAAGGGGGGGGAAGCTAGGGGGGAGCTGTTCTGGGATGATGGGGAGAGCC
TGGAGGTGCTGGAGAGGGGGGCCTATACCCAGGTGATCTTCCTGGCTAGGAACAACACCATTGTCAATGAGCTGG
TGAGGGTGACTTCTGAGGGGGCTGGGCTGCAGCTGCAGAAGGTGACTGTGCTGGGGGTGGCCACTGCTCCCCAGC
AGGTGCTGAGCAATGGGGTGCCTGTGAGCAACTTCACCTACAGCCCTGACACCAAGGTGCTGGACATCTGTGTGT
CTCTGCTGATGGGGGAGCAGTTCCTGGTGAGCTGGTGCTGACTCGAGAGATCTACCGGTGAATTCACCGCGGGTT
TAAACTGTGCCTTCTAGTTGCCAGCCATCTGTTGTTTGCCCCTCCCCCGTGCCTTCCTTGACCCTGGAAGGTGCC
ACTCCCACTGTCCTTTCCTAATAAAATGAGGAAATTGCATCGCATTGTCTGAGTAGGTGTCATTCTATTCTGGGG
GGTGGGGTGGGGGCTAGCTCTAGACTCGAGATCCACTAGGGCCGCAGGAACCCCTAGTGATGGAGTTGGCCACTC
CCTCTCTGCGCGCTCGCTCGCTCACTGAGGCCGGGCGACCAAAGGTCGCCCGACGCCCGGGCTTTGCCCGGGCGG
CCTCAGTGAGCGAGCGAGCGCGCAGCTGCCTGCAGG
>pAAV-ApoE/hAAT.GAA8.wtBGH
(SEQ ID NO: 23)
CCTGCAGGCAGCTGCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAACTCCATCACTAGGGGTTCCTGCGGCCGCA
GGCTCAGAGGCACACAGGAGTTTCTGGGCTCACCCTGCCCCCTTCCAACCCCTCAGTTCCCATCCTCCAGCAGCT
GTTTGTGTGCTGCCTCTGAAGTCCACACTGAACAAACTTCAGCCTACTCATGTCCCTAAAATGGGCAAACATTGC
AAGCAGCAAACAGCAAACACACAGCCCTCCCTGCCTGCTGACCTTGGAGCTGGGGCAGAGGTCAGAGACCTCTCT
GGGCCCATGCCACCTCCAACATCCACTCGACCCCTTGGAATTTCGGTGGAGAGGAGCAGAGGTTGTCCTGGCGTG
GTTTAGGTAGTGTGAGAGGGGTACCCGGGGATCTTGCTACCAGTGGAACAGCCACTAAGGATTCTGCAGTGAGAG
CAGAGGGCCAGCTAAGTGGTACTCTCCCAGAGACTGTCTGACTCACGCCACCCCCTCCACCTTGGACACAGGACG
CTGTGGTTTCTGAGCCAGGTACAATGACTCCTTTCGGTAAGTGCAGTGGAAGCTGTACACTGCCCAGGCAAAGCG
TCCGGGCAGCGTAGGCGGGCGACTCAGATCCCAGCCAGTGGACTTAGCCCCTGTTTGCTCCTCCGATAACTGGGG
TGACCTTGGTTAATATTCACCAGCAGCCTCCCCCGTTGCCCCTCTGGATCCACTGCTTAAATACGGACGAGGACA
GGGCCCTGTCTCCTCAGCTTCAGGCACCACCACTGACCTGGGACAGTGAATAGATCCTGAGAACTTCAGGGTGAG
TCTATGGGACCCTTGATGTTTTCTTTCCCCTTCTTTTCTATGGTTAAGTTCATGTCATAGGAAGGGGAGAAGTAA
CAGGGTACACATATTGACCAAATCAGGGTAATTTTGCATTTGTAATTTTAAAAAATGCTTTCTTCTTTTAATATA
CTTTTTTGTTTATCTTATTTCTAATACTTTCCCTAATCTCTTTCTTTCAGGGCAATAATGATACAATGTATCATG
CCTCTTTGCACCATTCTAAAGAATAACAGTGATAATTTCTGGGTTAAGGCAATAGCAATATTTCTGCATATAAAT
ATTTCTGCATATAAATTGTAACTGATGTAAGAGGTTTCATATTGCTAATAGCAGCTACAATCCAGCTACCATTCT
GCTTTTATTTTCTGGTTGGGATAAGGCTGGATTATTCTGAGTCCAAGCTAGGCCCTTTTGCTAATCTTGTTCATA
CCTCTTATCTTCCTCCCACAGCTCCTGGGCAACCTGCTGGTCTCTCTGCTGGCCCATCACTTTGGCAAAGCACGC
GTGCCACCATGGCCTTCCTGTGGCTGCTGTCTTGCTGGGCTCTGCTGGGGACCACCTTTGGCCTGCTGGTCCCCA
GGGAGCTGTCTGGCTCTTCTCCTGTCCTGGAGGAGACCCACCCTGCCCACCAGCAGGGGGCTAGCAGGCCTGGCC
CCAGGGATGCCCAGGCCCACCCTGGCAGGCCCAGGGCTGTGCCCACCCAGTGTGATGTGCCTCCCAACAGCAGGT
TTGACTGTGCCCCTGACAAGGCCATCACCCAGGAGCAGTGTGAGGCCAGGGGCTGCTGCTATATCCCTGCCAAGC
AGGGCCTGCAGGGGGCTCAGATGGGCCAGCCCTGGTGCTTCTTTCCCCCCTCTTATCCTAGCTATAAGCTGGAGA
ACCTGAGCAGCTCTGAGATGGGGTACACTGCCACCCTGACCAGGACCACCCCCACTTTCTTCCCTAAGGACATCC
TGACCCTGAGGCTGGATGTGATGATGGAGACTGAGAATAGGCTGCACTTTACTATCAAGGACCCTGCCAACAGGA
GGTATGAGGTGCCTCTGGAGACCCCCCATGTGCATTCTAGGGCCCCCAGCCCCCTGTACTCTGTGGAGTTCTCTG
AGGAGCCCTTTGGGGTGATTGTGAGGAGACAGCTGGATGGCAGGGTCCTGCTGAACACCACTGTGGCTCCCCTGT
TTTTTGCTGACCAGTTCCTGCAGCTGAGCACCAGCCTGCCCAGCCAGTACATCACTGGGCTGGCTGAGCACCTGA
GCCCCCTGATGCTGAGCACCAGCTGGACCAGGATCACCCTGTGGAACAGGGATCTGGCTCCTACCCCTGGGGCCA
ACCTGTATGGCTCTCACCCCTTTTACCTGGCCCTGGAGGATGGGGGCTCTGCCCATGGGGTGTTCCTGCTGAACA
GCAATGCTATGGATGTGGTGCTGCAGCCCAGCCCTGCCCTGAGCTGGAGGTCTACTGGGGGCATCCTGGATGTGT
ACATCTTTCTGGGGCCTGAGCCCAAGTCTGTGGTGCAGCAGTACCTGGATGTGGTGGGCTATCCTTTTATGCCCC
CCTATTGGGGCCTGGGCTTCCACCTGTGCAGGTGGGGCTACAGCAGCACTGCCATCACCAGACAGGTGGTGGAGA
ACATGACCAGGGCCCACTTCCCCCTGGATGTGCAGTGGAATGACCTGGACTACATGGACAGCAGGAGGGACTTCA
CCTTTAACAAGGATGGCTTTAGGGACTTCCCTGCCATGGTGCAGGAGCTGCATCAGGGGGGCAGGAGGTACATGA
TGATTGTGGACCCAGCCATCAGCAGCTCTGGGCCTGCTGGGTCTTACAGGCCCTATGATGAGGGCCTGAGGAGGG
GGGTGTTCATCACCAATGAGACTGGCCAGCCCCTGATTGGCAAGGTGTGGCCTGGGAGCACTGCCTTCCCTGATT
TTACCAACCCCACTGCCCTGGCCTGGTGGGAGGATATGGTGGCTGAGTTTCATGACCAGGTGCCCTTTGATGGCA
TGTGGATTGACATGAATGAGCCCAGCAATTTCATCAGGGGCTCTGAGGATGGCTGCCCCAACAATGAGCTGGAGA
ATCCTCCCTATGTGCCTGGGGTGGTGGGGGGCACCCTGCAGGCTGCCACCATCTGTGCCTCTAGCCACCAGTTCC
TGAGCACCCACTATAACCTGCATAACCTGTATGGCCTGACTGAGGCCATTGCCAGCCATAGAGCCCTGGTGAAGG
CCAGAGGGACCAGGCCCTTTGTGATCTCTAGGAGCACCTTTGCTGGCCATGGCAGGTATGCTGGCCACTGGACTG
GGGATGTGTGGAGCTCTTGGGAGCAGCTGGCCAGCTCTGTGCCAGAGATCCTGCAGTTCAACCTGCTGGGGGTGC
CTCTGGTGGGGGCTGATGTGTGTGGCTTCCTGGGCAATACCTCTGAAGAGCTGTGTGTGAGGTGGACTCAGCTGG
GGGCCTTCTATCCCTTCATGAGGAACCACAACAGCCTGCTGTCTCTGCCCCAGGAGCCCTACAGCTTCTCTGAGC
CTGCTCAGCAGGCTATGAGGAAGGCCCTGACCCTGAGGTATGCCCTGCTGCCCCATCTGTACACCCTGTTCCACC
AGGCCCATGTGGCTGGGGAGACTGTGGCCAGGCCCCTGTTTCTGGAGTTTCCCAAGGACAGCAGCACCTGGACTG
TGGACCATCAGCTGCTGTGGGGGGAGGCTCTGCTGATTACCCCTGTGCTGCAGGCTGGCAAGGCTGAGGTGACTG
GGTACTTCCCCCTGGGGACTTGGTATGACCTGCAGACTGTGCCTGTGGAAGCTCTGGGCAGCCTGCCCCCACCCC
CTGCTGCCCCTAGGGAGCCTGCCATCCACTCTGAGGGCCAGTGGGTGACCCTGCCTGCCCCTCTGGACACCATCA
ATGTGCACCTGAGGGCTGGCTATATCATCCCCCTGCAGGGCCCTGGGCTGACCACCACTGAGAGCAGGCAGCAGC
CCATGGCCCTGGCTGTGGCCCTGACTAAGGGGGGGGAGGCCAGGGGGGAGCTGTTCTGGGATGATGGGGAGAGCC
TGGAGGTGCTGGAGAGAGGGGCCTACACCCAGGTGATCTTTCTGGCCAGGAACAACACCATTGTGAATGAGCTGG
TGAGGGTGACTTCTGAGGGGGCTGGCCTGCAGCTGCAGAAGGTGACTGTGCTGGGGGTGGCCACTGCCCCCCAGC
AGGTGCTGAGCAATGGGGTGCCTGTGTCTAACTTCACCTACAGCCCTGATACTAAGGTGCTGGATATCTGTGTGA
GCCTGCTGATGGGGGAGCAGTTTCTGGTGAGCTGGTGCTGACTCGAGAGATCTACCGGTGAATTCACCGCGGGTT
TAAACTGTGCCTTCTAGTTGCCAGCCATCTGTTGTTTGCCCCTCCCCCGTGCCTTCCTTGACCCTGGAAGGTGCC
ACTCCCACTGTCCTTTCCTAATAAAATGAGGAAATTGCATCGCATTGTCTGAGTAGGTGTCATTCTATTCTGGGG
GGTGGGGTGGGGGCTAGCTCTAGACTCGAGATCCACTAGGGCCGCAGGAACCCCTAGTGATGGAGTTGGCCACTC
CCTCTCTGCGCGCTCGCTCGCTCACTGAGGCCGGGCGACCAAAGGTCGCCCGACGCCCGGGCTTTGCCCGGGCGG
CCTCAGTGAGCGAGCGAGCGCGCAGCTGCCTGCAGG
>pAAV-ApoE/hAAT.GAA13.wtBGH
(SEQ ID NO: 24)
CCTGCAGGCAGCTGCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAACTCCATCACTAGGGGTTCCTGCGGCCGCA
GGCTCAGAGGCACACAGGAGTTTCTGGGCTCACCCTGCCCCCTTCCAACCCCTCAGTTCCCATCCTCCAGCAGCT
GTTTGTGTGCTGCCTCTGAAGTCCACACTGAACAAACTTCAGCCTACTCATGTCCCTAAAATGGGCAAACATTGC
AAGCAGCAAACAGCAAACACACAGCCCTCCCTGCCTGCTGACCTTGGAGCTGGGGCAGAGGTCAGAGACCTCTCT
GGGCCCATGCCACCTCCAACATCCACTCGACCCCTTGGAATTTCGGTGGAGAGGAGCAGAGGTTGTCCTGGCGTG
GTTTAGGTAGTGTGAGAGGGGTACCCGGGGATCTTGCTACCAGTGGAACAGCCACTAAGGATTCTGCAGTGAGAG
CAGAGGGCCAGCTAAGTGGTACTCTCCCAGAGACTGTCTGACTCACGCCACCCCCTCCACCTTGGACACAGGACG
CTGTGGTTTCTGAGCCAGGTACAATGACTCCTTTCGGTAAGTGCAGTGGAAGCTGTACACTGCCCAGGCAAAGCG
TCCGGGCAGCGTAGGCGGGCGACTCAGATCCCAGCCAGTGGACTTAGCCCCTGTTTGCTCCTCCGATAACTGGGG
TGACCTTGGTTAATATTCACCAGCAGCCTCCCCCGTTGCCCCTCTGGATCCACTGCTTAAATACGGACGAGGACA
GGGCCCTGTCTCCTCAGCTTCAGGCACCACCACTGACCTGGGACAGTGAATAGATCCTGAGAACTTCAGGGTGAG
TCTATGGGACCCTTGATGTTTTCTTTCCCCTTCTTTTCTATGGTTAAGTTCATGTCATAGGAAGGGGAGAAGTAA
CAGGGTACACATATTGACCAAATCAGGGTAATTTTGCATTTGTAATTTTAAAAAATGCTTTCTTCTTTTAATATA
CTTTTTTGTTTATCTTATTTCTAATACTTTCCCTAATCTCTTTCTTTCAGGGCAATAATGATACAATGTATCATG
CCTCTTTGCACCATTCTAAAGAATAACAGTGATAATTTCTGGGTTAAGGCAATAGCAATATTTCTGCATATAAAT
ATTTCTGCATATAAATTGTAACTGATGTAAGAGGTTTCATATTGCTAATAGCAGCTACAATCCAGCTACCATTCT
GCTTTTATTTTCTGGTTGGGATAAGGCTGGATTATTCTGAGTCCAAGCTAGGCCCTTTTGCTAATCTTGTTCATA
CCTCTTATCTTCCTCCCACAGCTCCTGGGCAACCTGCTGGTCTCTCTGCTGGCCCATCACTTTGGCAAAGCACGC
GTGCCACCATGGCCTTTCTGTGGCTGCTGTCCTGCTGGGCCCTGCTGGGGACCACCTTTGGCCTGCTGGTGCCCA
GGGAGCTGTCTGGGAGCAGCCCAGTGCTGGAGGAGACCCACCCTGCCCACCAGCAGGGGGCCAGCAGGCCTGGCC
CTAGGGATGCCCAGGCCCACCCTGGCAGGCCCAGGGCTGTGCCTACCCAGTGTGATGTGCCACCCAATTCTAGGT
TTGACTGTGCTCCTGACAAGGCCATCACTCAGGAGCAGTGTGAAGCTAGGGGGTGCTGCTACATCCCAGCCAAGC
AGGGCCTGCAGGGGGCCCAGATGGGCCAGCCCTGGTGCTTCTTCCCCCCCAGCTACCCTAGCTACAAGCTGGAGA
ATCTGAGCAGCTCTGAGATGGGCTACACTGCTACCCTGACCAGGACCACTCCTACCTTCTTCCCCAAGGACATCC
TGACTCTGAGGCTGGATGTCATGATGGAGACTGAAAATAGGCTGCACTTCACCATCAAGGACCCTGCCAATAGGA
GGTATGAGGTGCCTCTGGAGACCCCCCATGTGCATAGCAGGGCTCCCAGCCCCCTGTATTCTGTGGAGTTCTCTG
AGGAGCCCTTTGGGGTCATTGTGAGGAGACAGCTGGATGGGAGGGTGCTGCTGAACACTACTGTGGCTCCCCTGT
TCTTTGCTGACCAGTTCCTGCAGCTGTCTACCAGCCTGCCCAGCCAGTACATCACTGGGCTGGCTGAGCATCTGA
GCCCCCTGATGCTGAGCACCAGCTGGACCAGGATCACTCTGTGGAACAGGGATCTGGCCCCCACTCCTGGGGCCA
ACCTGTATGGGAGCCATCCCTTCTACCTGGCCCTGGAGGATGGGGGCTCTGCCCATGGGGTGTTCCTGCTGAACA
GCAATGCCATGGATGTGGTGCTGCAGCCTAGCCCTGCCCTGAGCTGGAGGAGCACTGGGGGCATCCTGGATGTCT
ACATCTTCCTGGGGCCTGAGCCCAAGTCTGTGGTGCAGCAGTATCTGGATGTGGTGGGGTATCCCTTCATGCCCC
CCTACTGGGGCCTGGGCTTTCACCTGTGCAGGTGGGGCTACAGCAGCACTGCCATCACCAGGCAGGTGGTGGAGA
ACATGACCAGGGCCCACTTCCCTCTGGATGTGCAGTGGAATGACCTGGACTATATGGATTCTAGGAGAGACTTTA
CTTTTAACAAGGATGGCTTCAGGGATTTCCCTGCCATGGTGCAGGAGCTGCACCAGGGGGGCAGGAGGTACATGA
TGATTGTGGACCCTGCTATTAGCAGCTCTGGCCCTGCTGGGTCTTACAGGCCTTATGATGAGGGCCTGAGGAGGG
GGGTGTTCATCACCAATGAGACTGGCCAGCCCCTGATTGGCAAGGTGTGGCCTGGCAGCACTGCCTTCCCTGACT
TCACCAACCCCACTGCCCTGGCCTGGTGGGAGGACATGGTGGCTGAGTTCCATGACCAGGTGCCCTTTGATGGGA
TGTGGATTGACATGAATGAGCCCTCTAACTTCATCAGGGGGTCTGAGGATGGCTGCCCCAACAATGAGCTGGAGA
ACCCCCCCTATGTGCCTGGGGTGGTGGGGGGCACTCTGCAGGCTGCCACTATCTGTGCTTCTTCTCACCAGTTTC
TGAGCACCCACTATAATCTGCACAACCTGTATGGCCTGACTGAGGCCATTGCCAGCCATAGGGCCCTGGTGAAGG
CCAGGGGCACCAGGCCCTTTGTGATCAGCAGGTCTACCTTTGCTGGCCATGGCAGGTATGCTGGCCACTGGACTG
GGGATGTGTGGTCTTCTTGGGAGCAGCTGGCCAGCTCTGTGCCTGAGATCCTGCAGTTCAACCTGCTGGGGGTGC
CTCTGGTGGGGGCTGATGTGTGTGGCTTTCTGGGCAACACCTCTGAGGAGCTGTGTGTGAGGTGGACCCAGCTGG
GGGCCTTTTACCCCTTCATGAGGAACCACAATAGCCTGCTGAGCCTGCCCCAGGAGCCTTACTCTTTCTCTGAGC
CTGCCCAGCAGGCCATGAGGAAGGCCCTGACTCTGAGGTATGCCCTGCTGCCCCATCTGTATACCCTGTTTCACC
AGGCCCATGTGGCTGGGGAGACTGTGGCTAGGCCTCTGTTTCTGGAGTTCCCTAAGGACTCTAGCACCTGGACTG
TGGACCACCAGCTGCTGTGGGGGGAGGCCCTGCTGATCACCCCTGTGCTGCAGGCTGGCAAGGCTGAGGTGACTG
GCTACTTCCCCCTGGGCACCTGGTATGACCTGCAGACTGTGCCTGTGGAGGCCCTGGGGAGCCTGCCTCCCCCCC
CTGCTGCCCCCAGGGAGCCTGCCATTCATTCTGAGGGCCAGTGGGTGACCCTGCCTGCCCCTCTGGACACCATCA
ATGTGCACCTGAGGGCTGGGTACATCATCCCCCTGCAGGGCCCTGGCCTGACCACCACTGAGAGCAGGCAGCAGC
CCATGGCCCTGGCTGTGGCTCTGACCAAGGGGGGGGAGGCCAGGGGGGAGCTGTTCTGGGATGATGGGGAGTCTC
TGGAGGTGCTGGAGAGGGGGGCCTACACCCAGGTGATCTTTCTGGCCAGGAACAATACTATTGTGAATGAGCTGG
TGAGGGTGACCTCTGAGGGGGCTGGCCTGCAGCTGCAGAAGGTGACTGTGCTGGGGGTGGCCACTGCCCCCCAGC
AGGTCCTGAGCAATGGGGTGCCTGTGAGCAACTTCACCTACTCTCCTGACACCAAGGTGCTGGACATTTGTGTGT
CTCTGCTGATGGGGGAGCAGTTCCTGGTGAGCTGGTGCTGACTCGAGAGATCTACCGGTGAATTCACCGCGGGTT
TAAACTGTGCCTTCTAGTTGCCAGCCATCTGTTGTTTGCCCCTCCCCCGTGCCTTCCTTGACCCTGGAAGGTGCC
ACTCCCACTGTCCTTTCCTAATAAAATGAGGAAATTGCATCGCATTGTCTGAGTAGGTGTCATTCTATTCTGGGG
GGTGGGGTGGGGGCTAGCTCTAGACTCGAGATCCACTAGGGCCGCAGGAACCCCTAGTGATGGAGTTGGCCACTC
CCTCTCTGCGCGCTCGCTCGCTCACTGAGGCCGGGCGACCAAAGGTCGCCCGACGCCCGGGCTTTGCCCGGGCGG
CCTCAGTGAGCGAGCGAGCGCGCAGCTGCCTGCAGG
SEQ ID NO: 25: amino acid sequence for a secretable GAA protein
MAFLWLLSCWALLGTTFGLLVPRELSGSSPVLEETHPAHQQGASRPGPRDAQAHPGRPRAVPTQCDVPPNSREDC
APDKAITQEQCEARGCCYIPAKQGLQGAQMGQPWCFFPPSYPSYKLENLSSSEMGYTATLTRTTPTFFPKDILTL
RLDVMMETENRLHFTIKDPANRRYEVPLETPHVHSRAPSPLYSVEFSEEPFGVIVRRQLDGRVLLNTTVAPLFFA
DQFLQLSTSLPSQYITGLAEHLSPLMLSTSWTRITLWNRDLAPTPGANLYGSHPFYLALEDGGSAHGVFLLNSNA
MDVVLQPSPALSWRSTGGILDVYIFLGPEPKSVVQQYLDVVGYPFMPPYWGLGFHLCRWGYSSTAITRQVVENMT
RAHFPLDVQWNDLDYMDSRRDFTENKDGFRDFPAMVQELHQGGRRYMMIVDPAISSSGPAGSYRPYDEGLRRGVF
ITNETGQPLIGKVWPGSTAFPDFTNPTALAWWEDMVAEFHDQVPFDGMWIDMNEPSNFIRGSEDGCPNNELENPP
YVPGVVGGTLQAATICASSHQFLSTHYNLHNLYGLTEAIASHRALVKARGTRPFVISRSTFAGHGRYAGHWTGDV
WSSWEQLASSVPEILQFNLLGVPLVGADVCGFLGNTSEELCVRWTQLGAFYPFMRNHNSLLSLPQEPYSFSEPAQ
QAMRKALTLRYALLPHLYTLFHQAHVAGETVARPLFLEFPKDSSTWTVDHQLLWGEALLITPVLQAGKAEVTGYF
PLGTWYDLQTVPVEALGSLPPPPAAPREPAIHSEGQWVTLPAPLDTINVHLRAGYIIPLQGPGLTTTESRQQPMA
LAVALTKGGEARGELFWDDGESLEVLERGAYTQVIFLARNNTIVNELVRVTSEGAGLQLQKVTVLGVATAPQQVL
SNGVPVSNFTYSPDTKVLDICVSLLMGEQFLVSWC
SEQ ID NO: 26: CpG reduced BGH poly A nucleic acid sequence
AGATCTAGAGCTGAATTCCTGCAGCCAGGGGGATCAGCCTCTACTGTGCCTTCTAGTTGCCAGCCATCTGTTGTT
TGCCCCTCCCCCTTGCCTTCCTTGACCCTGGAAGGTGCCACTCCCACTGTCCTTTCCTAATAAAATGAGGAAATT
GCATCACATTGTCTGAGTAGGTGTCATTCTATTCTGGGGGGGGGGTGGGGCAGGACAGCAAGGGGGAGGATTGG
GAAGACAATAGCAGGCATGCTGGGGATGCAGTGGGCTCTATGG
SEQ ID NO: 27: wtBGH poly A nucleic acid sequence
AGATCTACCGGTGAATTCACCGCGGGTTTAAACTGTGCCTTCTAGTTGCCAGCCATCTGTTGTTTGCCCCTCCCC
CGTGCCTTCCTTGACCCTGGAAGGTGCCACTCCCACTGTCCTTTCCTAATAAAATGAGGAAATTGCATCGCATTG
TCTGAGTAGGTGTCATTCTATTCTGGGGGGTGGGGTGGGGGCTAGCTCTAGA
SEQ ID NO: 28: ApoE/hAAT sequence bordered by Apal restriction sites
(underlined).
GGGCCCATGCCACCTCCAACATCCACTCGACCCCTTGGAATTTCGGTGGAGAGGAGCAGAGGTTGTCCTGGCGTG
GTTTAGGTAGTGTGAGAGGGGTACCCGGGGATCTTGCTACCAGTGGAACAGCCACTAAGGATTCTGCAGTGAGAG
CAGAGGGCCAGCTAAGTGGTACTCTCCCAGAGACTGTCTGACTCACGCCACCCCCTCCACCTTGGACACAGGACG
CTGTGGTTTCTGAGCCAGGTACAATGACTCCTTTCGGTAAGTGCAGTGGAAGCTGTACACTGCCCAGGCAAAGCG
TCCGGGCAGCGTAGGCGGGCGACTCAGATCCCAGCCAGTGGACTTAGCCCCTGTTTGCTCCTCCGATAACTGGGG
TGACCTTGGTTAATATTCACCAGCAGCCTCCCCCGTTGCCCCTCTGGATCCACTGCTTAAATACGGACGAGGACA
GGGCCC
SEQ ID NO: 29: ApoE/hAAT sequence.
ATGCCACCTCCAACATCCACTCGACCCCTTGGAATTTCGGTGGAGAGGAGCAGAGGTTGTCCTGGCGTGGTTTAG
GTAGTGTGAGAGGGGTACCCGGGGATCTTGCTACCAGTGGAACAGCCACTAAGGATTCTGCAGTGAGAGCAGAGG
GCCAGCTAAGTGGTACTCTCCCAGAGACTGTCTGACTCACGCCACCCCCTCCACCTTGGACACAGGACGCTGTGG
TTTCTGAGCCAGGTACAATGACTCCTTTCGGTAAGTGCAGTGGAAGCTGTACACTGCCCAGGCAAAGCGTCCGGG
CAGCGTAGGCGGGCGACTCAGATCCCAGCCAGTGGACTTAGCCCCTGTTTGCTCCTCCGATAACTGGGGTGACCT
TGGTTAATATTCACCAGCAGCCTCCCCCGTTGCCCCTCTGGATCCACTGCTTAAATACGGACGAGGACA
AAV vector capsids:
VP1
(SEQ ID NO: 30)
MAADGYLPDWLEDNLSEGIREWWDLKPGAPKPKANQQKQDNGRGLVLPGYKYLGPENGLD
KGEPVNAADAAALEHDKAYDQQLQAGDNPYLRYNHADAEFQERLQEDTSEGGNLGRAVFQ
AKKRVLEPLGLVESPVKTAPGKKRPVEPSPQRSPDSSTGIGKKGQQPAKKRLNFGQTGDS
ESVPDPQPIGEPPAAPSGVGPNTMAAGGGAPMADNNEGADGVGSSSGNWHCDSTWLGDRV
ITTSTRTWALPTYNNHLYKQISNGTSGGSTNDNTYFGYSTPWGYFDFNRFHCHFSPRDWQ
RLINNNWGFRPKRLNFKLFNIQVKEVTQNEGTKTIANNLTSTIQVFTDSEYQLPYVLGSA
HQGCLPPFPADVEMIPQYGYLTLNNGSQAVGRSSFYCLEYFPSQMLRTGNNFEFSYNFED
VPFHSSYAHSQSLDRLMNPLIDQYLYYLSRTQSTGGTAGTQQLLFSQAGPNNMSAQAKNW
LPGPCYRQQRVSTTLSQNNNSNFAWTGATKYHLNGRDSLVNPGVAMATHKDDEERFFPSS
GVLMFGKQGAGKDNVDYSSVMLTSEEEIKTTNPVATEQYGVVADNLQQQNAAPIVGAVNS
QGALPGMVWQNRDVYLQGPIWAKIPHTDGNFHPSPLMGGFGLKHPPPQILIKNTPVPADP
PTTFNQAKLASFITQYSTGQVSVEIEWELQKENSKRWNPEIQYTSNYYKSTNVDFAVNTE
GTYSEPRPIGTRYLTRNL
VP2
(SEQ ID NO: 31)
TAPGKKRPVEPSPQRSPDSSTGIGKKGQQPAKKRLNFGQTGDSESVPDPQPIGEPPAGPS
GLGSGTMAAGGGAPMADNNEGADGVGSSSGNWHCDSTWLGDRVITTSTRTWALPTYNNHL
YKQISNGTSGGSTNDNTYFGYSTPWGYFDFNRFHCHFSPRDWQRLINNNWGFRPKRLNEK
LFNIQVKEVTQNEGTKTIANNLTSTIQVFTDSEYQLPYVLGSAHQGCLPPFPADVFMIPQ
YGYLTLNNGSQAVGRSSFYCLEYFPSQMLRTGNNFEFSYNFEDVPFHSSYAHSQSLDRLM
NPLIDQYLYYLSRTQSTGGTAGTQQLLFSQAGPNNMSAQAKNWLPGPCYRQQRVSTTLSQ
NNNSNFAWTGATKYHLNGRDSLVNPGVAMATHKDDEERFFPSSGVLMFGKQGAGKDNVDY
SSVMLTSEEEIKTTNPVATEQYGVVADNLQQQNAAPIVGAVNSQGALPGMVWQNRDVYLQ
GPIWAKIPHTDGNFHPSPLMGGFGLKHPPPQILIKNTPVPADPPTTENQAKLASFITQYS
TGQVSVEIEWELQKENSKRWNPEIQYTSNYYKSTNVDFAVNTEGTYSEPRPIGTRYLTRN
L
VP3
(SEQ ID NO: 32)
MAAGGGAPMADNNEGADGVGSSSGNWHCDSTWLGDRVITTSTRTWALPTYNNHLYKQISN
GTSGGSINDNTYFGYSTPWGYFDFNRFHCHFSPRDWQRLINNNWGFRPKRLNFKLFNIQV
KEVTQNEGTKTIANNLTSTIQVFTDSEYQLPYVLGSAHQGCLPPFPADVFMIPQYGYLTL
NNGSQAVGRSSFYCLEYFPSQMLRIGNNFEFSYNFEDVPFHSSYAHSQSLDRLMNPLIDQ
YLYYLSRTQSTGGTAGTQQLLFSQAGPNNMSAQAKNWLPGPCYRQQRVSTTLSQNNNSNF
AWTGATKYHLNGRDSLVNPGVAMATHKDDEERFFPSSGVLMFGKQGAGKDNVDYSSVMLT
SEEEIKTTNPVATEQYGVVADNLQQQNAAPIVGAVNSQGALPGMVWQNRDVYLQGPIWAK
IPHTDGNFHPSPLMGGFGLKHPPPQILIKNTPVPADPPTTFNQAKLASFITQYSTGQVSV
EIEWELQKENSKRWNPEIQYTSNYYKSTNVDFAVNTEGTYSEPRPIGTRYLTRNL
LK03-AAV VP1
(SEQ ID NO: 33)
MAADGYLPDWLEDNLSEGIREWWALQPGAPKPKANQQHQDNARGLVLPGYKYLGPGNGLDKGEPVNAADAAALEH
DKAYDQQLKAGDNPYLKYNHADAEFQERLKEDTSFGGNLGRAVFQAKKRLLEPLGLVEEAAKTAPGKKRPVDQSP
QEPDSSSGVGKSGKQPARKRLNFGQTGDSESVPDPQPLGEPPAAPTSLGSNTMASGGGAPMADNNEGADGVGNSS
GNWHCDSQWLGDRVITTSTRTWALPTYNNHLYKQISSQSGASNDNHYFGYSTPWGYFDFNRFHCHFSPRDWQRLI
NNNWGFRPKKLSFKLFNIQVKEVTQNDGTTTIANNLTSTVQVFTDSEYQLPYVLGSAHQGCLPPFPADVEMVPQY
GYLTLNNGSQAVGRSSFYCLEYFPSQMLRTGNNFQFSYTFEDVPFHSSYAHSQSLDRLMNPLIDQYLYYLNRTQG
TTSGTTNQSRLLFSQAGPQSMSLQARNWLPGPCYRQQRLSKTANDNNNSNFPWTAASKYHLNGRDSLVNPGPAMA
SHKDDEEKFFPMHGNLIFGKEGTTASNAELDNVMITDEEEIRTTNPVATEQYGTVANNLQSSNTAPTTRTVNDQG
ALPGMVWQDRDVYLQGPIWAKIPHTDGHFHPSPLMGGFGLKHPPPQIMIKNTPVPANPPTTFSPAKFASFITQYS
TGQVSVEIEWELQKENSKRWNPEIQYTSNYNKSVNVDFTVDINGVYSEPRPIGTRYLTRPL

### Example 2

### Study Design—Potency In Vitro (Huh7 Cells)

To evaluate potency of the GAA expression cassettes, 200 ng of plasmid DNA corresponding to each cassette was transfected into a human hepatocellular carcinoma cell line (Huh7) using Lipofectamine® 2000 (ThermoFisher). Following 48 hours of incubation, samples of culture medium from cells transfected with plasmids bearing each expression cassette were assessed for the levels of GAA by GAA activity assay and normalized to the intracellular protein content.

### Results

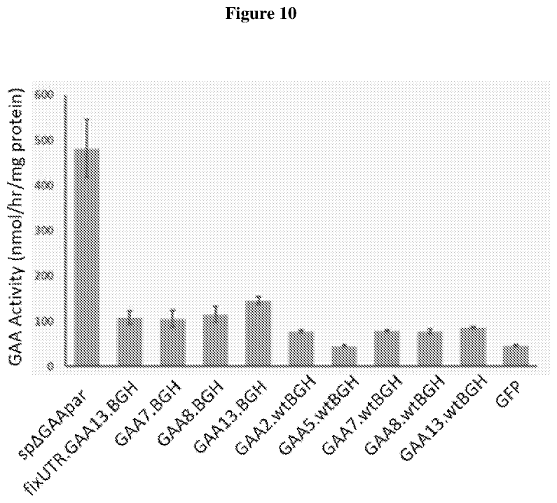

FIG. 10 shows evaluation of different GAA expression plasmids in vitro in Huh7 cells by GAA activity assay. Results are the average of biological replicates n=3. Error bars represent standard deviation.

With the exception of GAA5.wtBGH, all plasmids demonstrated increased expression levels of secreted GAA compared to a green fluorescent protein (GFP) control plasmid, indicating that these plasmids resulted in expression of the GAA protein product (FIG. 10). In general, “BGH” plasmids (harboring a CpG-reduced poly A sequence) outperformed “wtBGH” plasmids (with a wild-type bGH poly A). In addition, codon optimized sequences GAA2, GAA7, GAA 8, and GAA13 appeared to be relatively similar, with a trend towards higher expression from GAA13 plasmids. An overall decrease in expression levels was observed when compared with parental spAGAApar plasmid.

### Example 3

### Study Design—Cassette Potency by Hydrodynamic Injection in Mice

To evaluate the potency of different GAA expression cassettes in mammals, 25 micrograms of plasmid DNA corresponding to each cassette was introduced into male C57BL/6 mice (approximately 8 weeks of age) intravenously in the lateral tail vein by hydrodynamic injection. Plasma was collected at 72 hours post-injection and assessed for circulating GAA enzyme presence by GAA activity assay and western blot.

### Results

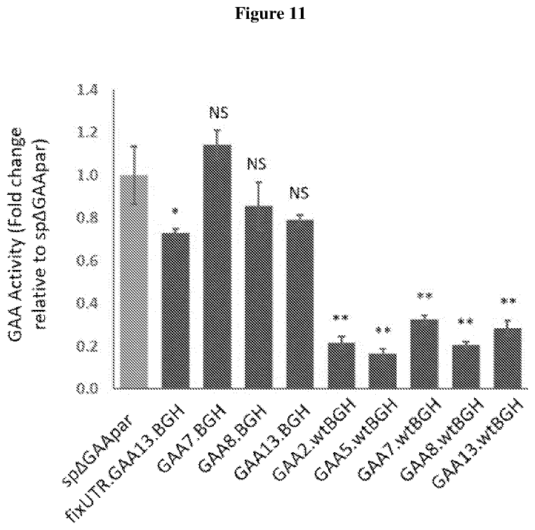

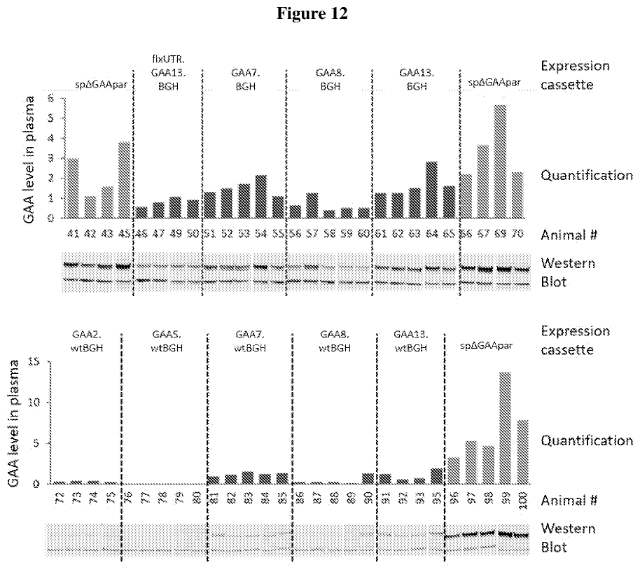

FIGS. 11 and 12 show evaluation of different GAA expression cassettes by hydrodynamic injection in male C57BL/6 mice by GAA activity assay and western blot, respectively, in plasma. For FIG. 11, plasma levels of GAA are normalized to the parental construct (spAGAApar). Results are the average of biological replicates n=4-5. Error bars represent standard deviation. NS, not significant. * p<0.05, ** p<0.01.

For FIG. 12, the GAA western blot analysis of plasma, the bar graph represents quantification of the upper band normalized to the bottom, non-specific band. Each of the biological replicates (n=4-5 per group) is shown.

Evaluation of codon-optimized GAA expression cassettes in mice by hydrodynamic injection revealed that these cassettes were capable of expression and secretion of the GAA transgene product from mouse liver, with levels similar to those observed with the parental GAA construct, spAGAApar (FIG. 11). As in the in vitro studies in Huh7 cells (Example 2), GAA expression cassettes harboring the CpG-reduced BGH poly A sequence outperformed those with a wild-type BGH poly A. Unexpectedly, in comparison to the results of Example 2, some of the codon-optimized GAA constructs performed as well as or better than the parental spAGAApar plasmid.

### Example 4

### Study Design—Vector Potency in Mice

To evaluate the potency of the codon-optimized GAA cassettes of the invention compared with parental cassette spAGAApar, spAGAApar and 9 codon-optimized cassettes were packaged into an AAV vector comprising SEQ ID NOs: 30-32 capsids (Table 1). Five male C57BL/6 mice per group were injected intravenously via the tail vein with 1 of 2 vector doses (5×10¹¹ vg/kg or 2×10¹² vg/kg). Circulating GAA activity was longitudinally measured following AAV vector administration to the mice. Plasma were tested bi-weekly for GAA activity. At day 70, 7 of the 10 test animal groups were sacrificed, and the remaining three groups (SPK-AAV-01, SPK-AAV-02, and the parental vector) were sacrificed at day 147.

### Results

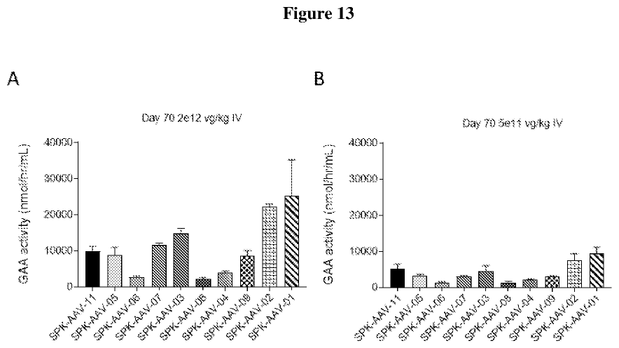

FIG. 13 shows evaluation of different codon-optimized GAA expression cassettes packaged into AAV (SEQ ID NOs: 30-32) vector in male C57BL/6 mice by GAA activity assay in plasma 70 days post vector infusion. Results are the average of biological replicates n=4-5. Error bars represent standard deviation. NS, not significant. * p<0.05, ** p<0.01.

Two vectors, SPK-AAV-01 and SPK-AAV-02, consistently showed significantly higher GAA activity in the plasma (approximately 2- to 3-fold higher in the high dose) when compared to the parental vector (designated herein as SPK-AAV-11).

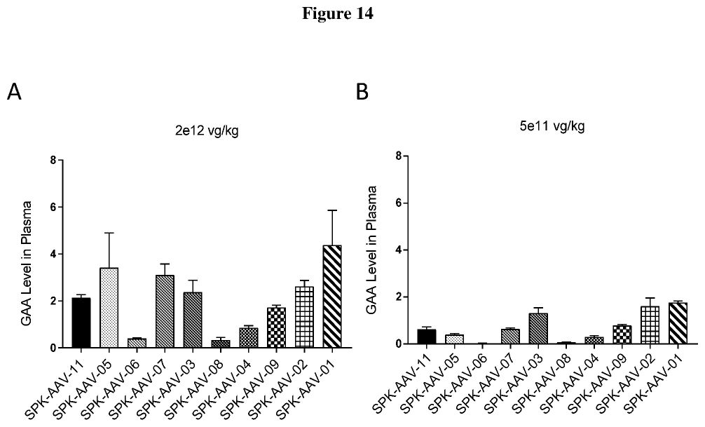

FIG. 14 shows evaluation of different codon-optimized GAA expression cassettes packaged into AAV (SEQ ID NOs: 30-32) vector in male C57BL/6 mice by GAA western blot in plasma 7 days post vector infusion. Results are the average of biological replicates n=4-5. Error bars represent standard deviation. NS, not significant. * p<0.05, ** p<0.01.

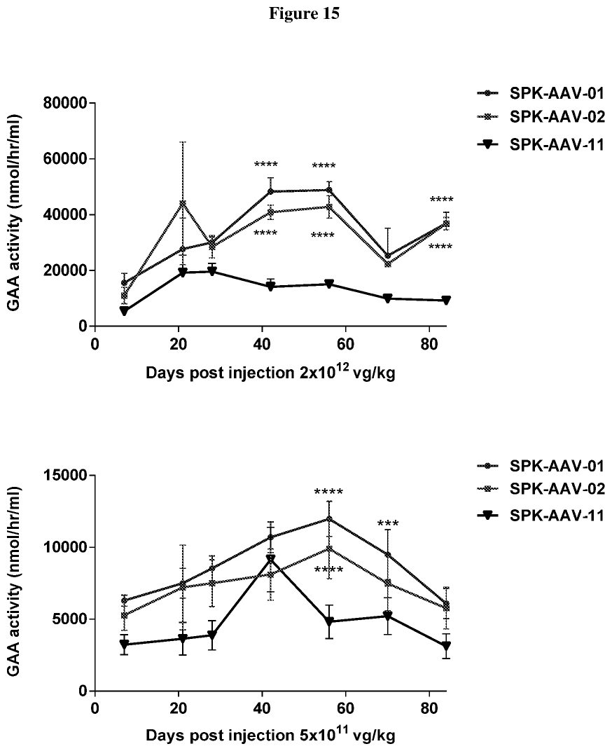

FIG. 15 shows GAA activity levels measured in plasma following administration of codon-optimized GAA expression cassettes packaged into AAV (SEQ ID NOs: 30-32) vector to mice. Male C57BL/6 mice were injected with a dose of 5×10¹¹ (upper panel) or 2×10¹² vg/kg (lower panel) of either of the two best performing GAA-expressing AAV (SEQ ID NOs: 30-32) vectors (n=5 animals/group). GAA activity measured in the plasma of mice that received SPK-AAV-11 vector (parental spAGAApar construct) is shown for comparison. Starting at day 42, GAA activity in plasma was significantly higher for SPK-AAV-01 and SPK-AAV-02 vectors than that for SPK-AAV-11 vector (*** p=<0.001, **** p=<0.0001 Tukey multiple comparisons test).

### Example 5

### Study Design—Potency in Rats

The purpose of this study was to evaluate the potency of SPK-AAV-02 in two different rat strains. Circulating GAA activity was measured in plasma following vector administration to Wistar Hanover (n=4) and Sprague Dawley (n=5) rats. Male rats were injected intravenously via the tail vein with 2×10¹³ vg/kg of vector.

### Results

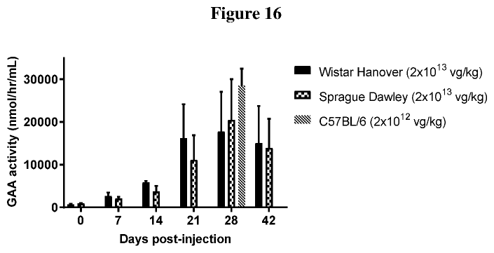

FIG. 16 shows evaluation of SPK-AAV-02 (pAAV-ApoE/hAAT.GAA13.BGH packaged into AAV (SEQ ID NOs: 30-32)) vector in male Wistar Hanover and Sprague Dawley rats compared to C57BL/6 mice by GAA activity assay in plasma. Results are the average of biological replicates n=4-5. Error bars represent standard deviation.

The levels of GAA activity started to plateau at day 28 with GAA activity measured around 15,000 nmol/hr/mL. Mice were injected with SPK-AAV-02 at a 10-fold lower dose (2×10¹² vg/kg of vector) than the rats.

### Example 6

### Study Design—Potency in Non-Human Primates (NHPs), Part 1

To evaluate the long-term effects of secretable GAA expression, a single dose of SPK-AAV-01, SPK-AAV-02 or SPK-AAV-10 vector was administered to a species phylogenetically close to human, rhesus macaques (Macaca mulatta), and animals were evaluated for GAA activity levels in plasma.

Male rhesus macaques (n=3/dose cohort) received a 30-minute IV infusion in the saphenous vein of a single dose of 6×10¹² vg/kg of SPK-AAV-01, SPK-AAV-02 or SPK-AAV-10 vector. Following AAV vector administration, animals were monitored daily for clinical observations, and blood was collected weekly.

### Results

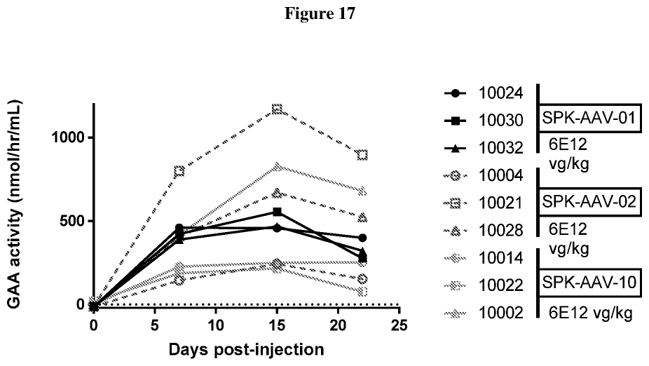

Rhesus macaques were injected with a dose of 6×10¹² vg/kg of GAA-expressing AAV (SEQ ID NOs: 30-32) vectors (n=3 animals/group). FIG. 17 shows GAA activity levels measured in plasma following administration of GAA expression cassettes packaged into AAV (SEQ ID NOs: 30-32) vector to male rhesus macaques. No significant differences were observed among the plasma GAA activity levels of SPK-AAV-01, SPK-AAV-02 and SPK-AAV-10 vector injected NHPs.

Preliminary data in the NHP study confirm the results observed in the mouse studies, and show that plasma GAA activity levels resulting from administration of SPK-AAV-01 and SPK-AAV-02 are similar (FIG. 17).

### Example 7

### Study Design—Potency in Non-Human Primates, Part 2

The main objective of this study was to evaluate the transduction profile of SPK-AAV-01 and SPK-AAV-02 in African green monkeys (AGM; Chlorocebus sabaeus). Male AGM (n=4/dose cohort) received a 30-minute IV infusion in the saphenous vein of a single dose of 6×10¹² vg/kg of SPK-AAV-01 or SPK-AAV-02 vector. Following AAV vector administration, animals were monitored daily for clinical observations, and blood was collected weekly.

### Results

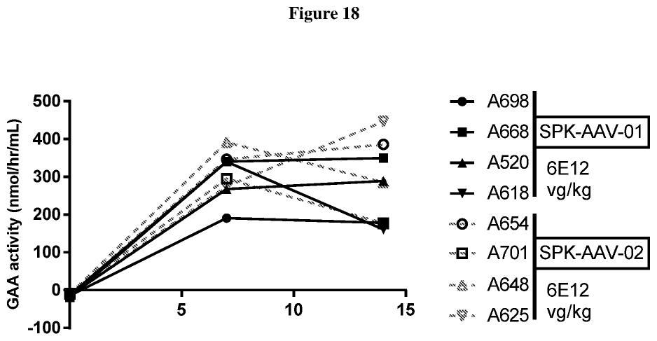

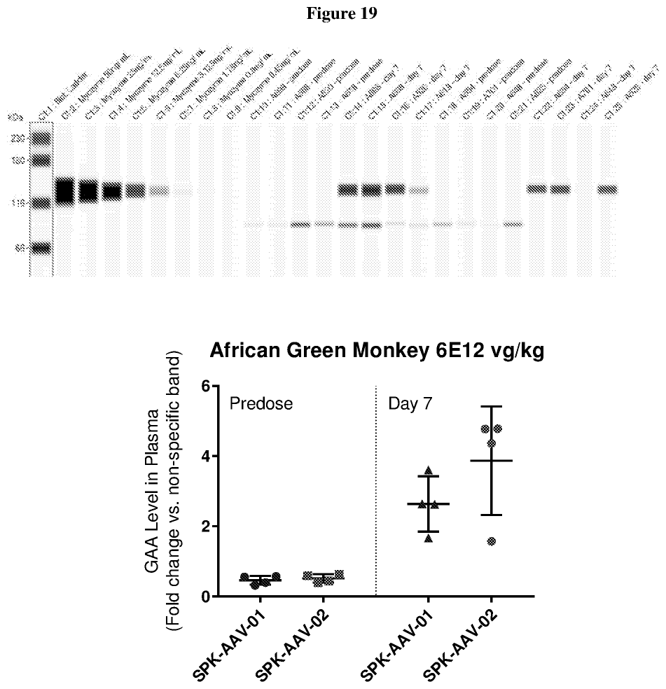

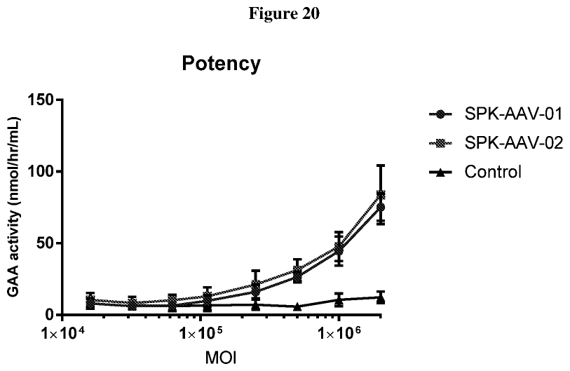

Preliminary data in African green monkeys confirm the results observed in mouse studies that plasma GAA activity levels resulting from administration of SPK-AAV-01 and SPK-AAV-02 are similar (FIG. 18).

### Example 8

### Conclusions

The codon-optimized GAA cassettes that express secretable human GAA represent a significant improvement over the parental spAGAApar construct. In particular, SPK-AAV-01 and SPK-AAV-02 exhibit excellent potency and expression of secretable human GAA in cell culture, rodent, and two non-human primate models.

### Example 9

### Study Design—Dose Ranging in NHPs

To evaluate the dose response of secretable GAA expression, three doses of SPK-AAV-02 vector were administered to a primate species phylogenetically close to humans, rhesus macaques (Macaca mulatta), and animals were evaluated for GAA activity levels over the course of one month.

Male rhesus macaques (n=4/dose cohort) received a 30-minute intravenous (IV) infusion in the saphenous vein of a dose of either 2×10¹², 6×10¹², 2×10¹³ vg/kg of SPK-AAV-02 vector. Female rhesus macaques (n=4/dose cohort) received a 30-minute IV infusion in the saphenous vein of a dose of 2×10¹³ vg/kg of SPK-AAV-02 vector. Following AAV vector administration, animals were monitored daily for clinical observations. Blood was collected weekly for 4 weeks to measure GAA activity, GAA antigen levels, anti-GAA IgG formation, hematology and clinical chemistries, including glucose levels to monitor potential alterations of glycemia.

### Results

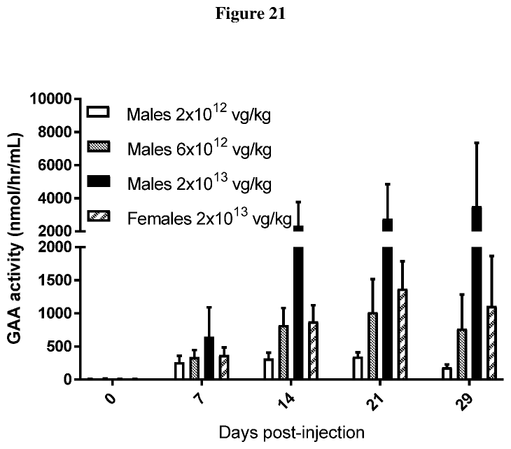

FIG. 21 shows GAA activity levels measured in plasma following administration SPK-AAV-02 vector to male and female rhesus macaques. Results of GAA activity measured in plasma 7, 14, 21 and 28 days post vector injection show a correlation between vector dose and plasma GAA activity level. The levels of GAA activity in the plasma of female NHPs injected at a dose of 2×10¹³ vg/kg were about 0.5-fold lower (49.6%) than those measured in male NHPs injected at an equal dose.

The data in this NHP study confirm the results observed in the mouse studies, namely that plasma GAA activity levels derived from AAV vectors for secretable GAA expression result in a dose dependent GAA activity in plasma following vector administration (FIG. 21). A single infusion of SPK-AAV-02 in NHPs at three ascending doses demonstrated dose-dependent expression of GAA in NHP plasma.

### Example 10

### Nine-Month NHP Study

Each of SPK-AAV-01, SPK-AAV-02 and SPK-AAV-10 were administered at a single dose (6×10¹² vg/kg) via intravenous infusion to male Rhesus macaques (Macaca mulatta). During the course of the study, a decrease in active GAA protein in plasma and concomitant rise in anti-GAA IgG antibody levels was observed in a portion of animals dosed with secretable GAA expressing vectors. Loss of GAA activity and antigen level in plasma was likely due to the development of an IgG-mediated humoral immune clearance of the human transgene protein product. In an effort to reduce the humoral immune response and possibly regain detectable circulating GAA enzyme levels, starting on day 183, certain animals were given an immunosuppression regimen of monthly rituximab in combination with daily cyclosporine A.

### Results

Levels of secreted and active GAA in plasma were detected in all animals, exhibiting peak mean activity levels on day 15 of 482.07±47.90, 664.43±417.55 and 427.833±307.94 nmol/mL/hr for SPK-AAV-01, SPK-AAV-02 and SPK-AAV-10, respectively. Detectable levels of circulating GAA antigen were observed with mean peak levels of 14.84 ±2.21, 19.11±23.23, 14.64±16.96 ng/mL for animals administered with SPK-AAV-01, SPK-AAV-02 and SPK-AAV-10, respectively. Concomitant with the observed decrease in circulating GAA, starting at day 15, an increase in circulating anti-GAA IgG levels was observed. Analysis of vector genomes, by qPCR of liver tissue, confirmed presence of vector in the liver, exhibiting mean vector genome copies of 6.39±2.72, 11.43±4.23 and 19.01±13.93 per haploid genome, for animals administered SPK-AAV-01, SPK-AAV-02 and SPK-AAV-10, respectively.

Liver samples were analyzed for both full length GAA and cleaved GAA (lysosomal form) protein levels. Study samples were compared to vector-naïve Rhesus liver samples serving as a negative control, which did not display detectable levels of either cleaved or intact GAA at the levels of protein loaded (n=3). The amount of full-length protein in study animal liver was 4.0±7.0, 5.4±9.1, 11.0±11.3 ng of GAA/mg of total protein in animals administered SPK-AAV-01, SPK-AAV-02 and SPK-AAV-10, respectively. Each cohort contained higher levels of cleaved GAA than full length GAA, exhibiting levels of 41.6±57.6, 44.4±74.5, 49.0±40.3 ng of GAA/mg of total protein in animals administered SPK-AAV-01, SPK-AAV-02 and SPK-AAV-10, respectively. Regardless of GAA processing, differences between groups were not significant (P=0.72 for the full-length (intact) form; P =0.83 for the lysosomal (cleaved) form by Kruskal-Wallis). At terminus, all groups administered AAV vectors of the invention displayed average detectable levels of GAA above baseline in the liver.

### Conclusion

Administration of each of SPK-AAV-01, SPK-AAV-02 and SPK-AAV-10 resulted in detectable and increasing plasma levels of GAA antigen, peaking over weeks 2-3 and correlating linearly with GAA activity, exhibiting similar kinetics regardless of vector genome. Both GAA activity and GAA antigen levels in plasma declined consecutively over the following timepoints in all but one animal. As previously observed in NHPs treated with AAV vectors, a humoral response to the human transgene product was observed beginning on day 43, which inversely correlated with GAA antigen levels in plasma in all animals that developed a GAA-specific IgG response. Suppression of anti-GAA IgG levels, via administration of an immunosuppressive agent targeting B-cell mediated IgG response (rituximab), resulted in a restoration of detectable GAA levels in plasma in a subset of animals. For all groups, vector genome copies were detectable in the liver at the end of the study, even in the absence of measurable circulating plasma levels of GAA. Correspondingly, GAA protein was detected in all liver tissues treated with vector at the end of the study, while GAA antigen was below the limit of detection in treatment-naïve liver tissue. Taken together, these results indicate that AAV vectors of the invention are able to mediate durable expression of secretable human GAA in the liver over 9 months, regardless of the development of an antibody response to the human transgene product.

### Example 11

### Nine-Week Rodent Immunosuppression Study

Expression levels of the GAA transgene product were measured for nine weeks following intravenous administration of a single dose of 2×10¹² vg/kg of SPK-AAV-02 particles, in the presence or absence of rapamycin and/or prednisolone, in male and female C57BL/6 mice. Mice were tested in 5 groups, as shown in Table 2.

TABLE 2
Group Designations and Dose Levels

| | No. of Animals | | SPK-AAV-02 | Rapamycin | Prednisolone |
|---|---|---|---|---|---|
| Group | Male | Female | (vg/kg) | (2-3 mg/kg)* | (1-0.25 mg/kg)** |
| 1 | 5 | 5 | Vehicle | | |
| 2 | 10 | 10 | 2 × 10¹² | | |
| 3 | 10 | 10 | | X | |
| 4 | 10 | 10 | | | X |
| 5 | 10 | 10 | | X | X |

vg = vector genomes, X = administered treatment
*Rapamycin was delivered daily at 2 mg/kg from day (−)7 through day 5. Starting at day 6, delivery was changed to 3 mg/kg every other day.
**Prednisolone was delivered at a concentration of 1 mg/kg from day (−)1 through day 15, 0.5 mg/kg from day 16 through day 22, and 0.25 mg/kg from day 23 through day 28.

In groups receiving immunosuppressive agent(s), agent(s) were administered prior to AAV particle dosing, and for the first 5 weeks of the study. Following completion of AAV dosing, animals were monitored for clinical observations and body weight for 9 weeks. At the end of the study, clinical chemistry and hematology was performed on blood samples, and histopathology was performed on select tissues. IgG antibodies against the rAAV capsid and plasma GAA activity were assessed weekly throughout the study.

### Results

Levels of GAA activity in plasma were detected above background in all animals administered vector. Vehicle-administered males in Group 1 showed background levels approximately between 300-400 nmol/mL′hr. Circulating levels of GAA activity rose following vector administration with slightly different kinetics depending on the immunosuppressive treatment group but exhibited equivalent peak mean levels of GAA activity. In particular, the peak mean levels of GAA activity were 16114±5411, 14875±6882, 14890±6882, and 21480±6340 nmol/mL′hr for males in Group 2 (SPK-AAV-02 alone), Group 3 (SPK-AAV-02, 2-3 mg/kg rapamycin), Group 4 (SPK-AAV-02, 1-0.25 mg/kg prednisolone), and Group 5 (SPK-AAV-02, 2-3 mg/kg rapamycin, 1-0.25 mg/kg prednisolone), respectively. No statistically significant differences were observed in peak GAA activity levels between any of the AAV treatment groups.

In female mice, peak mean levels of GAA activity were 7380±4034, 5912±3259, 6096±3249, and 9955±3104 nmol/mL′hr for Group 2, Group 3, Group 4, and Group 5, respectively. These levels of GAA activity were significantly above the background detected in vehicle-administered female animals. As seen with the male mice, peak levels of GAA activity in the female mice were not statistically significantly different between any of the AAV treatment groups. The observed differences in plasma levels of GAA activity between males and females are consistent with the well-documented phenomenon of decreased AAV transduction of hepatocytes in female mice (Davidoff et al., 2003, Blood, 102:480-488; DOI: 10.1182/blood-2002-09-2889).

Anti-rAAV capsid IgG in plasma was detected in all animals administered SPK-AAV-02, exhibiting peak mean levels of 86527±92140, 6695±3555, 64368±29635, and 11374 ±6053 ng/mL for males in Group 2, Group 3, Group 4, and Group 5, respectively, and peak mean levels of 182009±148148, 66141±77925, 182654±90161, and 57752±59192 ng/ml for females in Groups 2, 3, 4 and 5, respectively. Group 1 (vehicle) for both males and females had levels of anti-rAAV capsid IgG that were below the quantitative limit of the assay for all timepoints.

### Conclusion

Administration of SPK-AAV-02 resulted in detectable GAA activity levels in all animals. The combined treatment with rapamycin and prednisolone caused levels of GAA activity to be statistically increased (vs. rAAV vector alone) at days 22, 36, and 42 in male mice, and at day 43 in female animals. Of note, the difference in peak levels of GAA activity, regardless of when maximum expression was reached, was not statistically significant between any of the AAV treatment groups within sexes.

A humoral IgG response raised against the rAAV capsid was observed in all groups administered SPK-AAV-02. In both sexes, the presence of rapamycin (Groups 3 and 5) significantly reduced the formation of anti-capsid IgG when compared to vector alone. Treatment with prednisolone had more modest effects on anti-capsid IgG formation, with significant reduction at days 15, 43 and 60 in males and days 43 and 60 in females in Group 4.

Immunosuppression with rapamycin and/or prednisolone in mice, resulted in decreased humoral responses to the rAAV capsid, and did not significantly affect peak levels of GAA activity in plasma.

### Example 12

4-Week Single Dose Co-Administered with Rapamycin in NHP

This study evaluated circulating GAA activity (levels of GAA activity in plasma) over a period of four weeks following administration of a single dose of SPK-AAV-02 at 5.5×10¹³ vg/kg, in the presence of rapamycin, in three male African green monkeys (Chlorocebus sabaeus).

Animals were administered rapamycin (2 mg/kg daily) beginning 5 days prior to AAV dosing, and continuing throughout the four-week study. A single dose of 5.5×10¹³ vg/kg of SPK-AAV-02 was administered intravenously via the saphenous vein. Following completion of AAV dosing, animals were monitored for clinical observations and body weight for 4 weeks. At the conclusion of the study, clinical chemistry and hematology was performed on blood samples, and histopathology was performed on select tissues. Plasma GAA activity was measured weekly throughout the study.

### Results

Levels of GAA activity in plasma were above background in all animals following administration of SPK-AAV-02, with peak activity levels for each of the three animals reaching 1158.1, 711.9, and 623.8 nmol/mL/hr, at the final point measured (Table 3).

TABLE 3
Plasma GAA Activity for Individual NHPs Administered Rapamycin.
GAA Activity in Plasma (nmol/ml/hr)

| | Individual NHP Number | | | |
|---|---|---|---|---|
| | 1 | 2 | 3 | Mean ± SD |
| Baseline | 2.1 | 4.9 | 3.7 | 3.6 ± 1.4 |
| Day 7 | 777.2 | 331.3 | 187.4 | 432.0 ± 307.5 |
| Day 14 | 924.0 | 349.9 | 368.0 | 547.3 ± 326.4 |
| Day 21 | 973.2 | 289.6 | 490.0 | 584.3 ± 351.4 |
| Day 28 | 1158.1 | 711.9 | 623.8 | 831.3 ± 286.5 |

### Conclusion

Administration of SPK-AAV-02 resulted in detectable plasma GAA activity in all three NHPs. The combination of SPK-AAV-02 and rapamycin was well tolerated and had a favorable safety profile in all tests measured.

### Example 13

Therapeutic efficacy in Gaa^{-/-} mouse Pompe model

A 10-month follow-up study of 4-month old Gaa^{-/-} mice (an established mouse model of Pompe disease) administered SPK-AAV-02 at three doses (1.25×10¹¹, 5×10¹¹ and 2×10¹² vg/kg) was carried out. The study demonstrated: (1) dose-dependent increases in circulating GAA enzyme activity, (2) significant improvement of muscle strength, and (3) no significant anti-GAA humoral immune responses.

### Example 14

### Methods

### Cell-Based Assay to Measure the Protein Expression Efficiency of Plasmids Encoding Secretable Human GAA Transgenes

Huh7 cells were plated in 48-well dishes at 5×10⁴ cells per well overnight in DMEM+10% FBS+penicillin/streptomycin/L-glutamine. Plasmids prepared using Plasmid Giga Kit (Qiagen) were transfected into cells at 250 ng per well using Lipofectamine® 2000. Cells were maintained at 37 degrees Celsius and 5% CO₂ for 48-72 hours and supernatants were harvested and stored in low retention microtiter plates at −80 degrees Celsius until assayed for GAA activity. Supernatants were diluted 1:10 and incubated with 3 mM of fluorescent substrate 4-methylumbelliferyl α-D-glucopyranoside for 1 hour at 37 degrees Celsius. The reaction was stopped after 1 hour with carbonate buffer (pH 10.5) and compared to a standard curve generated by diluting 4-methylumbelliferone (4-MU) in the stop buffer, creating a 12-point standard curve starting at 250 pmol/μL and ending at 0 pmol/μL. The plate was read at λe 360 nm; λem 449 nm.

### Hydrodynamic Injection of Plasmid DNA to Assess Potency of Plasmids Encoding Secretable Human GAA Transgenes

Plasmids prepared using Plasmid Giga Kit (Qiagen) were diluted in PBS180+0.001% Pluronic. 25 micrograms of plasmid DNA corresponding to each cassette to be tested was introduced into male C57BL/6 mice of approximately 8 weeks of age intravenously in the lateral tail vein by hydrodynamic injection. 72 hours post injection, whole blood was collected into sodium citrate tubes via lancing the submandibular vein. The first drop of blood was discarded and 200 μL of blood was collected into sodium citrate, and the tube was inverted to avoid hemolysis and clotting. The sample was spun down at 10,000 RMP for 10 minutes at 4 degrees Celsius. Plasma was aliquoted into tubes and stored in a −80 degrees Celsius freezer until analyzed for GAA activity. Plasma samples were diluted to varying degrees and incubated with 3 mM of fluorescent substrate 4-methylumbelliferyl α-D-glucopyranoside for 1 hour at 37 degrees Celsius. The reaction was stopped after 1 hour with carbonate buffer (pH 10.5) and compared to a standard curve generated by diluting 4-MU in the stop buffer, creating a 12-point standard curve starting at 250 pmol/μL and ending at 0 pmol/μL. The plate was read at λex 360 nm; λem 449 nm.

### Cell-Based Assay to Measure the Potency of AAV Vectors Encoding Secretable Human GAA Transgenes

Huh7 cells were plated in 48-well dishes at 5×10⁴ cells per well overnight. SPK-AAV-01 and SPK-AAV-02 from stock vector were prepared in a dose curve (multiplicity of infection, MOI, range from 2×10⁶-1×10⁴) in Opti-MEM™ or DMEM+10% FBS+penicillin/streptomycin/L-glutamine. Existing medium was removed from Huh7 cells and replaced with virus particle containing medium. Cells were maintained at 37 degrees Celsius and 5% CO₂ for 72 hours, and supernatants were harvested and stored in low retention microtiter plates at −80 degrees Celsius until assayed for GAA activity. Supernatants were diluted 1:10 and incubated with 3 mM of fluorescent substrate 4-methylumbelliferyl α-D-glucopyranoside for 1 hour at 37 degrees Celsius. The reaction was stopped after 1 hour with carbonate buffer (pH 10.5) and compared to a standard curve generated by diluting 4-MU in the stop buffer, creating a 12-point standard curve starting at 250 pmol/μL and ending at 0 pmol/μL. The plate was read at λex 360 nm; λem 449 nm.

### GAA Activity Assay in Mouse/Rat Plasma

Whole blood was collected into sodium citrate tubes via lancing the submandibular vein. The first drop of blood was discarded and 200 μL of blood was collected into sodium citrate; the tube was inverted to avoid hemolysis and clotting. The sample was spun down at 10,000 rpm for 10 minutes at 4 degrees Celsius. Plasma was aliquoted into tubes and stored in a −80 degrees Celsius freezer until analyzed for GAA activity. Plasma samples were diluted to varying degrees and incubated with 3 mM of fluorescent substrate 4-methylumbelliferyl α-D-glucopyranoside for 1 hour at 37 degrees Celsius. The reaction is stopped after 1 hour with carbonate buffer (pH 10.5) and compared to a standard curve generated by diluting 4-MU in the stop buffer, creating a 12-point standard curve starting at 250 pmol/μL and ending at 0 pmol/μL. The plate was read at λex 360 nm; λem 449 nm.

### GAA Activity Assay in NHP (Rhesus/AGM) Plasma

Male rhesus macaques/African green monkeys were injected intravenously via the saphenous vein, and whole blood was collected into sodium citrate tubes, which were inverted to avoid hemolysis and clotting. The sample was spun down at 10,000 RMP for 10 minutes at 4 degrees Celsius. Plasma was aliquoted into tubes and stored in a −80 degrees Celsius freezer until analyzed for GAA activity. Plasma samples were diluted to varying degrees and incubated with 3 mM of fluorescent substrate 4-methylumbelliferyl α-D-glucopyranoside for 1 hour at 37 degrees Celsius. The reaction was stopped after 1 hour with carbonate buffer (pH 10.5) and compared to a standard curve generated by diluting 4-MU in the stop buffer, creating a 12-point standard curve starting at 250 pmol/μL and ending at 0 pmol/μL, or an 11-point standard curve (5 μM/mL-0.49 nM/mL). The plate is read at Aex 360 nm; λem 449 nm. GAA activity for tested samples were interpolated from the 4-MU standard curve and expressed as velocity (nanomole/milliliter/hour (nmol/mL/hr)).

### Protein Level Assessment of GAA in Huh7 Supernatants by Western Blot

Huh7 cells were plated in 48-well dishes at 5×10⁴ cells per well overnight. SPK-AAV-01 and SPK-AAV-02 from stock vector were prepared in a dose curve (multiplicity of infection, MOI, range from 2×10⁶-1×10⁴) in Opti-MEM™ or DMEM+10% FBS+penicillin/streptomycin/L-glutamine. Existing medium was removed from Huh7 cells and replaced with virus particle containing medium. Cells were maintained at 37 degrees Celsius and 5% CO₂ for 72 hours, and supernatants were harvested and stored in low retention microtiter plates at −80 degrees Celsius until assayed for GAA protein level. Supernatants were diluted 1:20 with RIPA buffer incubated at 95 degrees Celsius for 5 minutes and run on a 4-12% Bis-Tris NuPage gel with MOPS running buffer. The protein was transferred to polyvinylidene difluoride (pvdf) membrane using the iBlot® system (ThermoFisher), and blocked for 1 hour at room temperature in Odyssey® TBS buffer. The membrane was incubated overnight at 4 degrees Celsius with the primary antibody, rabbit anti-human GAA (Abcam) diluted 1:1000. The membrane was washed and incubated at room temperature for 2 hours with the fluorescent secondary anti-rabbit antibody diluted 1:10,000. The membrane was washed and imaged using the Li-Core® imaging system. The bands were compared to 10 ng of Myozyme® and the marker. Densitometry analysis was conducted using Li-Core® software and bands were normalized to the Myozyme® control.

### Protein Level Assessment of GAA in Mouse Plasma by Western Blot

Male and female C57BL/6 mice were injected intravenously via the tail vein, and whole blood was collected into sodium citrate tubes via lancing the submandibular vein. The first drop of blood was discarded and 200 μL of blood was collected into sodium citrate; the tube was inverted to avoid hemolysis and clotting. The sample was spun down at 10,000 RMP for 10 minutes at 4 degrees Celsius. Plasma was aliquoted into tubes and stored in a −80 degrees Celsius freezer until analyzed for GAA protein level. Plasma was diluted 1:20 with RIPA buffer, incubated at 95 degrees Celsius for 5 minutes, and run on a 4-12% Bis-Tris NuPage gel with MOPS running buffer. The protein was transferred to pvdf membrane using the iBlot® system (ThermoFisher), and blocked for 1 hour at room temperature in Odyssey® TBS buffer. The membrane was incubated overnight at 4 degrees Celsius with the primary antibody, rabbit anti-human GAA (Abcam) diluted 1:1000. The membrane was washed and incubated at room temperature for 2 hours with the fluorescent secondary anti-rabbit antibody diluted 1:10,000. The membrane was washed and imaged using the Li-Core® imaging system. The bands were compared to 10 ng of Myozyme® and the marker. Densitometry analysis was conducted using Li-Core® software and bands were normalized to the Myozyme® control.

### Protein Level Assessment of GAA in NHP Plasma by WES

Male rhesus macaques/African green monkeys were injected intravenously via the saphenous vein, and whole blood was collected into sodium citrate tubes, which were inverted to avoid hemolysis and clotting. The sample was spun down at 10,000 rpm for 10 minutes at 4 degrees Celsius. Plasma was aliquoted into tubes and stored in a −80 degrees Celsius freezer until analyzed for GAA protein levels. Plasma samples were diluted 1:6000 in sample diluent. An 8-point, two-fold standard curve was added to each chip with the Myozyme® starting at a concentration of 50 ng/mL. The samples were diluted in the buffer, denatured at 95 degrees Celsius for 5 minutes, and loaded into the WES™ chip (Protein Simple). The primary antibody was added at a 1:1000 dilution, and the secondary antibody was loaded at working dilution. The chip was run in accordance to the WES™ Protein Simple software, and the resulting bands are analyzed and compared to the Myozyme® standard curve and normalized to the non-specific second band.

### Anti-AAV Capsid IgG Antibodies

Anti-AAV capsid total IgG formation was measured with an ELISA capture assay. ELISA plate wells were coated with 50 μL of a solution containing 1 μg/mL of rAAV particles. Total human IgG (Southern Biotech, 0150-01) was diluted to generate a 10-point standard curve ranging from 10,000 ng/mL to 0.5 ng/mL and added to the plate. The limit of quantitation of the assay was 460 ng/ml after back-calculation. Three levels of quality control samples were prepared and included on each plate to assess assay performance. Capsid particles, standards, and quality controls were incubated overnight at 4 degrees Celsius. After washing, wells were blocked with 2% BSA, 0.05% Tween-20 in PBS for 2 hours at room temperature. Serial dilutions of samples in blocking buffer were loaded on the plate and incubated at room temperature for 2 hours. An HRP-conjugated sheep anti-human IgG antibody (GE Healthcare, NA933V) diluted 1:5000 in blocking buffer was used as detecting antibody and incubated on the plate for 1 hour at room temperature. Following washing, peroxidase activity was developed during a 10-minute incubation at room temperature with 3,3′,5,5′-tetramethylbenzidine substrate (TMB). The reaction was stopped with 1M sulfuric acid, and the plate was read by an absorbance plate reader for optical density (OD) at 450 nm. IgG concentration was determined against a standard curve made with serial dilution of purified human total IgG.

1. A nucleic acid encoding an acid α-glucosidase (GAA), wherein said nucleic acid has greater than 95% sequence identity to SEQ ID NO: 5.

2-88. (canceled)

89. The nucleic acid of claim 1, wherein said GAA comprises the sequence of SEQ ID NO: 25.

90. The nucleic acid of claim 89, wherein said nucleic acid has greater than 97% sequence identity to SEQ ID NO: 5.

91. The nucleic action of claim 90, wherein said nucleic acid has greater than 99% sequence identity to SEQ ID NO: 5.

92. The nucleic acid of claim 89, wherein said nucleic acid contains 0 to 5 CpG dinucleotides.

93. The nucleic acid of claim 1, wherein said nucleic acid comprises the sequence of SEQ ID NO: 5.

94. The nucleic acid of claim 92, wherein said nucleic acid contains 0 CpG dinucleotides.

95. An expression cassette comprising the nucleic acid of claim 89 operably linked to a 5′ expression control element and 3′ polyadenylation sequence.

96. The expression cassette of claim 95, wherein said polyadenylation sequence comprises the amino acid sequence of SEQ ID NO:26.

97. The expression cassette of claim 96, wherein said expression control element comprises a promoter.

98. The expression cassette of claim 96, wherein said expression control element comprises an ApoE/hAAT enhancer/promoter sequence promoter,

99. The expression cassette of claim 97, comprising a sequence having a sequence identity greater than 95% to SEQ ID NO: 15.

100. The expression cassette of claim 99, comprising the sequence of SEQ ID NO: 15.

101. A recombinant adeno-associated virus (rAAV) vector comprising the expression cassette of claim 97.

102. The rAAV vector of claim 101, where said rAAV vector comprises:
a) an AAV capsid, and
b) a 3′ inverted terminal repeats (ITR) and a 5′ ITR flanking the 3′ and 5′ terminus of said expression cassette.

103. The rAAV vector of claim 102, wherein said capsid is an AAV1, AAV2, AAV3, AAV4, AAV5, AAV6, AAV7, AAV8, AAV9, AAV10, AAV11, Rh10, Rh74, AAV3B, or AAV-218 capsid, or comprises a VP1 of SEQ ID NO:30, a VP2 of SEQ ID NO: 31 and a VP3 of SEQ ID NO: 31.

104. The rAAV vector of claim 103, wherein said 5′ and 3′ ITR comprise the 5′ and 3′ ITR provided by any of AAV1, AAV2, AAV3, AAV4, AAV5, AAV6, AAV7, AAV8, AAV9, AAV10, AAV11, Rh10, Rh74 or AAV3B serotypes.

105. The AAV vector of claim 102, comprising a nucleic acid sequence having a sequence identity greater than 95% to SEQ ID NO: 17, wherein said capsid comprises a VP1 of SEQ ID NO: 30, a VP2 of SEQ ID NO: 31, and a VP3 of SEQ ID NO: 32.

106. The rAAV vector of claim 105, comprising the nucleic acid sequence of SEQ ID NO: 17.

107. A pharmaceutical composition comprising the rAAV vector of claim 102, in a biologically compatible carrier or excipient.

108. A method of treating a human in need of GAA, comprising administering the vector of claim 102 to said human.

109. The method of claim 108, wherein said human has Pompe disease, infantile onset Pompe disease, or late onset Pompe disease.

110. The method of claim 108, wherein said human has a glycogen storage disease (GSD) selected from the group consisting of: GSD type I (von Gierke's disease), GSD type III (Forbes-Cori disease), GSD type IV (Anderson disease, amylopectinosis), GSD type V (McArdle disease), GSD type VI (Hers disease), GSD type VII (Tarui disease), and a lethal congenital GSD of the heart.
